(19) United States
(12) Patent Application Publication
(10) Pub. No.: US 2022/0364171 A1
(43) Pub. Date: Nov. 17, 2022

(54) PREDICTING A TREATMENT RESPONSE IN INFLAMMATORY BOWEL DISEASE

(71) Applicant: KATHOLIEKE UNIVERSITEIT LEUVEN, Leuven (BE)

(72) Inventors: Séverine VERMEIRE, Haasrode (BE); Bram VERSTOCKT, Kampenhout (BE); Marc FERRANTE, Sint-Katelijne-Waver (BE)

(21) Appl. No.: 17/296,415

(22) PCT Filed: Nov. 25, 2019
(86) PCT No.: PCT/EP2019/082483

§ 371 Date: May 24, 2021

Related U.S. Application Data
(60) Provisional application No. 62/888,987, filed on Aug. 19, 2019.

(30) Foreign Application Priority Data
Nov. 23, 2018 (GB) 1819065.2
May 9, 2019 (GB) 1906543.2
May 15, 2019 (GB) 1906848.5
May 15, 2019 (GB) 1906883.2

(51) Int. Cl. C12Q 1/6883; G01N 33/564; C07K 16/24; C07K 16/28

(52) U.S. Cl. CPC C12Q 1/6883; G01N 33/564; C07K 16/241; C07K 16/2839; C07K 16/244; C12Q 1/6851

(57) ABSTRACT

In general the present invention concerns a method for predicting the therapeutic outcome of a treatment of in inflammatory bowel disease for anti-TNF agents, anti-α₄β₇-integrin agents and/or anti-IL-12/23 agents. The method defines which the agents are likely to provide the best healing effect for a particular patients affected by an inflammatory bowel disease. In particular the method predicts the therapeutic outcome of a treatment of anti-TNF agents in inflammatory bowel disease.

### BACKGROUND OF THE INVENTION

### A. Field of the Invention

In general the present invention concerns a method for predicting the therapeutic outcome of a treatment of in inflammatory bowel disease for anti-TNF agents, anti-α₄β₇-integrin agents and/or anti-IL-12/23 agents. The method defines which the agents are likely to provide the best healing effect for a particular patients affected by an inflammatory bowel disease. In particular the method predicts the therapeutic outcome of a treatment of anti-TNF agents in inflammatory bowel disease.

Another general aspect of the present invention concerns a method for predicting the therapeutic outcome of a treatment of anti-TNF agents in inflammatory bowel disease. Furthermore in general the present invention concerns a method for predicting the therapeutic outcome of a treatment of anti-TNF and anti-integrin agents in inflammatory bowel disease.

The invention concerns method of determining the efficacy of an anti-TNF-agent for treatment of a gastrointestinal inflammatory disorder in a patient, the method comprising comparing the amount of a biomarker in a sample obtained from the patient after or during treatment with anti-TNF-agent, to an amount of the biomarker in a sample obtained from the patient before the treatment, wherein a change in the amount of the biomarker after or during the treatment, as compared to before the treatment, is indicative of the efficacy of the agent for treatment of the gastrointestinal disorder in the patient, and wherein the biomarker is triggering receptor expressed on myeloid cells 1 (TREM1), a protein that in humans is encoded by the TREM1 gene.

More particularly the invention concerns, a method of determining the efficacy of an anti-TNF-agent for treatment of an inflammatory bowel disease in a patient, the method comprising comparing the amount of a biomarker in a sample obtained from the patient after or during treatment with anti-TNF-agent, to an amount of the biomarker in a sample obtained from the patient before the treatment, wherein a change in the amount of the biomarker after or during the treatment, as compared to before the treatment, is indicative of the efficacy of the agent for treatment of the an inflammatory bowel disease in the patient, and wherein the biomarker is triggering receptor expressed on myeloid cells 1 (TREM1), a protein that in humans is encoded by the TREM1 gene, and whereby, low TREM1 expression in whole blood predicts anti-TNF response in inflammatory bowel disease.

Another aspect of present invention concerns a four-gene colonic signature that for a α₄β₇-integrine blocker treatment, predicts the endoscopic outcome in patients with inflammatory bowel disease. Present invention concerns more particularly a four-gene colonic signature that for a treatment with the monoclonal antibody, vedolizumab, predicts the endoscopic outcome in patients with inflammatory bowel disease.

Another aspect of present invention in general concerns a method of predicting the response in patients treated for immune bowel disorder with drug directed against interleukin 12 (IL12) and interleukin 23 (IL23), in particularly a drug that blocks or inhibits the IL-12/23 pathway (e.g. Apilimod (or STA-5326)). More specifically the present invention also concerns a method of predicting the response in patients treated with an antibody that specifically blocks interleukins IL-12 and IL-23 or that is a IL12 and IL23 inhibitor, for instance a monoclonal that selectively targets the P40 subunit of IL-23 and IL-12, for an immune bowel disorder (IBD), such as Crohn's disease. More specifically the present invention also concerns a method of predicting the response in patients treated with an Anti-P40 monoclonal antibody (e.g. a monoclonal antibody that selectively targets the P40 subunit of IL-23 and IL-12), such as ustekinumab (CNTO1275) or Briakinumab (ABT 874, for an immune bowel disorder, such as Crohn's disease.

### B. Description of the Related Art

Inflammatory bowel diseases (IBD) are complex gastro-intestinal diseases, driven by extrinsic environmental factors and intrinsic factors including host genetics, the immune system and the gut microbiome (de Souza, H. S. P., et al. Nat Rev Gastroenterol Hepatol 14, 739-749 (2017)). Due to the availability and accessibility of diseased tissues and cells (endoscopic biopsies, blood, faeces), IBD presents a unique opportunity to better understand its molecular biology and to aim for a more personalised treatment strategy (Weersma, R. K., et al; Gastroenterology 155, e1-e4 (2018)). Big data from various-omics layers such as genomics, transcriptomics and proteomics should therefore be integrated with clinical meta-data to achieve these goals. However, due to the high dimensionality of data and the many confounding factors, interpreting these data warrants novel approaches that can uncover hidden patterns in large and complex datasets (Gligorijevic, V & Przulj, N. Methods for biological data integration: perspectives and challenges. J R Soc Interface 12(2015)). Why patients respond better to one class of therapy over another, or to all classes, is unknown. Multiple factors are believed to contribute to a patient's response to a particular agent including clinical and/or demographic factors, host genomics, transcriptomics in addition to metagenomics, metabolomics and proteomics.

Anti-TNF agents (infliximab, adalimumab) have become the cornerstone in the management of moderate-to-severe IBD over the past two decades (Billiet, T., Expert Opin Biol Ther 14, 75-101 (2014). Despite their therapeutic success, the mode of action of tumour necrosis factor (TNF)-neutralising agents has not yet been fully unraveled (Levin, A. D., et al. J Crohns Colitis 10, 989-997 (2016)). Furthermore, endoscopic remission rates hardly exceed 30% in clinical trials, (Rutgeerts, P., et al. Gastroenterology 142, 1102-1111 e1102 (2012), Rutgeerts, P., et al. Gastrointest Endosc 63, 433-442; quiz 464 (2006) and Colombel, J F., et al. N Engl J Med 362, 1383-1395 (2010)) highlighting the therapeutic gap physicians are facing in daily clinical practice. Other compounds, targeting different processes in IBD pathophysiology are therefore absolutely warranted (Verstockt, B., et al. J Gastroenterol 53, 585-590 (2018), Sabino, J., et al; An update. Therap Adv Gastroenterol. (2019)). Among them are molecules affecting leucocyte trafficking (natalizumab, vedolizumab, etrolizumab, ontamalimab, ozanimod, etrasimod). Vedolizumab, a gut-specific anti-adhesion agent targeting the α4β7 integrin, has been approved for both Crohn's disease (CD) and ulcerative colitis (UC). However, similar to the anti-TNF agents, endoscopic remission in randomized trials can be achieved only in 20-40% of patients (Feagan, B. G., et al. N Engl J Med 369, 699-710 (2013). In order to further improve these endoscopic remission rates, as they significantly impact long-term patient outcome (Laharie, D., et al. Aliment Pharmacol Ther 37, 998-1004 (2013) and Schnitzler, F., et al. Inflamm Bowel Dis 15, 1295-1301 (2009)) patients should be better stratified at baseline using predictive biomarkers, reflecting the underlying biology driving their individual disease.

Only one vedolizumab biomarker and very few biomarkers predicting response to anti-TNF agents have been reported, all on a single-omic layer and the majority with a focus on just one single gene (Verstockt, B., et al. 14th Congress of ECCO, Vol. 13 S048-SO48 (Journal of Crohn s and Colitis Copenhagen, (2019), Arijs, I., et al. Gut 58, 1612-1619 (2009, Arijs, I., et al. Inflamm Bowel Dis 16, 2090-2098 (2010), West, N. R., et al. Nat Med 23, 579-589 (2017))). However, a single gene marker is presumably too simplistic, given the complexity of IBD pathophysiology. Efficient integration and interpretation of big datasets should therefore be prioritized (de Souza, H. S. P., Fiocchi, C. & Iliopoulos, D. The IBD interactome: an integrated view of aetiology, pathogenesis and therapy. Nat Rev Gastroenterol Hepatol 14, 739-749 (2017)) Also, in contrast to many hypothesis-driven studies, focus should go to unbiased data-driven strategies which reveal the key players in the underlying response networks and/or which are predictive of drug response (de Souza, H. S. P., Fiocchi, C. & Iliopoulos, D. The IBD interactome: an integrated view of aetiology, pathogenesis and therapy. Nat Rev Gastroenterol Hepatol 14, 739-749 (2017)).

We prospectively collected serum (proteomics), inflamed mucosal tissue (transcriptomics), sorted CD14⁺ monocytes and CD4⁺ T-cells (transcriptomics) and DNA (genomics) in IBD patients with active endoscopic disease initiating infliximab, adalimumab or vedolizumab therapy. By integrating the above mentioned multi-omic datasets, we studied differences in the underlying biology linked to therapeutic success, and identified predictive drug-specific biomarkers for endoscopic outcome in both CD and UC.

Antibodies against integrin α4β7 have been demonstrated to interfere with gut leukocyte trafficking. The monoclonal antibody, Vedolizumab, which interferes with gut leukocyte trafficking, has been approved for the treatment of inflammatory bowel disease (IBD). Due to the increasing availability of therapeutic compounds, predictive biomarkers are urgently awaited in order to stratify patients accordingly. Other α₄P₇ integrin inhibitors are known in the art and in development to therapy for IBD.

For present invention a vedolizumab-specific predictive 4-gene colonic expression signature was identified and validated. It provided additional insights in the mode of action of vedolizumab therapy.

For present work inflamed colonic biopsies from IBD patients initiating vedolizumab or anti-TNF therapy were collected, and processed through RNA-sequencing. Besides standard differential gene expression, pathway analysis and cell deconvolution, predictive modelling was applied in a training (n=20) and validation dataset (n=11). The identified model, predicting vedolizumab-induced endoscopic remission (absence of ulcerations at month 6 for Crohn's disease; Mayo endoscopic sub-score <1 at week 14 for ulcerative colitis) was subsequently validated in three independent datasets (n=66 in total).

The research of present invention demonstrated that forty-four genes were significantly differently expressed between vedolizumab endoscopic remitters and non-remitters, with a significant upregulation of leukocyte migration in non-remitters (p<0.006). Deconvolution methods identified a significant enrichment of monocytes (p=0.005), M1 macrophages (p=0.05) and effector memory CD4 T cells (p=0.008) in non-remitters, whereas remitters demonstrated a baseline enrichment of naïve B cells (p=0.05).

Present invention provides a 4-gene colonic signature (PIWIL1, MAATS1, RGS13, DCHS2) that accurately differentiates remitters from non-remitters in both the training and validation dataset (accuracy 80.0%; 100%), and in all 3 independent datasets, including qPCR validation (p=0.003).

In contrast, this 4-gene signature, though being predictive for anti-α₄β₇-integrin responsiveness was not predictive for anti-TNF responsiveness. The presence of the identified genes at protein level in inflamed colon, using immunohistochemistry was confirmed.

Furthermore multiple factors are believed to contribute to a patient's response to biological therapies. We here applied integrated omics analysis to understand and predict response of an IBD patient to an a IL12 and IL23 inhibitor, in particular an anti-IL12/23p40 monoclonal antibody. This has been shown to be particular suitable to predict the outcome of ustekinumab treatment of patients with Crohn's disease (CD).

### SUMMARY OF THE INVENTION

These results can aid in the selection of therapy in biologic-naïve patients.

The present invention solves the problems of the related art of predicting or of indicating that an anti-TNF therapy is or will heal an inflammatory bowel disease.

With the changed therapeutic armamentarium for Crohn's disease (CD) and ulcerative colitis (UC), biomarkers predicting treatment response are urgently needed. We studied whole blood and mucosal expression of genes previously reported to predict outcome to anti-TNF therapy, and investigated if the signature was specific for anti-TNF agents.

Methods: We prospectively included 54 active IBD patients (24CD, 30UC) initiating anti-TNF therapy, as well as 22 CD patients initiating ustekinumab and 51 patients initiating vedolizumab (25CD, 26UC). Whole blood expression of OSM, TREM1, TNF and TNFR2 was measured prior to start of therapy using qPCR, and mucosal gene expression in inflamed biopsies using RNA-sequencing. Response was defined as endoscopic remission (SES-CD≤2 at week 24 for CD and Mayo endoscopic sub-score <1 at week 10 for UC).

Findings: Baseline whole blood TREM1 was downregulated in future anti-TNF responders, both in UC (FC=0.53, p=0.001) and CD (FC=0.66, p=0.007), as well as in the complete cohort (FC=0.67, p b 0.001). Receiver operator characteristic statistics showed an area under the curve (AUC) of 0.78 (p=0.001). A similar accuracy could be achieved with mucosal TREM1 (AUC 0.77, p=0.003), which outperformed the accuracy of serum TREM1 (AUC 0.58, p=0.31). Although differentially expressed in tissue, OSM, TNF and TNFR2 were not differentially expressed in whole blood. The TREM1 predictive signal was anti-TNF specific, as no changes were seen in ustekinumab and vedolizumab treated patients.

Interpretation: We identified low TREM-1 as a specific biomarker for anti-TNF induced endoscopic remission.

The present invention provides in a first embodiment an in vitro method of determining if a subject suffering from an patient suffering of gastrointestinal inflammatory disorder will respond or not to anti-TNF, wherein the method comprises: obtaining a biological sample from the subject; analyzing the level of its TREM-1 expression or activity of expression product of TREM-1 in the biological sample, and comparing said level of expression or activity with the TREM-1 expression or activity from a control sample; wherein a different level of TREM-1 expression or activity relative to the control sample is an indication of response to anti-TNF or a propensity thereto in the subject.

The object of the present invention is also to provide the in vitro method according to any one of the first embodiment, wherein the inflammatory condition of the large intestine and/or small intestine is an inflammatory bowel disease.

The object of the present invention is also to provide the in vitro method according to any one of the first embodiment, further comprising predicting if the subject will respond to therapy for Crohn's disease.

The object of the present invention is also to provide the in vitro method according to any one of the first embodiment, further comprising predicting if the subject will respond to therapy for Ulcerative colitis.

The object of the present invention is also to provide the in vitro method according to the first embodiment, for predicting if the subject with Crohn's disease will respond to therapy of anti-TNF and/or anti-TNF.

The object of the present invention is also to provide the in vitro method according to the first embodiment, for predicting if the subject with Ulcerative colitis will respond to therapy of anti-TNF and/or anti-TNF.

The object of the present invention is also to provide the in vitro method according to the first embodiment, for predicting if the subject with gastrointestinal inflammatory disorder will respond to therapy of anti-TNF and/or anti-TNF, whereby downregulation of whole blood TREM1 expression indicates the future anti-TNF healers.

The object of the present invention is also to provide the in vitro method according to the first embodiment, for predicting if the subject with Crohn's disease will respond to therapy of anti-TNF and/or anti-TNF, whereby downregulation of whole blood TREM1 expression indicates the future anti-TNF healers.

The object of the present invention is also to provide the in vitro method according to the first embodiment, for predicting if the subject with Ulcerative colitis will respond to therapy of anti-TNF and/or anti-TNF, whereby downregulation of whole blood TREM1 expression indicates the future anti-TNF healers.

The object of the present invention is also to provide the in vitro method according to the first embodiment, for predicting if the subject with gastrointestinal inflammatory disorder will respond to therapy of anti-TNF and/or anti-TNF, whereby downregulation of whole blood TREM1 expression indicates the future healers on ustekinumab or vedolizumab therapy

The object of the present invention is also to provide the in vitro method according to the first embodiment, for predicting if the subject with Crohn's disease will respond to therapy of anti-TNF and/or anti-TNF, whereby downregulation of whole blood TREM1 expression indicates the future healers on ustekinumab or vedolizumab therapy.

The object of the present invention is also to provide the in vitro method according to the first embodiment, for predicting if the subject with Ulcerative colitis will respond to therapy of anti-TNF and/or anti-TNF, whereby downregulation of whole blood TREM1 expression indicates the future healers on ustekinumab or vedolizumab therapy.

The object of the present invention is also to provide the in vitro method according to the first embodiment, for predicting if the subject with gastrointestinal inflammatory disorder will respond to therapy of anti-TNF and/or anti-TNF, whereby downregulation of whole blood TREM1 expression or predicts future anti-TNF induced endoscopic remission.

The object of the present invention is also to provide the in vitro method according to the first embodiment, for predicting if the subject with Crohn's disease will respond to therapy of anti-TNF and/or anti-TNF, whereby downregulation of whole blood TREM1 expression or predicts future anti-TNF induced endoscopic remission.

The object of the present invention is also to provide the in vitro method according to the first embodiment, for predicting if the subject with Ulcerative colitis will respond to therapy of anti-TNF and/or anti-TNF, whereby downregulation of whole blood TREM1 expression indicates or predicts future anti-TNF induced endoscopic remission.

In another second aspect, the present invention provides an in vitro method of determining if a subject suffering from an patient suffering of gastrointestinal inflammatory disorder will respond or not to anti-TNF therapy, wherein the method comprises: obtaining a biological sample from the subject; analyzing the level of its TREM-1 expression or activity of expression product of TREM-1 in the biological sample, and comparing said level of expression or activity with the TREM-1 expression or activity from a control sample; wherein a different level of TREM-1 expression or activity relative to the control sample is an indication of response to anti-TNF or a propensity thereto in the subject

The object of the present invention is also to provide the in vitro method according to the second aspect, wherein the inflammatory condition of the large intestine and/or small intestine is an inflammatory bowel disease.

The object of the present invention is also to provide the in vitro method according to the second aspect, further comprising predicting if the subject will respond to therapy for Crohn's disease.

The object of the present invention is also to provide the in vitro method according to the second aspect, further comprising predicting if the subject will respond to therapy for Ulcerative colitis.

The object of the present invention is also to provide the in vitro method according to the second aspect, for predicting if the subject with Crohn's disease will respond to therapy of anti-TNF and/or anti-TNF.

The object of the present invention is also to provide the in vitro method according to the second aspect, for predicting if the subject with Ulcerative colitis will respond to therapy of anti-TNF and/or anti-TNF.

The object of the present invention is also to provide the in vitro method according to the second aspect, for predicting if the subject with gastrointestinal inflammatory disorder will respond to therapy of anti-TNF and/or anti-TNF, whereby downregulation of whole blood TREM1 expression indicates the future anti-TNF healers.

The object of the present invention is also to provide the in vitro method according to the second aspect, for predicting if the subject with Crohn's disease will respond to therapy of anti-TNF and/or anti-TNF, whereby downregulation of whole blood TREM1 expression indicates the future anti-TNF healers.

The object of the present invention is also to provide the in vitro method according to the second aspect, for predicting if the subject with Ulcerative colitis will respond to therapy of anti-TNF and/or anti-TNF, whereby downregulation of whole blood TREM1 expression indicates the future anti-TNF healers.

The object of the present invention is also to provide the in vitro method according to the second aspect, for predicting if the subject with gastrointestinal inflammatory disorder will respond to therapy of anti-TNF and/or anti-TNF, whereby downregulation of whole blood TREM1 expression indicates the future healers on ustekinumab or vedolizumab therapy

The object of the present invention is also to provide the in vitro method according to the second aspect, for predicting if the subject with Crohn's disease will respond to therapy of anti-TNF and/or anti-TNF, whereby downregulation of whole blood TREM1 expression indicates the future healers on ustekinumab or vedolizumab therapy

The object of the present invention is also to provide the in vitro method according to the second aspect, for predicting if the subject with Ulcerative colitis will respond to therapy of anti-TNF and/or anti-TNF, whereby downregulation of whole blood TREM1 expression indicates the future healers on ustekinumab or vedolizumab therapy

The object of the present invention is also to provide the in vitro method according to the second aspect, for predicting if the subject with gastrointestinal inflammatory disorder will respond to therapy of anti-TNF and/or anti-TNF, whereby downregulation of whole blood TREM1 expression or predicts future anti-TNF induced endoscopic remission.

The object of the present invention is also to provide the in vitro method according to the second aspect, for predicting if the subject with Crohn's disease will respond to therapy of anti-TNF and/or anti-TNF, whereby downregulation of whole blood TREM1 expression or predicts future anti-TNF induced endoscopic remission.

The object of the present invention is also to provide the in vitro method according to the second aspect, for predicting if the subject with Ulcerative colitis will respond to therapy of anti-TNF and/or anti-TNF, whereby downregulation of whole blood TREM1 expression indicates or predicts future anti-TNF induced endoscopic remission.

The present invention provides in a third embodiment an in vitro method of determining if a subject suffering from an patient suffering of gastrointestinal inflammatory disorder will respond or not to anti-TNF, wherein the method comprises: obtaining a biological sample from the subject; analyzing the level of its TREM-1 expression or activity of expression product of TREM-1 in the biological sample, and comparing said level of expression or activity with the TREM-1 expression or activity from a control sample; wherein a different level of TREM-1 expression or activity relative to the control sample is an indication of response to anti-TNF or a propensity thereto in the subject.

The object of the present invention is also to provide the in vitro method according to any one of the third embodiment, wherein the inflammatory condition of the large intestine and/or small intestine is an inflammatory bowel disease.

The object of the present invention is also to provide the in vitro method according to any one of the third embodiment, further comprising predicting if the subject will respond to therapy for Crohn's disease.

The object of the present invention is also to provide the in vitro method according to any one of the third embodiment, further comprising predicting if the subject will respond to therapy for Ulcerative colitis.

The object of the present invention is also to provide the in vitro method according to the third embodiment, for predicting if the subject with Crohn's disease will respond to therapy of anti-TNF and/or anti-TNF.

The object of the present invention is also to provide the in vitro method according to the third embodiment, for predicting if the subject with Ulcerative colitis will respond to therapy of anti-TNF and/or anti-TNF.

The object of the present invention is also to provide the in vitro method according to the third embodiment, for predicting if the subject with gastrointestinal inflammatory disorder will respond to therapy of anti-TNF and/or anti-TNF, whereby downregulation of whole blood TREM1 expression indicates the future anti-TNF healers.

The object of the present invention is also to provide the in vitro method according to the third embodiment, for predicting if the subject with Crohn's disease will respond to therapy of anti-TNF and/or anti-TNF, whereby downregulation of whole blood TREM1 expression indicates the future anti-TNF healers.

The object of the present invention is also to provide the in vitro method according to the third embodiment, for predicting if the subject with Ulcerative colitis will respond to therapy of anti-TNF and/or anti-TNF, whereby downregulation of whole blood TREM1 expression indicates the future anti-TNF healers.

The object of the present invention is also to provide the in vitro method according to the third embodiment, for predicting if the subject with gastrointestinal inflammatory disorder will respond to therapy of anti-TNF and/or anti-TNF, whereby downregulation of whole blood TREM1 expression indicates the future healers on ustekinumab or vedolizumab therapy

The object of the present invention is also to provide the in vitro method according to the third embodiment, for predicting if the subject with Crohn's disease will respond to therapy of anti-TNF and/or anti-TNF, whereby downregulation of whole blood TREM1 expression indicates the future healers on ustekinumab or vedolizumab therapy

The object of the present invention is also to provide the in vitro method according to the third embodiment, for predicting if the subject with Ulcerative colitis will respond to therapy of anti-TNF and/or anti-TNF, whereby downregulation of whole blood TREM1 expression indicates the future healers on ustekinumab or vedolizumab therapy

The object of the present invention is also to provide the in vitro method according to the third embodiment, for predicting if the subject with gastrointestinal inflammatory disorder will respond to therapy of anti-TNF and/or anti-TNF, whereby downregulation of whole blood TREM1 expression or predicts future anti-TNF induced endoscopic remission.

The object of the present invention is also to provide the in vitro method according to the third embodiment, for predicting if the subject with Crohn's disease will respond to therapy of anti-TNF and/or anti-TNF, whereby downregulation of whole blood TREM1 expression or predicts future anti-TNF induced endoscopic remission.

The object of the present invention is also to provide the in vitro method according to the third embodiment, for predicting if the subject with Ulcerative colitis will respond to therapy of anti-TNF and/or anti-TNF, whereby downregulation of whole blood TREM1 expression indicates or predicts future anti-TNF induced endoscopic remission.

In some embodiments, the invention concerns a method of determining the efficacy of a TNF antagonist for treatment of a gastrointestinal inflammatory disorder in a patient, the method comprising comparing the amount of a biomarker in a sample obtained from the patient after or during treatment with the TNF antagonist, to an amount of the biomarker in a sample obtained from the patient before the treatment, wherein a change in the amount of the biomarker after or during the treatment, as compared to before the treatment, is indicative of the efficacy of the antagonist for treatment of the gastrointestinal disorder in the patient, and wherein the biomarker is triggering receptor expressed on myeloid cells 1 (TREM1) in the patient's peripheral blood.

In another embodiment the invention concerns a method of predicting the responsiveness of a patient having a gastrointestinal inflammatory disorder to treatment with an TNF antagonist, the method comprising comparing the amount of a biomarker in a sample obtained from the patient after or during treatment with the TNF antagonist, to the amount of the biomarker in a sample obtained from the patient before the treatment, wherein a change in the amount of the biomarker after or during the treatment, as compared to before the treatment is indicative of the responsiveness of said patient to treatment with said antagonist, and wherein the biomarker is TREM1 in the patient's peripheral blood.

In yet another embodiment the invention concerns a method of determining the dosing of a TNF antagonist for treatment of a gastrointestinal inflammatory disorder in a patient, the method comprising adjusting the dose of the TNF antagonist based on a comparison of the amount of a biomarker in a sample obtained from the patient after or during treatment with a dose or dosing regimen of the TNF antagonist, to an amount of the biomarker in a sample obtained from the patient before the treatment, wherein a change in the amount of the biomarker after or during the treatment, as compared to before the treatment, is indicative of the efficacy of or responsiveness to the dose or dosing regimen of the TNF antagonist for treatment of the gastrointestinal disorder in the patient, and wherein the biomarker is TREM1 in the patient's peripheral blood.

A method of determining the dosing regimen of a TNF antagonist for treatment of a gastrointestinal inflammatory disorder in a patient, the method comprising adjusting the dose regimen of the TNF antagonist based on a comparison of the amount of a biomarker in a sample obtained from the patient after or during treatment with a dosing regimen of the TNF antagonist, to an amount of the biomarker in a sample obtained from the patient before the treatment, wherein a change in the amount of the biomarker after or during the treatment, as compared to before the treatment, is indicative of the efficacy of or responsiveness to the dose or dosing regimen of the TNF antagonist for treatment of the gastrointestinal disorder in the patient, and wherein the biomarker is TREM1 in the patient's peripheral blood. Such change in amount is preferably a that the biomarker is decreased, for instance when the amount of said biomarker is measured within 100 days after receiving a first dose of the agent or when the amount of said biomarker is measured at least about 24 hours after administering the agent. This biomarker can be indicative for the inflammatory bowel disease, Crohn's disease (CD) or ulcerative colitis (UC). The TNF antagonist used can be an anti-TNF antibody, for instance monoclonal antibody, for instance a chimeric, human or humanized antibody or a fragment of such antibody. This method is particular suitable for determining the dosing regimen of ustekinumab or vedolizumab. Suitable samples are a peripheral blood sample of said patient.

In an particular embodiment, the method according to the present invention further comprises a treatment with a candidate agent for a human patient diagnosed with a gastrointestinal inflammatory disorder, comprising determining an effective dosage for the human patient based on a dosage that effectively decreases the amount of the biomarker TREM1 in peripheral blood of a non-human subject in response to a treatment with said candidate agent, wherein the biomarker is in the patient's peripheral blood. The non-human subject is preferably a monkey and the agent is preferably an anti-TNF antibody.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

Some embodiments of the invention are set forth in embodiment format directly below:
1. An in vitro method of determining if a subject suffering from gastrointestinal inflammatory disorder will respond or not to anti-TNF, wherein the method comprises: obtaining a biological sample from the subject; analyzing the level of its TREM-1 expression or activity of expression product of TREM-1 in the biological sample, and comparing said level of expression or activity with the TREM-1 expression or activity from a control sample; wherein a different level of TREM-1 expression or activity relative to the control sample is an indication of response to anti-TNF or a propensity thereto in the subject.
2. An in vitro method of determining the efficacy of a TNF antagonist for treatment of a gastrointestinal inflammatory disorder in a subject, the method comprising comparing the amount of a biomarker in a sample obtained from the subject after or during treatment with the TNF antagonist, to an amount of the biomarker in a sample obtained from the subject before the treatment, wherein a change in the amount of the biomarker after or during the treatment, as compared to before the treatment, is indicative of the response or efficacy of the antagonist for treatment of the gastrointestinal disorder in the subject, and wherein the biomarker is triggering receptor expressed on myeloid cells 1 (TREM1) in the subject's peripheral blood.
3. The in vitro method according to any one of the embodiments 1 or 2, wherein the inflammatory condition of the large intestine and/or small intestine is an inflammatory bowel disease.
4. The in vitro method according to any one of the embodiments 1 or 2, further comprising predicting if the subject will respond to therapy for Crohn's disease.
5. The in vitro method according to any one of the embodiments 1 or 2, further comprising predicting if the subject will respond to therapy for Ulcerative colitis.
6. The in vitro method according to the embodiments 1 or 2, for predicting if the subject with Crohn's disease will respond to therapy of anti-TNF and/or anti-TNF.
7. The in vitro method according to the embodiments 1 or 2, for predicting if the subject with Ulcerative colitis will respond to therapy of anti-TNF and/or anti-TNF.
8. The in vitro method according to the embodiments 1 or 2, for predicting if the subject with gastrointestinal inflammatory disorder will respond to therapy of anti-TNF and/or anti-TNF, whereby downregulation of whole blood TREM1 expression indicates the future anti-TNF healers.
9. The in vitro method according to the embodiments 1 or 2, for predicting if the subject with Crohn's disease will respond to therapy of anti-TNF and/or anti-TNF, whereby downregulation of whole blood TREM1 expression indicates the future anti-TNF healers.
10. The in vitro method according to the embodiments 1 or 2, for predicting if the subject with Ulcerative colitis will respond to therapy of anti-TNF and/or anti-TNF, whereby downregulation of whole blood TREM1 expression indicates the future anti-TNF healers.
11. The in vitro method according to the embodiments 1 or 2, for predicting if the subject with gastrointestinal inflammatory disorder will respond to therapy of anti-TNF and/or anti-TNF, whereby downregulation of whole blood TREM1 expression indicates the future healers on ustekinumab or vedolizumab therapy
12. The in vitro method according to the embodiments 1 or 2, for predicting if the subject with Crohn's disease will respond to therapy of anti-TNF and/or anti-TNF, whereby downregulation of whole blood TREM1 expression indicates the future healers on ustekinumab or vedolizumab therapy
13. The in vitro method according to the embodiments 1 or 2, for predicting if the subject with Ulcerative colitis will respond to therapy of anti-TNF and/or anti-TNF, whereby downregulation of whole blood TREM1 expression indicates the future healers on ustekinumab or vedolizumab therapy
14. The in vitro method according to the embodiments 1 or 2, for predicting if the subject with gastrointestinal inflammatory disorder will respond to therapy of anti-TNF and/or anti-TNF, whereby downregulation of whole blood TREM1 expression or predicts future anti-TNF induced endoscopic remission.
15. The in vitro method according to the embodiments 1 or 2, for predicting if the subject with Crohn's disease will respond to therapy of anti-TNF and/or anti-TNF, whereby downregulation of whole blood TREM1 expression or predicts future anti-TNF induced endoscopic remission.
16. The in vitro method according to the embodiments 1 or 2, for predicting if the subject with Ulcerative colitis will respond to therapy of anti-TNF and/or anti-TNF, whereby downregulation of whole blood TREM1 expression indicates or predicts future anti-TNF induced endoscopic remission.
Yet some embodiments of the invention are set forth in embodiment format directly below:
1. An in vitro method of determining if a subject suffering from an patient suffering of inflammatory bowel diseases will respond or not to anti-α₄β₇-integrin therapy, wherein the method comprises: obtaining a biological sample from the subject; analyzing the level of its FAM129A, ELM01, TRIP13, PTAR1, ASAH1, SKAP2, HAUS1, C3orf67, SEC14L6, ATP6V0D1, ABCG1, ERAP1, ERV3_1, APOL6 and STON2 expression or activity of expression product of FAM129A, ELM01, TRIP13, PTAR1, ASAH1, SKAP2, HAUS1, C3orf67, SEC14L6, ATP6V0D1, ABCG1, ERAP1, ERV3_1, APOL6 and STON2 in the biological sample, and comparing said level of expression or activity with the FAM129A, ELM01, TRIP13, PTAR1, ASAH1, SKAP2, HAUS1, C3orf67, SEC14L6, ATP6V0D1, ABCG1, ERAP1, ERV3_1, APOL6 and STON2 expression or activity from a control sample; wherein a different level of FAM129A, ELM01, TRIP13, PTAR1, ASAH1, SKAP2, HAUS1, C3orf67, SEC14L6, ATP6V0D1, ABCG1, ERAP1, ERV31, APOL6 and STON2 expression or activity relative to the control sample is an indication of response to anti-α₄β₇-integrin or a propensity thereto in the subject.
2. An in vitro method of determining if a subject suffering from an patient suffering of inflammatory bowel diseases will respond or not to anti-TNF therapy, wherein the method comprises: obtaining a biological sample from the subject; analyzing the level of its ELOVL4, FGL2, CTSW, DDX11, LYZ, TRAPPC4, CDKAL1, ACVRL1, TSPAN14, PCNP, CITED4, CLEC5A, SGK1, ALOX5AP and SGK223 expression or activity of expression product of ELOVL4, FGL2, CTSW, DDX11, LYZ, TRAPPC4, CDKAL1, ACVRL1, TSPAN14, PCNP, CITED4, CLEC5A, SGK1, ALOX5AP and SGK223 in the biological sample, and comparing said level of expression or activity with the ELOVL4, FGL2, CTSW, DDX11, LYZ, TRAPPC4, CDKAL1, ACVRL1, TSPAN14, PCNP, CITED4, CLEC5A, SGK1, ALOX5AP and SGK223 expression or activity from a control sample; wherein a different level of ELOVL4, FGL2, CTSW, DDX11, LYZ, TRAPPC4, CDKAL1, ACVRL1, TSPAN14, PCNP, CITED4, CLEC5A, SGK1, ALOX5AP and SGK223 expression or activity relative to the control sample is an indication of response to anti-TNF or a propensity thereto in the subject
3. The in vitro method according to any one of the embodiments 1 to 2, wherein the inflammatory condition of the large intestine and/or small intestine is an inflammatory bowel disease.
4. The in vitro method according to any one of the embodiments 1 to 2, further comprising predicting if the subject will respond to therapy for Crohn's disease.
5. The in vitro method according to any one of the embodiments 1 to 2, further comprising predicting if the subject will respond to therapy for Ulcerative colitis.
6. The in vitro method according to the embodiments 1 and 2, for predicting if the subject with Crohn's disease will respond to therapy of anti-TNF and/or anti-α₄β₇-integrin.
7. The in vitro method according to the embodiments 1 and 2, for predicting if the subject with Ulcerative colitis will respond to therapy of anti-TNF and/or anti-α₄β₇-integrin.

Through multi-omic data integration, we discovered pathways contributing to ustekinumab response, and identified cell-type specific multi-gene panels which could discern biological and endoscopic ustekinumab-induced response. This type of integrated analyses will further aid in understanding the complexity of therapeutic response to biological agents, but needs further confirmation in independent datasets.

Prior to ustekinumab initiation, RNA sequencing was performed on inflamed colonic and ileal biopsies, as well as on circulating monocytes and CD4⁺ T-cells of patients with active CD. Proteomic analysis was done on baseline serum samples using proximity extension technology. Genotyping data were generated using Immunochip. The 6 above described-omic layers were then integrated using Multi-Omics Factor Analysis (MOFA). Feature selection and predictive modelling were used to accurately predict a 50% drop in faecal calprotectin (fCal) at week 8 and an endoscopic response (>50% in SES-CD) at week 24. Pathway and network analysis were subsequently performed to understand the mode of ustekinumab action.

MOFA identified 20 latent factors (LF, minimum explained variance 2%), with 2 LFs explaining the reduction in fCal (r=−0.25, p=0.05; r=−0.22, p=0.09) and 1 LF explaining endoscopic response (r=0.21,p=0.09). The colonic and monocyte transcriptomic layers contributed to the 2 LFs explaining the fCal reduction, while the circulating monocyte transcriptomic layer contributed significantly to the LF explaining the endoscopic response. Feature selection resulted in the identification of two 10-feature panels with high discriminatory power to discern the clinical groups based on their fCal or endoscopic responses. Identified pathways and networks contributing to ustekinumab response were clearly linked to the IL-12/IL-23/IL-17 axis, but also to antigen presentation, OX40 signalling and activation of T-cells.
Yet some embodiments of the invention are set forth in embodiment format directly below:
1. A method to predict the response of an immune bowel disorder patient to a drug directed against interleukin 12 (IL12) and interleukin 23 (IL23), characterised in that the method comprising the steps of: (a) determining the expression or activity of genes in a biological sample taken from the patient prior to treatment with the drug, and (b) comparing the expression profile of genes in said biological samples of patient with the expression or activity of genes obtained from a control sample, for instance of patients having progressive disease and/or of patients having stable disease or better after the drug treatment, wherein the genes comprise the group consisting of genes of table 4′ and wherein a different level of gene expression or activity relative to the control sample is an indication of response to the treatment or a propensity thereto in the patient.
2. A method to predict the response of an immune bowel disorder patient to a drug that blocks or inhibits the IL-12/23 pathway comprising the steps of: (a) determining the expression or activity of genes in a biological sample taken from the patient prior to treatment with the drug that blocks or inhibits the IL-12/23 pathway, and (b) comparing the expression profile of genes in said biological samples of patient with the expression or activity of genes obtained from a control sample, for instance of patients having progressive disease and/or of patients having stable disease or better after the treatment with the drug that blocks or inhibits the IL-12/23 pathway, wherein the genes comprise the group consisting of genes of table 4′ and wherein a different level of gene expression or activity relative to the control sample is an indication of response to the treatment or a propensity thereto in the patient.
3. A method to predict the response of an immune bowel disorder patient to an anti-P40 antibody comprising the steps of: (a) determining the expression or activity of genes in a biological sample taken from the patient prior to treatment with an anti-P40 antibody, and (b) comparing the expression profile of genes in said biological samples of patient with the expression or activity of genes obtained from a control sample, for instance of patients having progressive disease and/or of patients having stable disease or better after the anti-P40 antibody treatment, wherein the genes comprise the group consisting of genes of table 4′ and wherein a different level of gene expression or activity relative to the control sample is an indication of response to the treatment or a propensity thereto in the patient.
4. A method to predict the response of a Crohn's disease patient to ustekinumab (CNTO1275) comprising the steps of: (a) determining the expression or activity of genes in a biologic sample taken from the patient prior to treatment with ustekinumab, and (b) comparing the expression profile of genes in said biological samples of patient with the expression or activity of genes obtained from a control sample, for instance of patients having progressive disease and/or of patients having stable disease or better after the ustekinumab treatment, wherein the genes comprise the group consisting of genes of table 4′ and wherein a different level of gene expression or activity relative to the control sample is an indication of response to the treatment or a propensity thereto in the patient.
Yet some embodiments of the invention are set forth in embodiment format directly below:
1. A method to predict the response of an immune bowel disorder patient to a drug directed against interleukin 12 (IL12) and interleukin 23 (IL23), characterised in that the method comprising the steps of: (a) determining the expression or activity of genes in a biological sample taken from the patient prior to treatment with the drug, and (b) comparing the expression profile of genes in said biological samples of patient with the expression or activity of genes obtained from a control sample, for instance of patients having progressive disease and/or of patients having stable disease or better after the drug treatment, wherein the genes comprise the group consisting of genes of table 4′ and wherein a different level of gene expression or activity relative to the control sample is an indication of response to the treatment or a propensity thereto in the patient.
2. A method to predict the response of an immune bowel disorder patient to a drug that blocks or inhibits the IL-12/23 pathway comprising the steps of: (a) determining the expression or activity of genes in a biological sample taken from the patient prior to treatment with the drug that blocks or inhibits the IL-12/23 pathway, and (b) comparing the expression profile of genes in said biological samples of patient with the expression or activity of genes obtained from a control sample, for instance of patients having progressive disease and/or of patients having stable disease or better after the treatment with the drug that blocks or inhibits the IL-12/23 pathway, wherein the genes comprise the group consisting of genes of table 4′ and wherein a different level of gene expression or activity relative to the control sample is an indication of response to the treatment or a propensity thereto in the patient.
3. A method to predict the response of an immune bowel disorder patient to an anti-P40 antibody comprising the steps of: (a) determining the expression or activity of genes in a biological sample taken from the patient prior to treatment with an anti-P40 antibody, and (b) comparing the expression profile of genes in said biological samples of patient with the expression or activity of genes obtained from a control sample, for instance of patients having progressive disease and/or of patients having stable disease or better after the anti-P40 antibody treatment, wherein the genes comprise the group consisting of genes of table 4′ and wherein a different level of gene expression or activity relative to the control sample is an indication of response to the treatment or a propensity thereto in the patient.
4. A method to predict the response of a Crohn's disease patient to ustekinumab (CNTO1275) comprising the steps of: (a) determining the expression or activity of genes in a biologic sample taken from the patient prior to treatment with ustekinumab, and (b) comparing the expression profile of genes in said biological samples of patient with the expression or activity of genes obtained from a control sample, for instance of patients having progressive disease and/or of patients having stable disease or better after the ustekinumab treatment, wherein the genes comprise the group consisting of genes of table 4′ and wherein a different level of gene expression or activity relative to the control sample is an indication of response to the treatment or a propensity thereto in the patient.
5. The method according to any one of the previous embodiments where by the activity or the expression of the genes of the group consisting of CELSR3, HAAO, FAM135B, F2RL2, CMPK2, SLC28A2, RET, CHP2, PITX1 and GSTT1 are measured on colonic samples and the FCER2, CTSL, PTGFRN, GPRC5C, SLAMF7, NR4A2, GNG2, RHOC, SULT1A1, DSC2, NEDD4L, ENGASE, GSN, GNLY, CLEC10A, HLA_DRB5, BAG3, ASGR2, HLA_DRB1 and PTK2 are measured on CD14+ monocytes.
6. The method according to any one of the previous embodiments where by the activity or the expression of the genes of the group consisting of CELSR3, HAAO, FAM135B, F2RL2, CMPK2, SLC28A2, RET, CHP2, PITX1 and GSTT1 are measured on colonic samples and the FCER2, CTSL, PTGFRN, GPRC5C, SLAMF7, NR4A2, GNG2, RHOC, SULT1A1, DSC2, NEDD4L, ENGASE, GSN, GNLY, CLEC10A, HLA_DRB5, BAG3, ASGR2, HLA_DRB1 and PTK2 are measured on CD14+ monocytes.
Yet some embodiments of the invention are set forth in embodiment format directly below:
1. An in vitro method of determining if a subject suffering from gastrointestinal inflammatory disorder will respond or not to anti-TNF, wherein the method comprises: obtaining a biological sample from the subject; analyzing the level of its TREM-1 expression or activity of expression product of TREM-1 in the biological sample, and comparing said level of expression or activity with the TREM-1 expression or activity from a control sample; wherein a different level of TREM-1 expression or activity relative to the control sample is an indication of response to anti-TNF or a propensity thereto in the subject.
2. An in vitro method of determining the efficacy of a TNF antagonist for treatment of a gastrointestinal inflammatory disorder in a subject, the method comprising comparing the amount of a biomarker in a sample obtained from the subject after or during treatment with the TNF antagonist, to an amount of the biomarker in a sample obtained from the subject before the treatment, wherein a change in the amount of the biomarker after or during the treatment, as compared to before the treatment, is indicative of the response or efficacy of the antagonist for treatment of the gastrointestinal disorder in the subject, and wherein the biomarker is triggering receptor expressed on myeloid cells 1 (TREM1) in the subject's peripheral blood.
3. The in vitro method according to any one of the embodiments 1 or 2, wherein the inflammatory condition of the large intestine and/or small intestine is an inflammatory bowel disease.
4. The in vitro method according to any one of the embodiments 1 or 2, further comprising predicting if the subject will respond to therapy for Crohn's disease.
5. The in vitro method according to any one of the embodiments 1 or 2, further comprising predicting if the subject will respond to therapy for Ulcerative colitis.
6. The in vitro method according to the embodiments 1 or 2, for predicting if the subject with Crohn's disease will respond to therapy of anti-TNF and/or anti-TNF.
7. The in vitro method according to the embodiments 1 or 2, for predicting if the subject with Ulcerative colitis will respond to therapy of anti-TNF and/or anti-TNF.
8. The in vitro method according to the embodiments 1 or 2, for predicting if the subject with gastrointestinal inflammatory disorder will respond to therapy of anti-TNF and/or anti-TNF, whereby downregulation of whole blood TREM1 expression indicates the future anti-TNF healers.
9. The in vitro method according to the embodiments 1 or 2, for predicting if the subject with Crohn's disease will respond to therapy of anti-TNF and/or anti-TNF, whereby downregulation of whole blood TREM1 expression indicates the future anti-TNF healers.
10. The in vitro method according to the embodiments 1 or 2, for predicting if the subject with Ulcerative colitis will respond to therapy of anti-TNF and/or anti-TNF, whereby downregulation of whole blood TREM1 expression indicates the future anti-TNF healers.
11. The in vitro method according to the embodiments 1 or 2, for predicting if the subject with gastrointestinal inflammatory disorder will respond to therapy of anti-TNF and/or anti-TNF, whereby downregulation of whole blood TREM1 expression indicates the future healers on ustekinumab or vedolizumab therapy
12. The in vitro method according to the embodiments 1 or 2, for predicting if the subject with Crohn's disease will respond to therapy of anti-TNF and/or anti-TNF, whereby downregulation of whole blood TREM1 expression indicates the future healers on ustekinumab or vedolizumab therapy
13. The in vitro method according to the embodiments 1 or 2, for predicting if the subject with Ulcerative colitis will respond to therapy of anti-TNF and/or anti-TNF, whereby downregulation of whole blood TREM1 expression indicates the future healers on ustekinumab or vedolizumab therapy
14. The in vitro method according to the embodiments 1 or 2, for predicting if the subject with gastrointestinal inflammatory disorder will respond to therapy of anti-TNF and/or anti-TNF, whereby downregulation of whole blood TREM1 expression or predicts future anti-TNF induced endoscopic remission.
15. The in vitro method according to the embodiments 1 or 2, for predicting if the subject with Crohn's disease will respond to therapy of anti-TNF and/or anti-TNF, whereby downregulation of whole blood TREM1 expression or predicts future anti-TNF induced endoscopic remission.
16. The in vitro method according to the embodiments 1 or 2, for predicting if the subject with Ulcerative colitis will respond to therapy of anti-TNF and/or anti-TNF, whereby downregulation of whole blood TREM1 expression indicates or predicts future anti-TNF induced endoscopic remission.
Yet some embodiments of the invention are set forth in embodiment format directly below:
1. A method of determining the efficacy of a TNF antagonist for treatment of a gastrointestinal inflammatory disorder in a patient, the method comprising comparing the amount of a biomarker in a sample obtained from the patient after or during treatment with the TNF antagonist, to an amount of the biomarker in a sample obtained from the patient before the treatment, wherein a change in the amount of the biomarker after or during the treatment, as compared to before the treatment, is indicative of the efficacy of the antagonist for treatment of the gastrointestinal disorder in the patient, and wherein the biomarker is triggering receptor expressed on myeloid cells 1 (TREM1) in the patient's peripheral blood.
2. A method of predicting the responsiveness of a patient having a gastrointestinal inflammatory disorder to treatment with an TNF antagonist, the method comprising comparing the amount of a biomarker in a sample obtained from the patient after or during treatment with the TNF antagonist, to the amount of the biomarker in a sample obtained from the patient before the treatment, wherein a change in the amount of the biomarker after or during the treatment, as compared to before the treatment is indicative of the responsiveness of said patient to treatment with said antagonist, and wherein the biomarker is TREM1 in the patient's peripheral blood.
3. A method of determining the dosing of a TNF antagonist for treatment of a gastrointestinal inflammatory disorder in a patient, the method comprising adjusting the dose of the TNF antagonist based on a comparison of the amount of a biomarker in a sample obtained from the patient after or during treatment with a dose or dosing regimen of the TNF antagonist, to an amount of the biomarker in a sample obtained from the patient before the treatment, wherein a change in the amount of the biomarker after or during the treatment, as compared to before the treatment, is indicative of the efficacy of or responsiveness to the dose or dosing regimen of the TNF antagonist for treatment of the gastrointestinal disorder in the patient, and wherein the biomarker is TREM1 in the patient's peripheral blood.
4. A method of determining the dosing regimen of a TNF antagonist for treatment of a gastrointestinal inflammatory disorder in a patient, the method comprising adjusting the dose regimen of the TNF antagonist based on a comparison of the amount of a biomarker in a sample obtained from the patient after or during treatment with a dosing regimen of the TNF antagonist, to an amount of the biomarker in a sample obtained from the patient before the treatment, wherein a change in the amount of the biomarker after or during the treatment, as compared to before the treatment, is indicative of the efficacy of or responsiveness to the dose or dosing regimen of the TNF antagonist for treatment of the gastrointestinal disorder in the patient, and wherein the biomarker is TREM1 in the patient's peripheral blood.
5. The method of embodiment 1 or 2, wherein said change in the amount of the biomarker is an increase or decrease.
6. The method of embodiment 1 or 2, wherein said change in the amount of the biomarker is decrease.
7. The method of embodiment 6, wherein the amount of said biomarker is measured within 100 days after receiving a first dose of the agent.
8. The method of embodiment 6, wherein the amount of said biomarker is measured at least about 24 hours after administering the agent.
9. The method of any one of embodiments 1-8, wherein said gastrointestinal inflammatory disorder is an inflammatory bowel disease.
10. The method of embodiment 9, wherein said inflammatory bowel disease is Crohn's disease (CD) or ulcerative colitis (UC).
11. The method of embodiment 10, wherein said patient is a human.
12. The method of embodiment 10, wherein said TNF antagonist is an anti-TNF antibody.
13. The method of embodiment 12, wherein said antibody is monoclonal.
14. The method of embodiment 12, wherein said antibody is a chimeric, human or humanized antibody.
15. The method of embodiment 12, wherein said antibody is an antibody fragment.
16. The method of embodiment 12, wherein said agent is ustekinumab or vedolizumab
17. A method of embodiment 1, 2, 3 or 4, wherein said sample is a peripheral blood sample of said patient.
18. A method of designing a treatment with a candidate agent for a human patient diagnosed with a gastrointestinal inflammatory disorder, comprising determining an effective dosage for the human patient based on a dosage that effectively decreases the amount of the biomarker TREM1 in peripheral blood of a non-human subject in response to a treatment with said candidate agent, wherein the biomarker is in the patient's peripheral blood.
19. A method of embodiment 18, wherein said non-human subject is a monkey.
20. The method of embodiment 19, wherein said agent is an anti-TNF antibody.

The present invention also solves the problems of the related art by determining if a subject suffering from an inflammatory condition of the large intestine and/or small intestine will or will not respond to anti-α₄β₇-integrin therapy.

In accordance with a purpose of an aspect if present invention, as embodied and broadly described herein, the invention is broadly drawn to an in vitro method of determining if a patient suffering of an inflammatory bowel diseases will respond to anti-α₄β₇-integrin therapy, wherein the method comprises: obtaining a biological sample from the subject, preferably a colonic biopsy; analyzing the level of its PIWIL1, MAATS1, RGS13 and DCHS2 expression (mRNA) or activity of expression product of PIWIL1, MAATS1, RGS13 and DCHS2 in the biological sample, and comparing the determined expression level of PIWIL1, MAATS1, RGS13 and DCHS2 with a predetermined reference level, wherein a different level of PIWIL1, MAATS1, RGS13 and DCHS2 expression or activity relative to the control sample is an indication of response to anti-α₄β₇-integrin or a propensity thereto in the subject

In accordance with another purpose of the invention, as embodied and broadly described herein, the invention is broadly drawn to an in vitro method of determining if a patient suffering of an inflammatory bowel diseases will respond to anti-α₄β₇-integrin therapy, wherein the method comprises: obtaining a biological sample from the subject, preferably a colonic biopsy; analyzing the level of its PIWIL1, MAATS1, RGS13 and DCHS2 expression (mRNA) or activity of expression product of PIWIL1, MAATS1, RGS13 and DCHS2 in the biological sample, and comparing said level of expression or activity with the PIWIL1, MAATS1, RGS13 and DCHS2 expression or activity from remitters and/or non-remitters control sample; wherein a different level of PIWIL1, MAATS1, RGS13 and DCHS2 expression or activity relative to the control sample is an indication of response to anti-α₄β₇-integrin or a propensity thereto in the subject.

In one aspect of the invention, the an in vitro method of determining if a a patient suffering of an inflammatory bowel diseases will respond to anti-α₄β₇-integrin therapy, wherein the method comprises: obtaining a biological sample from the subject; analyzing the level of it PIWIL1, MAATS1, RGS13 and DCHS2 expression or activity of expression product of PIWIL1, MAATS1, RGS13 and DCHS2 in the biological sample, and comparing said level of expression or activity with the PIWIL1, MAATS1, RGS13 and DCHS2 expression or activity from a control sample; wherein a different level of PIWIL1, MAATS1, RGS13 and DCHS2 expression or activity relative to the control sample is an indication of response to anti-α₄β₇-integrin or a propensity thereto in the subject wherein a decreased level of PIWIL1, MAATS1, RGS13 and DCHS2 in comparison to the control sample is indicative of a positive response to the anti-α₄β₇-integrin therapy in the subject.

Another aspect of the invention is the inflammatory condition of the large intestine and/or small intestine is an inflammatory bowel disease.

In still another aspect of the invention, is an in vitro method of determining if a s a patient suffering of an inflammatory bowel diseases will respond to anti-α₄β₇-integrin therapy, wherein the method comprises: obtaining a biological sample from the subject; analyzing the level of it PIWIL1, MAATS1, RGS13 and DCHS2 expression or activity of expression product of PIWIL1, MAATS1, RGS13 and DCHS2 in the biological sample, and comparing said level of expression or activity with the PIWIL1, MAATS1, RGS13 and DCHS2 expression or activity from a control sample; wherein a decreased level of PIWIL1, MAATS1, RGS13 and DCHS2 is indicative of a positive response thereto and is predictive of a responder.

By using an inventive system it is possible of predicting if the subject will respond to anti-α₄β₇-integrin therapy for Crohn's disease or ulcerative colitis.

By using an inventive system it is possible to predict if the subject will respond to an anti-α₄β₇-integrin antibody therapy that blocks action of α₄β₇-integrin by preventing Integrin α₄β₇ on CD4 T cells forming a complex with MadCam-1 on endothelial cells.

By using an inventive system it is possible to predict if the subject suffering from an inflammatory bowel disease will respond to an anti-α₄β₇-integrin antibody therapy that blocks the action of α₄β₇-integrin by preventing α₄β₇-integrin of interacting addressin MadCAM-1.

In a practical embodiment, the device according to the present invention comprises measuring the level of PIWIL1, MAATS1, RGS13 and DCHS2 as an indication of a positive response thereto and is indicative of a responder, wherein the expression product is a nucleic acid molecule selected from the group consisting of mRNA and cDNA mRNA or polypeptides derived therefrom.

In a particular embodiment of present invention the sample isolated form the subject is from a colonic mucosal biopsy.

In a practical embodiment, the device according to the present invention comprises measuring the level of PIWIL1, MAATS1, RGS13 and DCHS2 as an indication of a positive response thereto and is indicative of a responder, wherein the method comprises the detection of the level of the nucleic acids or polypeptides carried out utilizing at least one binding agent specifically binding to the nucleic acids or polypeptides to be detected. Such binding agent can be detectably labelled, for instance with a label is selected from the group consisting of a radioisotope, a bioluminescent compound, a chemiluminescent compound, a fluorescent compound, a metal chelate, biotin, digoxigenin, and an enzyme. In another embodiment of the invention, the at least one binding agent is an aptamer or an antibody selected from the group consisting of a monoclonal antibody; a polyclonal antibody; a fab-fragment; a single chain antibody; and an antibody variable domain sequence. In yet another embodiment of the invention, the at least one binding agent being a nucleic acid hybridising to a nucleic acid utilized for the detection of marker molecules, PIWIL1, MAATS1, RGS13 and DCHS2 expression. Moreover the detection reaction can comprise a nucleic acid amplification reaction.

The object of the present invention is also to use the in-vitro method according to present for in-situ detection.

In another aspect, the present invention provides a diagnostic test kit for use in diagnosing a subject for responsiveness to an anti-α₄β₇-integrin treatment of inflammatory bowel disease and/or Crohn's disease (cd), or for use in monitoring the effectiveness of therapy of inflammatory bowel disease in patients receiving an anti-α₄P₇-integrin therapy, the diagnostic kit comprising: a predetermined amount of an antibody specific for PIWIL1, MAATS1, RGS13 and DCHS2; a predetermined amount of a specific binding partner to said antibody; buffers and other reagents necessary for monitoring detection of antibody bound to PIWIL1, MAATS1, RGS13 and DCHS2; and wherein either said antibody or said specific binding partner is detectably labelled.

In yet another aspect, the present invention provides a diagnostic test kit for use in diagnosing a subject for responsiveness to anti-α₄β₇-integrin treatment of inflammatory bowel disease (ibd) or for use in monitoring the effectiveness of therapy of inflammatory bowel disease in patients receiving an to anti-α₄β₇-integrin therapy, the diagnostic kit comprising: a) a nucleic acid encoding the PIWIL1, MAATS1, RGS13 and DCHS2 protein; b) reagents useful for monitoring the expression level of the one or more nucleic acids or proteins encoded by the nucleic acids of step a); and c) instructions for use of the kit.

We recruited inflamed colonic biopsies from 31 patients (20 UC, 11 CD) prior to initiation of vedolizumab. Similarly, inflamed colonic biopsies (15 UC, 9 CD) were collected from 24 patients initiating anti-TNF therapy (Table 1′″). RNA was extracted and single-end RNA sequencing was performed using Illumina HiSeq4000. Normalization and differential expression was done using DESeq2 R package. Pathways were analysed with Ingenuity Pathway Analysis (IPA). Using randomized generalized linear modelling (RGLM), a predictor for vedolizumab-induced endoscopic remission (absence of ulcerations at month 6 for CD; Mayo endoscopic sub-score <1 at week 14 for UC) was identified in a randomly generated test cohort (n=20) and validated in 11 independent samples. Through unsupervised consensus clustering, we validated the marker in a publically available microarray dataset (GSE73661), and studied vedolizumab specificity in the anti-TNF treated cohort.

Forty-four genes (25 down, 19 up) were significantly differently expressed between future vedolizumab remitters and non-remitters. Involved pathways included glucocorticoid receptor signalling, differential regulation of cytokines in intestinal epithelial cells, granulocyte adhesions and diapedesis. Using these 44 differentially expressed genes as input for the RGLM modelling, we identified a 4-gene signature which could accurately split remitters and non-remitters in both the discovery (accuracy 80.2%, p=0.02) and validation (100%, p=0.006) set. Using the same 4-gene signature we could accurately discriminate prospective future remitters from non-remitters in a publically available microarray data set of 13 open-label vedolizumab treated UC patients (84.6%, p=0.02). In contrast, this 4-gene signature was not predictive for anti-TNF induced endoscopic remission (62.5%, p=0.65).

We identified and validated the first, vedolizumab-specific predictive 4-gene expression signature which may guide treatment strategy in IBD patients with colonic involvement.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

Yet some embodiments of the invention are set forth in embodiment format directly below:
1. A method of testing whether a patient suffering of inflammatory bowel diseases will respond or not to a treatment with α₄β₇-integrin inhibition, comprising determining the expression level of a plurality of biomarkers in a cell or tissue or body fluid sample obtained of said subject, whereby the plurality of biomarkers comprises the IWIL1, MAATS1, RGS13 and DCHS2 genes.
2. The method of embodiment 1, comprising determining the expression level of RNA transcripts or their products in a biological sample obtained of said subject, wherein the RNA transcript is the RNA transcript of the PIWIL1, MAATS1, RGS13 and DCHS2 genes, wherein increased expression of based on genomic features of the four signature genes PIWIL1, MAATS1, RGS13 and DCHS2 in the cell or tissue or body fluid sample, or their corresponding product, indicates an increased likelihood of a positive response to the α₄β₇-integrin inhibition treatment.
3. A method of predicting sensitivity to α₄β₇-integrin inhibition by a α₄β₇-integrin inhibitor in a cell or tissue or body fluid sample from a subject, comprising: assigning a sensitivity score to α₄β₇-integrin inhibition based on genomic features of the four signature genes PIWIL1, MAATS1, RGS13 and DCHS2 in the cell or tissue or body fluid sample.
4. A method for selecting a subject for treatment of a disease or condition with a therapy comprising α₄β₇-integrin inhibitor, comprising: (a) assigning a sensitivity score to α₄β₇-integrin inhibition based on genomic features of four signature genes PIWIL1, MAATS1, RGS13 and DCHS2 in a cell or tissue or body fluid sample from the subject according to the method of embodiment 1, and (b) selecting the subject for treatment with a therapy comprising a α₄β₇-integrin inhibitor based on the assigned sensitivity score.
5. A method of prognosis of a disease or condition suitable for treatment with a therapy comprising aα₄β₇-integrin inhibitor in a patient, comprising: assigning a sensitivity score to α₄β₇-integrin inhibition based on genomic features of four signature genes PIWIL1, MAATS1, RGS13 and DCHS2 in a cell or tissue or body fluid sample from the subject according to the method of embodiment 1; wherein the prognosis of patient with the disease or condition is based on the assigned sensitivity score.
6. A method of predicting a response to aα₄β₇-integrin inhibitor therapy in a patient, comprising: assigning a sensitivity score to α₄β₇-integrin inhibition based on genomic features of four signature genes PIWIL1, MAATS1, RGS13 and DCHS2 in a cell or tissue or body fluid sample from the subject according to the method of embodiment 1; wherein the patient is predicted to respond to or not respond to aα₄β₇-integrin inhibitor therapy based on the assigned sensitivity score.
7. A method for predicting efficacy of, or monitoring treatment with a therapy comprising aα₄β₇-integrin inhibitor in a subject having a disease or condition, comprising: assigning a sensitivity score to α₄β₇-integrin inhibition based on genomic features of four signature genes PIWIL1, MAATS1, RGS13 and DCHS2 according to the method of embodiment 1 in a cell or tissue or body fluid sample from a subject who is or has been treated with the therapy comprising the α₄β₇-integrin inhibitor; wherein the assigned sensitivity score indicates whether the treatment is effective or is likely to be effective, or is an indicator of the progress of treatment.
8. A method for improving clinical outcome of treatment with a therapy comprising aα₄β₇-integrin inhibitor in a subject having a disease or condition, comprising assigning a sensitivity score to α₄β₇-integrin inhibition based on genomic features of four signature genes PIWIL1, MAATS1, RGS13 and DCHS2 in a cell or tissue or body fluid sample from the subject according to the method of embodiment 1; and developing appropriate treatment based on the assigned sensitivity score thereby improving clinical outcome.
9. The method according to any of embodiments 4 to 8, further comprising: altering treatment based on the assigned sensitivity score.
10. The method according to embodiment 1, further comprising: obtaining the cell or tissue or body fluid sample from the subject; and analyzing the cell or tissue or body fluid sample from the subject for genomic features of the four signature genes PIWIL1, MAATS1, RGS13 and DCHS2.
11. The method according to embodiment 1, wherein the assigning the sensitivity score comprises determining expression levels of four signature genes PIWIL1, MAATS1, RGS13 and DCHS2.
12. The method according to embodiment 1-11, further comprising: comparing the assigned sensitivity score to a reference sensitivity score.
13. The method according to embodiment 12, wherein the reference sensitivity is determined from a reference sample, wherein the reference sample is a sample from a healthy subject, is a sample from an individual not having the disease or condition, is a baseline sample from the subject prior to treatment with a therapy comprising aα₄β₇-integrin inhibitor or is a sample from a subject prior to the last dose of a therapy comprising aα₄β₇-integrin inhibitor.
14. The method according to any of embodiments 1-13, wherein the disease or condition is inflammatory condition of the large intestine and/or small intestine is an inflammatory bowel disease.
15. The method according to any of embodiments 1-13, wherein the disease or condition is Crohn's disease.
16. The method according to any of embodiments 1-13, wherein the assigning the sensitivity score comprises applying a linear regression model to the genomic features of four signature genes PIWIL1, MAATS1, RGS13 and DCHS2; and optionally combining the genomic features into a predictive model using a multivariate algorithm.
17. The method of embodiment 16, wherein the linear regression model is a multivariate linear regression model.
18. The method according to any of embodiments 1-17, wherein the genomic features comprise a feature selected from the group consisting of gene expression (mRNA expression or protein expression), gene copy number, and activating or deactivating point mutation.
19. The method according to any of embodiments 1-18, wherein the α₄β₇-integrin inhibitor is a small molecule.
20. The method of embodiment 21, wherein the small molecule α₄β₇ integrin inhibitors are of the group consisting of TR-14035, Chemical Structure

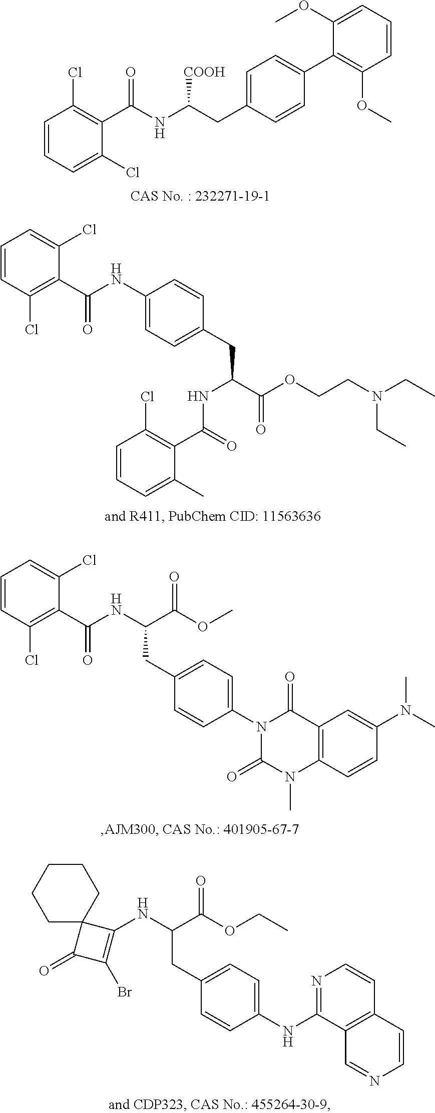 [SMILES: COc1cccc(OC)c1-c1ccc(C[C@H](NC(=O)c2c(Cl)cccc2Cl)C(=O)O)cc1 ; CCN(CC)CCOC(=O)[C@H](Cc1ccc(NC(=O)c2c(Cl)cccc2Cl)cc1)NC(=O)c1c(C)cccc1Cl ; COC(=O)[C@H](Cc1ccc(-n2c(=O)c3cc(N(C)C)ccc3n(C)c2=O)cc1)NC(=O)c1c(Cl)cccc1Cl ; CCOC(=O)C(Cc1ccc(Nc2nccc3ccncc23)cc1)NC1=C(Br)C(=O)C12CCCCC2]

21. The method according to any of embodiments 1-18, wherein the α₄β₇-integrin inhibitor is an antibody.
22. The method of embodiment 21, wherein the antibody is of the group consisting of an antibody of α4-subunit blocking both α4β1 and α4β7 integrin, an antibody that selectively targets the α4β7 integrin, an antibody that is selectively against the β7-subunit of α4β7 integrin and a human monoclonal antibody specifically against the α4β7 integrin.
23. The method of embodiment 21, wherein the antibody is of the group consisting of an Natalizumab, Vedolizumab, Etrolizumab and AMG-18.

Yet some other embodiments of the invention are set forth in embodiment format directly below:
1. A method of predicting the likelihood of positive response to treatment with vedolizumab of a subject diagnosed with inflammatory bowel diseases, comprising determining the expression level of a signature of the four PIWIL1, MAATS1, RGS13 and DCHS2 genes in a cell or tissue or body fluid sample obtained of said subject.
2. The method of embodiment 1, comprising determining the expression level of RNA transcripts or their products in a biological sample obtained of said subject, wherein the RNA transcript is the RNA transcript of the PIWIL1, MAATS1, RGS13 and DCHS2 genes, wherein increased expression of based on genomic features of the four signature genes PIWIL1, MAATS1, RGS13 and DCHS2 in the cell or tissue or body fluid sample, or their corresponding product, indicates an increased likelihood of a positive response to the vedolizumab treatment.
3. A method of predicting sensitivity to vedolizumab treatment, comprising: assigning a sensitivity score to vedolizumab based on genomic features of the four signature genes PIWIL1, MAATS1, RGS13 and DCHS2 in the cell or tissue or body fluid sample.
4. A method for selecting a subject for treatment of a disease or condition with a vedolizumab therapy, comprising: (a) assigning a sensitivity score to vedolizumab based on genomic features of four signature genes PIWIL1, MAATS1, RGS13 and DCHS2 in a cell or tissue or body fluid sample from the subject according to the method of embodiment 1, and (b) selecting the subject for treatment with vedolizumab based on the assigned sensitivity score.
5. A method of prognosis of a disease or condition suitable for treatment with a vedolizumab therapy in a patient, comprising: assigning a sensitivity score to vedolizumab based on genomic features of four signature genes PIWIL1, MAATS1, RGS13 and DCHS2 in a cell or tissue or body fluid sample from the subject according to the method of embodiment 1; wherein the prognosis of patient with the disease or condition is based on the assigned sensitivity score.
6. A method of predicting a response to a vedolizumab therapy in a patient, comprising: assigning a sensitivity score to vedolizumab based on genomic features of four signature genes PIWIL1, MAATS1, RGS13 and DCHS2 in a cell or tissue or body fluid sample from the subject according to the method of embodiment 1; wherein the patient is predicted to respond to or not respond to vedolizumab therapy based on the assigned sensitivity score.
7. A method for predicting efficacy of, or monitoring treatment with a vedolizumab therapy in a subject having a disease or condition, comprising: assigning a sensitivity score to vedolizumab based on genomic features of four signature genes PIWIL1, MAATS1, RGS13 and DCHS2 according to the method of embodiment 1 in a cell or tissue or body fluid sample from a subject who is or has been treated with the vedolizumab therapy; wherein the assigned sensitivity score indicates whether the treatment is effective or is likely to be effective, or is an indicator of the progress of treatment.
8. A method for improving clinical outcome of treatment with a vedolizumab therapy in a subject having a disease or condition, comprising assigning a sensitivity score to vedolizumab based on genomic features of four signature genes PIWIL1, MAATS1, RGS13 and DCHS2 in a cell or tissue or body fluid sample from the subject according to the method of embodiment 1; and developing appropriate treatment based on the assigned sensitivity score thereby improving clinical outcome.
9. The method according to any of embodiments 4 to 8, further comprising: altering treatment based on the assigned sensitivity score.
10. The method according to embodiment 1, further comprising: obtaining the cell or tissue or body fluid sample from the subject; and analyzing the cell or tissue or body fluid sample from the subject for genomic features of the four signature genes PIWIL1, MAATS1, RGS13 and DCHS2.
11. The method according to embodiment 1, wherein the assigning the sensitivity score comprises determining expression levels of four signature genes PIWIL1, MAATS1, RGS13 and DCHS2.
12. The method according to embodiment 1-11, further comprising: comparing the assigned sensitivity score to a reference sensitivity score.
13. The method according to embodiment 12, wherein the reference sensitivity is determined from a reference sample, wherein the reference sample is a sample from a healthy subject, is a sample from an individual not having the disease or condition, is a baseline sample from the subject prior to treatment with a vedolizumab therapy or is a sample from a subject prior to the last dose of a therapy comprising vedolizumab.
14. The method according to any of embodiments 1-13, wherein the disease or condition is inflammatory condition of the large intestine and/or small intestine is an inflammatory bowel disease.
15. The method according to any of embodiments 1-13, wherein the disease or condition is Crohn's disease.
16. The method according to any of embodiments 1-13, wherein the assigning the sensitivity score comprises applying a linear regression model to the genomic features of four signature genes PIWIL1, MAATS1, RGS13 and DCHS2; and optionally combining the genomic features into a predictive model using a multivariate algorithm.
17. The method of embodiment 16, wherein the linear regression model is a multivariate linear regression model.

Yet some embodiments of the invention are set forth in embodiment format directly below:
1. An in vitro method of determining if a subject suffering from an patient suffering of inflammatory bowel diseases will respond or not to anti-α₄β₇-integrin therapy, wherein the method comprises: obtaining a biological sample from the subject; analyzing the level of it PIWIL1, MAATS1, RGS13 and DCHS2 expression or activity of expression product of PIWIL1, MAATS1, RGS13 and DCHS2 in the biological sample, and comparing said level of expression or activity with the PIWIL1, MAATS1, RGS13 and DCHS2 expression or activity from a control sample; wherein a different level of PIWIL1, MAATS1, RGS13 and DCHS2 expression or activity relative to the control sample is an indication of response to anti-α₄β₇-integrin or a propensity thereto in the subject.
2. The in vitro method of embodiment 1, wherein a decreased level of PIWIL1, MAATS1, RGS13 and DCHS2 in comparison to the control sample is indicative of a positive response to the anti-α₄β₇-integrin therapy in the subject.
3. The in vitro method according to any one of the embodiments 1 to 2, wherein the inflammatory condition of the large intestine and/or small intestine is an inflammatory bowel disease.
4. The in vitro method according to any one of the embodiments 1 to 2, further comprising predicting if the subject will respond to anti-α₄β₇-integrin therapy for Crohn's disease.
5. The in vitro method according to any one of the embodiments 1 to 2, further comprising predicting if the subject will respond to an anti-α₄β₇-integrin antibody therapy that blocks action of α₄β₇-integrin by preventing Integrin α₄β₇ forming a complex with the T-cell surface Cd4.
6. The in vitro method of to any one of the embodiments 1 to 5, wherein a decreased level of PIWIL1, MAATS1, RGS13 and DCHS2 is indicative of a positive response thereto and is predictive of a responder.
7. The in vitro method according to embodiment 5, further comprising predicting if the subject suffering from an inflammatory bowel disease will respond to an anti-α₄β₇-integrin antibody therapy that blocks the action of α₄β₇-integrin by preventing α₄β₇-integrin of interacting addressin MadCAM-1.
8. The in vitro method of embodiment 7, wherein a decreased level of PIWIL1, MAATS1, RGS13 and DCHS2 is indicative of a positive response thereto and is indicative of a responder.
9. The in vitro method according to any one of the embodiments 1 to 8, wherein the expression product is a nucleic acid molecule selected from the group consisting of mRNA and cDNA mRNA or polypeptides derived therefrom.
10. The in vitro method according to any one of the embodiments 1 to 9, wherein the sample isolated form the subject is from a colonic mucosal biopsy.
11. The in-vitro method according to any one of the embodiments 1 to 10, comprising the detection of the level of the nucleic acids or polypeptides carried out utilizing at least one binding agent specifically binding to the nucleic acids or polypeptides to be detected.
12. The in-vitro method according to any one of the embodiments 1 to 11, wherein the binding agent is detectably labelled.
13. The in-vitro method according to embodiment 12, wherein the label is selected from the group consisting of a radioisotope, a bioluminescent compound, a chemiluminescent compound, a fluorescent compound, a metal chelate, biotin, digoxigenin, and an enzyme.
14. The in-vitro method according to any one of the embodiments 1 to 13, wherein at least one binding agent is an aptamer or an antibody selected from the group consisting of a monoclonal antibody; a polyclonal antibody; a fab-fragment; a single chain antibody; and an antibody variable domain sequence.
15. The in-vitro method according to any one of the embodiments 1 to 13, with at least one binding agent being a nucleic acid hybridising to a nucleic acid utilized for the detection of marker molecules, PIWIL1, MAATS1, RGS13 and DCHS2 expression.
16. The in-vitro method according to embodiment 15, wherein the detection reaction comprises a nucleic acid amplification reaction.
17. The in-vitro method according to any one of the embodiments 1 to 16, the method wherein the method is be utilized for in-situ detection.
18. The in vitro method according to any one of the embodiments 1 to 17, wherein the samples are inflamed colonic biopsies from inflammatory bowel disease (IBD) patients
19. The in vitro method according to any one of the embodiments 1 to 18, wherein the wherein the α₄β₇-integrin inhibitor is an antibody of the group consisting of an antibody of α4-subunit blocking both α4β1 and α4β7 integrin, an antibody that selectively targets the α4β7 integrin, an antibody that is selectively against the β7-subunit of α4β7 integrin and a human monoclonal antibody specifically against the α4β7 integrin.
20. The in vitro method according to any one of the embodiments 1 to 18, wherein the wherein the α₄β₇-integrin inhibitor is an antibody of the group consisting of an Natalizumab, Vedolizumab, Etrolizumab and AMG-18.
21. A diagnostic test kit for use in diagnosing a subject for responsiveness to an anti-α₄β₇-integrin treatment of inflammatory bowel disease and/or Crohn's disease (cd), or for use in monitoring the effectiveness of therapy of inflammatory bowel disease in patients receiving an anti-β₄β₇-integrin therapy, the diagnostic kit comprising: a predetermined amount of an antibody specific for PIWIL1, MAATS1, RGS13 and DCHS2; a predetermined amount of a specific binding partner to said antibody; buffers and other reagents necessary for monitoring detection of antibody bound to PIWIL1, MAATS1, RGS13 and DCHS2; and wherein either said antibody or said specific binding partner is detectably labelled.
22. A diagnostic test kit for use in diagnosing a subject for responsiveness to anti-α₄β₇-integrin treatment of inflammatory bowel disease (ibd) or for use in monitoring the effectiveness of therapy of inflammatory bowel disease in patients receiving an to anti-α₄β₇-integrin therapy, the diagnostic kit comprising: a) a nucleic acid encoding the PIWIL1, MAATS1, RGS13 and DCHS2 protein; b) reagents useful for monitoring the expression level of the one or more nucleic acids or proteins encoded by the nucleic acids of step a); and c) instructions for use of the kit.

### DETAILED DESCRIPTION

### Detailed Description of Embodiments of the Invention

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof.

The following detailed description of the invention refers to the accompanying drawings. The same reference numbers in different drawings identify the same or similar elements. Also, the following detailed description does not limit the invention. Instead, the scope of the invention is defined by the appended claims and equivalents thereof.

Several documents are cited throughout the text of this specification. Each of the documents herein (including any manufacturer's specifications, instructions etc.) are hereby incorporated by reference; however, there is no admission that any document cited is indeed prior art of the present invention.

The present invention will be described with respect to particular embodiments and with reference to certain drawings but the invention is not limited thereto but only by the claims.

The drawings described are only schematic and are non-limiting. In the drawings, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to the devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details.

In other instances, well-known methods, structures and techniques have not been shown in detail in order not to obscure an understanding of this description.

Other embodiments of the invention will be apparent to those skilled in the art from consideration of the specification and practice of the invention disclosed herein.

It is intended that the specification and examples be considered as exemplary only.

Each and every claim is incorporated into the specification as an embodiment of the present invention. Thus, the claims are part of the description and are a further description and are in addition to the preferred embodiments of the present invention.

Each of the claims set out a particular embodiment of the invention.

The following terms are provided solely to aid in the understanding of the invention.

We prospectively included 35 (discovery) and 19 (validation) consecutive, active IBD patients (both Crohn's disease and ulcerative colitis), initiating anti-TNF therapy, as well as 22 patients initiating ustekinumab and 51 patients initiating vedolizumab. Whole blood expression levels of OSM, TNF, TNFR2 and TREM1 (total and all individual transcripts separately) were measured prior to start of therapy using qPCR, and mucosal gene expression in inflamed biopsies using RNA-sequencing. Endoscopic remission was defined as an SES-CD≤2 at week 24 for Crohn's disease and a Mayo endoscopic sub-score 1 at week 8-14 for ulcerative colitis. Baseline whole blood TREM1 expression was significantly downregulated in future anti-TNF healers (p<0.001, both discovery and validation cohort). Receiver operator characteristic statistics showed an area under the curve (AUC) of 0.78 (p=0.001), resulting in post-test probabilities of 77.1% and 90.0% for endoscopic remission and non-remission, respectively. A similar accuracy could be observed in mucosal TREM1 expression (AUC 0.77, p=0.003), which outperformed the accuracy of serum TREM1 at the protein level (AUC 0.58, p=0.31). Whole blood TREM1 expression did not significantly correlate with CRP (spearman ρ=−0.08, p=0.38), faecal calprotectin (spearman p=−0.06, p=0.64) or serum TNFα (spearman ρ=−0.15, p=0.63). OSM, TNF and TNFR2 were not differentially expressed in whole blood (p=0.09, p=0.13, p=0.24 respectively), whereas they were at the mucosal level (p=0.007, p=0.02, p=0.008 respectively). The whole blood TREM1 predictive signal was anti-TNF specific, as no changes in expression were seen in ustekinumab and vedolizumab treated patients, neither in whole blood (p=0.82, p=0.53 respectively), nor in tissue (p=0.24, p=^{0.10}, respectively). We identified and validated low TREM-1 as a specific biomarker for anti-TNF induced endoscopic remission. These results can aid in the selection of therapy in biologic-naïve patients, but should be confirmed in a randomized trial prior to translation into daily clinical practice.

In certain embodiments, the present invention provides a compound of formula I-a or I-b or a pharmaceutically acceptable salt thereof.

### I. Definitions

Tumor necrosis factor (TNF, tumor necrosis factor alpha, TNFα, cachexin, or cachectin) is a cell signaling protein (cytokine) involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons. [Levin A D, et al. J Crohns Colitis 2016; 10(8):989-97] TNFα is a member of the TNF superfamily, consisting of various transmembrane proteins with a homologous TNF domain

As used herein, with respect to patient response or patient responsiveness, the term “prediction” or “predicting” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive or improve following treatment, for example treatment with a particular therapeutic agent, for instance, anti-TNF antibody, and for a certain period of time without disease recurrence. For example, the predictive methods of the invention can be used clinically to make treatment decisions by choosing the most appropriate treatment modalities for any particular patient. The predictive methods of the present invention are valuable tools in predicting whether a patient is likely to respond favorably to a treatment regimen, including for example, administration of a given therapeutic agent or combination, surgical intervention, steroid treatment, etc., or whether long-term survival of the patient, following a therapeutic regimen is likely.

“Treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis.

“Treatment regimen” refers to a combination of dosage, frequency of administration, or duration of treatment, with or without addition of a second medication.

“Effective treatment regimen” refers to a treatment regimen that will offer beneficial response to a patient receiving the treatment.

“Modifying a treatment” refers to changing the treatment regimen including, changing dosage, frequency of administration, or duration of treatment, and/or addition of a second medication.

“Patient response” or “patient responsiveness” can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e., reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment. The term “responsiveness” refers to a measurable response, including complete response (CR) and partial response (PR).

By “complete response” or “CR” is intended the disappearance of all signs of inflammation or remission in response to treatment. This does not always mean the disease has been cured.

“Partial response” or “PR” refers to a decrease of at least 50% in the severity of inflammation, in response to treatment.

A “beneficial response” of a patient to treatment with an integrin beta7 antagonist and similar wording refers to the clinical or therapeutic benefit imparted to a patient at risk for or suffering from a gastrointestinal inflammatory disorder from or as a result of the treatment with the antagonist, such as an anti-TNF antibody. Such benefit includes cellular or biological responses, a complete response, a partial response, a stable disease (without progression or relapse), or a response with a later relapse of the patient from or as a result of the treatment with the antagonist.

“A patient maintains responsiveness to a treatment” when the patient′ responsiveness does not decrease with time during the course of a treatment.

The term “diagnosis” is used herein to refer to the identification or classification of a molecular or pathological state, disease or condition. For example, “diagnosis” may refer to identification of a particular type of gastrointestinal inflammatory disorder, and more particularly, the classification of a particular sub-type of gastrointestinal inflammatory disorder, by tissue/organ involvement (e.g., inflammatory bowel disease), or by other features (e.g., a patient subpopulation characterized by responsiveness to a treatment, such as to a treatment with an integrin beta7 antagonist).

The term “prognosis” is used herein to refer to the prediction of the likelihood of disease symptoms, including, for example, recurrence, flaring, and drug resistance, of a gastrointestinal inflammatory disorder.

The term “sample”, as used herein, refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. The sample can be obtained from a tissue for the subject of interest or from peripheral blood of the subject.

“Anti-TNF agent” or “A TNF antagonist” or “TNFalpa antagonist” refers to any molecule that inhibits one or more biological activities or blocking binding of TNF with one or more of its associated molecules. Antagonists of the invention can be used to modulate one or more aspects of TNF associated effects. In one embodiment of the invention, the anti-TNF agent is an anti-TNF antibody. In one embodiment, the anti-TNF antibody is a humanized anti-TNF antibody and more particularly a recombinant humanized monoclonal anti-TNF antibody (or rhuMAb TNF).

An “amount” or “level” of biomarker can be determined using methods known in the art. In some embodiments, an “elevated” or “increased” amount or level of a biomarker, is as compared to a reference/comparator amount of the biomarker. The increase is preferably greater than about 10%, preferably greater than about 30%, preferably greater than about 50%, preferably greater than about 100%, preferably greater than about 300% as a function of the value for the reference or comparator amount. For example, a reference or comparator amount can be the amount of a biomarker before treatment and more particularly, can be the baseline or pre-dose amount.

In some embodiments, a “decreased” amount or level of a biomarker, is as compared to a reference/comparator amount of the biomarker. The decrease is preferably less than about 10%, preferably less than about 30%, preferably less than about 50%, preferably less than about 100%, preferably less than about 300% as a function of the value for the reference or comparator amount. For example, a reference or comparator amount can be the amount of a biomarker before treatment and more particularly, can be the baseline or pre-dose amount.

The phrase “does not substantially change” as used herein, denotes a insignificant degree of change such that one of skill in the art would not consider the change to be of statistical significance within the context of the biological characteristic measured by said values. The change is preferably less than about 10%, preferably less than about 5%, preferably less than about 1%.

“Gastrointestinal inflammatory disorders” are a group of chronic disorders that cause inflammation and/or ulceration in the mucous membrane. These disorders include, for example, inflammatory bowel disease (e.g., Crohn's disease, ulcerative colitis, indeterminate colitis and infectious colitis), mucositis (e.g., oral mucositis, gastrointestinal mucositis, nasal mucositis and proctitis), necrotizing enterocolitis and esophagitis. In a preferred embodiment, the gastrointestinal inflammatory disorder is a inflammatory bowel disease.

“Inflammatory Bowel Disease” or “IBD” is used interchangeably herein to refer to diseases of the bowel that cause inflammation and/or ulceration and includes without limitation Crohn's disease and ulcerative colitis.

“Crohn's disease (CD)” or “ulcerative colitis (UC)” are chronic gastrointestinal inflammatory disorder of unknown etiology. Crohn's disease, unlike ulcerative colitis, can affect any part of the bowel. The most prominent feature Crohn's disease is the granular, reddish-purple edematous thickening of the bowel wall. With the development of inflammation, these granulomas often lose their circumscribed borders and integrate with the surrounding tissue. Diarrhea and obstruction of the bowel are the predominant clinical features. As with ulcerative colitis, the course of Crohn's disease may be continuous or relapsing, mild or severe, but unlike ulcerative colitis, Crohn's disease is not curable by resection of the involved segment of bowel. Most patients with Crohn's disease require surgery at some point, but subsequent relapse is common and continuous medical treatment is usual.

Crohn's disease may involve any part of the alimentary tract from the mouth to the anus, although typically it appears in the ileocolic, small-intestinal or colonic-anorectal regions. Histopathologically, the disease manifests by discontinuous granulomatomas, crypt abscesses, fissures and aphthous ulcers. The inflammatory infiltrate is mixed, consisting of lymphocytes (both T and B cells), plasma cells, macrophages, and neutrophils. There is a disproportionate increase in IgM- and IgG-secreting plasma cells, macrophages and neutrophils.

Anti-inflammatory drugs sulfasalazine and 5-aminosalisylic acid (5-ASA) are useful for treating mildly active colonic Crohn's disease and are commonly prescribed to maintain remission of the disease. Metroidazole and ciprofloxacin are similar in efficacy to sulfasalazine and appear to be particularly useful for treating perianal disease. In more severe cases, corticosteroids are effective in treating active exacerbations and can even maintain remission. Azathioprine and 6-mercaptopurine have also shown success in patients who require chronic administration of cortico steroids. It is also possible that these drugs may play a role in the long-term prophylaxis. Unfortunately, there can be a very long delay (up to six months) before onset of action in some patients. Antidiarrheal drugs can also provide symptomatic relief in some patients. Nutritional therapy or elemental diet can improve the nutritional status of patients and induce symptomatic improvement of acute disease, but it does not induce sustained clinical remissions. Antibiotics are used in treating secondary small bowel bacterial overgrowth and in treatment of pyogenic complications.

“Ulcerative colitis (UC)” afflicts the large intestine. The course of the disease may be continuous or relapsing, mild or severe. The earliest lesion is an inflammatory infiltration with abscess formation at the base of the crypts of Lieberkuhn. Coalescence of these distended and ruptured crypts tends to separate the overlying mucosa from its blood supply, leading to ulceration. Symptoms of the disease include cramping, lower abdominal pain, rectal bleeding, and frequent, loose discharges consisting mainly of blood, pus and mucus with scanty fecal particles. A total colectomy may be required for acute, severe or chronic, unremitting ulcerative colitis.

The clinical features of UC are highly variable, and the onset may be insidious or abrupt, and may include diarrhea, tenesmus and relapsing rectal bleeding. With fulminant involvement of the entire colon, toxic megacolon, a life-threatening emergency, may occur. Extraintestinal manifestations include arthritis, pyoderma gangrenoum, uveitis, and erythema nodosum.

Treatment for UC includes sulfasalazine and related salicylate-containing drugs for mild cases and corticosteroid drugs in severe cases. Topical administration of either salicylates or corticosteroids is sometimes effective, particularly when the disease is limited to the distal bowel, and is associated with decreased side effects compared with systemic use. Supportive measures such as administration of iron and antidiarrheal agents are sometimes indicated. Azathioprine, 6-mercaptopurine and methotrexate are sometimes also prescribed for use in refractory corticosteroid-dependent cases.

An “effective dosage” refers to an amount effective, at dosages and for periods of time necessary, to achieve the desired therapeutic or prophylactic result.

As used herein, the term “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “non-human subject” refers to any single non-human animal, more preferably a mammal (including such non-human animals as, for example, dogs, cats, horses, rabbits, zoo animals, cows, pigs, sheep, and non-human primates).

The terms “antibody” and “immunoglobulin” are used interchangeably in the broadest sense and include monoclonal antibodies (for example, full length or intact monoclonal antibodies), polyclonal antibodies, multivalent antibodies, multispecific antibodies (e.g., bispecific antibodies so long as they exhibit the desired biological activity) and may also include certain antibody fragments (as described in greater detail herein). An antibody can be human, humanized and/or affinity matured.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise on antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994).

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol., 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al., Monoclonal Antibody Production Techniques and Applications, pp. 55-93 (Marcel Dekker, Inc., New York, 1987); and Boerner et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci. USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “hypervariable region,” “HVR,” or “HV,” when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures.

The phrase “substantially similar,” or “substantially the same,” as used herein, denotes a sufficiently high degree of similarity between two numeric values (generally one associated with an antibody of the invention and the other associated with a reference/comparator antibody) such that one of skill in the art would consider the difference between the two values to be of little or no biological and/or statistical significance within the context of the biological characteristic measured by said values (e.g., Kd values). The difference between said two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a n-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (x) and lambda (k), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (W. B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

A “naked antibody” for the purposes herein is an antibody that is not conjugated to a cytotoxic moiety or radiolabel.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays as herein disclosed, for example.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)), and regulates homeostasis of immunoglobulins. Antibodies with improved binding to the neonatal Fc receptor (FcRn), and increased half-lives, are described in WO00/42072 (Presta, L.) and US2005/0014934A1 (Hinton et al.). These antibodies comprise an Fc region with one or more substitutions therein which improve binding of the Fc region to FcRn. For example, the Fc region may have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region-comprising antibody variant with improved FcRn binding comprises amino acid substitutions at one, two or three of positions 307, 380 and 434 of the Fc region thereof (Eu numbering of residues).

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. Nos. 5,571,894; and 5,587,458.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “amino acid sequence variant” antibody herein is an antibody with an amino acid sequence which differs from a main species antibody. Ordinarily, amino acid sequence variants will possess at least about 70% homology with the main species antibody, and preferably, they will be at least about 80%, more preferably at least about 90% homologous with the main species antibody. The amino acid sequence variants possess substitutions, deletions, and/or additions at certain positions within or adjacent to the amino acid sequence of the main species antibody.

Examples of amino acid sequence variants herein include an acidic variant (e.g., deamidated antibody variant), a basic variant, an antibody with an amino-terminal leader extension (e.g. VHS−) on one or two light chains thereof, an antibody with a C-terminal lysine residue on one or two heavy chains thereof, etc., and includes combinations of variations to the amino acid sequences of heavy and/or light chains. The antibody variant of particular interest herein is the antibody comprising an amino-terminal leader extension on one or two light chains thereof, optionally further comprising other amino acid sequence and/or glycosylation differences relative to the main species antibody.

A “glycosylation variant” antibody herein is an antibody with one or more carbohydrate moieties attached thereto which differ from one or more carbohydrate moieties attached to a main species antibody. Examples of glycosylation variants herein include antibody with a G1 or G2 oligosaccharide structure, instead a GO oligosaccharide structure, attached to an Fc region thereof, antibody with one or two carbohydrate moieties attached to one or two light chains thereof, antibody with no carbohydrate attached to one or two heavy chains of the antibody, etc., and combinations of glycosylation alterations. Where the antibody has an Fc region, an oligosaccharide structure may be attached to one or two heavy chains of the antibody, e.g. at residue 299 (298, Eu numbering of residues).

The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32 and radioactive isotopes of Lu), chemotherapeutic agents, and toxins such as small molecule toxins or enzymatically active toxins of bacterial, fungal, plant or animal origin, including fragments and/or variants thereof.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “immunosuppressive agent” as used herein for adjunct therapy refers to substances that act to suppress or mask the immune system of the subject being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir; tacrolimus; glucocorticoids such as cortisol or aldosterone; anti-inflammatory agents such as a cyclooxygenase inhibitor; a 5-lipoxygenase inhibitor; or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporine; 6 mercaptopurine; steroids such as corticosteroids or glucocorticosteroids or glucocorticoid analogs, e.g., prednisone, methylprednisolone, including SOLU-MEDROL® methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies or antagonists including anti-interferon-alpha, -beta, or -gamma antibodies, anti-tumor necrosis factor (TNF)-alpha antibodies (infliximab (REMICADE®) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists; anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodomase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF or BR3 antibodies or immunoadhesins and zTNF4 antagonists (for review, see Mackay and Mackay, Trends Immunol., 23:113-5 (2002) and see also definition below); biologic agents that interfere with T cell helper signals, such as anti-CD40 receptor or anti-CD40 ligand (CD154), including blocking antibodies to CD40-CD40 ligand. (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9.

“Compounds” of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

Apilimod,

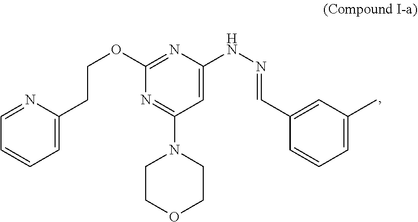 [SMILES: Cc1cccc(/C=N/Nc2cc(N3CCOCC3)nc(OCCc3ccccn3)n2)c1]

and Apilimod mesylate,

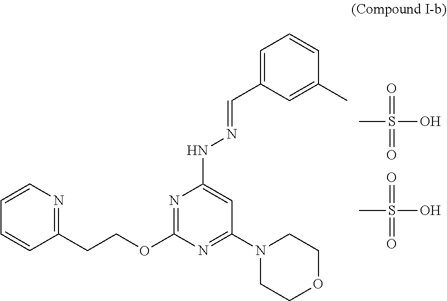

are inhibitors of IL-12/23, they inhibit the IL-12 and IL-23 production
α₄β₇ integrin inhibitors are known in the art. For instance Natalizumab is a mAbs of α₄-subunit blocking both α₄β₁ and α₄β₇ integrin. Vedolizumab selectively targets the α₄β₇ integrin. Etrolizumab is selectively against the β₇-subunit of α₄β₇ integrin and AMG-181 is a human monoclonal antibody specifically against the α₄β₇ integrin are the most promising anti-α₄β₇ integrin antibodies. Small molecule α₄β₇ integrin inhibitors are for instance TR-14035,

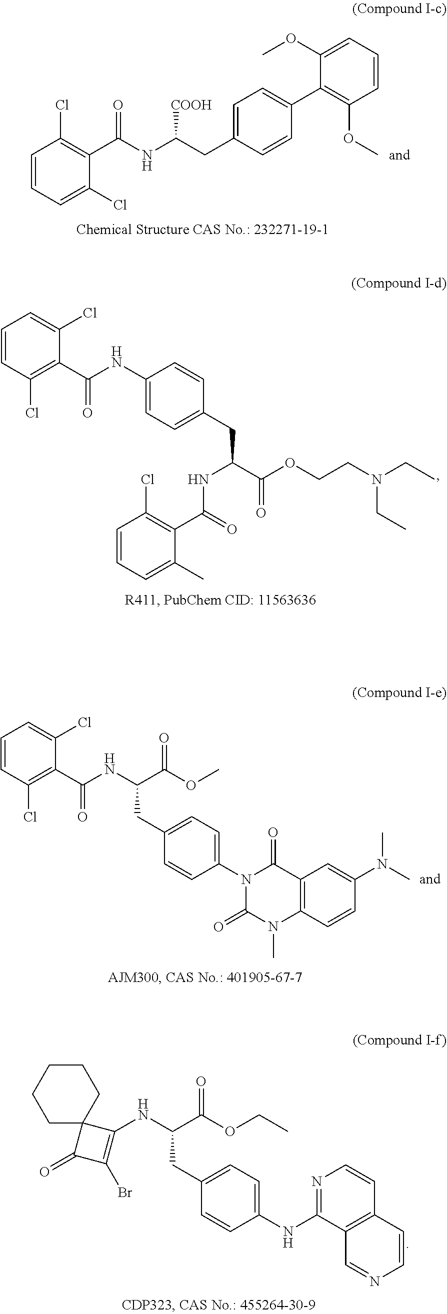 [SMILES: COc1cccc(OC)c1-c1ccc(C[C@H](NC(=O)c2c(Cl)cccc2Cl)C(=O)O)cc1 ; CCN(CC)CCOC(=O)[C@H](Cc1ccc(NC(=O)c2c(Cl)cccc2Cl)cc1)NC(=O)c1c(C)cccc1Cl ; COC(=O)[C@H](Cc1ccc(-n2c(=O)c3cc(N(C)C)ccc3n(C)c2=O)cc1)NC(=O)c1c(Cl)cccc1Cl ; CCOC(=O)[C@H](Cc1ccc(Nc2nccc3ccncc23)cc1)NC1=C(Br)C(=O)C12CCCCC2]

Also peptide α₄β₇ integrin inhibitors inhibitors are known in the art.

Vedolizumab, a monoclonal antibody targeting alpha4beta7 integrin and mainly inhibiting gut lymphocyte trafficking, has been approved for the treatment of both Crohn's disease (CD) and ulcerative colitis (UC). Due to the increasing availability of therapeutic compounds in inflammatory bowel disease (IBD), predictive biomarkers are urgently awaited in order to help in deciding for anti-TNF, vedolizumab or other therapy.

We obtained inflamed colonic biopsies from IBD patients initiating vedolizumab or anti-TNF therapy, and performed RNA-sequencing. Besides standard differential gene expression, pathway analysis and cell deconvolution, we applied predictive modelling in a training (n=20) and validation dataset (n=11). The identified model, predicting vedolizumab-induced endoscopic remission (absence of ulcerations at month 6 for Crohn's disease; Mayo endoscopic sub-score ≤1 at week 14 for ulcerative colitis) was subsequently validated in three independent datasets (n=66 in total).

Forty-four genes were significantly differently expressed between vedolizumab endoscopic remitters and non-remitters, with a significant upregulation of leukocyte migration in non-remitters (p<0.006). Deconvolution methods identified a significant enrichment of monocytes (p=0.005), M1 macrophages (p=0.05) and effector memory CD4 T cells (p=0.008) in non-remitters, whereas remitters demonstrated a baseline enrichment of naïve B cells (p=0.05).

We identified a 4-gene colonic signature (PIWIL1, MAATS1, RGS13, DCHS2) accurately differentiating remitters from non-remitters in both the training and validation dataset (accuracy 80.0%; 100%), and in all 3 independent datasets, including qPCR validation (p=0.003). In contrast, this 4-gene signature was not predictive for anti-TNF responsiveness. Finally, we confirmed the presence of the identified genes at protein level in inflamed colon, using immunohistochemistry.

ABCG1 concerns the gene_assignment ABCG1—ATP binding cassette subfamily G member 1 with GeneID: 9619 and with mRNA Genbank No. NM 004915, NM 016818, NM_207174, NM_207627 and NM_207628 where under the versions NM_016818.2, NM_004915.3, NM_207174.1, NM_207627.1 and NM_207628.1 and RefSeq (protein) NP_004906, NP 058198, NP_997057, NP 997510 and NP_997511 where under the versions NP 058198.2, NP 004906.3, NP_997057.1, NP_997510.1 and NP_997511.1 ACVRL1 concerns the gene_assignment ACVRL1—activin A receptor like type 1 with GeneID: 94 and with mRNA Genbank No. NM_000020 and NM_001077401 where under the versions NM_000020.3 and NM_001077401.2 and RefSeq (protein) NP_000011 and NP_001070869 where under the versions NP_000011.2 and NP_001070869.1 ALOX5AP concerns the gene_assignment ALOX5AP arachidonate 5-lipoxygenase activating protein with Gene ID: 241 and with mRNA Genbank No. NM_001204406 and NM_001629 where under the versions NM_001204406.1 and NM_001629.3 and RefSeq (protein) NP_001191335 and NP_001620 where under the versions NP_001191335.1 and NP_001620.2. APOL6 concerns the gene_assignment APOL6—apolipoprotein L6 with GeneID: 80830 and with mRNA Genbank No. NM_030641 where under the version NM_030641.4 and RefSeq (protein) NP_085144 where under the version NP_085144.1

ASAH1 concerns the gene_assignment ASAH1—N-acylsphingosine amidohydrolase 1 with GeneID: 427 and with mRNA Genbank No. NM_001127505, NM 004315, NM_177924 and NM_001363743 where under the versions NM 177924.5, NM_004315.6, NM_001127505.3, NM_001363743.2 and RefSeq (protein) NP_001120977, NP_004306, NP_808592, NP_001350672 where under the versions NP 808592.2, NP 004306.3, NP_001120977.1 and NP_001350672.1

ASGR2 concerns the gene_assignment ASGR2—asialoglycoprotein receptor 2 with GeneID: 433 and with mRNA Genbank No. NM_080914 where under the versions NM_001201352.1, NM 001181.4, NM_080912.3, NM_080913.3 and NM_080914.2 and RefSeq (protein) P07307 where under the versions NP_001188281.1, NP_001172.1, NP_550434.1, NP_550435.1 and NP_550436.1

ATP6V0D1 concerns the gene_assignment ATP6V0D1—ATPase H+ transporting V0 subunit d1 with GeneID: 9114 and with mRNA Genbank No. NM_004691 where under the version NM_004691.5 and RefSeq (protein) NP_004682 where under the versions NP_004682.2

BAG3 concerns the gene_assignment BAG3—BAG cochaperone 3 with GeneID: 9531 and with mRNA Genbank No. NM_004281 where under the versions NM_004281.3 and RefSeq (protein) NP_004272 where under the versions NP_004272.2

C3orf67 concerns the gene_assignment C3orf67—chromosome 3 open reading frame 67 with GeneID: 200844 and with mRNA Genbank No. NM_198463, NM_001351530, NM_001351531, NM_001351532 and NM_001351533 where under the versions NM_001351530.2, NM_198463.4, NM_001351531.2, NM_001351532.2 and NM_001351533.1 and RefSeq (protein) NP_940865, NP_001338459, NP_001338460, NP_001338461 and NP_001338462 where under the versions NP_001338459.1, NP_940865.1, NP_001338460.1, NP_001338461.1 and NP_001338462.1

CDKAL1 concerns the gene_assignment CDKAL1 CDK5 regulatory subunit associated protein 1 like 1 with Gene ID: 54901 and with mRNA Genbank No. NM_017774 where under the versions NM_017774.3 and RefSeq (protein) NP_060244 where under the versions NP_060244.2

CELSR3 concerns the gene_assignment CELSR3—cadherin EGF LAG seven-pass G-type receptor 3 with GeneID: 1951 and with mRNA Genbank No. NM_001407 where under the version NM_001407.3 and RefSeq (protein) NP_001398 where under the version NP_001398.2 CHP2 concerns the gene_assignment CHP2 calcineurin like EF-hand protein 2 with Gene ID: 63928 and with mRNA Genbank No. NM_022097.4 and RefSeq (protein) NP_071380.1 CITED4 concerns the gene_assignment CITED4 Cbp/p300 interacting transactivator with Glu/Asp rich carboxy-terminal domain 4 with Gene ID: 163732 and with mRNA Genbank No. NM_133467.3 and RefSeq (protein) NP_597724.1

CLEC10A concerns the gene_assignment CLEC10A—C-type lectin domain containing 10A with GeneID: 10462 and with mRNA Genbank No. NM_006344, NM_182906 and NM_001330070 where under the versions NM_001330070.2, NM_006344.4 and NM_182906.4 and RefSeq (protein) NP_001316999, NP_006335 and NP_878910 where under the versions NP_001316999.1, NP_006335.2 and NP_878910.1

CLEC5A concerns the gene_assignment CLEC5A—C-type lectin domain containing 5A with GeneID: 23601 and with mRNA Genbank No. NM_001301167 and NM_013252 where under the versions NM_013252.3, NM_001301167.2 and XM_011515995.2 and RefSeq (protein) NP_001288096 and NP_037384 where under the versions NP_037384.1, NP_001288096.1 CMPK2 concerns the gene_assignment CMPK2—cytidine/uridine monophosphate kinase 2 with GeneID: 129607 and with mRNA Genbank No. NM_207315 where under the versions NM_207315.3, NM_001256477.1, NM_001256478.1 and NR_046236.2 and RefSeq (protein) NP_997198.2, NP_001243406.1 and NP_001243407.1.

CTSL concerns the gene assignment CTSL—cathepsin L with GeneID: 1514 and with mRNA Genbank No. NM_001335 where under the versions NM_001257971.2, NM_001257972.2, NM_001257973.2, NM_001912.5 and NM_145918.3 and RefSeq (protein) NP_001326 where under the versions NP_666023.1, NP_001903.1, NP_001244902.1, NP_001244901.1 and NP_001244900.1

CTSW concerns the gene_assignment CTSW cathepsin W with Gene ID: 1521 and with mRNA Genbank No. NM_001335 where under the versions NM_001335.4 and RefSeq (protein) NP_001326 where under the versions NP_001326.3

DCHS2 concerns the gene_assignment DCHS2—dachsous cadherin-related 2 with GeneID: 54798 and with mRNA Genbank No. NM_001142552, NM 001142553, NM_017639, NM_199348 and NM_001358235 where under the versions NM_001358235.2 and NM_001142552.2 and RefSeq (protein) NP_001136024 and NP_001345164 where under the versions NP_001345164.1 and NP_001136024.1

DDX11 concerns the gene_assignment DDX11—DEAD/H-box helicase 11 with GeneID: 1663 and with mRNA Genbank No. NM 001257144, NM 001257145, NM_004399, NM 030653, NM_030655 where under the versions NM_030653.4, NM_004399.3, NM_152438.2, NM_001257144.2 and NM_001257145.2 and RefSeq (protein) NP_001244073, NP_001244074, NP_004390, NP_085911 and NP_689651 where under the versions NP_085911.2, NP_004390.3, NP_689651.1, NP_001244073.1 and NP_001244074.1 DSC2 concerns the gene_assignment DSC2 desmocollin 2 with Gene ID: 1824 and with mRNA Genbank No. NM_004949 and NM_024422 and RefSeq (protein) NP_004940 and NP_077740

ELM01 concerns the gene_assignment ELMO1—engulfment and cell motility 1 with GeneID: 9844 and with mRNA Genbank No. NM_001039459, NM_001206480, NM 001206482, NM_014800 and NM_130442 where under the versions NM_001206480.2, NM_014800.10, NM_130442.3, NM_001039459.2 and NM_001206482.1 and RefSeq (protein) NP_001034548, NP_001193409, NP_001193411, NP_055615 and NP_569709 where under the versions NP_001193409.1, NP_055615.8, NP_569709.1, NP_001034548.1 and NP_001193411.1

ELOVL4 concerns the gene_assignment ELOVL4—ELOVL fatty acid elongase 4 with GeneID: 6785 and with mRNA Genbank No. NM_022726 where under the version NM_022726.4 and RefSeq (protein) NP_073563 where under the version NP_073563.1

ENGASE concerns the gene_assignment ENGASE—endo-beta-N-acetylglucosaminidase with GeneID: 64772 and with mRNA Genbank No. NM_001042573 and NM_022759 where under versions NM_001042573.3 and RefSeq (protein) NP_001036038 where under the versions NP_001036038.1

ERAP1 concerns the gene_assignment ERAP1—endoplasmic reticulum aminopeptidase 1 with GeneID: 51752 and with mRNA Genbank No. NM_001040458, NM_001198541, NM 016442 and NM 001349244 where under the versions NM 001040458.3, NM_016442.4, NM_001198541.2 and NM_001349244.1 and RefSeq (protein) NP_001035548, NP_001185470, NP_057526, NP_001336173 where under the versions NP_001035548.1, NP_057526.3, NP_001185470.1 and NP_001336173.1

ERV3_1 concerns the gene_assignment ERV3-1—endogenous retrovirus group 3 member 1, envelope with GeneID: 2086 and with mRNA Genbank No. NM_001007253, where under the versions NM_001007253.4, NR_145414.2 and NR_145415.2 and RefSeq (protein) NP_001007254 where under the version NP_001007254.2

F2RL2 concerns the gene_assignment F2RL2 coagulation factor II thrombin receptor like 2 with Gene ID: 2151 and with mRNA Genbank No. NM_004101 and NM_001256566, where under version NM_001256566.2 and version NM_004101.4 and RefSeq (protein) NP_001243495 and NP_004092

FAM129A concerns the gene assignment Fam129a—family with sequence similarity 129, member A with GeneID: 63913, including version NM_022018.3 and with mRNA Genbank No. NM_052966 and RefSeq (protein) NP_443198 and including version NP_071301.2 FAM135B concerns the gene_assignment FAM135B—family with sequence similarity 135 member B with GeneID: 51059 with mRNA Genbank No. NM_015912 and NM_001362965, including the versions NM_015912.4 and NM_001362965.1 and RefSeq (protein) NP_056996 and NP_001349894, including NP_056996.2 and NP_001349894.1

FCER2 concerns the gene_assignment FCER2—Fc fragment of IgE receptor II with GeneID: 2208 and with mRNA Genbank No. NM 001207019, NM_001220500 and NM_002002 where under the versions NM_001220500.2, NM_002002.4 and NM_001207019.2 and RefSeq (protein) NP_001193948, NP_001207429 and NP_001993 where under the versions NP_001207429.1, NP_001993.2 and NP_001193948.2

FGL2 concerns the gene_assignment FGL2—fibrinogen like 2 with GeneID: 10875 and with mRNA Genbank No. NM_006682 where under version NM_006682.3 and RefSeq (protein) NP_006673, where under version NP_006673.1

GNG2 concerns the gene_assignment GNG2—G protein subunit gamma 2 with GeneID: 54331 and with mRNA Genbank No. NM 001243773, NM_001243774 and NM_053064 including the versions NM_053064.5, NM_001243773.2 and NM_001243774.2 and RefSeq (protein) NP_001230702, NP_001230703 and NP_444292 including the versions NP_444292.1, NP_001230702.1 and NP_001230703.1

GNLY concerns the gene assignment GNLY—granulysin with and with mRNA Genbank No. NM_001302758, NM_006433 and NM_012483, including the versions NM_006433.5 NM_012483.4 and RefSeq (protein) NP_001289687, NP_006424 and NP_036615, including the versions NP_006424.2 and NP_036615.2

GPRC5C concerns the gene_assignment GPRC5C—G protein-coupled receptor class C group 5 member C with GeneID: 55890 and with mRNA Genbank No. NM 018653, NM_022036, NM_001366261 and NM_001366262, including the versions NM_022036.4 NM_018653.4, NM_001366261.2, NM_001366262.2 and RefSeq (protein) NP_061123n NP_071319, NP_001353190 and NP_001353191, including the versions NP_071319.3, NP_061123.4, NP_001353190.1 and NP_001353191.1

GSN concerns the gene_assignment GSN—gelsolin with GeneID: 2934 and with mRNA Genbank No. NM_000177, NM_001127662, NM_001127663, NM_001127664 and NM_001127665 including the versions NM_198252.3, NM_000177.5, NM_001127662.2, NM_001127663.2 and NM_001127664.2 and RefSeq (protein) NP_000168, NP_001121134, NP_001121135, NP_001121136 and NP_001121137 and including the versions and NP_937895.1, NP_000168.1, NP_001121134.1, NP_001121135.2 and NP_001121136.1

GSTT1 concerns the gene assignment GSTT1—glutathione S-transferase theta 1 with GeneID: 2952 and with mRNA Genbank No. NM 000853, NM_001293807, NM_001293808 NM_001293809 and NM_001293810 including the versions NM_000853.3, NM_001293807.1, NM_001293808.1, NM_001293809.1 and NM_001293810.1 and RefSeq (protein) NP_000844, NP_001280736, NP_001280737, NP_001280738 and NP_001280739 including the versions NP_000844.2, NP_001280736.1, NP_001280737.1, NP_001280738.1 and NP_001280739.1

HAAO concerns the gene_assignment HAAO—3-hydroxyanthranilate 3,4-dioxygenase with GeneID: 23498 and with mRNA Genbank No. NM_012205.3 and RefSeq (protein) NP_036337.2

HAUS1 concerns the gene_assignment HAUS1—HAUS augmin like complex subunit 1 with GeneID: 115106 and with mRNA GenbankNo. NM_138443 including versions NM_138443.4 and NR_026978.2 and RefSeq (protein) NP_612452 and including version NP_612452.1

HLA_DRB1 concerns the gene_assignment HLA-DRB1—major histocompatibility complex, class II, DR beta 1 with GeneID: 3123 and with mRNA Genbank No. NM_001243965, NM_002124, NM_001359193 and NM_001359194 where under the version NM_001243965.1, NM_002124.3, NM_001359193.1 and NM_001359194.1 and RefSeq (protein) NP_001230894, NP_002115, NP_001346122, NP_001346123 and NP_001230894.1 where under the version NP_001230894.1, NP_002115.2, NP_001346122.1 and NP_001346123.1

HLA_DRB5 concerns the gene_assignment HLA-DRB5—major histocompatibility complex, class II, DR beta 5 with GeneID: 3127 and with mRNA Genbank No. NM_002125 including version NM_002125.4 and RefSeq (protein) NP_002116 including version NP_002116.2 LYZ concerns the gene_assignment LYZ—lysozyme with GeneID: 4069 and with mRNA Genbank No. NM_000239 where under the version NM_000239.3 and RefSeq (protein) NM_013590 where under the version NP_000230.1

MAATS1 concerns the gene_assignment MAATS1—MYCBP associated and testis expressed 1 with GeneID: 89876 and with mRNA Genbank No. NM 033364, NM_001320316, NM 001320317 and NM 001320318 where under the versions NM 033364.4, NM_001320316.1, NM_001320317.1 and NM_001320318.1 and RefSeq (protein) NP_001307245, NP_001307246, NP_001307247 and NP_203528, where under the versions NP_203528.3, NP_001307245.1, NP_001307246.1 and NP_001307247.1

NEDD4L concerns the gene_assignment NEDD4L—NEDD4 like E3 ubiquitin protein ligase with GeneID: 23327 and with mRNA Genbank No. NM_001144964, NM_001144965, NM_001144966, NM_001144967 and NM_001144968 including the versions NM_001144967.3, NM_015277.6, NM_001144964.1, NM_001144965.2 and NM_001144966.3 and RefSeq (protein) NP_001138436 including NP_001138437, NP_001138438, NP_001138439 and NP_001138440 including the versions NP_001138439.1 NP_056092.2, NP_001138436.1, NP_001138437.1 and NP_001138438.1

NR4A2 concerns the gene_assignment NR4A2—nuclear receptor subfamily 4 group A member 2 with GeneID: 4929 and with mRNA Genbank No. NM 006186, NM_173171, NM_173172 and NM_173173 and RefSeq (protein) NP_006177, NP_775265 and NP_006177.1

PCNP concerns the gene_assignment PCNP—PEST proteolytic signal containing nuclear protein with GeneID: 57092 and with mRNA Genbank No. NM_020357.3, NM_001320395.1 NM_001320397.1, NM_001320398.1 NM_001320399.1 and RefSeq (protein) NP_065090.1 NP_001307324.1, NP_001307326.1, NP_001307327.1 and NP_001307328.1 PITX1 concerns the gene_assignment PITX1—paired like homeodomain 1 with GeneID: 5307 and with mRNA Genbank No. NM_002653, where under the version NM_002653.5 and RefSeq (protein) NP_002644, where under the version NP_002644.4

PIWIL1 concerns the gene_assignment PIWIL1—piwi like RNA-mediated gene silencing 1 with GeneID: 9271 and with mRNA Genbank No. NM 001190971 and NM 004764 where under the versions NM_004764.5 and NM_001190971.2 and RefSeq (protein) NP_001177900 and NP_004755 where under the versions NP_004755.2 and NP_001177900.1

PTAR1 concerns the gene_assignment PTAR1—protein prenyltransferase alpha subunit repeat containing 1 with GeneID: 375743 and with mRNA Genbank No. NM_001099666.2, NM_001366935.1, NM_001366936.1, NM_001366937.1 and NM_001366938.1 and RefSeq (protein) NP_001093136.1, NP_001353864.1, NP_001353865.1, NP_001353866.1 and NP_001353867.1

PTGFRN concerns the gene_assignment PTGFRN—prostaglandin F2 receptor inhibitor with GeneID: 5738 and with mRNA Genbank No. xxxxx and RefSeq (protein) xxxxxx

PTK2 concerns the gene_assignment PTK2—protein tyrosine kinase 2 with GeneID: 5747 and with mRNA Genbank No. NM_001199649, NM_005607, NM_153831 and NM_001316342, where under versions NM_001352701.2, NM 005607.5, NM_153831.4, NM_001199649.2 and NM_001316342.2 and RefSeq (protein) NP_001186578, NP_001303271, NP_005598, NP_722560 and NP_001339623, where under versions NP_001339630.1, NP_005598.3, NP_722560.1, NP_001186578.1 and NP_001303271.1

RET concerns the gene_assignment RET—ret proto-oncogene with GeneID: 5979 and with mRNA Genbank No. NM_000323, NM_020629, NM 020630, NM_020975 and NM_001355216 and RefSeq (protein) NP_065681, NP_066124, NP_001342145 and NP_066124.1

RGS13 concerns the gene_assignment RGS13—regulator of G protein signaling 13 with GeneID: 6003 and with mRNA Genbank No. NM 144766 and NM 002927 where under the specific transcripts NM_002927.5 and NM_144766.3 and RefSeq (protein) NP_002918 and NP_658912, where under versions NP_002918.1 and NP_658912.1

RHOC concerns the gene_assignment RHOC—ras homolog family member C with GeneID: 389 and with mRNA Genbank No. NM_175744, NM_001042678, where under versions NM_175744.4, NM_001042678.1 and NM_001042679.1 and NM_001042679 and RefSeq (protein) NP_001036143, NP_001036144 and NP_786886, where under versions NP_786886.1, NP_786886.1 and NP_001036144.1

SEC14L6 concerns the gene_assignment SEC14L6—SEC14 like lipid binding 6 with GeneID: 730005 and with mRNA Genbank No. NM_001193336.3, NM_001353441.2 and NM_001353443.2 and RefSeq (protein) NP_001180265.2, NP_001340370.1 and NP_001340372.1

SGK1 concerns the gene_assignment SGK1—serum/glucocorticoid regulated kinase 1 with GeneID: 6446 and with mRNA Genbank No. NM_001143676, NM_001143677, NM_001143678, NM_001291995 and NM_005627 and RefSeq (protein) NP_001137148, NP_001137149, NP_001137150, NP_001278924 and NP_005618

SGK223 concerns the gene_assignment PRAG1 PEAK1 related, kinase-activating pseudokinase 1 with Gene ID: 157285 and with mRNA Genbank No. NM_001080826.3 and NM_001369759.1 and RefSeq (protein) NP_001074295.2 and NP_001356688.1

SKAP2 concerns the gene_assignment SKAP2—src kinase associated phosphoprotein 2 with GeneID: 8935 and with mRNA Genbank No. NM 003930 where under NM 003930.5 and NM_001303468 where under NM_001303468.1 and RefSeq (protein) NP_003921.2 and NP_001290397.1

SLAMF7 concerns the gene assignment SLAMF7—SLAM family member 7 with GeneID: 57823 and with mRNA Genbank No. NM 021181.5, NM_001282588, NM_001282589, NM_001282590, NM_001282591 and NM_001282592 and RefSeq (protein) NP_067004.3, NP_001269517, NP_001269518, NP_001269519, NP_001269520 and NP_001269521

SLC28A2 concerns the gene_assignment SLC28A2—solute carrier family 28 member 2 with GeneID: 9153 and with RefSeq (mRNA) NM_004212 and RefSeq (protein) NP_004203 STON2 concerns the gene_assignment STON2—stonin 2 with GeneID: 85439 and with mRNA Genbank No. NM_001366850.2, NM_001256430.2 and NM_001366849.2 and RefSeq (protein) NP_001353779.1, NP_001243359.1 and NP_001353778.1

SULT1A1 concerns the gene assignment SULT1A1—sulfotransferase family 1A member 1 with GeneID: 6817 and with mRNA Genbank No. NM 177536, NM 001055, NM_177529, NM_177530 and NM_177534 and RefSeq (protein), NP_001046, NP_803565, NP_803566, NP_803878 and NP_803880

TRAPPC4 concerns the gene_assignment TREM1—triggering receptor expressed on myeloid cells 1 with GeneID: 54210 with Genbank No. for mRNA of NM_016146 including the versions NM 001318486, NM_001318488, NM_001318489 and NM_001318490 and for proteins Genbank No. NP_001305415, NP_001305417, NP_001305418, NP_001305419 and NP_001305421

TREM1 concerns the gene_assignment TREM1—triggering receptor expressed on myeloid cells 1 with GeneID: 54210 and RefSeq (mRNA) Genbank No. NM_001242589, NM_001242590 and NM_018643 with versions NM_018643.5, NM_001242589.3, NM_001242590.3, NR_136332.1 and with protein RefSeq RefSeq (protein) NP_001229518, NP_001229519 and NP_06111

TRIP13 (in GenBank Accession #NM-001166260 or Affymetrix Accession #204033) concerns the gene_assignment TRIP13—thyroid hormone receptor interactor 13 with NCBI GeneID: 9319 and with Genbank No. Accession: NM_004237.4 for transcript version 1, mRNA (protein NP_004228 version NP_004228.1), Accession: NM_001166260.2 for transcript version 2, mRNA (protein NP_001159732 version NP_001159732.1.

TSPAN14 concerns the NCBI gene_assignment: TSPAN14 tetraspanin 14, with Gene ID: 81619 and with Genbank No. for the tetraspanin-14 isoform 2 (mRNA & Protein): NM_001128309.2 & NP_001121781.1 and for tetraspanin-14 isoform 1 (mRNA & Protein): NM_001351266.1 & NP_001338195.1, NM_001351267.3 & NP_001338196.1, NM_001351268.1 & NP_001338197.1, NM_001351269.1 & NP_001338198.1, NM_001351270.1 & NP_001338199.1, NM_001351271.1 & NP_001338200.1, NM_001351272.1 & NP_001338201.1 and NM_030927.3 & NP_112189.2

We identified and validated the first, vedolizumab-specific predictive 4-gene colonic expression signature, and provided additional insights in the mode of action of vedolizumab therapy.

The landscape of inflammatory bowel diseases (IBD) treatment has extensively changed over the past decade, with the advent of anti-TNF agents, anti-adhesion molecules and anti-IL12/23 compounds inducing and maintaining clinical and endoscopic remission (Verstockt B, Ferrante M, Vermeire S, et al. J Gastroenterol 2018; 53:585-590). Nevertheless, primary non-response and secondary loss-of-response compromise the efficacy of the current available therapies on the long term. Hence, novel therapies are eagerly awaited, (Sabino J, Verstockt B, Vermeire S, et al. New biologics and small molecules in inflammatory bowel disease. An update. Therap Adv Gastroenterol. 2019) as well as predictive biomarkers which can improve the likelihood of successful treatment.

Biomarkers predicting response to anti-TNF therapy (including Oncostatin M, IL13RA2 and TREM-1) are slowly emerging, (West N R, Hegazy A N, Owens B M J, et al. Nat Med 2017; 23:579-589; Gaujoux R, Starosvetsky E, Maimon N, et al. Gut 2018; Verstockt B, Verstockt S, Blevi H, et al. Gut 2018; Verstockt B, Verstockt S, Dehairs J, et al. EBioMedicine 2019; Verstockt B, Verstockt S, Creyns B, et al. M Aliment Pharmacol Ther 2019) but need further validation prior to translation into daily clinical practice. In contrast, vedolizumab-specific biomarkers are even more limited, with studies focusing only on prediction of clinical response (Boden E K, Shows D M, Chiorean M V, et al. Dig Dis Sci 2018; 63:2419-2429 and Soendergaard C, Seidelin J B, Steenholdt C, et al. BMJ Open Gastroenterol 2018; 5:e000208). As targets in IBD treatment are evolving from clinical to endoscopic remission, (Peyrin-Biroulet L, Sandborn W, Sands B E, et al. Am J Gastroenterol 2015; 110:1324-38). biomarker development should also focus on the prediction of endoscopic remission. Previous attempts to identify a predictive mucosal signature in vedolizumab treated patients with ulcerative colitis (UC) were unsuccessful (Arjs I, De Hertogh G, Lemmens B, et al. Gut 2016).

Vedolizumab, a humanized monoclonal antibody targeting the α4β7 integrin, has proven to be a safe and efficacious drug to induce and maintain clinical remission in patients with Crohn's disease (CD) and UC (Sandborn W J, Feagan B G, Rutgeerts P, et al. N Engl J Med 2013; 369:711-21 and Feagan B G, Rutgeerts P, Sands B E, et al. N Engl J Med 2013; 369:699-710). By disturbing the interaction between mucosal addressin cell adhesion molecule-1 (MadCAM-1) on the intestinal endothelial cells and α4β7 integrin, expressed on a variety of circulating leukocytes, including T-cells, B cells, eosinophils, natural killer cells and macrophages, vedolizumab is primarily a gut-focused drug. Although it has always been considered to interfere mainly with lymphocyte trafficking to the gut, a detailed characterization of its immunological mode of action recently pointed primarily towards its influence on the innate, rather than on the adaptive immune system (Zeissig S, Rosati E, Dowds C M, et al. Gut 2019; 68:25-39).

In order to identify the most suitable patients for vedolizumab therapy, we here studied colonic transcriptomic data of IBD patients initiating vedolizumab therapy, performed pathway analysis and deconvolution, and searched for predictive markers of vedolizumab-specific endoscopic remission.

The introduction of biological therapies in the treatment of inflammatory bowel disease (IBD) has significantly improved disease outcome and altered the natural history of the disease, including less steroid exposure, less hospitalizations, and less major surgeries [Mandel M D, et al Dig Dis 2014; 32(4):351-9]. Novel insights in IBD pathogenesis led to the development of new compounds with a different mode of action, including anti-adhesion molecules (vedolizumab, VDZ) and interleukin (IL) 12/23 antibodies (ustekinumab, UST) [Verstockt B, et al J Gastroenterol 2018; 53(5):585-90]. However, some patients never respond to a particular therapy. For anti-TNF therapy in particular, primary nonresponse rates vary from 10 to 30%, and the annual risk of secondary loss of response ranges from 13% for infliximab (IFX) to 20% for adalimumab (ADM) [FlamantM et al Ther Adv Gastroenterol 2018; 11:1756283X17745029]. Both from a patient perspective as from a socio-economic perspective, identifying the most suitable therapy for a given patient is key. With many more compounds being tested in phase II and III clinical trials [Argollo M, et al. J Autoimmun 2017; 85:103-16], personalised medicine will become even more necessary in future. During recent years, researchers focused on a better understanding of the working mechanisms of anti-TNF agents [Levin A D, et al. J Crohns Colitis 2016; 10(8):989-97]. This not only contributed to the development of novel targeted therapies, but also paved the way for biomarker development predicting response to anti-TNF. Gene expression analysis of inflamed biopsies of Crohn's disease (CD) and ulcerative colitis patients (UC) prior to IFX therapy, identified several genes differentially expressed between responders and non-responders [Arijs I, Li K, Toedter G, et al. Gut 2009; 58(12):1612-9, Arijs I, Quintens R, Van Lommel L, et al. Inflamm Bowel Dis 2010; 16 (12):2090-8 and Toedter G, Li K, Marano C, et al. Am J Gastroenterol 2011; 106(7):1272-80.]. Among these, IL13RA2 was the highest ranked common gene for both CD and UC analyses. Co-expression network analysis of the same dataset concluded that TNF-driven pathways are significantly increased at baseline in future non-responders [Verstockt B, Verstockt S, Creyns B, et al. Aliment Pharmacol Ther 2018 Aliment Pharmacol Ther. 2018 October; 48(7):731-739]. Recently, expansion of apoptosis-resistant intestinal TNFR2+IL-23R+ T-cells has been associated with resistance to anti-TNF therapy in CD [Schmitt H, et al. Gut 2018 May 30—Gut 2019; 68:814-828. doi:10.1136/gutjnl-2017-315671]. Finally, advanced bioinformatic techniques integrated all publically available datasets and identified colonic expression of both oncostatin M (OSM) and Triggering Receptor Expressed on Myeloid cells 1 (TREM1) as key players in and predictors of anti-TNF (non-)responsiveness [West N R, Hegazy A N, Owens B M J, et al. Oncostatin M drives intestinal inflammation and predicts response to tumor necrosis factor-neutralizing therapy in patients with inflammatory bowel disease. NatMed 2017; 23(5):579-89, Gaujoux R, Starosvetsky E, Maimon N, et al. Gut. 2019 April; 68(4):604-614. Epub 2018 Apr. 4 and Verstockt B, Verstockt S, Blevi H, et al. Gut. 2019 August; 68(8):1531-1533. Epub 2018 Jul. 14.]. However, their specificity for anti-TNF agents has not yet been investigated, and therefore it remains to be clarified if these markers are true anti-TNF-specific predictors or just bystanders of inflammation.

So far, no predictive biomarker has found its way into IBD clinical practice yet. Potentially because markers based on gene expression of intestinal biopsies are more complex to translate to clinical practice. In contrast, whole blood biomarkers may be more applicable. Whole blood TREM1 expression looks a promising predictive biomarker for anti-TNF therapy in CD, although conflicting results are currently reported [Gaujoux R, Starosvetsky E, Maimon N, et al. Gut. 2019 April; 68(4):604-614. Epub 2018 Apr. 4. and Verstockt B, Verstockt S, Blevi H, et al. Gut. 2019 August; 68(8):1531-1533. Epub 2018 Jul. 14.]. We here studied mucosal biopsies and whole blood expression of IL13RA2, TNF-alpha, TNFR2, OSM, TREM1 and its transcripts in a prospectively collected cohort of CD and UC patients prior to initiation of biological therapy (ADM, IFX, UST, or VDZ) and assessed endoscopic remission as outcome.

### EXAMPLES

### Example 1 on Anti-α₄β₇-Integrin Agents and/or Anti-IL-12/23 Agents

### Example 1 a Methods & Patient Selection

This prospective study was conducted at the IBD center of the University Hospitals Leuven (Leuven, Belgium). We collected whole blood of 127 IBD patients initiating biologic therapy: 54 CD and UC patients initiating IFX or ADM, 22 CD patients initiating UST and 51 CD and UC patients initiating VDZ (Table 1). All patients had endoscopy-proven active disease (Mayo endoscopic sub score 2-3 in case of UC; presence of ileal and/or colonic ulcerations in case of CD) and had to be naïve for the drug that was initiated at inclusion.

All anti-TNF treated patients had to have persistent endoscopic lesions with sufficient drug exposure, defined as a maintenance trough level N 3.0 μg/mL for infliximab or N 5.0 μg/mL for adalimumab before being defined as non-responder. Due the lack of agreement on the targeted threshold for ustekinumab and vedolizumab, if any, we did not include an exposure requirement in the definition of nonresponse for both drugs.

Whole blood (PAXgene blood RNA tubes, Qiagen, Benelux, Netherlands) samples were collected at baseline, prior to the first infusion/injection, stored overnight at room temperature whereafter they were preserved at −80° C. according to the manufacturer's instructions.

Biopsies at the edge of an ulcer in the most inflamed area were taken during endoscopy prior to the start of therapy, stored in RNALater buffer (Ambion, Austin, Tex., USA) and preserved at −80° C. Similarly, serum of all patients initiating anti-TNF therapy was taken prior to first administration, centrifuged and stored at −20° C.

### Example 1 b Outcomes

Response was defined based on endoscopic findings as an objective parameter In CD patients, endoscopic remission was assessed after 6 months and defined as a Simple Endoscopic Disease (SES-CD) score ≤2 [Sturm A, Maaser C, Calabrese E, et al. ECCO-ESGAR guideline for diagnostic assessment in inflammatory bowel disease. J Crohns Colitis 2018 Aug. 23.-J Crohns Colitis. 2019 Mar. 26; 13(3):273-284 and Maaser C, Sturm A, Vavricka S R, et al. ECCO-ESGAR guideline for diagnostic assessment in inflammatory bowel disease. J Crohns Colitis 2018 Aug. 23.—J Crohns Colitis. 2019 Feb. 1; 13(2):144-164.]. In UC patients, a Mayo endoscopic sub-score of ≤1 was considered as endoscopic remission. Due to national reimbursement criteria, all UC patients were endoscopically evaluated at week 8 (ADM) or week 14 (IFX and VDZ). All endoscopies were performed by the same 3 experienced IBD staff members (GVA, SV, MF).

Isolation of RNA

Total whole blood RNA was extracted using the PAXgene Blood RNA Kit (Qiagen, Benelux, Netherlands) according to the manufacturer's instructions.

Total RNA from inflamed biopsies was extracted using the AllPrep DNA/RNA Mini kit (Qiagen, Hilden, Germany) according to the manufacturer's instructions. The integrity and quantity of all RNA was assessed with a 2100 Bioanalyzer (Agilent, Waldbronn, Germany) and a Nanodrop ND-1000 spectrophotometer (Thermo Scientific, Waltham, Mass., USA). Extracted RNA was stored at −80° C. until further processing.

### Example 1 c Quantitative RT-PCR

Gene expression (TREM1, OSM, TNF, TNFR2, IL13RA2) in whole blood was studied trough quantitative real-time polymerase chain reaction (qPCR) analysis. To further unravel the TREM1 predictive signal, the expression of all known TREM1 transcripts, TREM1 transcript variant x1 (TREM1-mb), TREM1 transcript variant x2 (TREM1-x2) and TREM1 transcript variant x3 (TREM1-sv), was studied too. cDNA was synthesized from 0.25 μg of total RNA using the RevertAid H Minus First Strand cDNA synthesis kit (Fermentas, St. Leon-Rot, Germany) according to the manufacturer's protocol. The primers were synthesized by Sigma-Genosys (Haverhill, UK) and 10 Mstock solutions were used to make the reaction mixture (5 μL SybrGreen, 0.2 μM FW& RV primer, 2 μL cDNAsample, 2.8 μL RNAse-freeH2O). All samples were amplified in duplicate reactions. Samples were analysed with the Lightcycler 480 (Roche, Basel, Switzerland). The following amplification program was used: 5′ 95° C., 45×(10″ 95° C., 15″ 60° C., 15″ 72° C.), 5″ 95° C., 1′ 60° C., 4° C. mRNA-levels were normalized to the housekeeping gene β-actin and quantified using the comparative (AA) Ct method.

Example 1 d RNA Sequencing Next-generation single-end sequencing was performed using the Illumina HiSeq 4000NGS, after library preparation using the TruSeq Stranded mRNA protocol (Illumina, San Diego, USA) according to the manufacturer's instructions. Raw RNA-sequencing data were aligned to the reference genome using Hisat2 version 2.1.0 [Kim D, Langmead B, Salzberg S L. HISAT: a fast spliced aligner with low memory requirements. Nat Methods 2015; 12(4):357-60], absolute counts generated using HTSeq [Anders S, Pyl P T, Huber W HTSeq-a Python framework to work with high throughput sequencing data. Bioinformatics 2015; 31(2):166-9], whereafter counts were normalized and differential gene expression assessed using the DESeq2 package [Love M I, Huber W, Anders S. Moderated estimation of fold change and dispersion for RNA-seq data with DESeq2. Genome Biol 2014; 15(12):550].

RNA-seq data have been deposited in the ArrayExpress database at EMBL-EBI (www.ebi.ac.uk/arrayexpress) under accession number EMTAB-7604.

Serum Proteins

Serum TREM1 (soluble TREM1, sTREM1, CD 354) was measured using the Human sTREM-1 ELISA kit (HK348, Hycult Biotech, Uden, the Netherlands). Serum TNF was measured using the MesoScale Discovery electrochemiluminescence technology (MSD, Rockville, USA).

### Example 1 e Statistical Analysis

All analyses were carried out using IBMSPSS Statistics 24 (IBMSPSS, Costa Mesa, Calif., USA) and R version 3.5.0 (R Development Core Team, Vienna, Austria). Continuous variables are expressed as median and interquartile range (IQR). Unpaired data were compared using the Mann-Whitney U test for continuous variables, and with Fisher's exact test for categorical variables. Correlations were assessed using the Spearman r correlation coefficient. Stepwise forward and backward elimination logistic regression modelling was performed to identify independent predictors of the outcome of anti-TNF therapy. Final model selection was based on the most optimal second-order Akaike information criterion.

Diagnostic performance was assessed with receiver operating characteristics (ROC) curve analysis. A relevant threshold value was chosen on the ROC curve, based on the performance of the Youden's J statistic and closest top-left method. A two-tailed p-value b.05 was considered significant.

### Example 1 Results

Patient Characteristics

Fifty-four actively inflamed patients (24 CD, 30 UC) with a median (IQR) disease duration of 6.8 (1.7-19.6) years were included in this prospective study, prior to their first IFX or ADM administration (Table 1). At time of induction, 16 patients (29.6%) were on corticosteroids and 18 patients (33.3%) received immunomodulatory agents (IFX treated patients only). CD patients were endoscopically evaluated after 27.1 (25.0-29.0) weeks, with an overall endoscopic remission rate of 54.2% (30.0% ADM, 71.4% IFX). In UC patients, an endoscopic remission rate of 33.3% (27.8% ADM, 41.7% IFX) was observed after a median of 8.4 (8.0-10.0) weeks. Similar response rates were observed in patients who were naïve to anti-TNF therapy and in patients who previously failed another anti-TNF agent (43.9% vs. 38.5%, p=0.73).

Additionally, whole blood was collected in 51 actively inflamed patients (25 CD, 26 UC) initiating vedolizumab therapy, of whom 9 (17.6%) were entirely anti-TNF naïve. After 6 months (CD) and 14 weeks (UC), vedolizumab induced endoscopic remission in 48.0%, 61.5% of patients respectively. Finally, 22 active CD patients initiated ustekinumab with an endoscopic remission rate of 22.3% after 6 months

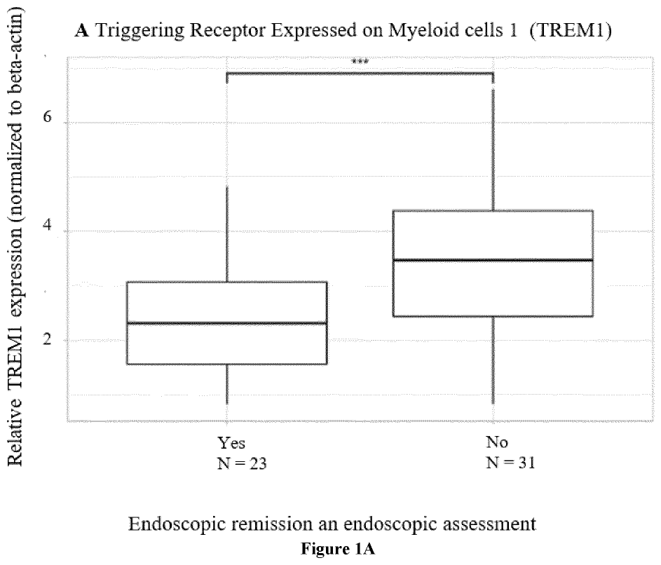

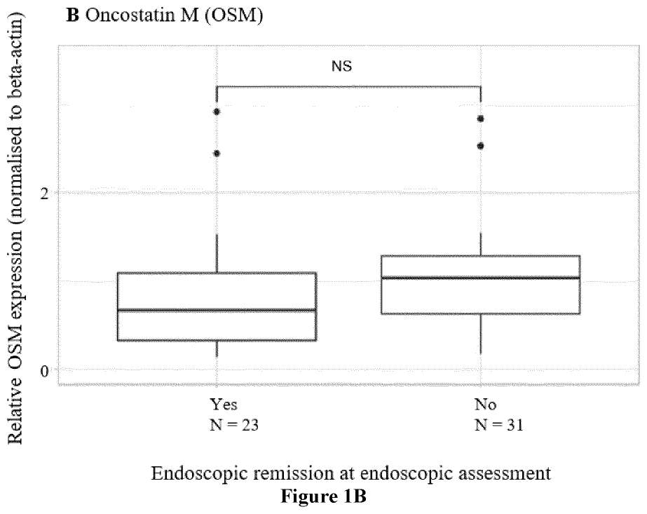

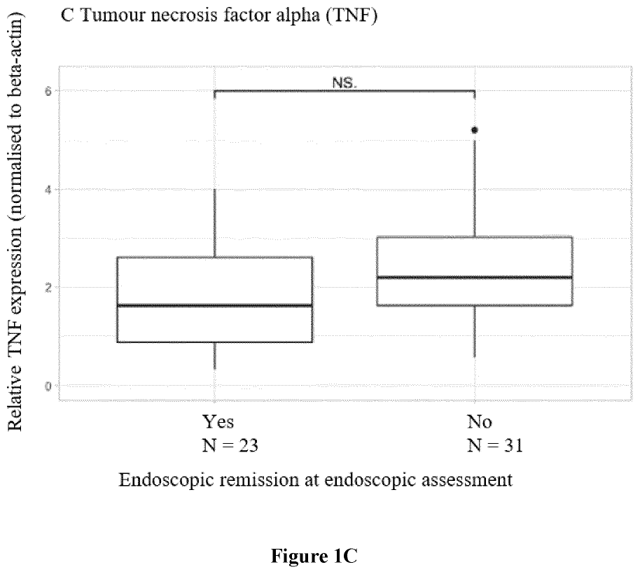

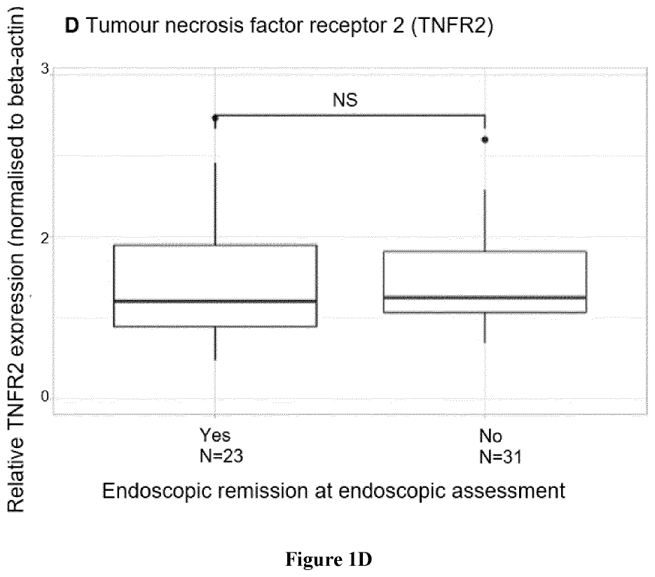

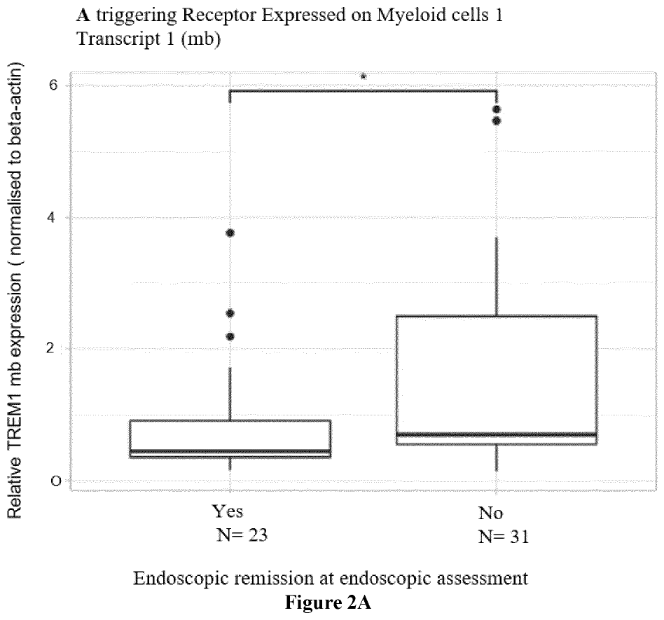

Whole blood, comparative analysis between responders and nonresponders In the anti-TNF cohort, TREM1 was significantly downregulated at baseline in patients achieving endoscopic remission (fold change (FC)=0.67, p b 0.001) (FIG. 1A). In contrast, OSM, TNF and TNFR2 expression was not significantly different between future responders and non-responders (FC=0.61, p=0.09; FC=0.74, p=0.13; FC=0.94, p=0.24 respectively) (FIG. 1B-D). Baseline sTREM1 was only numerically lower in future responders (0.28 ng/mL, IQR 0.16-0.56 ng/mL) compared to non-responders (0.40 ng/mL, IQR 0.30-0.83 ng/mL) (FC=0.70, p=0.09). Whole blood IL13RA2 mRNA could not be detected in both the discovery and validation cohort using 2 different pairs of primers and using different dilutions of cDNA.

Whole blood TREM1 expression did not significantly correlate with CRP (spearman p=−0.08, p=0.38), faecal calprotectin (spearman ρ=−0.06, p=0.64) or serum TNFα (spearman ρ=−0.15, p=0.63).

However, it did significantly increase with increasing age (spearman ρ=0.24, p=0.007) and tended to increase with longer disease duration (spearman ρ=0.17, p=0.06).

No correlation with the total number of previous biological agents previously exposed to could be observed (spearman ρ=0.08, p=0.55).

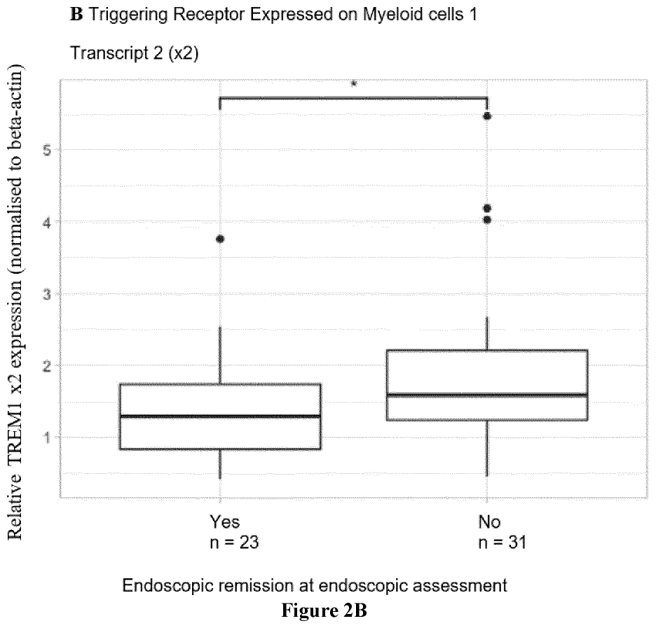

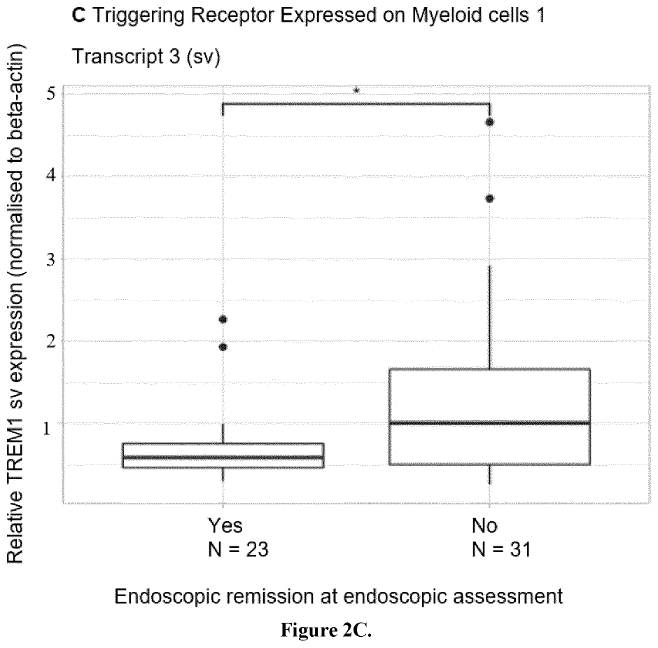

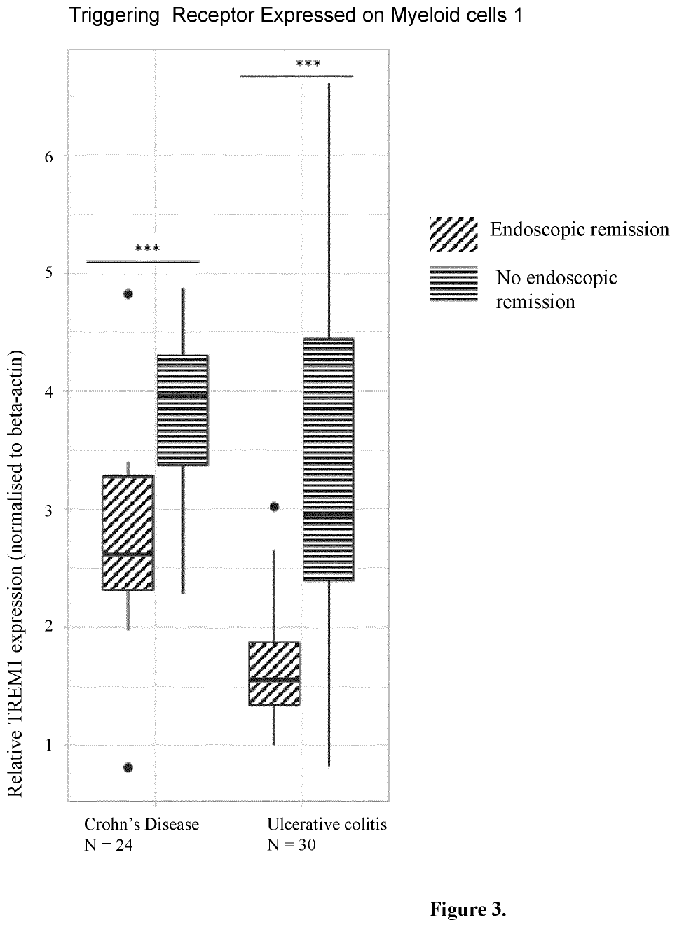

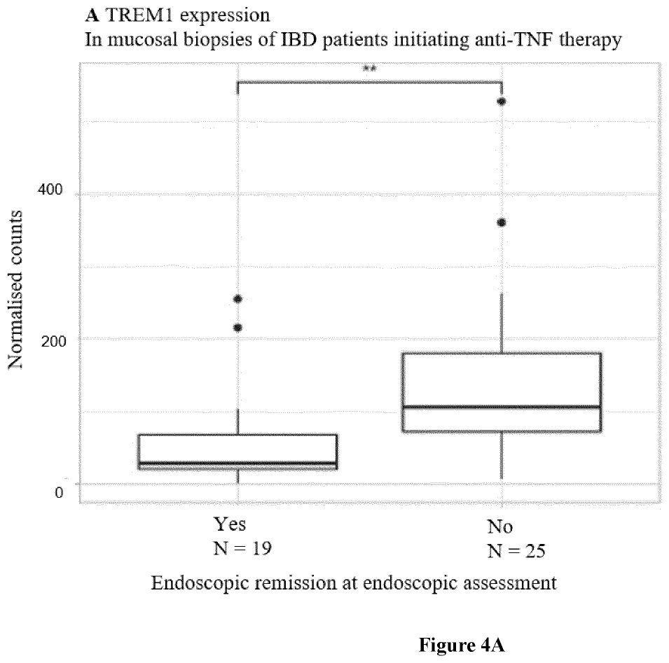

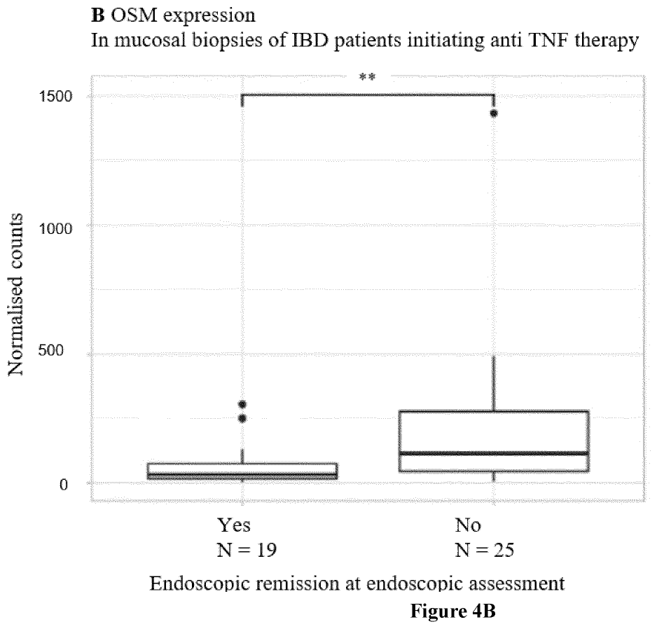

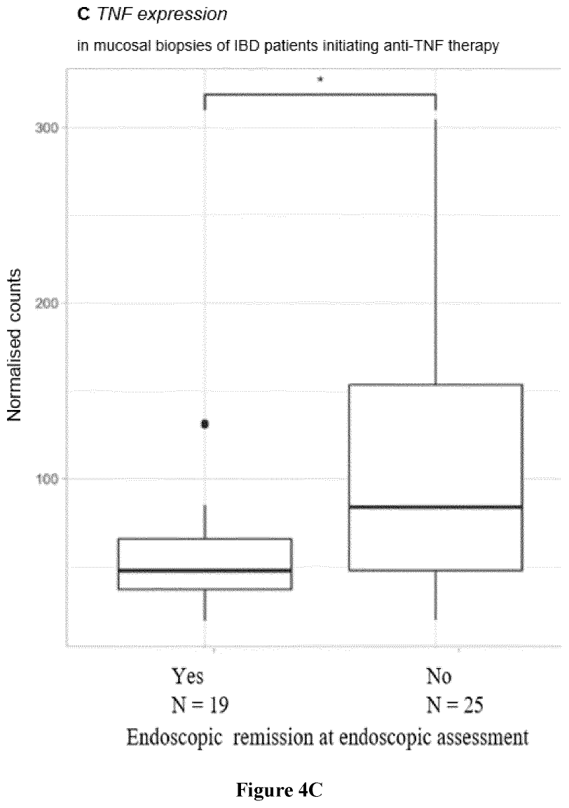

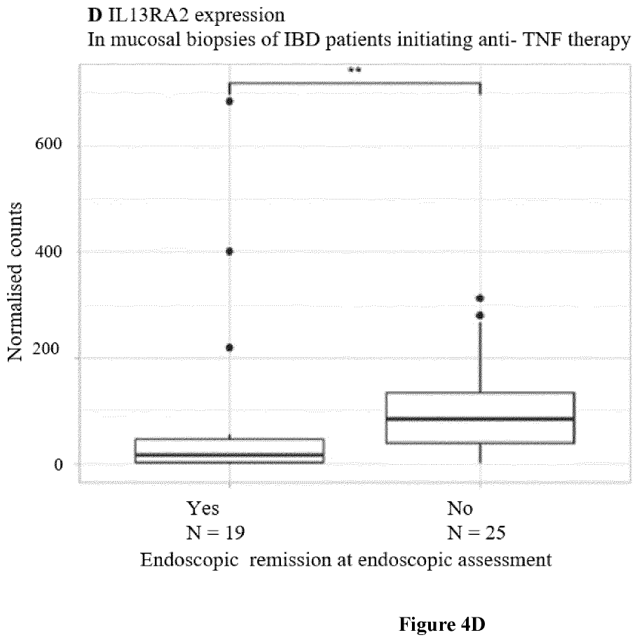

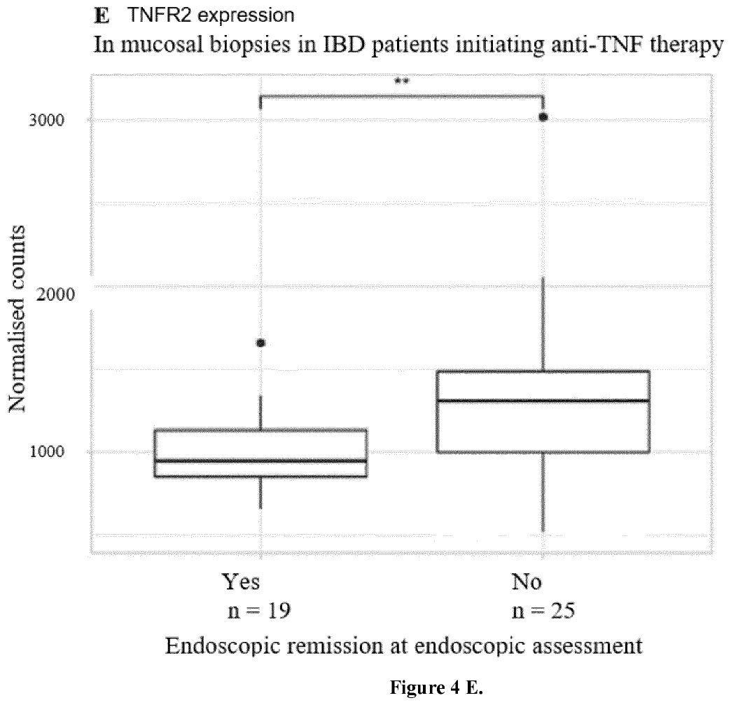

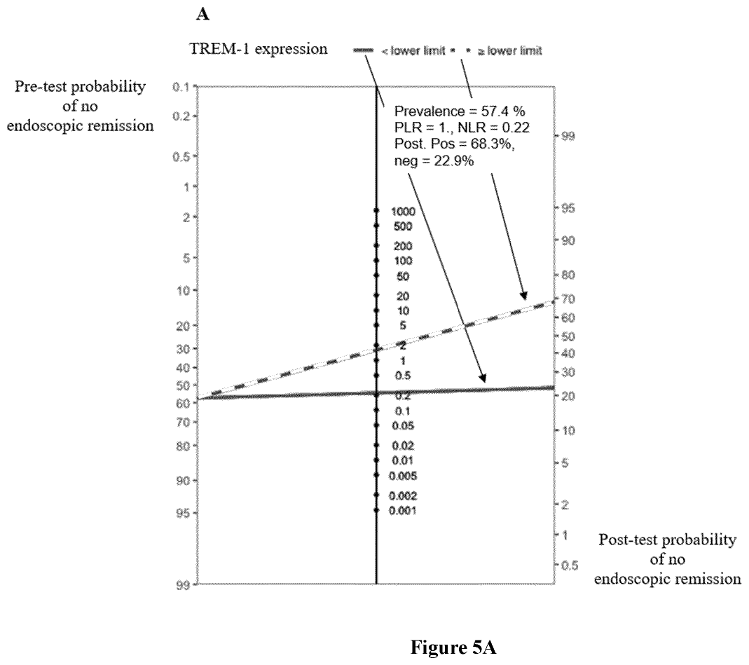

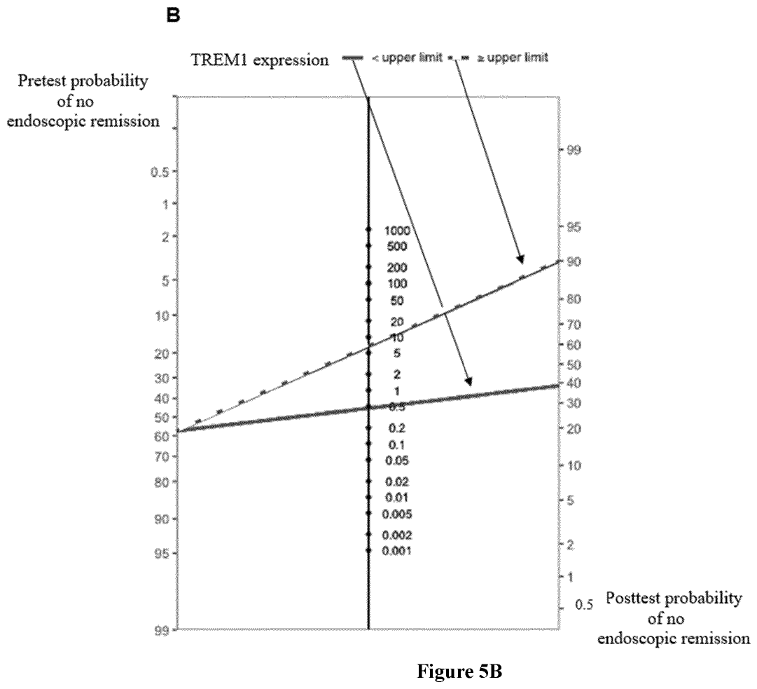

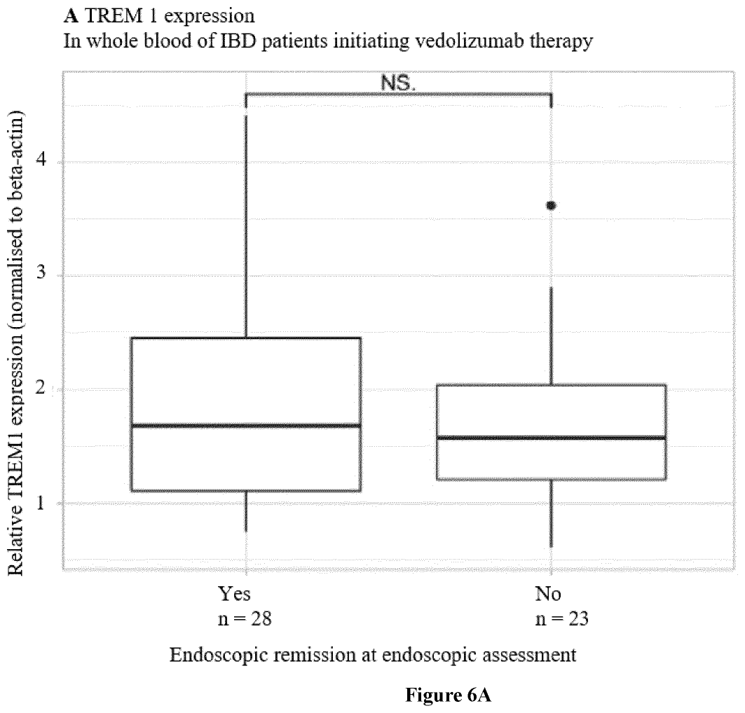

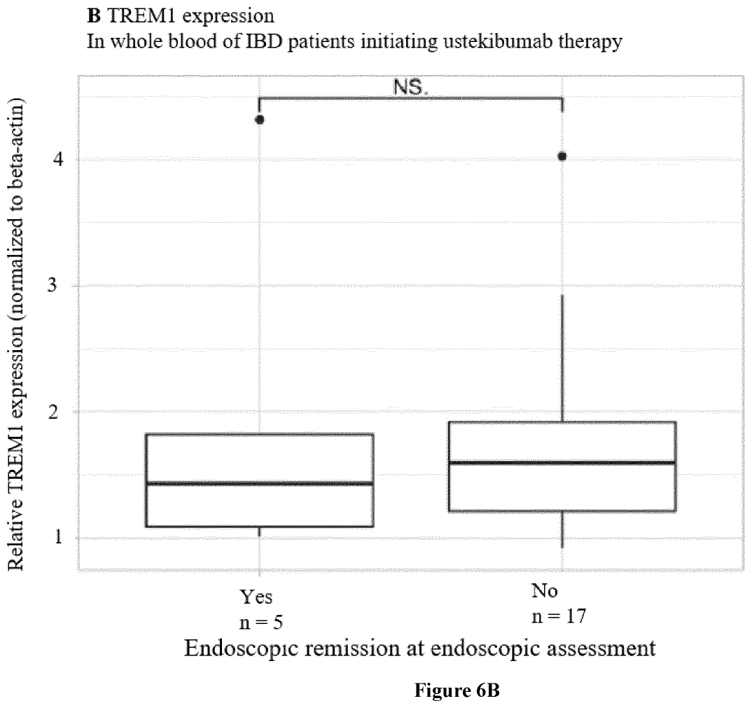

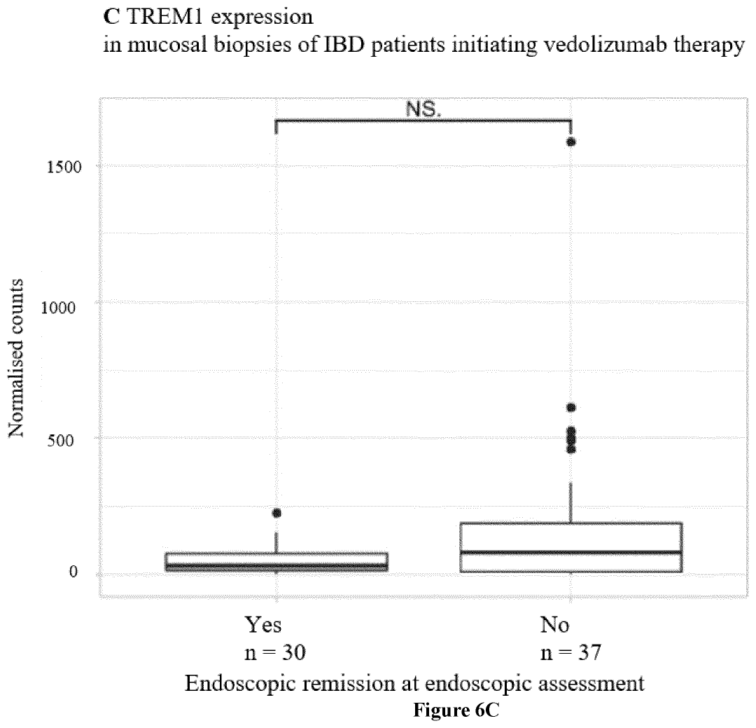

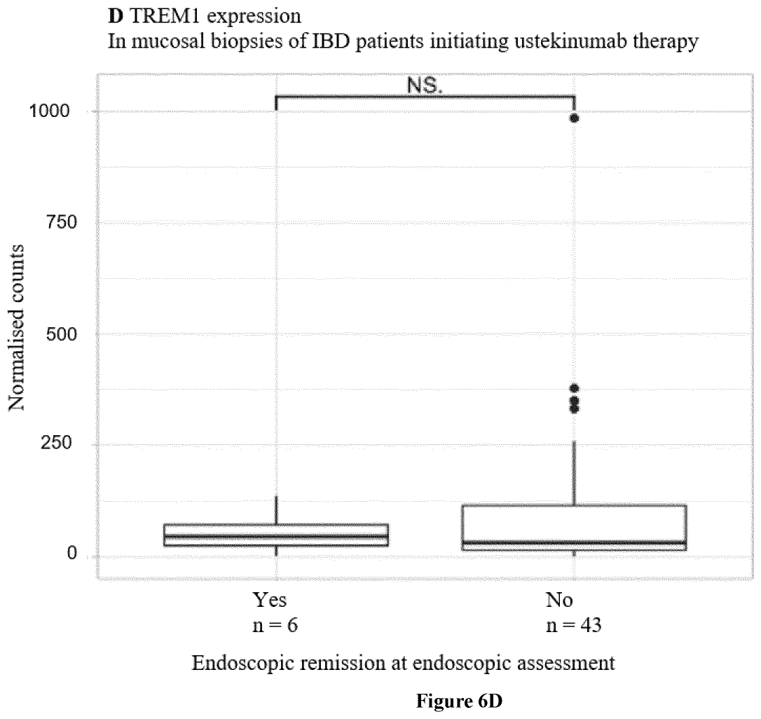

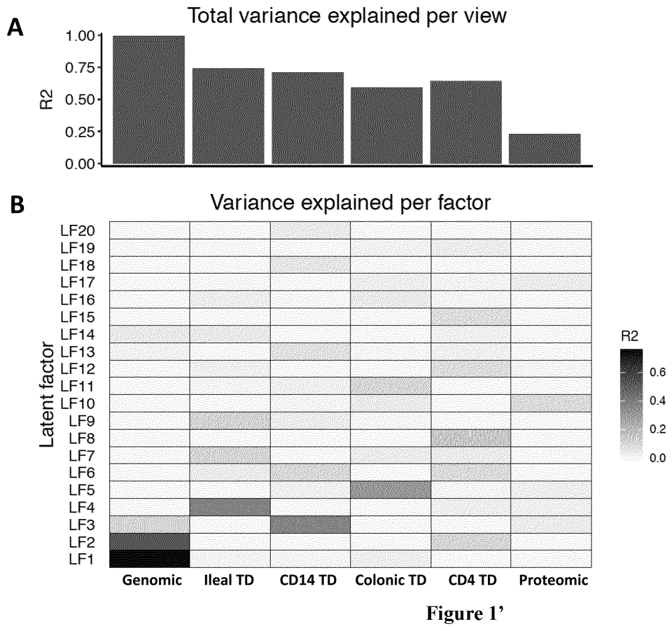

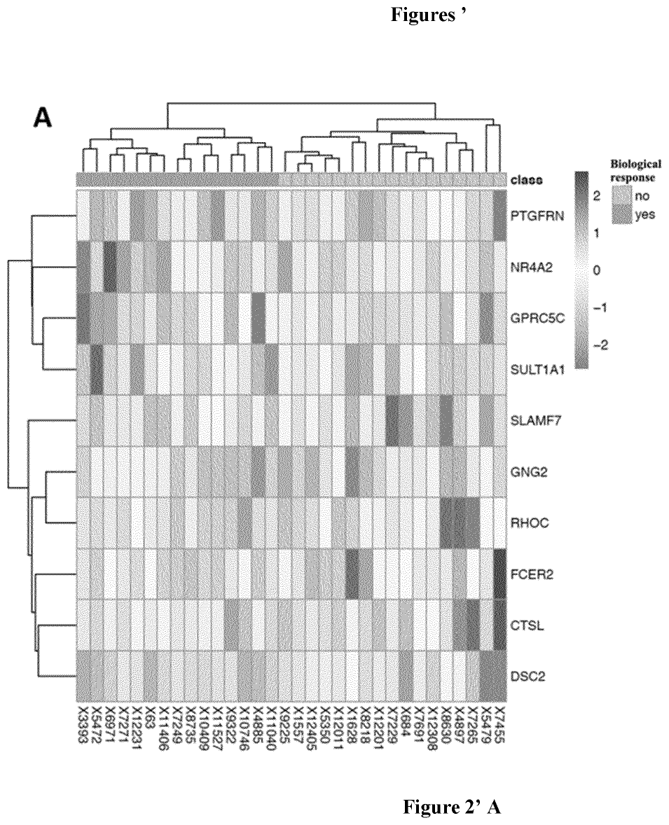

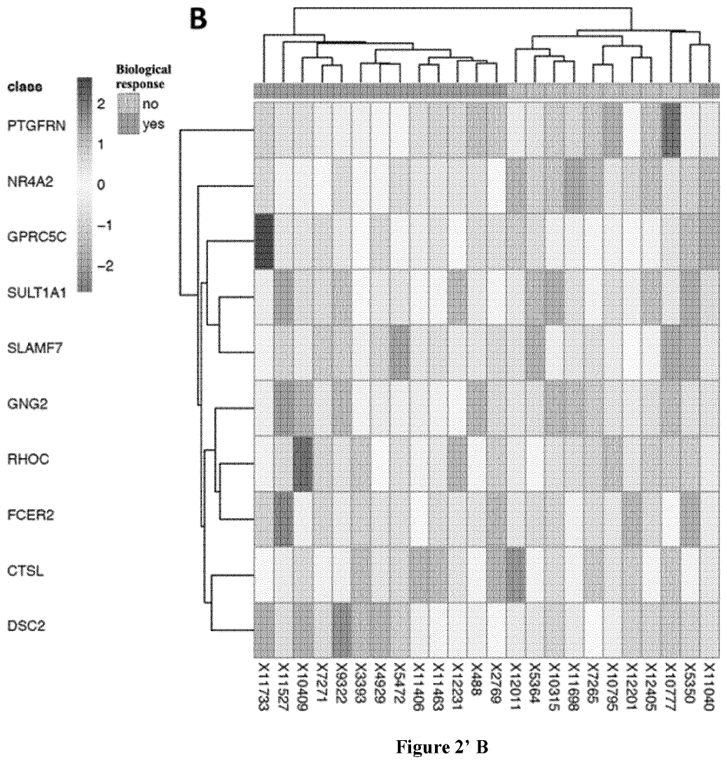

TREM1 expression did not differ between anti-TNF exposed and anti-TNF naïve patients (FC 30=1.24, p=0.46). Additionally, both in anti-TNF exposed and in anti-TNF naïve patients, future responders had lower TREM1 levels (FC=0.48, p=0.01; FC=0.69, p=0.01 respectively). The expression levels of all individual transcripts significantly correlated with each other and with the overall TREM1 expression level (Table 2), suggesting that they all contribute to the overall anti-TNF predictive signature (FIG. 2A-C). Furthermore, total TREM1 mRNA levels in whole blood correlated significantly with sTREM1 protein levels (spearman ρ=0.36, p=0.01), which did not hold true for the individual transcript levels (p=0.43, p=0.13, p=0.15 respectively).

Similar Findings in Both Crohn's Disease and Ulcerative Colitis

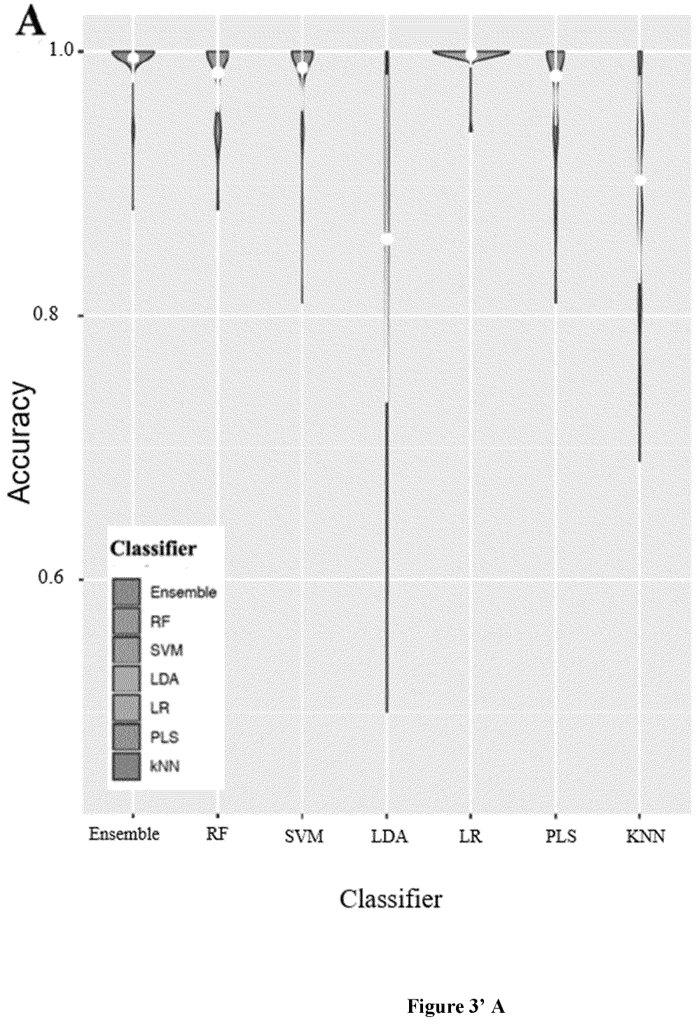

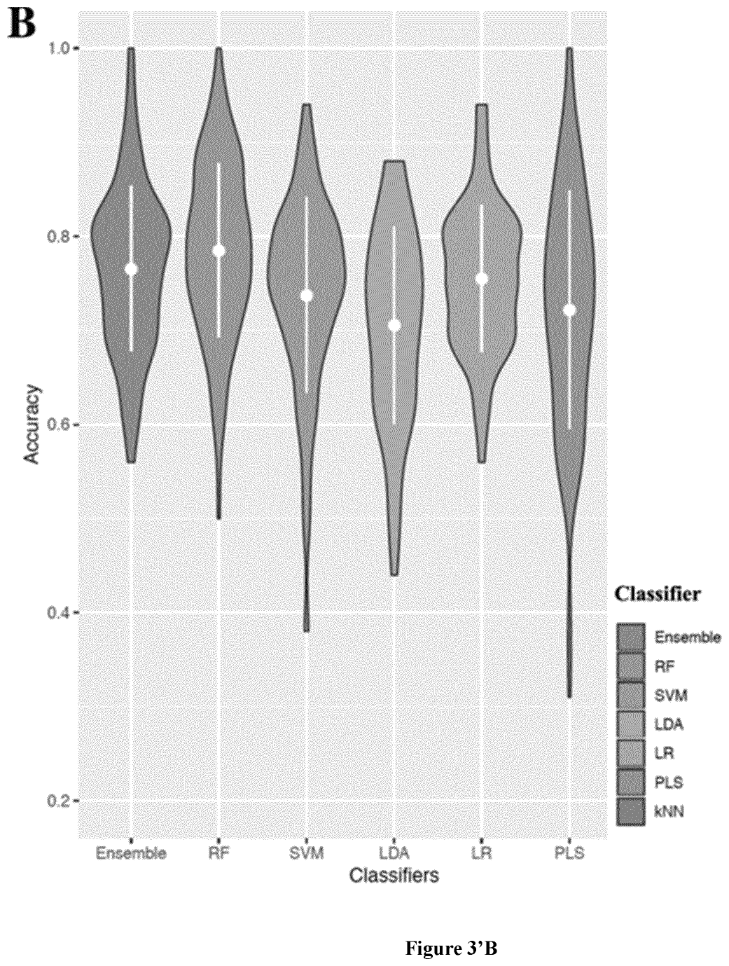

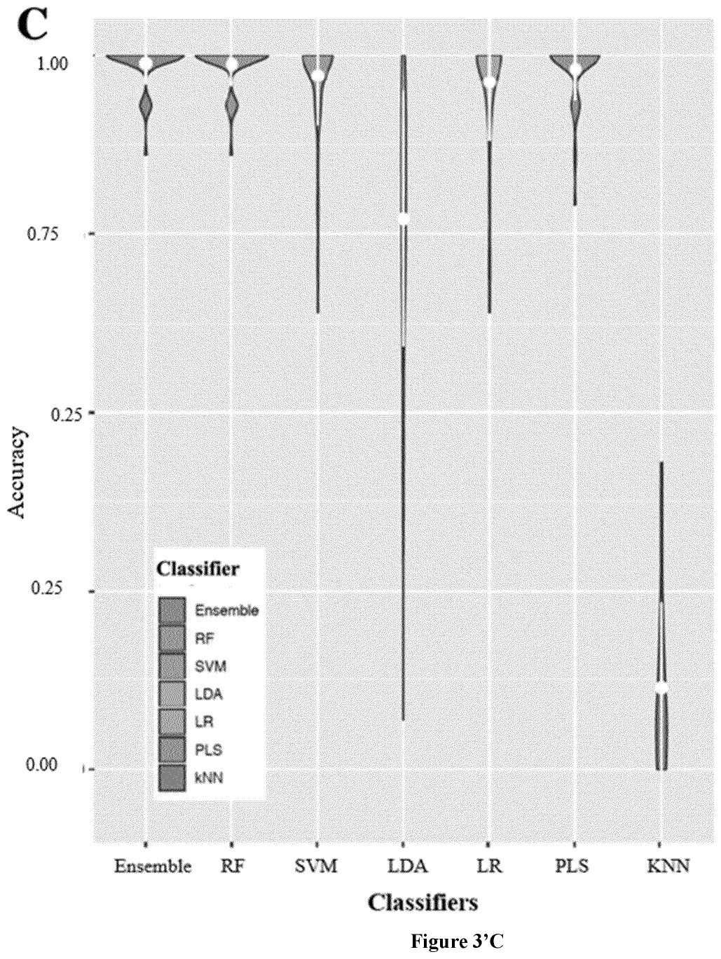

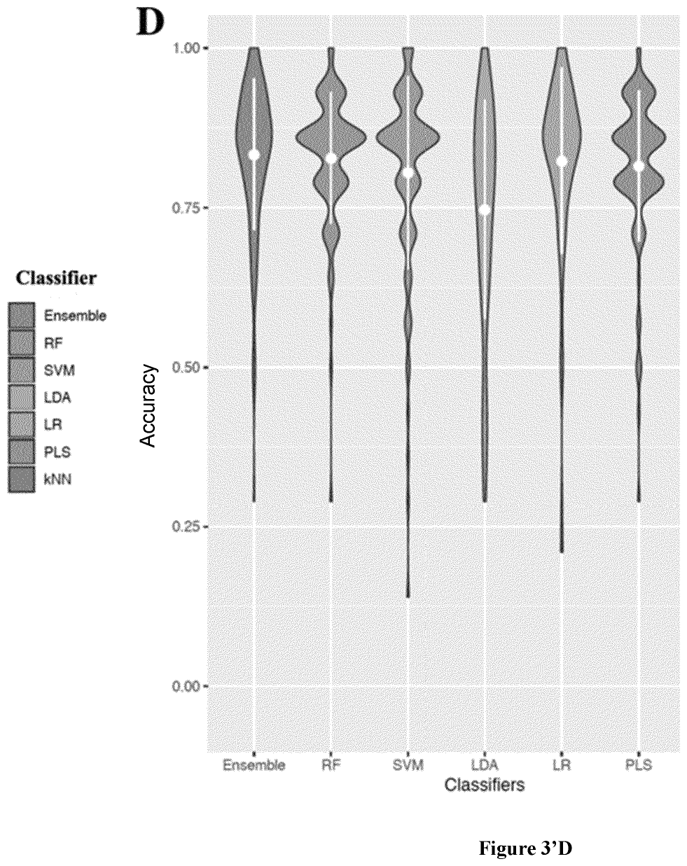

No significant differences in whole blood TREM1 expression between CD and UC patients could be observed (p=0.19). However, the association with endoscopic remission seemed stronger in UC patients than in CD patients (FC=0.53, p=0.001; FC=0.66, p=0.007 respectively) (FIG. 3). Similarly, CD and UC patients did not significantly differ in OSM, TNF or TNFR2 expression (FC=0.62, p=0.06; FC=1.2, p=0.58; FC=1.25, p=0.25 respectively). Although future CD responders did not differ from non-responders in OSM, TNF and TNFR2 expression (FC=0.97, p=0.36; FC=0.79, p=0.41; FC=1.18, p=0.83 respectively), UC responders did for TNFR2 (FC=0.66, p=0.03) but not for OSM or TNF FC=0.55, p=0.34; FC=0.65, p=0.08 respectively).

Intestinal Tissue, Comparative Analysis Between Responders and Nonresponders

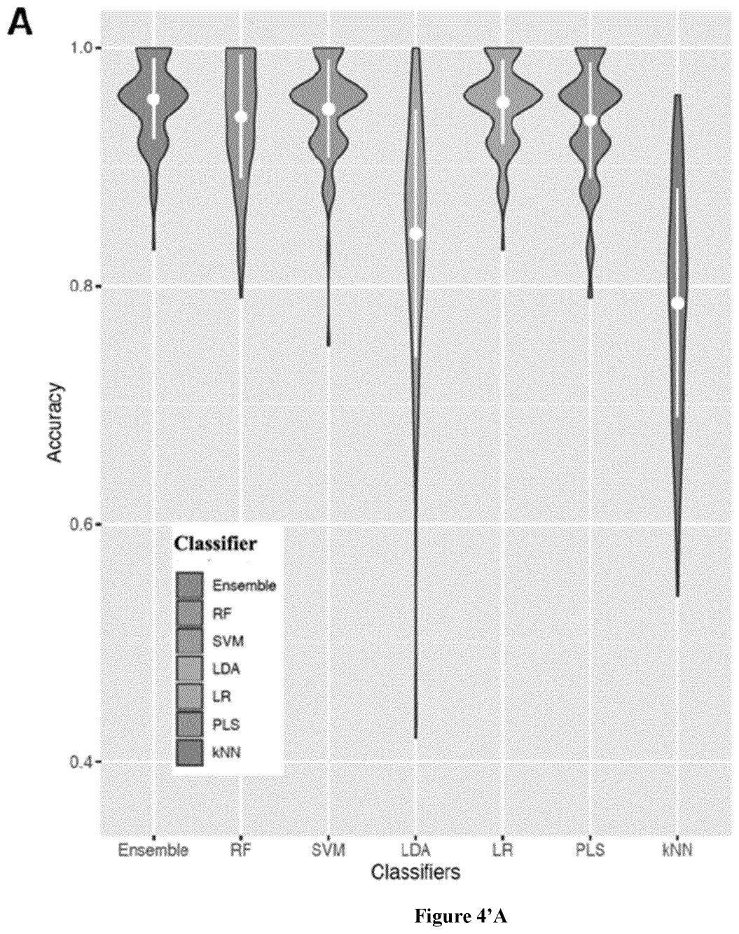

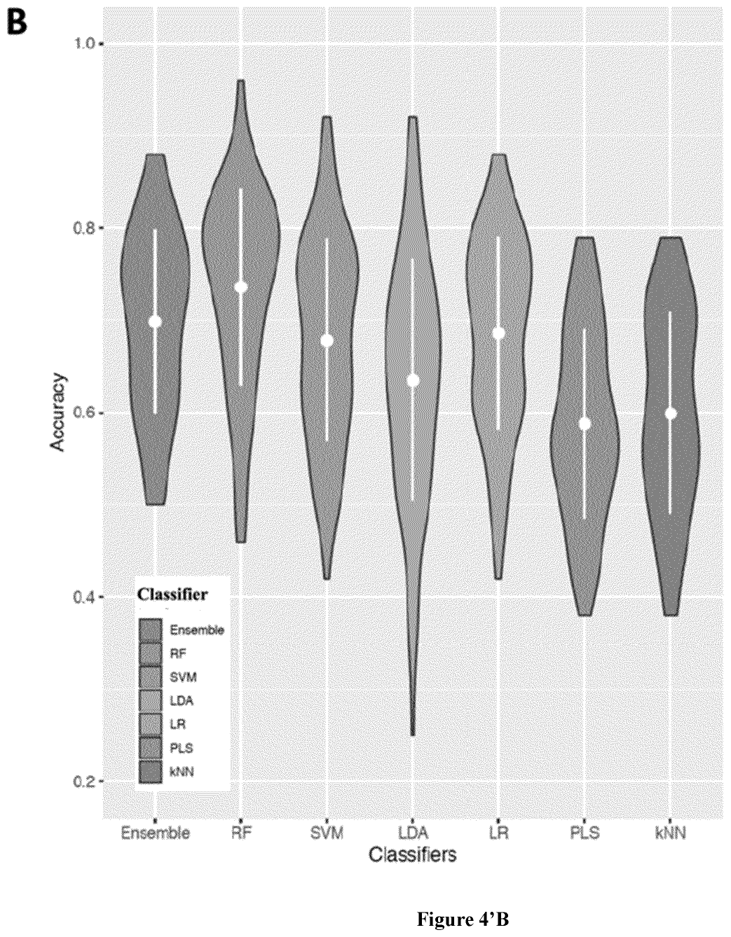

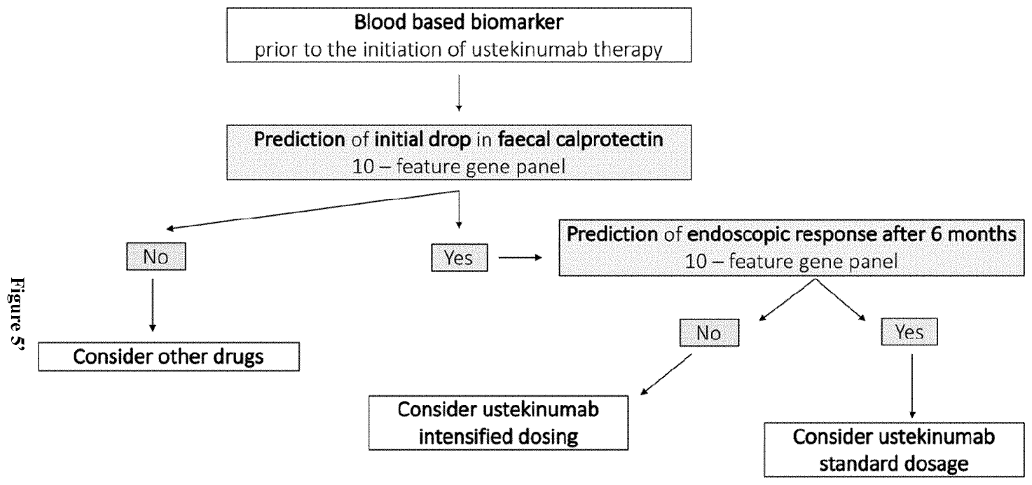

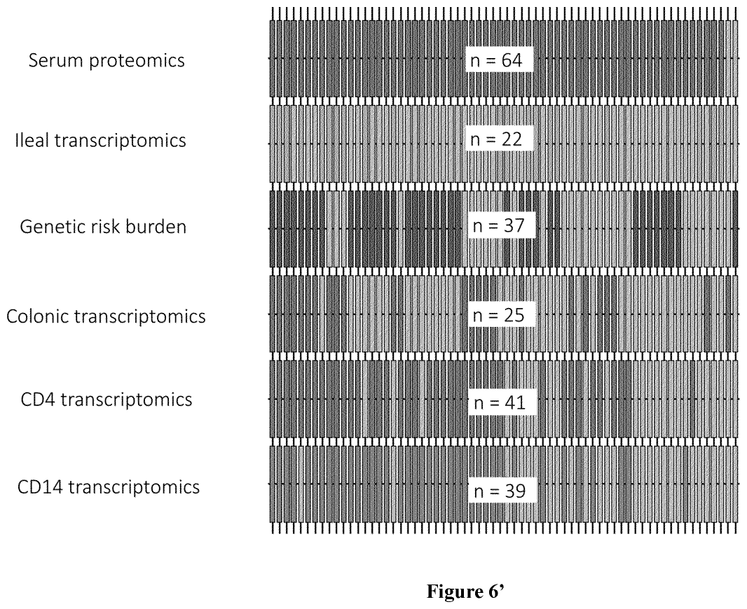

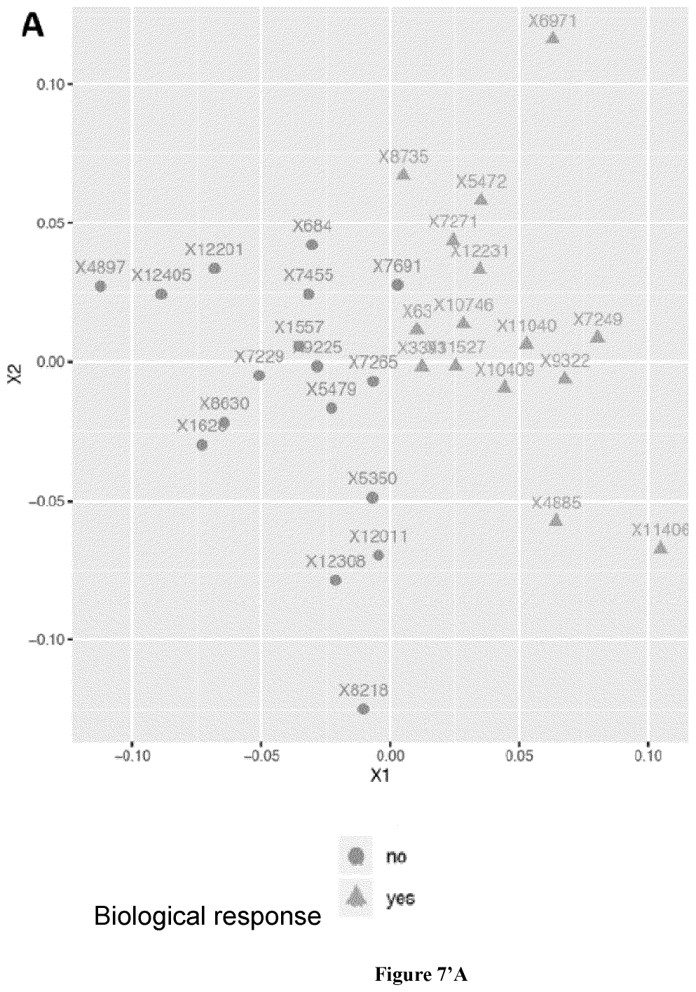

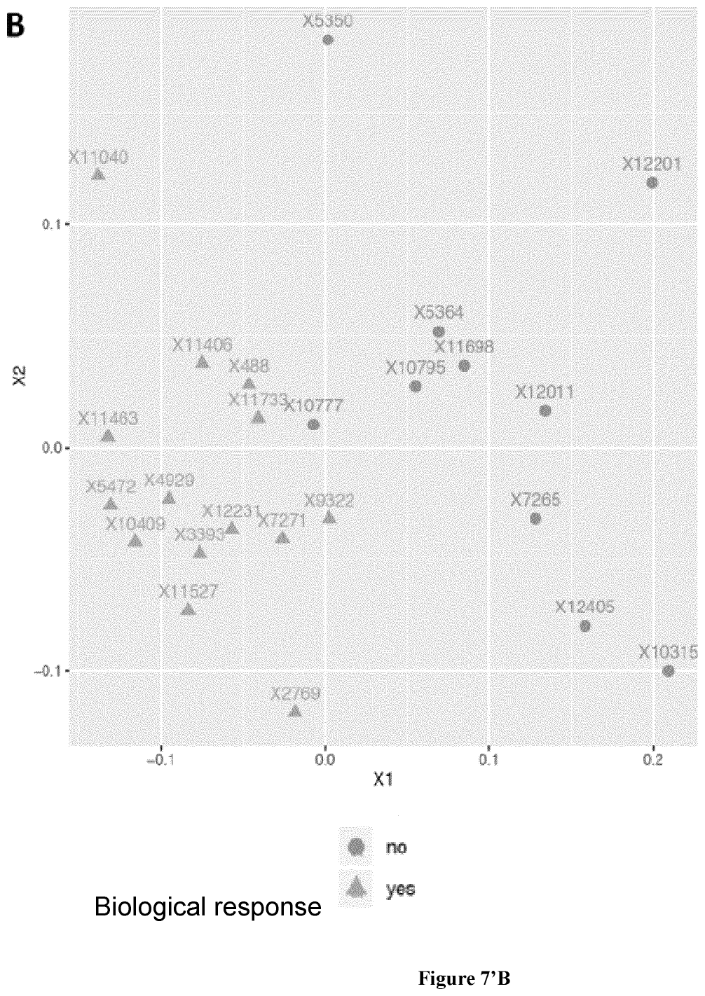

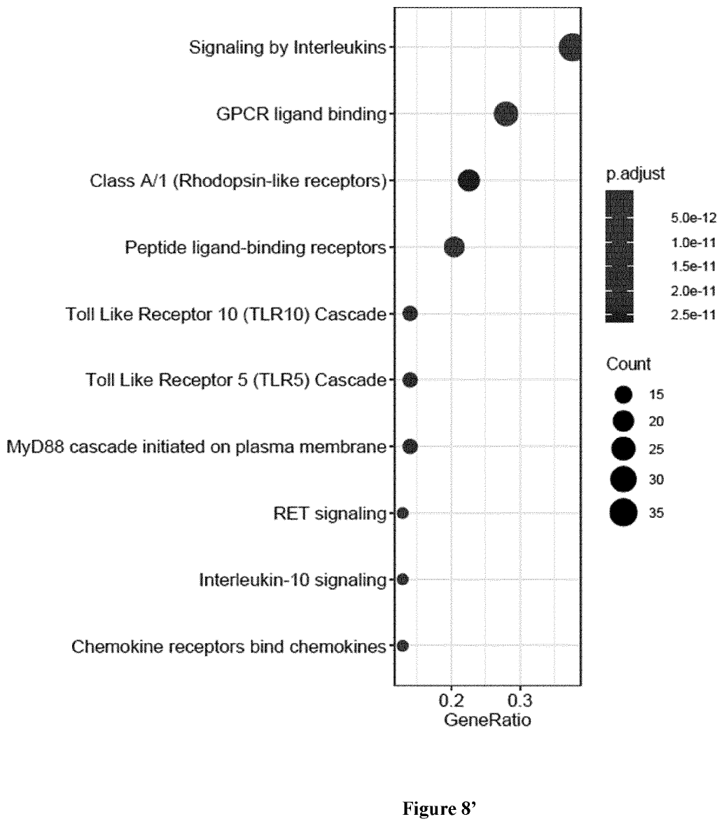

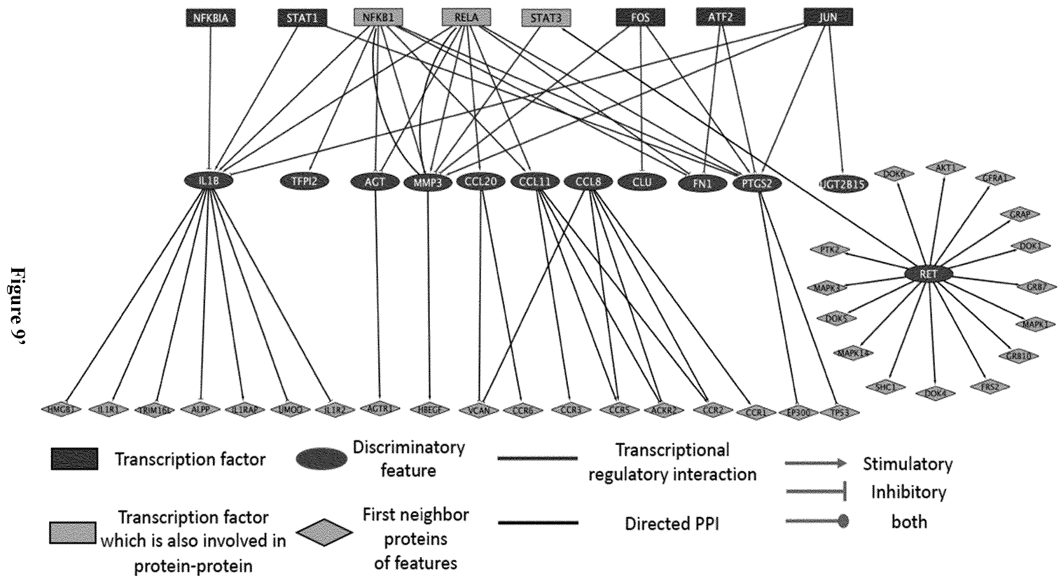

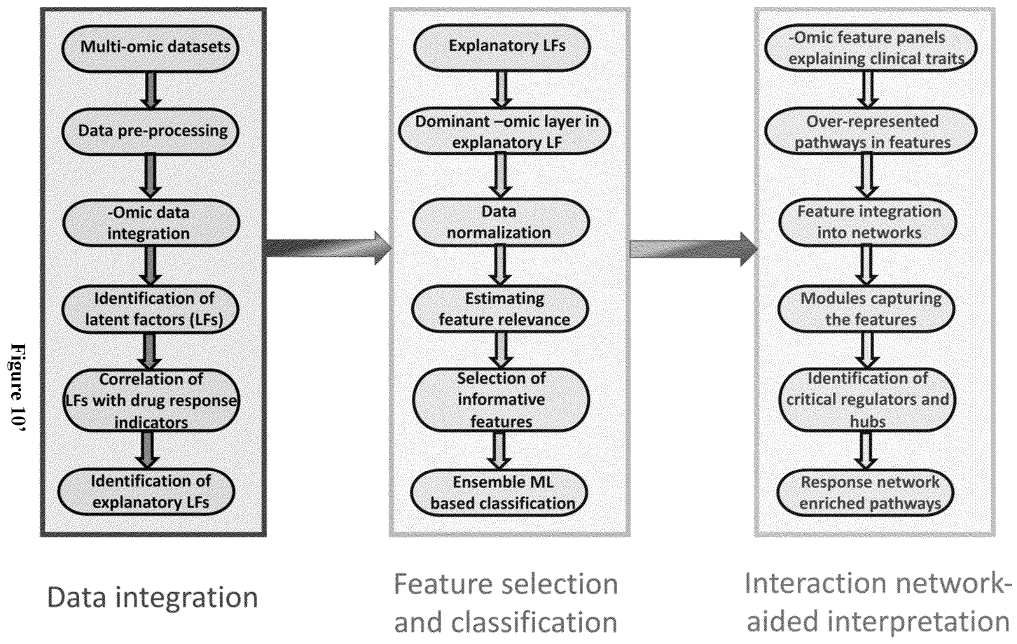

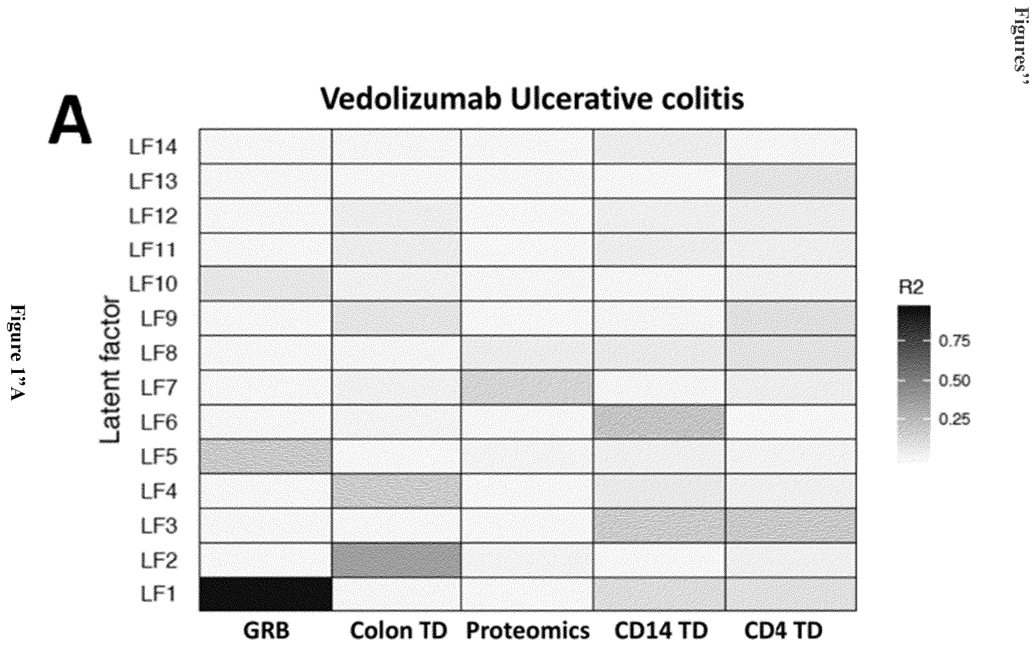

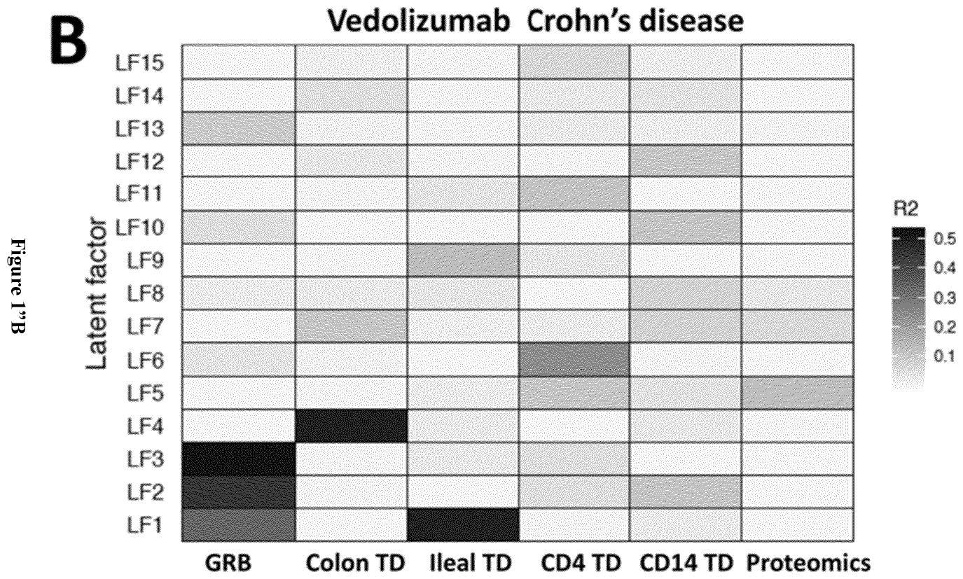

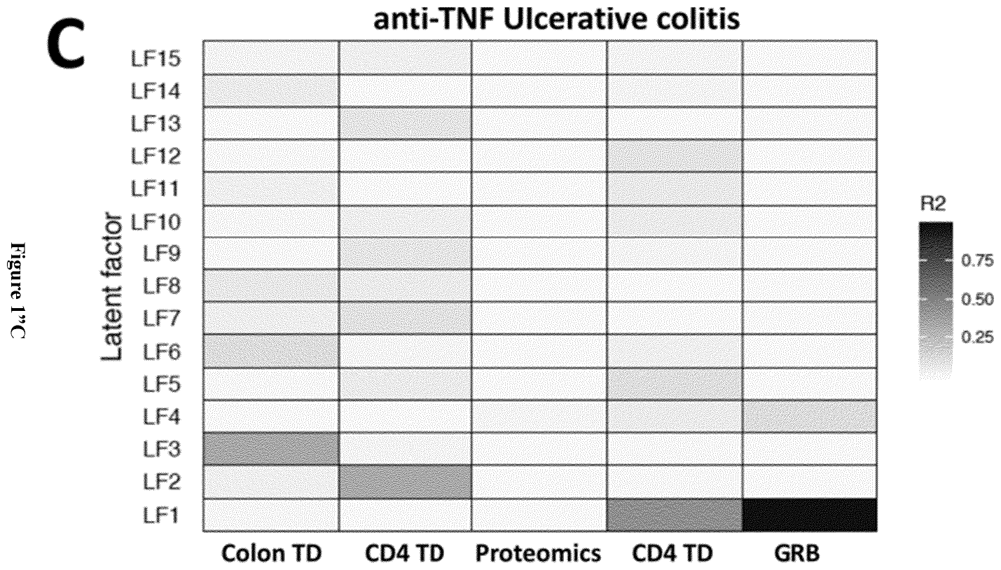

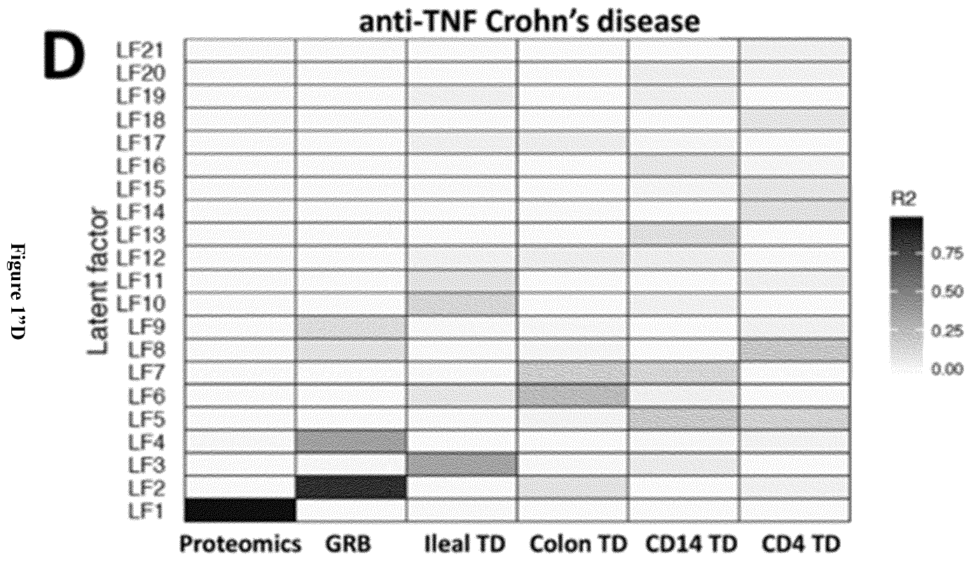

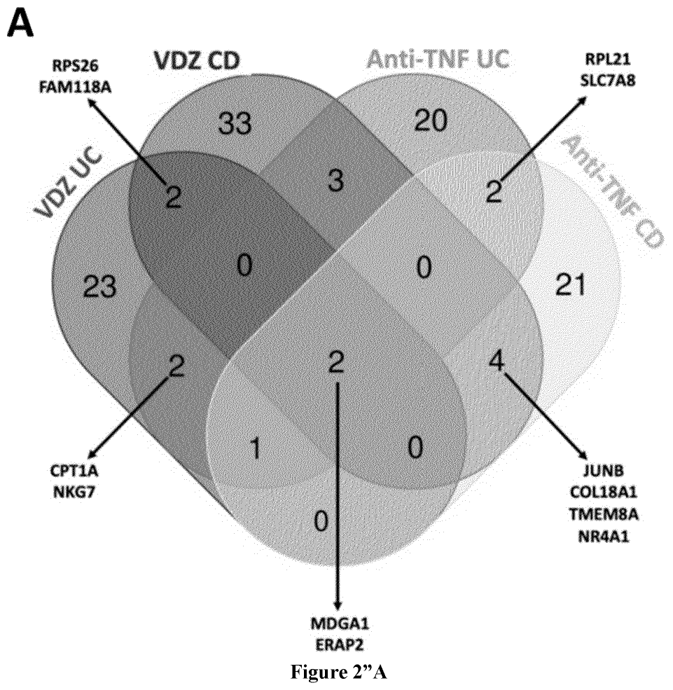

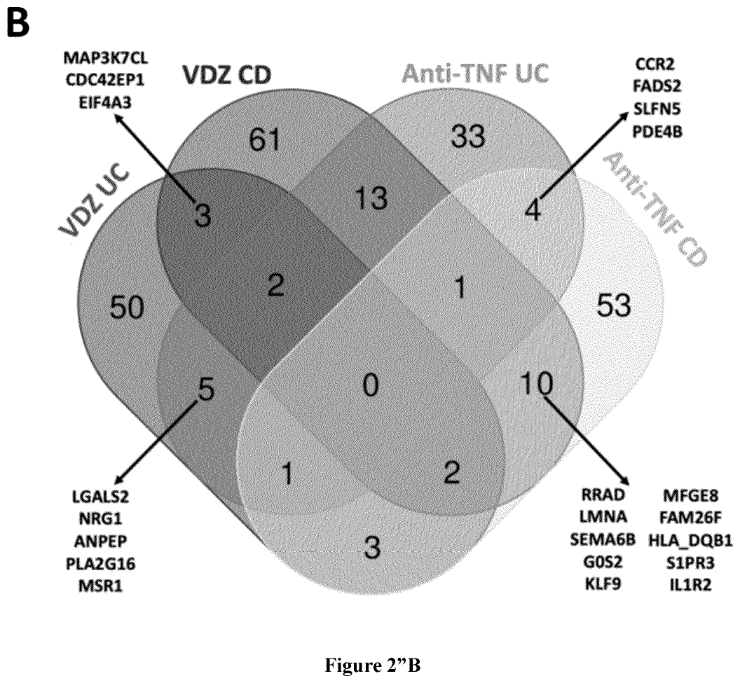

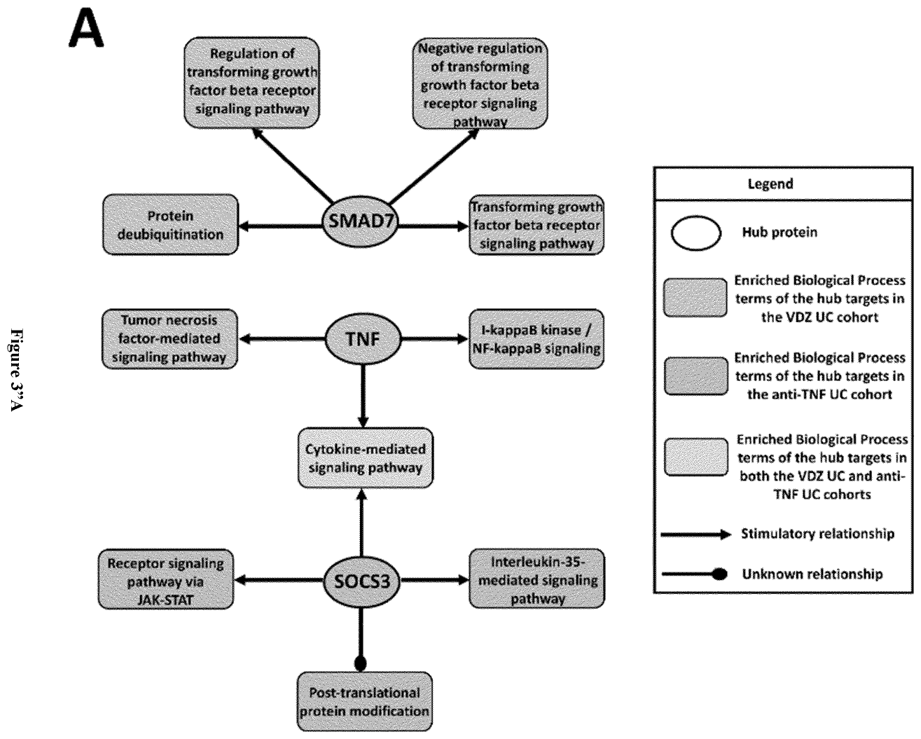

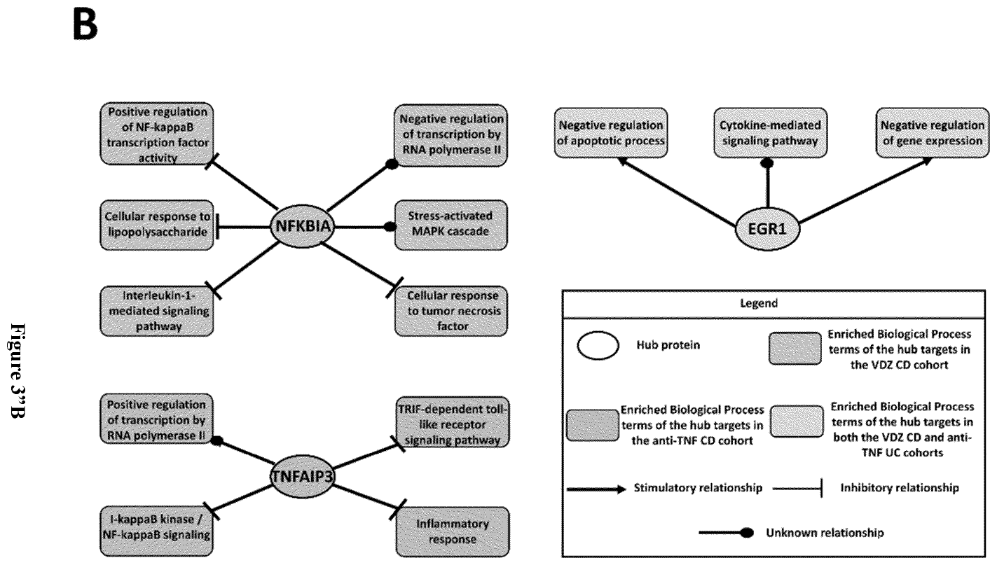

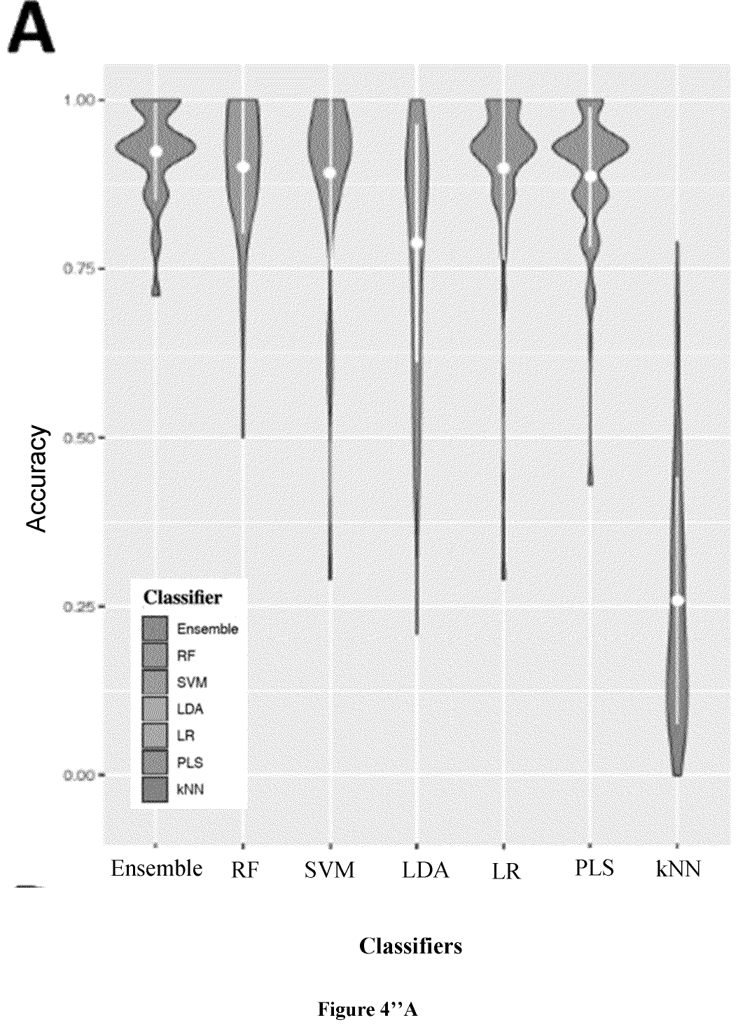

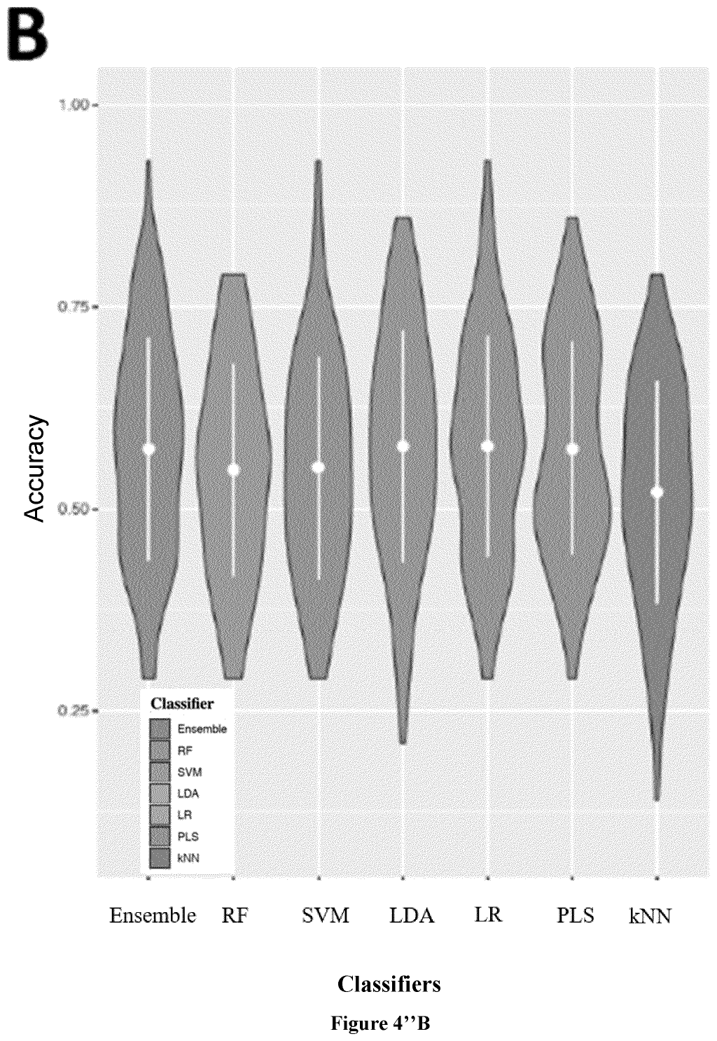

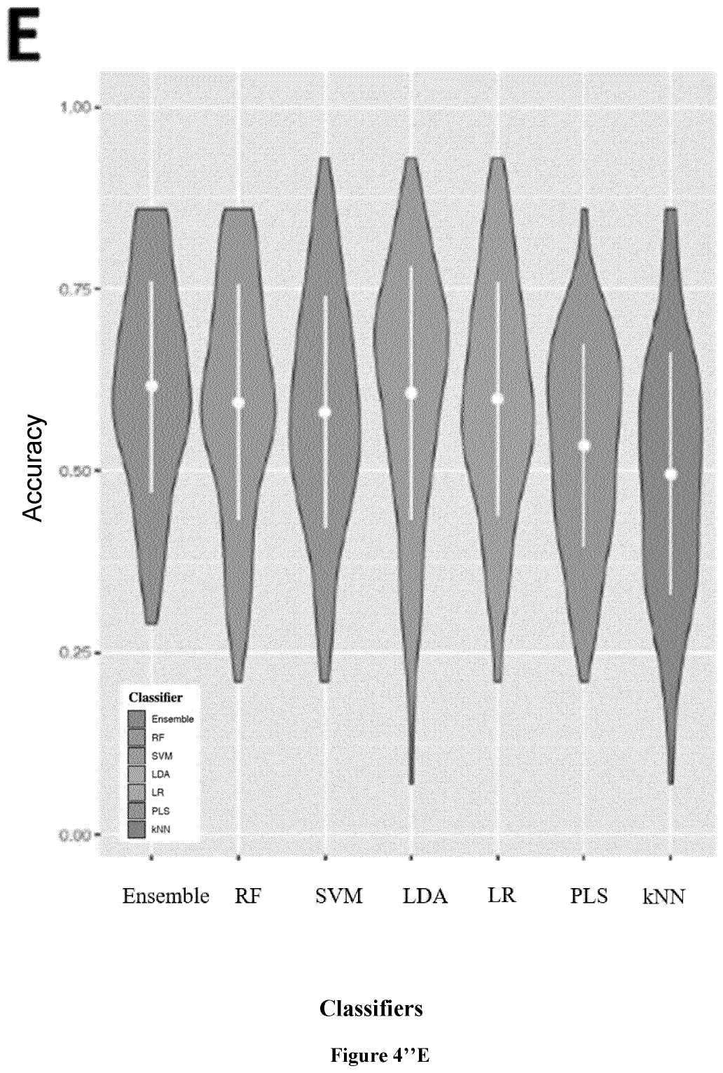

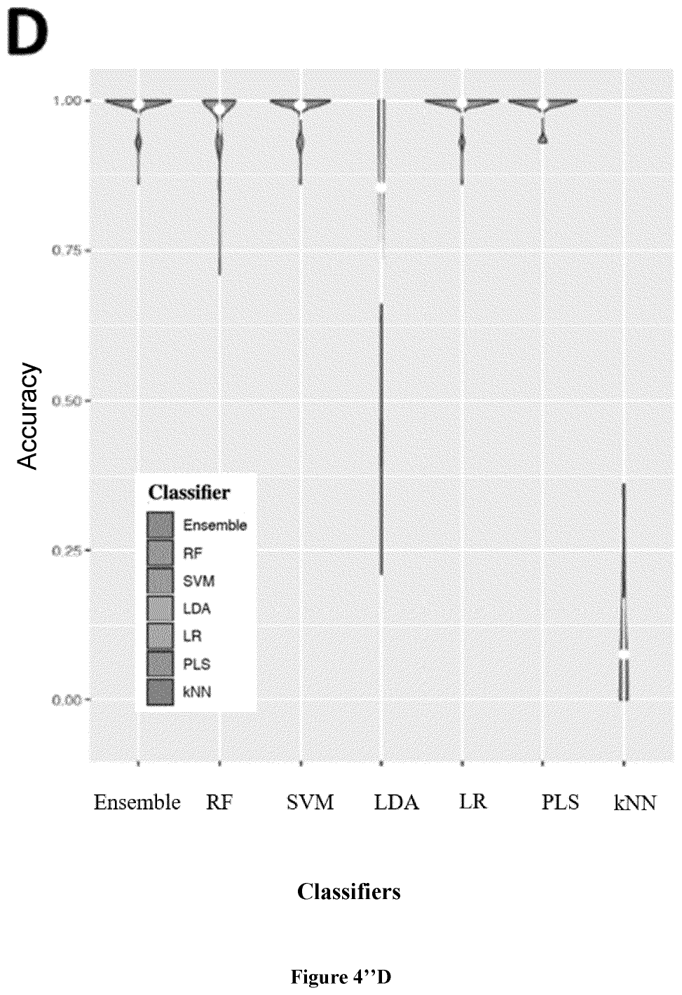

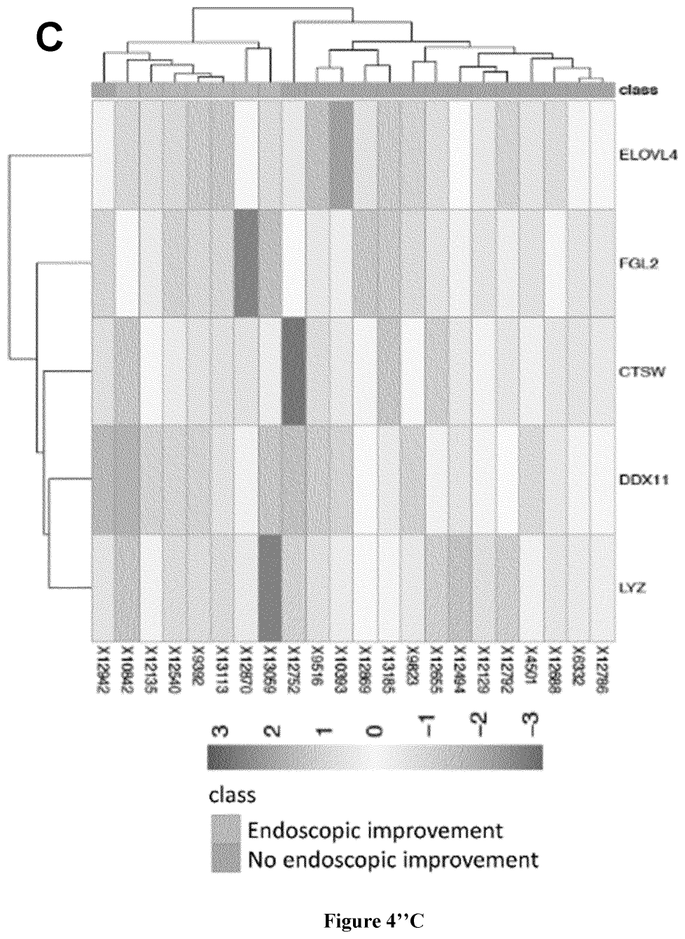

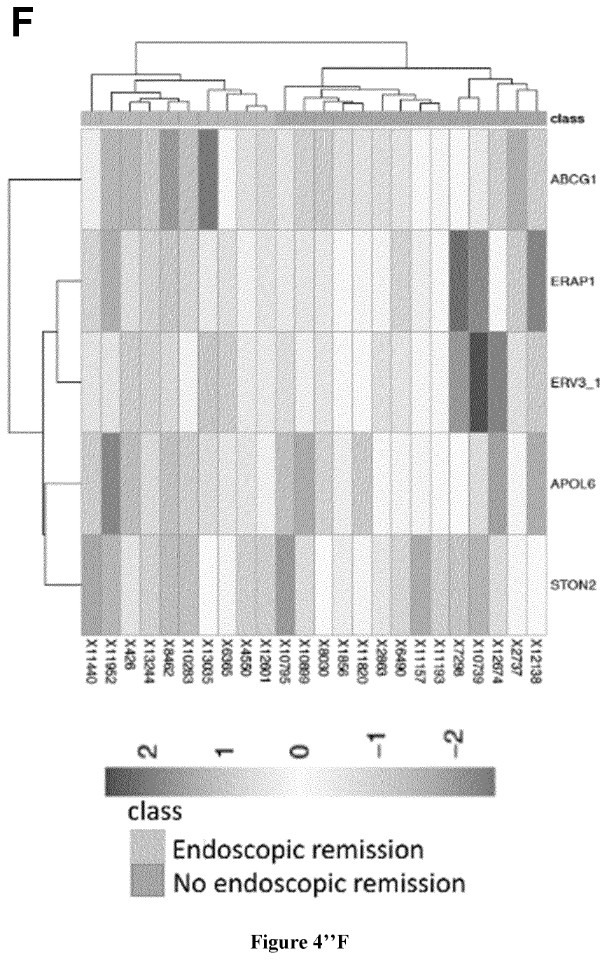

To validate previous findings and study the relationship between mucosal and whole blood gene expression levels, we performed RNAsequencing on 44 inflamed mucosal biopsies of IBD patients prior to the first anti-TNF administration, including 20 patients of the whole blood cohort. TREM1 was significantly decreased in future responders (FC=0.27, p=0.002), as well as OSM (FC=0.27, p=0.007), TNF (FC=0.57, p=0.02), IL13RA2 (FC=0.20, p=0.01) and TNFR2 (FC=0.72, p=0.008) (FIG. 4). Whole blood TREM1 levels correlated significantly with mucosal TREM1 levels (Spearman p=0.79, p=0.01, n=20).

Prediction of Response to Anti-TNF Therapy

Logistic regression analysis identified total whole blood TREM1 mRNA expression as the only significant predictor of anti-TNF induced endoscopic remission (p=0.02). ROC analysis based on baseline TREM1 mRNA levels in the anti-TNF cohort, gave an area under the curve (AUC) of 77.7% (95% CI 65.2-90.1%, p=0.001). Similar, mucosal TREM1 mRNA levels seemed to have good predictive accuracy with an AUC of 76.8% (95% CI 61.6-91.9%, p=0.003). In contrast, sTREM1 could not accurately predict anti-TNF induced endoscopic remission (AUC 58.3%, p=0.31). In the anti-TNF cohort with an overall pre-test probability for response and non-response of 42.6%, 57.4% respectively, predictive cutoffs were determined. Based on either 90.0% sensitivity or 90.0% specificity

(the latter also representing the Youden statistic), a post-test probability of 77.1% for achieving endoscopic remission (for values below the lower limit) (FIG. 5A) and a post-test probability of 90.0% for non-response (for values above the upper limit) (FIG. 5B) could be achieved. Only 1 out of 5 patients (20.4%) had an intermediate TREM1 level in between both thresholds.

An Anti-TNF Specific Marker in IBD Therapy

Baseline whole blood TREM1 expression was not associated with endoscopic remission in patients treated with either vedolizumab (n=51, p=0.53) or ustekinumab (n=22, p=0.82) (FIG. 6). Similarly, no association between baseline mucosal TREM1 expression and endoscopic remission could be observed in vedolizumab (n=67, p=0.24) or ustekinumab (n=51, p=1.0) treated patients (FIG. 6). Finally, no difference in TREM1 could be observed in patients with (82.4%) or without (17.6%) prior anti-TNF exposure (p=0.78) at the mucosal level.

Discussion

This is the first prospective study examining the predictive value of whole blood mRNA transcripts from genes previously identified in tissue as key in the prediction of anti-TNF therapy in patients with IBD.

We validated whole blood TREM1 expression as an accurate anti-TNF specific predictor for endoscopic remission in patients with CD and UC. In contrast to the observed moderate endoscopic remission rates with anti-TNF agents in current clinical practice, remission rates may be improved by prioritizing anti-TNF therapy to those patients with low TREM1 expression. Based on our results, pre-test probabilities for primary (non-)response to anti-TNF therapy could be optimized using TREM1 expression, resulting in post-test probabilities of 77.1% for endoscopic remission in the patients with lowTREM-1 expression (34.5% increase compared to pre-test probability) and 90.0% for non-response in the patients with high TREM-1 expression (32.6% increase compared to pre-test probability) respectively.

TREM1 is a receptor expressed on innate immune cells, known to amplify inflammatory signals that are initially triggered by Toll-like receptors and thus contributing to the pathophysiology of many acute and chronic inflammatory conditions [Carrasco K, Boufenzer A, Jolly L, et al. Cell Mol Immunol. 2019 May; 16(5):460-472]. Elevated serum levels of TREM1 have been documented in IBD patients, but sTREM1 does not correlate with the degree of endoscopic disease activity [Saurer L, et al. J Crohns Colitis 2012; 6 (9):913-23]. Similarly, TREM1 mRNA and sTREM1 protein levels did not correlate with CRP or faecal calprotectin in our cohort, suggesting that the TREM1 signal we observed is not purely reflecting a higher inflammatory state.

Increased TREM1 levels have been linked earlier to anti-TNF induced clinical response in a retrospective Israelian cohort of 28 patients with CD [Gaujoux R, Starosvetsky E, Maimon N, et al. Gut. 2019 April; 68(4):604-614. Epub 2018 Apr. 4]. We here observed the opposite signal, namely a significant increase in TREM1 in whole blood, both at the protein as at the mRNA level, in non-responders. Because our response criteria were based on more stringent endoscopic criteria and in view of the poor association between clinical symptoms and endoscopic disease activity [Saurer L, et al. J Crohns Colitis 2012; 6 (9):913-23 and Peyrin-Biroulet L, et al; Gut 2014; 63(1):88-95], these opposite results may not come as a surprise. As different transcripts could also contribute to the discrepancies between both studies, we not only focused on the overall TREM1 signal but also measured all known protein coding transcripts individually. Interestingly, all transcripts were significantly upregulated in future non-responders, and thus confirmed our belief of a true biological and clinically relevant signal. The limited number of patients in the original cohort by Gaujoux et al. and the different ethnicity could also contribute to this conflicting observation. However, the current validation of our previous findings (with increased TREM1 expression in future non-responders) [Verstockt B, Verstockt S, Blevi H, et al. Gut. 2019 August; 68(8):1531-1533. Epub 2018 Jul. 14.] in the current extended cohort, together with the absence of the same signal in an UST and VDZ treated cohort, raises the potential clinical applicability of measuring whole blood TREM1 as an anti-TNF specific biomarker in IBD.

Tissue biomarkers may be perceived as a better reflection of what is really going on in patients from a pathophysiological point of view. But, when it comes down to the translation to daily practice, a simple blood sample is less invasive than colonoscopy and easier to implement on a broader scale. In this study we showed that the accuracy of mucosal TREM1 expression is similar to the accuracy of whole blood TREM1 levels. In homeostatic conditions, the vast majority of resident intestinal macrophages completely lack TREM1 expression. In contrast, in patients with active IBD, TREM1 expression is mainly upregulated on intestinal macrophages with only limited TREM1-expressing intestinal neutrophils [Schenk M, et al; J Clin Invest 2007; 117(10):3097-106]. Immunophenotyping revealed a higher number of recruited TREM1+ CD14+ HLA-DRint macrophages, and not resident CD14+ HLA-DRhi lamina propria macrophages (LP), among CD45+LP cells in the inflamed mucosa of patients with IBD (compared to uninflamed regions) [Brynjolfsson S F, et al. Inflamm Bowel Dis 2016; 22(8):1803-11], explaining why the TREM1 mucosal signal could be picked up in whole blood as a (surrogate) biomarker. In mice, inhibition of TREM1 attenuated the severity of colitis clinically, endoscopically and histologically by restoring impaired autophagy and endoplasmic reticulum stress [Kokten T, Gibot S, Lepage P, et al. J Crohns Colitis 2018; 12(2):230-44]. As anti-TNF induced macrophages (Mϕind), have increased levels of autophagy and an intact autophagy pathway seems crucial for an optimal response to anti-TNF therapy [Levin A D, et al. J Crohns Colitis 2016; 10(3):323-9], we hypothesize that the lower TREM1 levels observed in

future anti-TNF induced responders are indeed associated with a better functioning autophagy pathway and thus a higher chance to achieve endoscopic remission after anti-TNF exposure. The lower serum TREM1 levels in responders to anti-TNF are not reflecting a higher membrane TREM1 expression. In contrast, future responders have significantly lower membrane bound TREM1, suggesting that their downstream proinflammatory TNF burden is lower, as has been reported earlier [Verstockt B, Verstockt S, Creyns B, et al. Aliment Pharmacol Ther 2018 Aliment Pharmacol Ther. 2018 October; 48(7):731-739]. Although there exist a specific splicing variant coding sTREM1 (TREM1-sv) [Gingras M C, et al Mol Immunol 2002; 38(11): 817-24 and Baruah S et al J Immunol 2015; 195(12):5725-31], sTREMI can also originate after cleavage of the membrane bound protein, as membrane TREM1 contains a matrix metalloproteinase 9 (MMP-9) cleavage site [Weiss G, et al Mucosal Immunol 2017; 10(4):1021-30 and Kumar S, et al. PLoS One 2015; 10 (5):e0127877]. As MMP-9 is known to be significantly upregulated in serum of active IBD patients [de Bruyn M, Arijs I, De Hertogh G, et al. J Crohns Colitis 2015; 9(12):1079-87], an increased cleavage of TREM1-mb is plausible, resulting in higher sTREM1 level than expected based on the TREM1-sv transcript alone and explaining why only the overall TREM1 expression level, but not the individual transcript levels, are correlating with sTREM1.

Using an unbiased, genome wide approach through RNA-sequencing of whole blood may therefore be even better to detect novel, outstanding predictive biomarkers in blood.

In conclusion, we validated baseline whole blood and mucosal TREM1 expression in IBD patients as an anti-TNF specific predictive biomarker for endoscopic remission.

Particular and preferred aspects of the invention are set out in the accompanying independent and dependent claims. Features from the dependent claims may be combined with features of the independent claims and with features of other dependent claims as appropriate and not merely as explicitly set out in the claims.

Thus, the claims following the detailed description are hereby expressly incorporated into this detailed description, with each claim standing on its own as a separate embodiment of this invention.

Examples ′2 on prediction of outcome for medicaments directed against interleukin 12 (IL12) and interleukin 23 (IL23)

The therapeutic landscape for inflammatory bowel disease (IBD) has expanded significantly over the past decade. Since the introduction of anti-TNF agents almost 20 years ago (T. Billiet, P. et al Expert Opin Biol Ther 14, 75-101 (2014)), anti-integrins (vedolizumab) and compounds blocking the IL-12/23 pathway (ustekinumab) have recently been licensed for the treatment of Crohn's disease (CD) (B. Verstockt, et al. J Gastroenterol 53, 585-590 (2018).). Despite the overall therapeutic successes, we still face an enormous therapeutic gap with endoscopic remission rates not exceeding 30% (P. Rutgeerts, et al. Gastroenterology 142, 1102-1111 e1102 (2012), P. Rutgeerts, et al. Gastrointest Endosc 63, 433-442; quiz 464 (2006), P. Rutgeerts, et al. Gastroenterology. 2018 October; 155(4):1045-1058.). Hence, many more agents are currently under development (J. Sabino, et al. An update. Therap Adv Gastroenterol., (2019). Additionally, remission rates of currently available therapies should be further optimized, taking into consideration early initiation, drug monitoring (allowing sufficient exposure in individual patients) and predictive biomarkers, allowing a more personalized treatment strategy.

Mechanisms of drug response are poorly understood, and why some patients respond better to one class over another, and others respond well to all classes, is unknown. Multiple factors are believed to contribute to a patient's response to biological therapies including host genomics, transcriptomics and possibly also metagenomics, metabolomics and proteomics. So far, there are limited studies that looked at prediction of drug response, and most focused on single omic layers. A number of (transcriptomic) biomarkers predicting response to anti-TNF therapy have been reported. However, because of the complexity of IBD, one can question the simplicity of a single-gene biomarker. As artificial intelligence, machine learning and systems biology have now opened the avenue for the efficient integration and interpretation of big datasets, technological advancements including next-generation sequencing, high-throughput omic data generation and molecular networks should be applied to dissect the underlying complexity and predict response to therapy, ultimately unravelling the “IBD interactome” (H S. P. de Souza, eNat Rev Gastroenterol Hepatol 14, 739-749 (2017).). This systems biology approach is not only about high-throughput omics measurements, but also a different philosophy of performing research. Compared with a standard hypothesis-driven approach, a systems approach is an unbiased data-driven strategy in which data integration reveals the key players of the network ((H. S. P. de Souza, eNat Rev Gastroenterol Hepatol 14, 739-749 (2017)). Stratifying patients into subgroups from identified predictive indicators is a crucial obstacle to deliver precision medicine.

In this study, we prospectively collected serum (proteomics), inflamed mucosal tissue from both ileum and colon (transcriptomics), sorted CD14+ and CD4+ T-cells (transcriptomics) and DNA (genomics) in CD patients with active endoscopic disease initiating ustekinumab therapy. Ustekinumab (Janssen Biotech, Inc.), a fully human IgG1 monoclonal antibody targeting the IL-12/IL-23p40 subunit, has been the latest approved biological agent for CD. All different omic datasets were subsequently integrated, in order to better understand the mode of action and identify potential biomarkers predictive for therapeutic success.

### Example 2 a Patient Characteristics

Sixty-four CD patients with active disease and a median (IQR) disease duration of 16.5 (9.7-23.6) years were included in this prospective study, prior to their first ustekinumab administration (Table 1′). At time of induction, 20 patients (31.2%) were on corticosteroids and just one patient (1.6%) received an immunomodulator. Almost all patients (98.4%) had failed or were intolerant to anti-TNF therapy, with 81.3% of them also having failed vedolizumab. After 8 weeks of ustekinumab therapy, 40.7% of patients experienced a 50% decrease in faecal calprotectin. After 6 months only 17.2% achieved endoscopic response with ustekinumab standard maintenance dosing of 90 mg q8 weeks SC.

### Example 2 b Variance Decomposition and Annotation of Latent Factors

By integrating the datasets from different omic layers (FIG. 6′), MOFA identified 20 latent factors (minimum explained variance 2% in at least one data type) (FIG. 1′). Among those, LF 1 almost exclusively explained all variance in the genetic layer (74.1%), whereas LF4, LF3 and LF5 contributed only to the variance in the ileal (38.0%), CD14+(37.0%), and colonic (31.0%) transcriptomic layers respectively. Using multiple regression modelling, we identified the explanatory LFs (LF3 and LF5) which contributed to the decrease in faecal calprotectin at week 8, with the CD14 and colonic transcriptomic datasets being the dominant layers within those LFs. Interestingly, those calprotectin-related LFs were different from the LF related to the endoscopic response after 6 months therapy (LF11). Nevertheless, this LF11 was again dominated by the colonic and CD14 transcriptomic layers, similar to LF3 and LF5 which were linked to the faecal calprotectin decrease.

Predicting an 8-Week Biological and 24-Week Endoscopic Ustekinumab Response

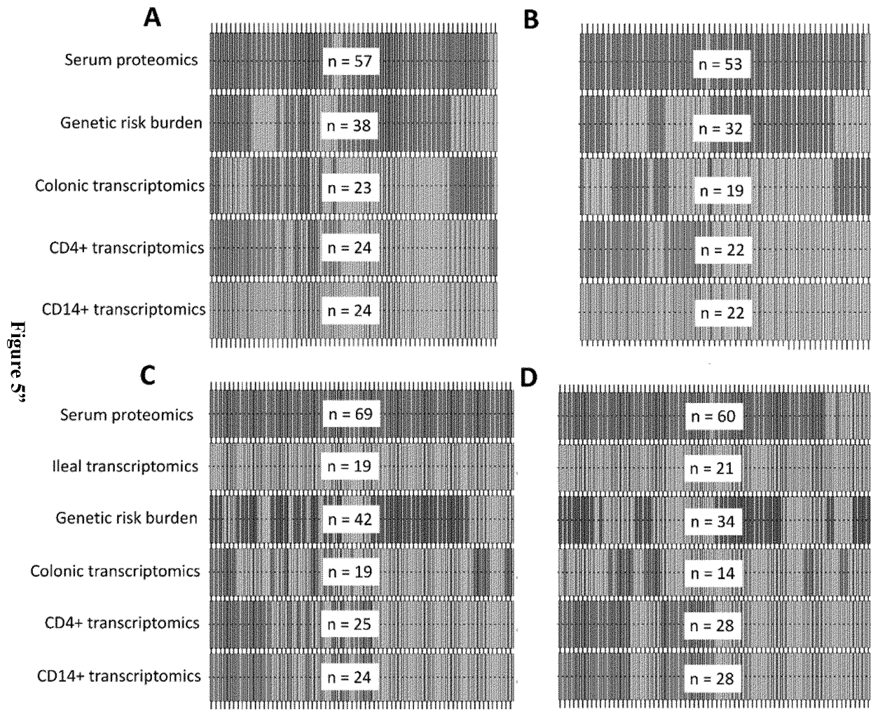

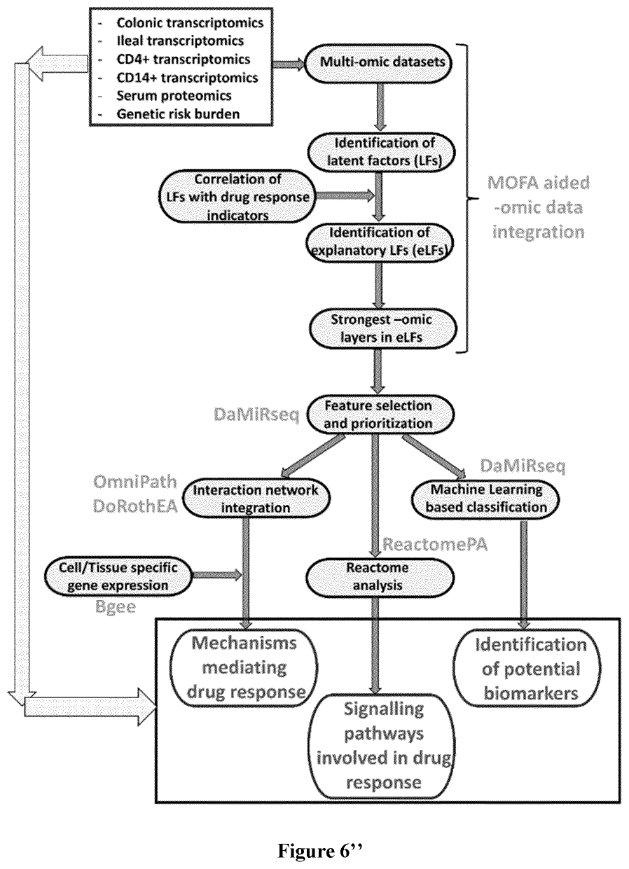

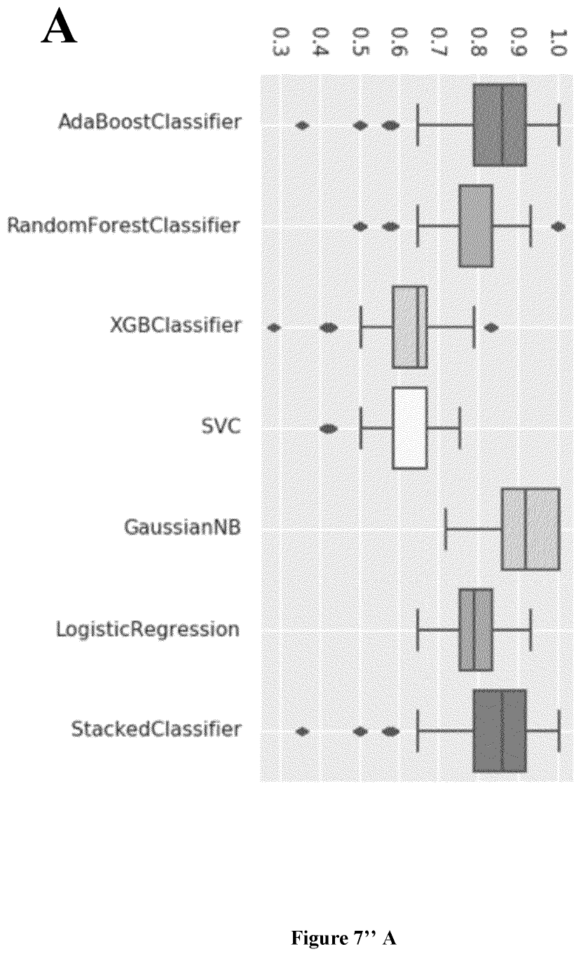

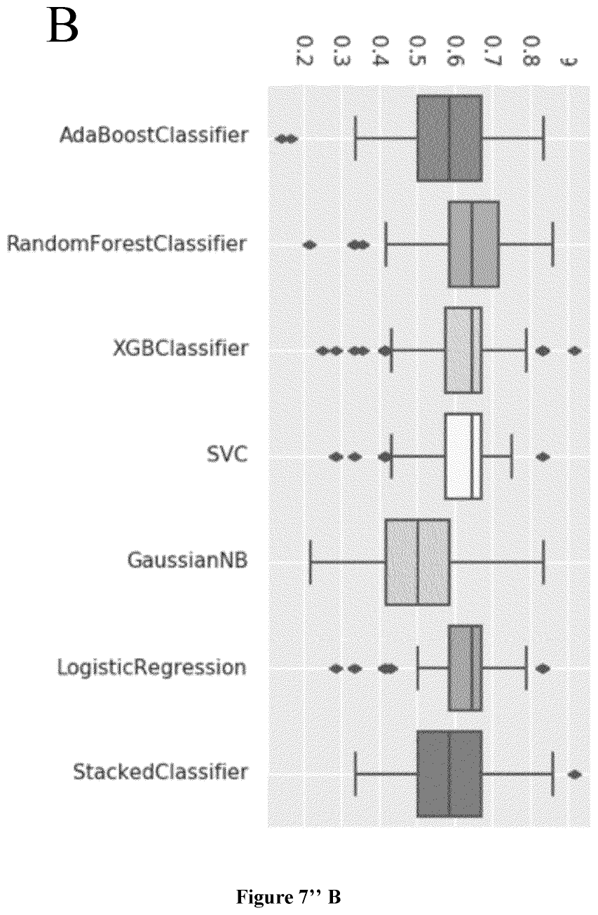

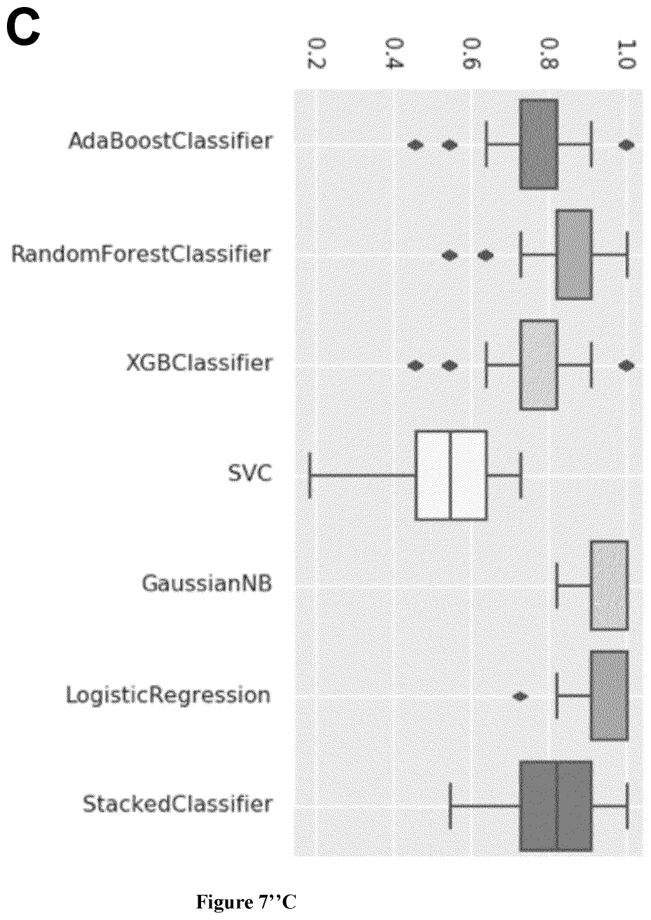

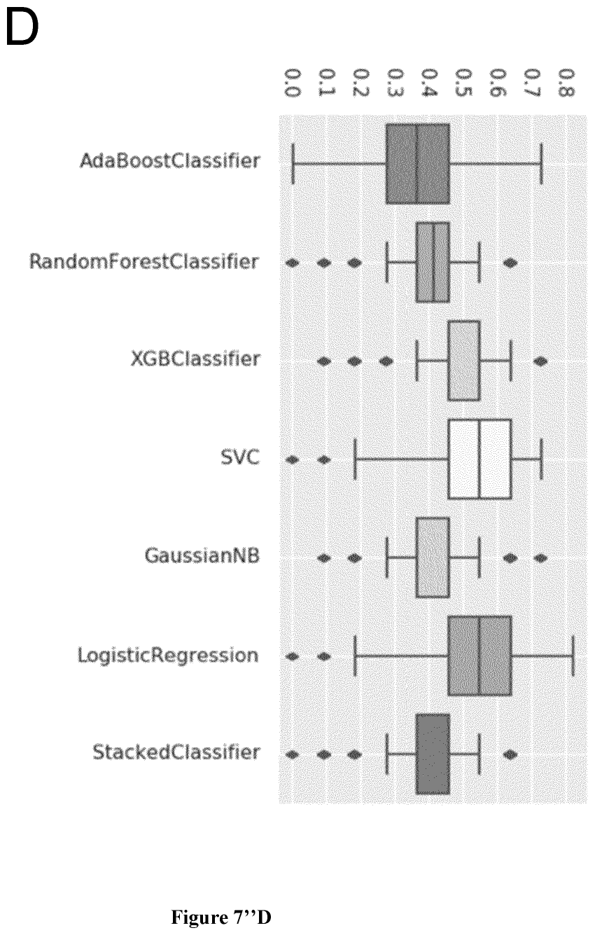

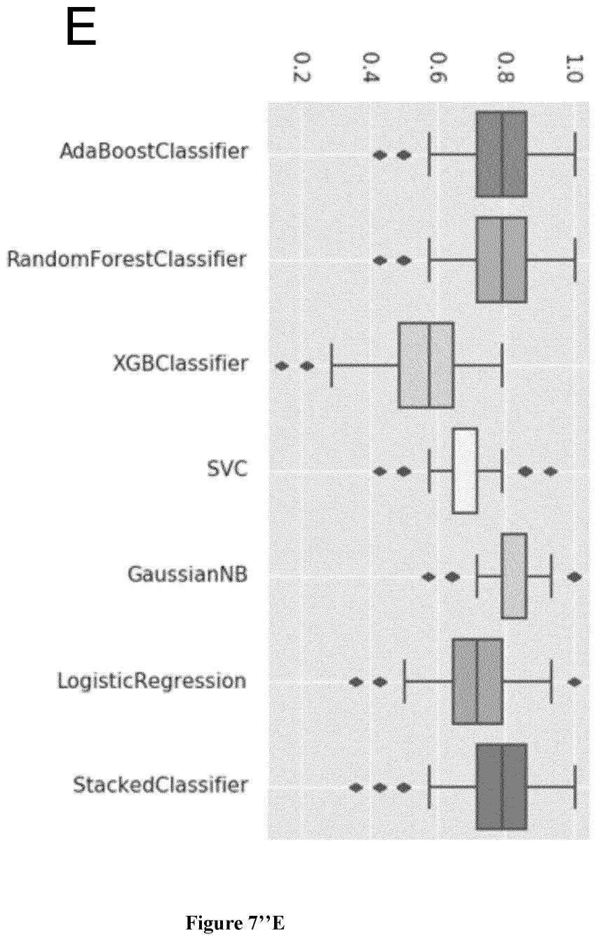

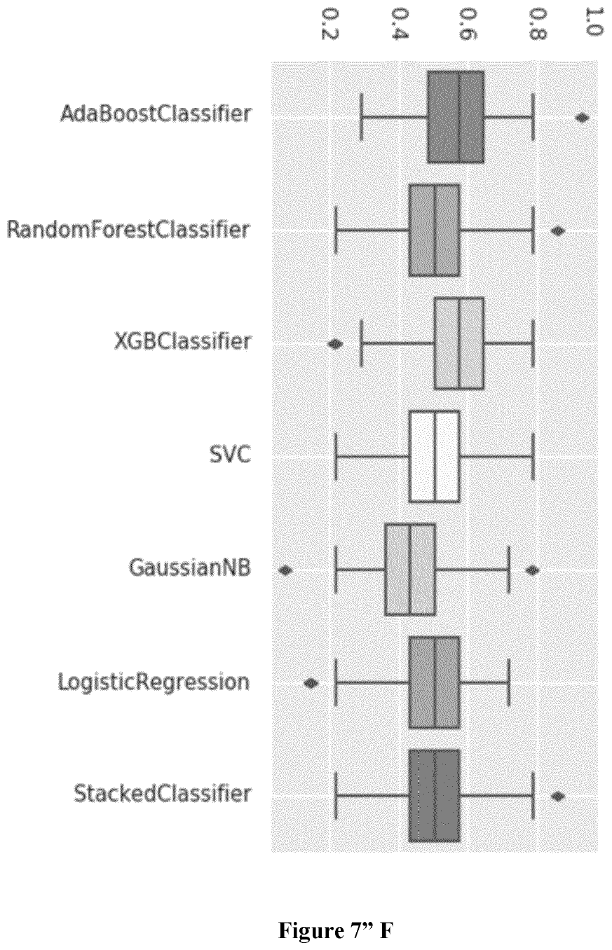

Both the CD14 and colonic transcriptional signatures were inferred as potential predictors of the reduction in faecal calprotectin. To test and verify this, we used feature reduction techniques to infer genes within the particular gene expression layers which could properly differentiate the subjects who did and did not experience a 50% reduction in faecal calprotectin 8 weeks after IV ustekinumab induction. After dimensionality reduction, we identified 77 and 124 genes from the CD14 and colonic transcriptional signatures respectively (FIG. 2′, which could explain the faecal calprotectin response-based segregation of patients (FIG. 7′). Importantly, this segregation is not reflecting a difference in baseline inflammatory burden between responders and non-responders. For each of the two transcriptomic datasets, the top 10 features (FIG. 2′), selected based on the score generated by the multi-variate filter RReliefF, were used for performing the Machine-Learning based classification. Mean accuracy rates for the 10 selected features from the CD14 and colonic transcriptomic datasets using a 6-stack ensemble classifier were reported at 100% and 99% respectively (FIG. 3′). On the other hand, the mean accuracy rates for 10 randomly selected features from the same dataset post normalization was around 77%.

Similarly, for the ustekinumab induced endoscopic response after 24 weeks we identified the CD14 transcriptomic dataset to be associated. Interestingly, the 10-feature CD14 panel predicting the initial drop in faecal calprotectin did not overlap with the 10-feature CD14 panel predicting an endoscopic ustekinumab response after 6 months. The CD14-calprotectin and CD14-endoscopic panels shared only 17 features, suggesting that different genes and processes in CD14 cells contribute to the initial biological and mid-term endoscopic response. We identified a 74-panel gene signature which explained the segregation of endoscopic responders from non-responders. The mean accuracy rates for the ensemble classification using the 10 relevant and 10 randomly selected features were determined to be 96% and 70% respectively (FIG. 4′).

### Example 2 c Pathway Analysis of the Panel Features

Monocyte-expressed genes predictive for the initial drop in faecal calprotectin and genes predictive for endoscopic response after 6 months were involved in many common pathways, including OX40 signalling, antigen presentation, Cdc42 signalling and Th1/Th2 activation pathway (Table 2′). In contrast, CXCR4 signalling was uniquely linked to the initial drop in faecal calprotectin (BH adjusted p-value=0.03), whereas, IL-4 signalling (BH adjusted p-value=0.008) and dendritic cell maturation (BH adjusted p-value=0.01) among many others, were uniquely linked to endoscopic response (Table 2′). At the colonic transcriptomic level, (a)granulocyte adhesion and diapedesis (BH adjusted p-value=5.7×10⁻⁶, p=0.0007 respectively), as well as IL-17 signalling (BH adjusted p-value=0.04) contributed significantly to the initial drop in faecal calprotectin (Table 3′).

### Example 2 d Cell-Type Specific Modulation of Discriminatory Features by Inflammatory-Pathway Related Transcription Factors

We identified a set of 11 TFs which may modulate the expression of individual features identified in the three panels (CD14—calprotectin, CD14—endoscopy, colon—calprotectin). While 6 (RELA, NFKB1, STAT1, JUN, FOS, ATF2) of the 11 identified TFs could regulate the expression of at least one feature in all three panels, three (NFKB2, RELB, NFKB1A) were specific to only one of the panel. Unsurprisingly, TFs such as NFKB1 and STAT1, which play major roles in mediating inflammatory effects (T. Lawrence, Cold Spring Harb Perspect Biol 1, a001651 (2009) and M. H. Kaplan, STAT signaling in inflammation. JAKSTAT. 2013 Jan. 1; 2(1):e24198 (2013).), were identified as regulators of many chemokines and interleukins whose expression differentiate the responders from non-responders.

### Example 2 e Network Analysis Reveals Distinct Signalling Pathways and Modules Involved in the Colonic Treatment Response

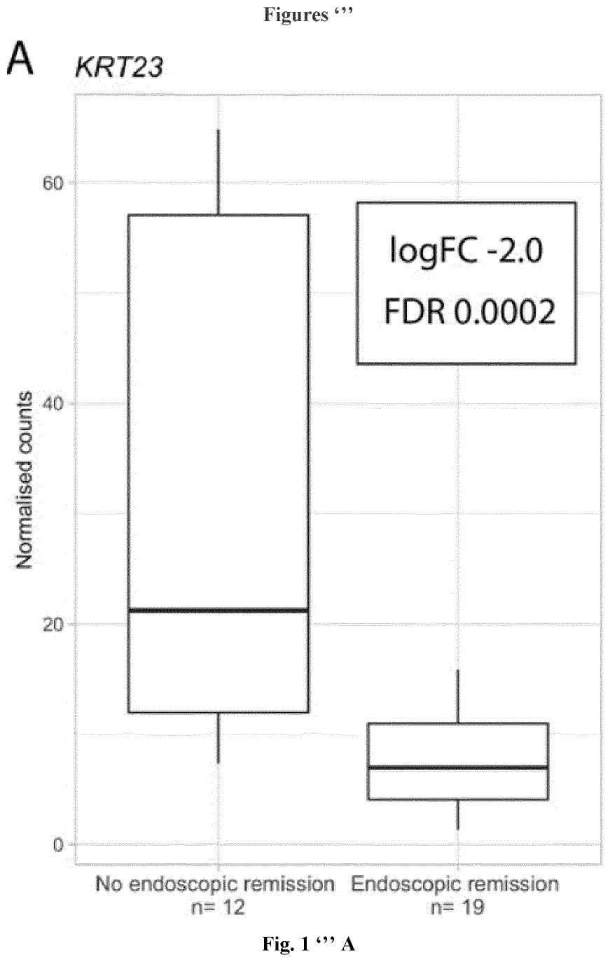

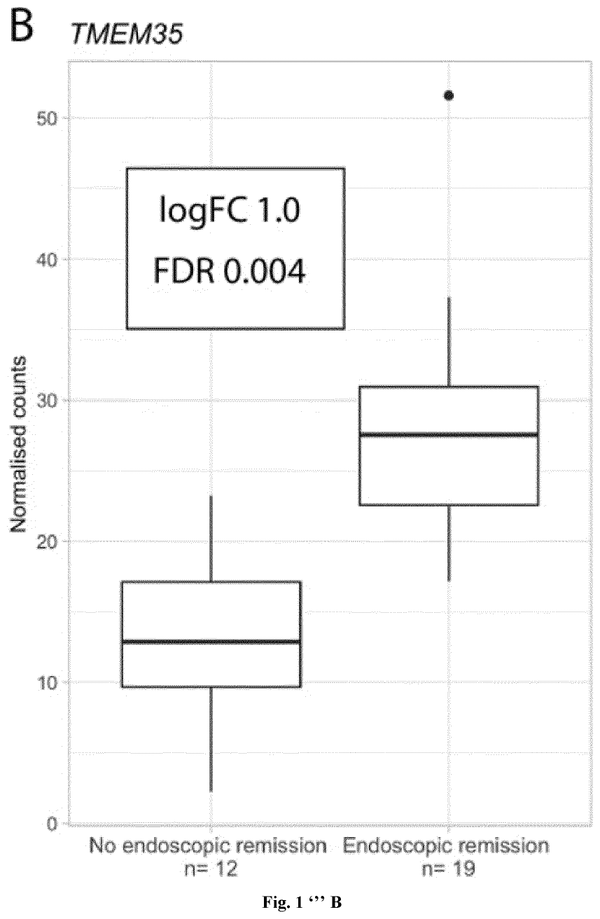

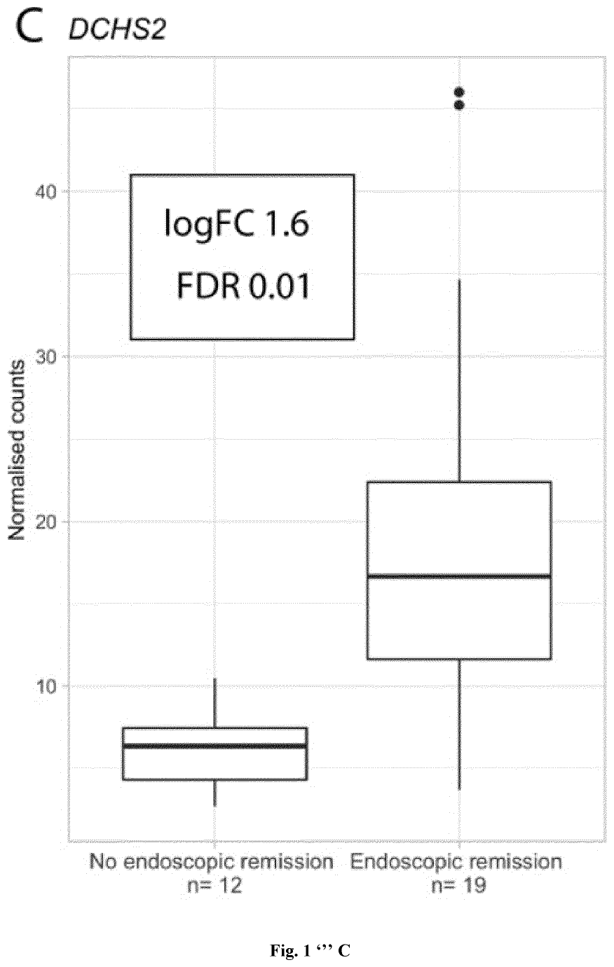

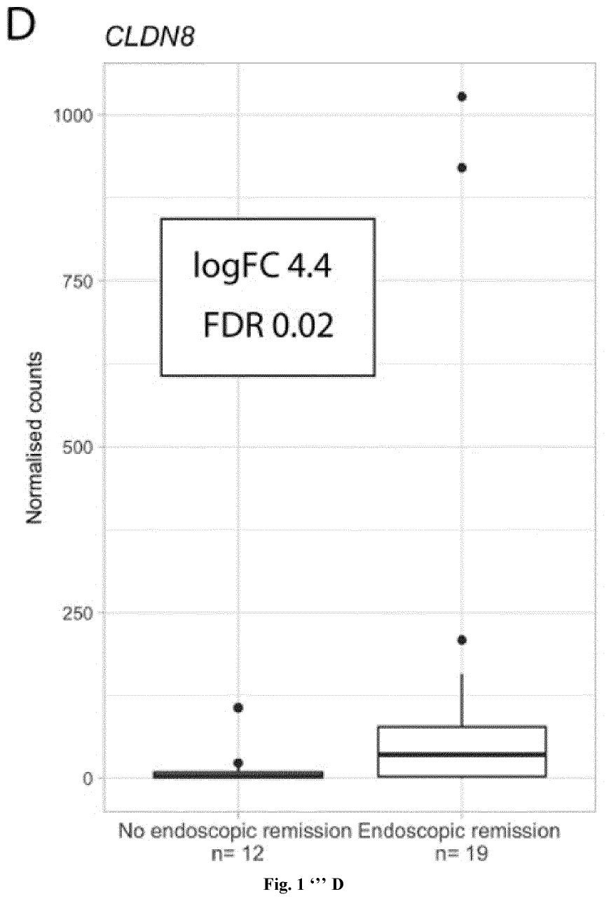

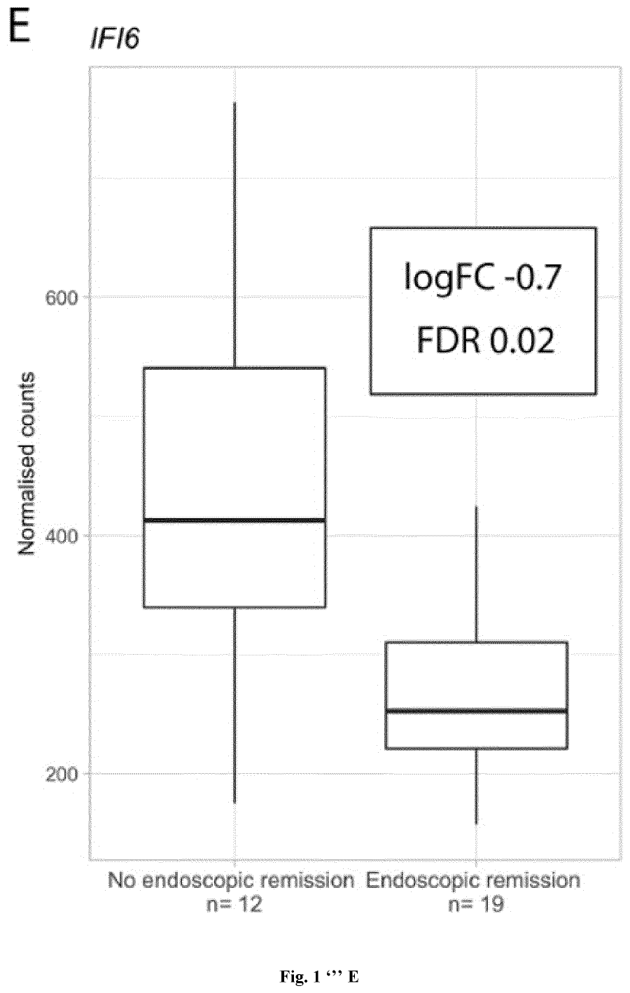

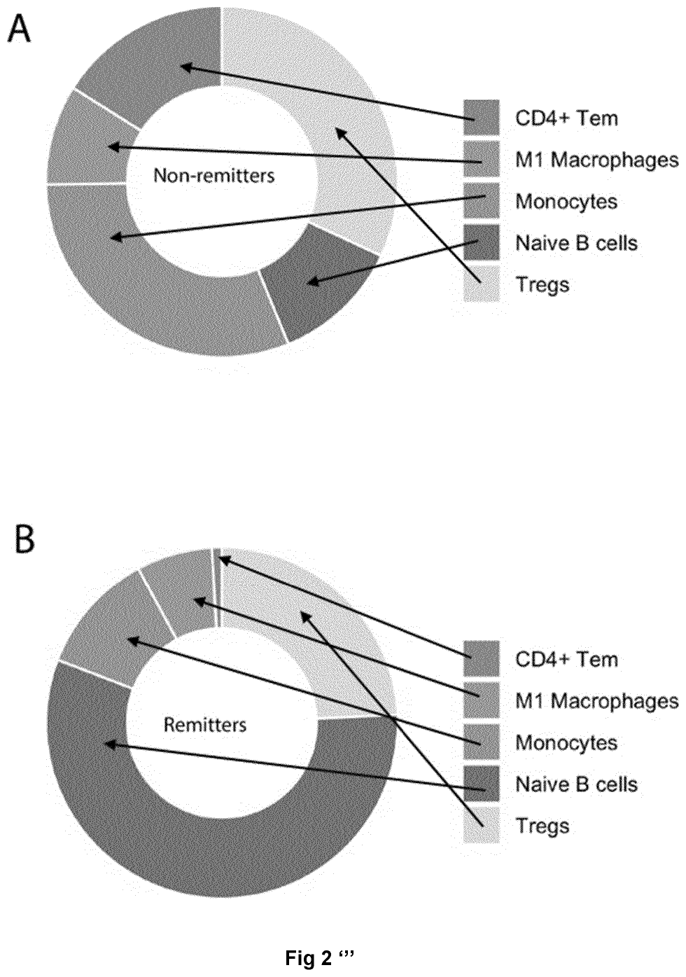

Functional enrichment analysis of the entire colonic response network revealed the over-representation of the network with Reactome pathways such as Interleukin-10 signalling, signalling cascades mediating TLRs 5 and 10, MyD88 signalling cascade among others (FIG. 8′). To gain insights into the functional context of the features from a regulatory and signalling perspective in the colonic tissue, we reconstructed the interaction network corresponding to the colonic features predictive of reduction in faecal calprotectin. The colonic response network consisted of 101 proteins distributed in 15 modules, the largest of which contained 53 proteins (FIG. 9′). The 53 proteins included 43% (12/28) of the discriminating features present in the entire colonic response network, thus indicating the critical function of this module in mediating the response to ustekinumab

### Example 2 f Materials and Methods

Patient Selection

This study was conducted at the University Hospitals of Leuven (Leuven, Belgium). We prospectively collected DNA, RNA (inflamed colonic and ileal tissue) and peripheral blood mononuclear cells (PBMC) as well as serum in 64 consecutive Crohn's disease patients initiating ustekinumab therapy (Table 1′). All recruited patients had endoscopy-proven active disease (Simple endoscopic score for Crohn's disease (SES-CD) >7, and >4 in patients with pure ileal involvement) and all were ustekinumab naïve. Proteomic data were available in all patients, whereas tissue transcriptomics, peripheral blood transcriptomics and genomics were available in 62.5%, 64.0% and 57.8% of patients respectively (FIG. 6′).

Outcomes

Biological response was assessed at week 8 and defined as a minimal 50% decrease in faecal calprotectin compared to baseline. Endoscopic response was defined as a minimal 50% SES-CD compared to baseline (M. Ferrante, et al; Gastroenterology 145, 978-986 e975 (2013)), and assessed at week 24 (A. Sturm, et al; European Society of R. Abdominal, ECCO-ESGAR Guideline for Diagnostic Assessment in Inflammatory Bowel Disease. J Crohns Colitis, (2018) and C. Maaser, et al. European Society of R. Abdominal, ECCO-ESGAR Guideline for Diagnostic Assessment in Inflammatory Bowel Disease. J Crohns Colitis, (2018).). All endoscopies were performed by the same 3 experienced IBD staff members (GVA, SV, MF).

Cell Separation

A 20 ml blood sample was taken, and PBMCs were isolated by density centrifugation. (37) After cell isolation, samples were cryopreserved with dimethyl sulfoxide (DMSO) using Mr Frosty (Thermo Fisher Scientific, Waltham, Mass., USA) for 24 hours and afterwards stored in liquid nitrogen. Frozen PBMC samples were subsequently thawed, and CD14+ monocytes and CD4+ T-cells sorted using fluorescence activated cell sorting (FACS) (median purity, 99.9% and 99.4% respectively).

Isolation of RNA

Total RNA from inflamed biopsies and sorted CD4⁺ and CD14⁺ cells was extracted using the AllPrep DNA/RNA Mini kit (Qiagen, Hilden, Germany) according to the manufacturer's instructions. The integrity and quantity of RNA was assessed with a 2100 Bioanalyzer (Agilent, Waldbronn, Germany) and a Nanodrop ND-1000 spectrophotometer (Thermo Scientific, Waltham, Mass., USA). Extracted RNA was stored at −80° C. until further processing.

RNA Sequencing

Next-generation single-end sequencing was performed using the Illumina HiSeq 4000NGS, after library preparation using the TruSeq Stranded mRNA protocol (Illumina, San Diego, USA) according to the manufacturer's instructions. Raw RNA-sequencing data were aligned to the reference genome using Hisat2 version 2.1.0 and absolute counts generated using HTSeq (D. Kim, et al. Nat Methods 12, 357-360 (2015) and S. Anders, et al. Bioinformatics 31, 166-169 (2015)). Raw RNA-seq data have been deposited in the ArrayExpress database at EMBL-EBI (www.ebi.ac.uk/arravexpress) under accession number E-MTAB-7799. Gene expression data were pre-processed, with only genes having at least 10 normalised read counts in at least 70% of the samples were considered for further analysis. Genes contributing to less than 1% of the variance across all samples were discarded. The resulting data were normalized using the varianceStabilizingTransformation function of the DESeq2 R package.

Serum Proteomics

Five hundred seventy-six unique proteins were measured using the Olink® CELL-REG, DEV, INF I, IO, MET, OD and IR panels (Olink Proteomics AB, Uppsala, Sweden) according to the manufacturer's instructions. The Proximity Extension Assay (PEA) technology used for the Olink protocol has been well described (E. Assarsson, et al; PLoS One 9, e95192 (2014), and enables 92 analytes to be analyzed simultaneously, using 1 μL of each sample. Data were quality controlled and normalized using an internal extension control and an inter-plate control, to adjust for intra- and inter-run variation. The final assay read-out is presented in Normalized Protein eXpression (NPX) values, which is an arbitrary unit on a log 2-scale where a high value corresponds to a higher protein expression. All assay validation data (detection limits, intra- and inter-assay precision data, etc.) are available on manufacturer's website (www.olink.com).

Genotyping Data

In a subset of patients (n=37, 57.8%) Immunochip genotype data were available (L. Jostins, et al; Nature 491, 119-124 (2012), from which the global mutation profiles were generated and transformed into a “genetic risk burden” matrix by mapping the mutations onto the protein coding genes. The “genetic risk burden” represents the degree to which a particular gene is affected in a patient by mutations which fall within its exonic regions. The mutation-gene mapping associations based on the genomic co-ordinates were retrieved from the chromosomal report files from the dbSNP database (S. T. Sherry, et al; Nucleic Acids Res 29, 308-311 (2001)).

MOFA Aided-Omic Data Integration

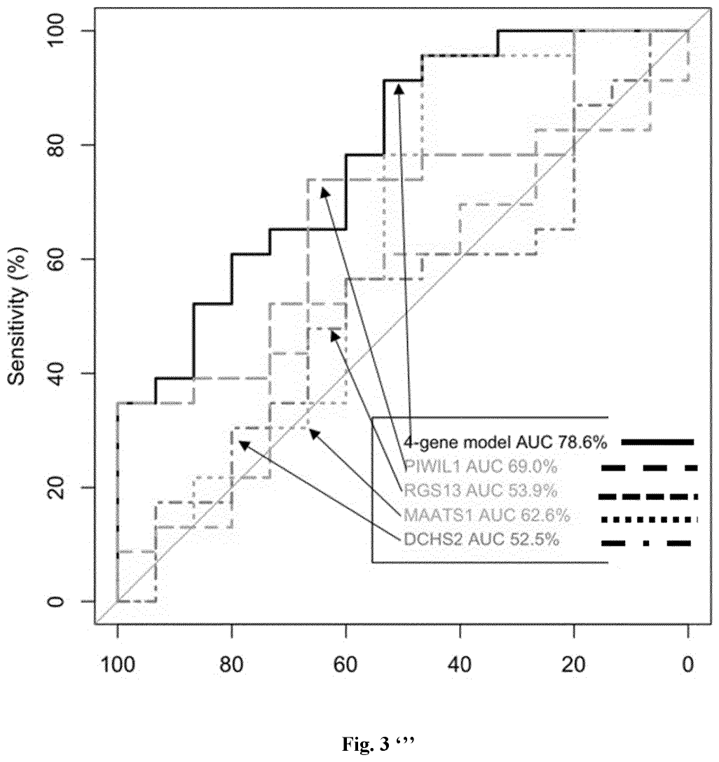

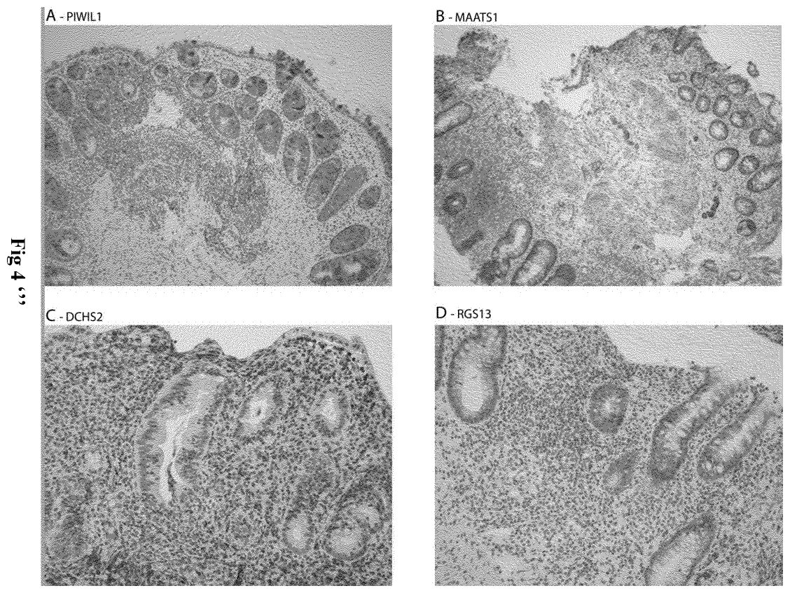

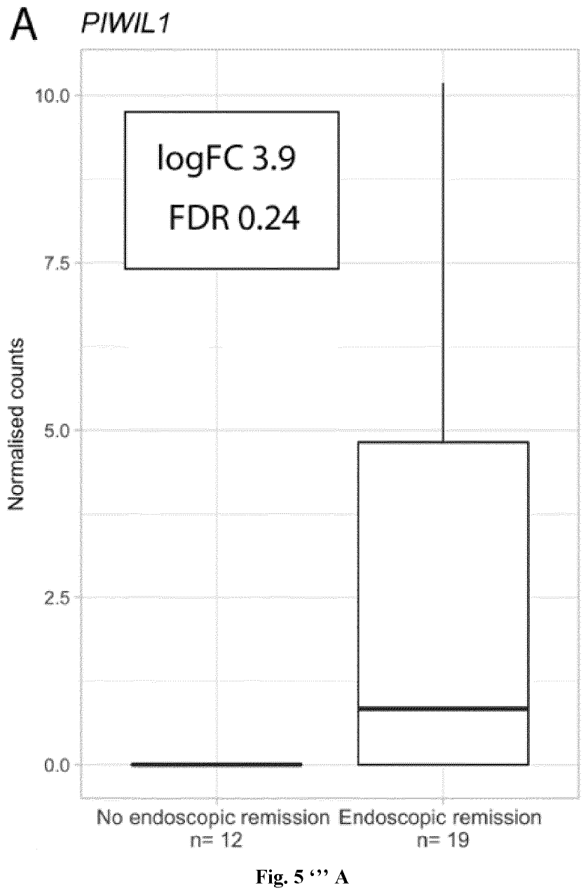

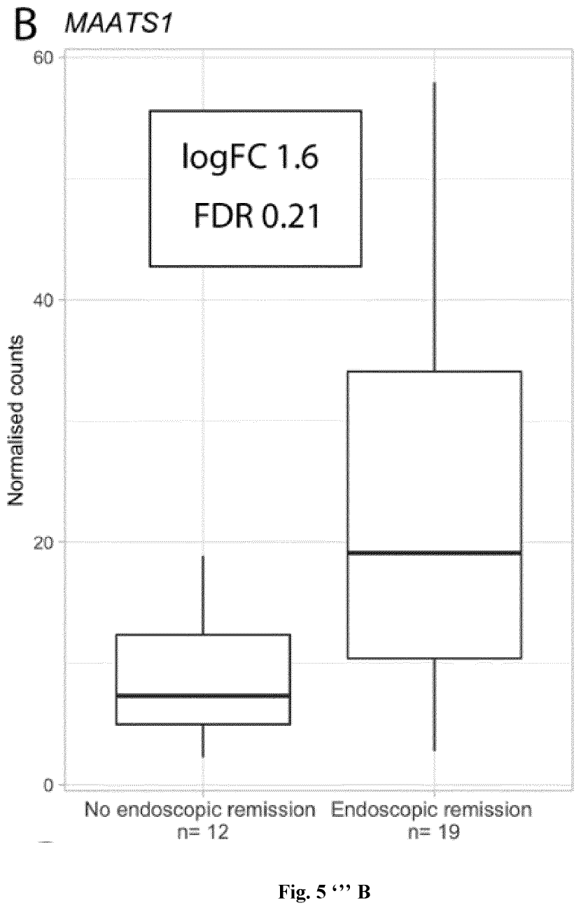

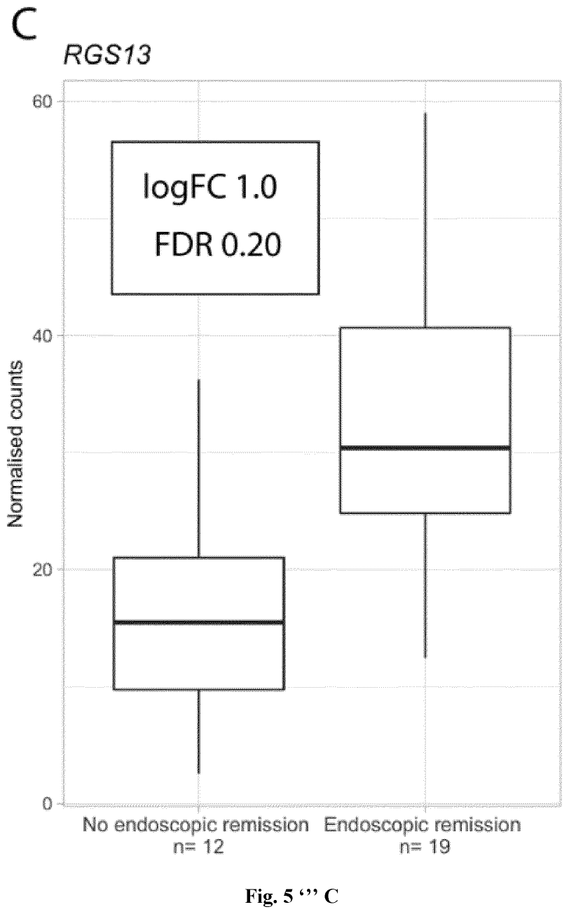

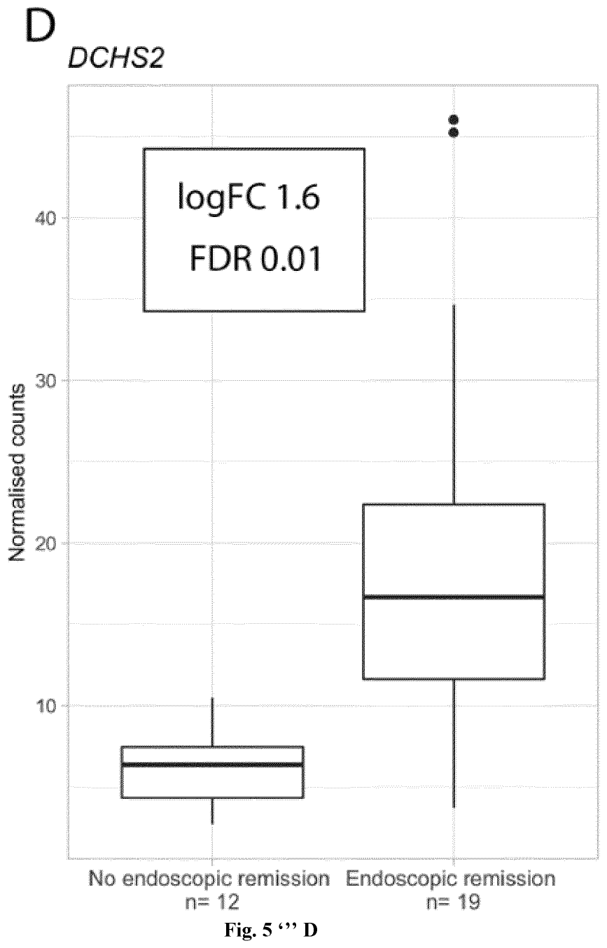

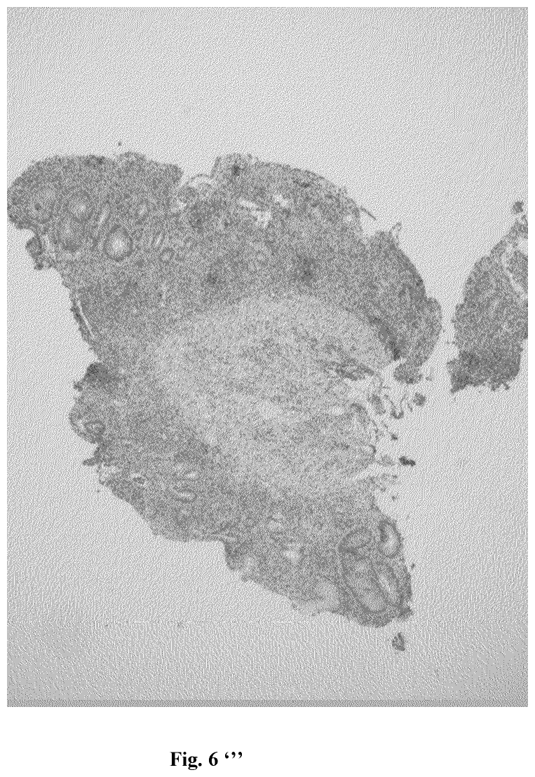

The Multi Omic Factor Analysis (MOFA) tool was used to analyse and evaluate the principle sources of variation in the multiple-omic datasets (normalized next-generation sequencing data from blood derived PBMCs (monocytes, CD4 T cells), colonic and ileal tissue, blood proteomics and the mutation derived genetic risk burden profiles) generated in our study (R. Argelaguet, et al. Mol Syst Biol 14, e8124 (2018)). The MOFA model object was compiled using the default model and training options with the exception of the DropFactorThreshold (representative of variance cut off) and maxiter (number of iterations) which were set at 0.02 and 15000 respectively. The compiled MOFA model object was executed using the runMOFA function to analyse and integrate the multi-omic datasets. The weights corresponding to each of the patients for every identified Latent Factor (LF) were retrieved using the getFactors function. The calculateVarianceExplained function was used to determine the contribution of the LFs towards the variance corresponding to each of the six different-omic datasets. Only LFs for which weights were assigned to all of the samples were considered. Using a multiple regression model, we identified the explanatory LFs which contribute to the desired traits (reduction in faecal calprotectin by week 8 or endoscopic response by week 24). LF-trait relationships with a p-value of <=0.1 were considered to be significant. The most dominant omic data layers in the explanatory LFs were determined from the variance contribution of the omic layers towards the LFs (FIG. 10′).

Feature Selection and Predictive Modelling

The gene expression data layers identified as the contributing -omic layers were subjected to feature selection and machine learning based predictive modelling using the DaMirSeq R package (M. Chiesa, et al. Bioinformatics 34, 1416-1418 (2018)), to identify genes whose expression signatures can discern patient groups (patients with/without endoscopic response, patients with/without reduction of faecal calprotectin). In order to avoid over-fitting, the samples were first split into a training (TR1) and test set (TS1) by bootstrap sampling. Another pair of training (TR2) and test set (TS2) were obtained from TR1. While TR2 is used to train the six individual classifiers, TS2 was used to test their accuracies.

Pathway and Network Analysis

Ingenuity Pathway Analysis (IPA, Qiagen Inc.) was used to identify canonical pathways linked (Benjamini-Hochberg correction <=0.05) to the genes discovered during feature selection and predictive modelling. Members from the IL12/IL23 related signalling pathways were downloaded from InnateDB (K. Breuer, et al; Nucleic Acids Res 41, D1228-1233 (2013)). Molecular interactions for the network analysis were retrieved from publicly available resources. Protein-protein interactions were retrieved from InnateDB, SIGNOR, and SignaLink2 via the OmniPath webserver while transcriptional regulatory interactions corresponding to the confidence levels A, B and C were downloaded from DoRothEA (K. Breuer, et al. Nucleic Acids Res 44, D548-554 (2016), D. Fazekas, et al. BMC Syst Biol 7, 7 (2013), D. Turei, et al; NatMethods 13, 966-967 (2016) and L. Garcia-Alonso, et aL; Cancer Res 78, 769-780 (2018)). Upstream transcription factors (TFs) which could potentially regulate the expression of the recovered features were identified. In addition, using the protein-protein interactions, the upstream post translational regulators of the TFs were also identified. The interaction analysis was confined to the TFs identified as belonging to the IL12/IL23 related signalling pathways from InnateDB (K. Breuer, et al. Nucleic Acids Res 41, D1228-1233 (2013)).

### Example 2 g Discussion

This is, to our knowledge, the first multi-omics data integration study in inflammatory bowel disease, integrating genomic, proteomic and transcriptomic (tissue, CD4 T-cells and monocytes) data to understand and identify molecular pathways and biomarkers involved in the response to ustekinumab therapy.

Through variance decomposition aided factor analysis, we identified different multi-omic subsets contributing to either an early decrease in faecal calprotectin or to endoscopic response after 6 months of ustekinumab therapy. Interestingly, our results point towards an important role for the innate immune system, as the CD14 transcriptomic dataset was the only omic layer which accurately predicted both short-term biological and mid-term endoscopic ustekinumab outcomes. Previous studies have highlighted the importance of the innate immune system with regard to disease outcome and therapy response (S Zeissig, E. et al Gut 68, 25-39 (2019).).

In contrast to 10 random features within the CD14 transcriptomic dataset which very poorly predicted response, we identified two relevance-filtered 10-feature transcriptomic panels which accurately predicted early biological response and endoscopic response. We hypothesize that the 10-feature panel predictive of a 50% drop in faecal calprotectin by week 8 could be a biomarker panel to select suitable patients for ustekinumab therapy. The additional 10-feature panel predicting endoscopic response, may subsequently be used to select patients in whom standard maintenance dosing is sufficient to induce endoscopic response (FIG. 5′). Patients indeed—after successful induction—may lose response to biological agents, including ustekinumab, over time (B. Verstockt, et al. J Crohns Colitis. 2019 Jul. 25; 13(7):864-872.), which is why we may have picked up different panels for different outcomes over time. Similar to previous studies focusing on prediction of disease prognosis (J. C. Lee, et al. J Clin Invest 121, 4170-4179 (2011), E. F. McKinney, et al. Nat Med 16, 586-591, 581p following 591 (2010) and D. Biasci, at al. Gut. 2019 August; 68(8):1386-1395.), our identified immune cell biomarker panel should now be translated in a whole blood, surrogate biomarker panel prior to validation in an independent cohort.

An integrated multi-omics framework not only provides a great opportunity to identify biomarkers with clinical applicability (R. K. Weersma, et al. Gastroenterology. 2018 November; 155(5):e1-e4.), but may also give detailed insights into the mechanistic actions of a particular drug. Common pathways involved in ustekinumab response both at week 8 and at month 6 included antigen presentation, OX40 signalling and activation of T-cells. This connects ustekinumab response with microbial sensing, and raises the question whether a specific microbial antigen/signature, sensed by innate immune cells influences ustekinumab response, as recently suggested (M. K. Doherty, et al. MBio. 2018 Mar. 13; 9(2). pii: e02120-17.).

Based on our findings also NFAT signalling was linked to ustekinumab response, which seems plausible as NFAT is known to regulate IL-12p40 expression in innate immune cells (C. Zhu, et al. J Biol Chem 278, 39372-39382 (2003)). CXCR4 signalling, known to mitigate the IL-23 effect in a mice model for psoriasiform dermatitis (T. Takekoshi, et al. J Invest Dermatol 133, 2530-2537 (2013)), uniquely contributed to the initial drop in faecal calprotectin. Many more pathways were involved in the endoscopic response after 6 months, including IL-4 signalling. Interestingly, IL-4 promotes IL-12 production but abrogates Th17 cell-mediated inflammation by the selective silencing of IL-23 in antigen-presenting cells (E. Guenova, Y. et al; Proc Natl Acad Sci USA 112, 2163-2168 (2015)). Also HMGB1 signalling which was significantly linked to ustekinumab response at the colonic level, is another pathway previously linked to IL-17 production in a IL-23 dependent manner (Q. Tang, et al. Mediators Inflamm 2013, 713859 (2013), X Chen, et al; Innate Immun 22, 696-705 (2016) and Z. He, et al. Scand J Immunol 76, 483-490 (2012).). Nevertheless, our findings highlight the need for future functional experiments in future, validating the observed pathways and how they may affect ustekinumab response, as hypothesised based on current literature.

Similar to the overlapping and unique pathways linked to the different endpoints studied, analysis at a network level also revealed overlapping and unique transcription factors regulating the different outcomes. Several transcription factors, such as NFKB1 among many others, were identified as regulators of unique markers in each data layer. JUN, which contributes to therapeutic resistance in cancer (M. H. Kaplan, STAT signaling in inflammation. JAKSTAT. 2013 Jan. 1; 2(1):e24198 (2013)), was also identified as a feature in the CD14—calprotectin and CD14—endoscopy panels, suggesting that it may play a critical role in mediating the response to ustekinumab.

We do acknowledge the issue of missing data in the current study. Missing data is not uncommon especially if many different specimens (tissue, serum, blood) have to be collected and processed from the same individual. The MOFA tool has been designed to cope with this real-life limitation, as demonstrated in the landmark paper in which at least one omic layers was missing in 40% of all included individuals (R. Argelaguet, et al; Mol Syst Biol 14, e8124 (2018)). Additionally, we acknowledge the lack of an independent validation cohort for our identified biomarkers. But to reduce the risk of over-fitting, we used multiple classifiers and an ensemble classifier over multiple iterations, with training and test sets generated by bootstrapping. Instead of sorted cells, surrogate whole blood markers are absolutely warranted to confirm our findings in an independent cohort and to make translation into clinical practice feasible. Finally, this biomarker should in future be tested also in patients initiating vedolizumab or anti-TNF therapy to study its specificity for ustekinumab. However, the identified pathways and networks contributing to ustekinumab response are clearly linked to the IL-12/IL-23/IL-17 axis, which indirectly suggests its ustekinumab specific character.

In conclusion, this is the first multi-omics data integration study in any sub-type of inflammatory bowel diseases. We identified two CD14 transcriptomic panels which accurately predicted a biological and endoscopic response to ustekinumab, and provide additional insights into biological pathways which either mediate the mode of action of ustekinumab and/or the response to ustekinumab in Crohn's disease. Although further validation in independent cohorts is absolutely warranted, this systems biology approach brings personalised medicine in IBD one step closer.

### Example″ 3 on Anti-α₄β₇-Integrin Agents and/or Anti-IL-12/23 Agents

### Example 3-a Patient Characteristics

Two hundred fifty-five individual patients with IBD (139 CD, 116 UC) were included in this prospective study, prior to the initiation of anti-TNF (n=124) or vedolizumab (n=131) therapy (Table″ 3). Endoscopic remission rates varied from 36.4% (anti-TNF) to 62.3% (vedolizumab) in UC, whereas anti-TNF and vedolizumab-induced endoscopic remission rates in CD were 55.1% and 47.1% respectively. Proteomic data were available in almost all patients (93.7%), whereas genomics (56.1%), tissue transcriptomics (46.3%) and CD4⁺ T cells/monocyte transcriptomics (38.8%) were available in subsets of patients (FIG. 5″). At least two distinct -omic layers were available in all patients.

### Example 3-b Variance Decomposition and Annotation of Latent Factors

By integrating the different -omic layers (FIG. 6″) in anti-TNF treated UC patients, Multi-Omics Factor Analysis (MOFA) identified 15 latent factors (LF) (minimum explained variance 2%), whereas 14 LF were found in the vedolizumab treated UC cohort (FIG. 1″). In both CD cohorts, we identified 21 (anti-TNF) and 15 (vedolizumab) LF, respectively (FIG. 1″). Multiple regression modelling identified the explanatory LFs which significantly contributed to endoscopic outcome (Table 4-5″). Subsequently, we determined the dominant -omic layers within those identified LFs from the variance contributions (Table 8-9″). Interestingly, genetics significantly contributed in terms of variance to the LFs correlating with the endoscopic outcome in 3 out the 4 datasets (anti-TNF UC, anti-TNF CD, vedolizumab UC and vedolizumab CD) (Table 8-9″). In addition, gene expression from the CD4+ T cells and CD14′ monocytes data sets were observed to have higher contributions to the variance within the explanatory LFs in each of the cohorts, as compared to tissue transcriptomics (Table 12″).

### Example 3-c Identification of Cell-Type Specific Features Contributing to Anti-TNF and Vedolizumab Outcome in Ulcerative Colitis and Crohn's Disease

After having identified the dominant -omic layers in the explanatory LFs, we zoomed into those layers by using supervised techniques to point out features which distinguish remitters and non-remitters for every drug-disease combination. At the gene expression level, we identified 457 features (genes) cumulatively across cell and tissue-types, drugs and disease (Table 13″). While 180 (39.4%) features were unique to vedolizumab remission across all datasets, 225 were specific for anti-TNF remission (49.2%). The remaining 52 (11.4%) were found to be features distinguishing remitters and non-remitters in both the vedolizumab and anti-TNF cohorts, potentially pointing towards a more refractory phenotype. Also, at a cell type specific level, characteristic features for vedolizumab or anti-TNF remission could be identified. For example, 86% and 82% of the CD4+ and CD14′ associated features respectively were found to be unique to particular drug-disease combinations (FIG. 2″), with only a small number of genes shared by at least 2 drug-disease combinations. None of the discovered markers significantly correlated (p value <=0.05) to the C-reactive protein levels (Table 14″).

### Example 3-d Network Analysis Reveals Divergent Functional Effects Mediated by Protein Hubs Distinguishing Responders and Non-Responders in a Cell-Type and Cohort-Specific Manner

Using curated interaction networks, we wanted to infer the functional effects of the identified cell-type specific features, as discussed above, in the different cohorts. We retrieved the immediate downstream targets of the protein-encoding genes whose expression discriminates remitters and non-remitters in a cohort-wise and cell-/tissue-type specific manner (Table 15-14″), and evaluated the functions of such targets which are known to be expressed in the corresponding cell- and tissue-type. Our network analyses revealed divergence of effects mediated by these features via their downstream targets (Table 17″, Table 1″).

In the CD4⁺ T cell layer of the vedolizumab UC cohort, we identified Smad family member 7 (SMAD7) as the top hub (defined herein as a protein with a high number of downstream targets) among the features distinguishing remitters and non-remitters. The downstream targets of SMAD7, which was upregulated in remitters, were enriched with categories such as signalling functions associated with transforming growth factor beta (TGF-β) and protein deubiquitination (FIG. 3″). Interestingly, TNF represented a second hub within the vedolizumab UC cohort, upregulated in vedolizumab responders. In the anti-TNF UC cohort however, the targets of the main protein hub suppressor of cytokine signalling protein 3 (SOCS3) distinguishing responders and non-responders with respect to the CD4⁺ cells were associated with biological processes such as the JAK-STAT pathway and interleukin-35 signalling (FIG. 3″).

In monocytes though, our findings were more intriguing since we observed not only cohort specific divergent effects mediated by hubs, but also particular hubs playing roles in UC and CD. For instance, in CD patients, TNF alpha induced protein 3 (TNFAIP3 or A20), NFKB Inhibitor Alpha (NFKBIA) and Early Growth Response 1 (EGR1) were found to be the top hubs with significant enrichment of distinct functional categories in the anti-TNF and vedolizumab cohorts respectively (FIG. 3″). However, EGR1 was also identified as the top hub mediating the same biological processes (negative regulation of apoptotic process, cytokine-mediated signalling pathway and negative regulation of gene expression) in both the anti-TNF UC and VDZ CD cohorts. At the expression level though, in contrast to vedolizumab remitters, EGR1 was found to be down-regulated in anti-TNF non-responders (Table 17″, Table 1″).

### Example 3-e Predicting Endoscopic Remission in Ulcerative Colitis

The top -omic layer in terms of the variance contribution to the explanatory LF identified for each dataset was used for the predictive modelling. The CD4⁺ transcriptomic dataset was inferred as the top potential predictor of endoscopic remission in anti-TNF treated UC patients. To test and verify this, we used feature reduction techniques to infer genes within this particular gene expression layer which could properly differentiate patients who did and did not endoscopically respond to anti-TNF. After dimensionality reduction using feature prioritization, we identified 30 CD4⁺ expressed genes, which could explain the endoscopic remission-based segregation of patients. The top 5 features selected based on the score generated by the multi-variate filter RReliefF (ELOVL4, FGL2, CTSW, DDX11, LYZ), were used for performing the Machine-Learning based classification using multiple classifiers including a stacked ensemble classifier. Mean accuracy rates for the 5 selected features from the CD4⁺ transcriptomic datasets using a 6-stack ensemble classifier were reported at 92% (Table 12″, FIG. 4A″). On the other hand, the mean accuracy rates for 5 randomly selected features post normalization was around 57.5% (Table 12″, FIG. 4B″), suggesting that the expression (FIG. 4C″) of the features used for the classification post feature prioritization were indeed predictive in nature. Additionally, applying this predictive 5-gene CD4⁺ panel to a vedolizumab treated UC cohort resulted in an overall 61.1% accuracy. Similarly, this 5 gene biomarker did not predict anti-TNF induced endoscopic remission in anti-TNF treated CD patients (56.4% accuracy).

In contrast to the anti-TNF treated cohort, genetics significantly contributed to the prediction of vedolizumab remission in UC patients. A 5-marker panel with single nucleotide polymorphisms (SNP) in FAM129A, ELMO1, TRIP13, PTAR1 and ASAH1 accurately predicted vedolizumab remission in UC patients (accuracy 84.2%) (FIG. 7A″, Table 2″). In contrast, a randomly chosen panel of 5 SNPs could predict vedolizumab-induced endoscopic remission clearly less accurately (59.9% accuracy) (FIG. 7B″, Table 2″). The vedolizumab UC genetic marker panel performed poorly (36.4% accuracy) in classifying responders and non-responders in the anti-TNF UC cohort. Similarly, the genetic marker panel for the anti-TNF UC cohort had a poor classification performance (51.4% accuracy) on the VDZ UC cohort.

### Example 3-f Predicting Endoscopic Remission in Crohn's Disease

In both CD cohorts, the genetic layer contributed the most to the explanatory LF associated with endoscopic remission. Hence, two genetic models were built predicting therapeutic success of anti-TNF agents and vedolizumab respectively. SNPs within TRAPPC4, CDKAL1, ACVRL1, TSPAN14 and PCNP accurately predicted remission to anti-TNF agents (accuracy 81.3%, as opposed to 40.7% in case 5 random genes were chosen) (FIG. 7C-D″, Table 2″). Similarly, SNPs within SKAP2, HAUS1, C3orf67, SEC14L6 and ATP6V0D1 predicted remission to vedolizumab therapy (accuracy 77.2%, instead of 52.8% in case of 5 random genes) (FIG. 7E-F″, Table 2″). We also checked the performance of cohort specific markers on other cohorts to evaluate cross-performance: the marker panel predicting VDZ and anti-TNF remission in CD had lower accuracy rates in the anti-TNF (41.7%) and VDZ cohorts (58.5%) respectively. In addition, we identified a 5 gene panel in monocytes predictive of the vedolizumab remission in CD patients (FIG. 4D, F″) with an accuracy rate of 98% as against 62% for 5 random features (FIG. 4E″, Table 2″).

### Example 3-g Ulcerative Colitis and Crohn's Disease, Two Different Molecular Entities

Pathway analysis of the various sets of features revealed both cell-type-, cohort- and disease-specific signalling pathways (Table 18″) which were found to be enriched (adjusted p-value <=0.05) in the feature sets. Most (35 out of 38) of the signalling pathways were unique to particular cell-/tissue-types in specific cohorts. Interestingly, pathways involved in the drug responses were distinct to UC and CD with only 3 of the 38 pathways being common and 19 of the pathways unique to CD. Pathways such as activation of matrix metalloproteinases, signalling related to interleukins-4, -6 and -13, activation of AP-1 class of transcription factors such as JUN among others were active in the UC cohorts. However, in the CD cohorts, a different set of pathways such as those associated with IL-10 signalling, interferon signalling, co-stimulation by the CD28 protein family, PD-1 signalling, ZAP-70 translocation to the immunological synapse among others were identified (Table 18″). We also observed distinct functional differences between UC and CD from a network perspective. UC and CD shared only one protein hub (distinguishing remitters and non-remitters), EGR1, with enriched functional terms among its targets (Table 17″).

### Example 3-g Methods

Patient Selection

This study was conducted at the tertiary IBD referral centre of the University Hospitals Leuven (Leuven, Belgium). We prospectively collected baseline inflamed colonic and ileal tissue, peripheral blood mononuclear cells (PBMC), whole blood DNA and serum in consecutive CD and UC patients initiating infliximab, adalimumab or vedolizumab therapy (Table 1″). The IBD diagnosis was made in line with current ECCO guidelines (Maaser, C., et al. J Crohns Colitis 13, 144-164 (2019)) All recruited patients had endoscopy-proven active disease, and all were naïve for the drug initiated at inclusion.

All included patients had given written consent to participate in the Institutional Review Board approved IBD Biobank of University Hospitals Leuven, Belgium (B322201213950/S53684).

Outcomes

Endoscopic remission was assessed at week 8 (adalimumab) or week 14 (infliximab, vedolizumab) (as per national reimbursement criteria) for UC patients, and defined as a Mayo endoscopic sub-score ≤1. According to current ECCO guidelines, (Sturm, A., et al. ECCO-ESGAR Guideline for Diagnostic Assessment in Inflammatory Bowel Disease. J Crohns Colitis (2018) 26 Aug. 2018) CD patients were endoscopically assessed 6 months after therapy initiation, with endoscopic remission, being defined as the complete absence of ulcerations (Schnitzler, F., et al. Gut 58, 492-500 (2009)).

Cell Separation

Using density centrifugation, PBMCs were isolated from a 20 ml blood samples.⁶⁹ Isolated PBMCs were subsequently cryopreserved with dimethyl sulfoxide (DMSO) using Mr Frosty (Thermo Fisher Scientific, Waltham, Mass., USA) for 24 hours at −80° C., and afterwards stored in liquid nitrogen. Frozen PBMCs were subsequently thawed in batches, and CD14⁺ and CD4⁺ T-cells sorted using fluorescence activated cell sorting (FACS) (median purity, 99.8% and 99.0% respectively)

Fluorescence Activated Cell Sorting

Vials with cryopreserved PBMCs were removed from liquid nitrogen and transferred to a waterbath at 37° C. Vials were allowed to warm completely, and left at 37° C. until further processing. One ml of prewarmed cHBSS-CM (Thermo Scientific, Waltham, Mass., USA) was slowly added to each vial and mixed by gently pipetting. Contents of the vial were transferred to a 15 ml conical polypropylene tube, containing 9 ml of prewarmed cHBSS-CM by dropwise addition, followed by gentle mixing. Tubes were centrifuged for 10 minutes at 300 g, supernatant removed and cell pellet resuspended in 5 ml cHBSS-CM containing 100 μg/ml DNaseI, where after incubated at room temperature for 10 minutes. Next, cell concentrations were determined using an ABX Diagnostics Micros 60. After addition of 5 ml cHBSS, tubes were centrifuged for 5 minutes at 400 g and supernatant completely removed. Cell pellet was then resuspended in cHBSS with FcR Blocking Reagent at 200 μl/ml to a concentration of 4.4×10⁷ cells/ml, and incubated at 4° C. for 10 minutes. A titrated amount of antibody cocktail (Table 17″) was then mixed into the cell suspension and incubated at 4° C. for 30 minutes. Cells were washed with 3 ml of cHBSS, centrifuged for 10 minutes at 300 g and resuspended at 5×10⁶ cells/ml. Before acquisition, samples were filtered through a 40 μm mesh cell strainer, and DAPI added to a final concentration of 0.1 μg/ml. Cells were sorted (BD FACSAria III) into 5 ml polypropylene tubes precoated with cHBSS, containing 250 μl of HBSS/8% foetal bovine serum. During sorting, sample and collection tubes were kept at 4° C. Sorted cells were lysed (QIAshredder, Qiagen, Hilden, Germany), and stored at −80° C. till further processing.

Isolation of RNA

Frozen inflamed biopsies, stored at −80° C. in RNALater buffer (Ambion, Austin, Tex., USA), were lysed using the FastPrep Lysing Matrix D tubes (MP Biomedicals, Brussels, Belgium) with RLT lysis buffer (Qiagen, Hilden, Germany) after thawing. Total RNA was extracted from lysed tissue and sorted cells using the AllPrep DNA/RNA Mini kit (Qiagen, Hilden, Germany), according to the manufacturer's instructions. The quantity and integrity of all RNA was assessed with a Nanodrop ND-1000 spectrophotometer (Thermo Scientific, Waltham, Mass., USA) and a 2100 Bioanalyzer (Agilent, Waldbronn, Germany). Extracted RNA was stored at −80° C. until further processing.

RNA Sequencing

After library preparation using the TruSeq Stranded mRNA protocol (Illumina, San Diego, USA), next-generation 75 base pairs single-end sequencing was performed using the Illumina HiSeq 4000NGS. Raw RNA-sequencing data were aligned to the reference genome (Hisat2 version 2.1.0) and absolute counts generated using HTSeq (Kim, D., et al. Nat Methods 12, 357-360 (2015) and Anders, S., Pyl, P. T. & Huber, W. Bioinformatics 31, 166-169 (2015)). Gene expression data were pre-processed, with only genes having at least 10 normalised read counts in at least 70% of the samples were considered for further analysis. Genes contributing to less than 1% of the variance across all samples were discarded. The resulting data were normalized using the varianceStabilizingTransformation function of the DESeq2 R package.

Serum Proteomics

Ninety-two unique proteins were measured using the Olink® INF I panel (Olink Proteomics AB, Uppsala, Sweden) (Table 18″).⁷² Proteins contributing to less than 1% of the variance across all samples were discarded. Data were quality controlled and normalized using an inter-plate control and an internal extension control, to adjust for inter- and intra-run variation. The final assay read-out is presented in Normalized Protein eXpression (NPX) values, which is an arbitrary relative unit on a log 2-scale, where a high value corresponds to a higher protein expression. Assay validation data are available on the manufacturer's website (www.olink.com).

Genotyping Data

Immunochip genotype data were available in a subset of patients (55.9%) (Jostins, L., et al. Nature 491, 119-124 (2012)), from which the global mutation profiles were transformed into a “genetic risk burden” matrix by mapping the mutations onto the protein coding genes. This “genetic risk burden” represents the degree to which a particular gene is affected in a patient by mutations which fall within its exonic regions. The mutation-gene mapping associations based on the genomic co-ordinates were retrieved from the chromosomal report files from the dbSNP database (Sherry, S. T., et al. Nucleic Acids Res 29, 308-311 (2001)).

MOFA Aided-Omics Data Integration MOFA was used to integrate and evaluate the principle sources of variation in the multiple -omic layers available in this study (Argelaguet, R., et al. Mol Syst Biol 14, e8124 (2018)). The DropFactorThreshold and maxiter parameters which determine the variance allowance of the LFs and the number of iterations were set at 0.02 and 10000 respectively. Only LFs with weights assigned to all the samples were considered. Multiple regression (P-value <=0.2) was used to identify the explanatory LFs which are LFs which correlated with the endoscopic outcomes. The most dominant -omic layers corresponding to the explanatory LFs were identified from the variance contributions of the -omic layers to the LFs.

The MOFA model object was compiled using the default model and training options with the exception of the DropFactorThreshold (representative of variance cut off) and maxiter (number of iterations) which were set at 0.02 and 15000 respectively. The compiled MOFA model object was executed using the runMOFA function to analyse and integrate the multi-omic datasets. The weights corresponding to each of the different omic layers for every identified Latent Factor (LF) were retrieved using the getFactors function. The calculateVarianceExplained function was used to determine the contribution of the LFs towards the variance corresponding to each of the different -omic datasets. LFs with no weight contributions from any of the patients were discarded. Using a multiple regression model, we identified the explanatory LFs which contribute to endoscopic outcome. LF-trait relationships with a p-value of <=0.2 were considered to be significant. The most dominant omic data layers in the explanatory LFs were determined from the variance contribution of the omic layers towards the LFs (FIG. 6″).

Feature Selection and Predictive Modelling

The gene expression data layers identified as the contributing -omic layers were subjected to feature selection and machine learning based predictive modelling using the DaMirSeq R package, (Chiesa, M. et al. Bioinformatics 34, 1416-1418 (2018)) to identify genes whose expression signatures can discern patient groups (patients with/without endoscopic remission). The count filtered version of the gene expression files were used as input for DaMirSeq. Features were selected from the normalized and adjusted expression matrix using a Principal Component Analysis which identified principle components (PC) that correlated (R²>=0.2) with the clinical co-variates. The features corresponding to the correlating PCs were further reduced using pair-wise absolute correlation which removed the features with the largest mean absolute correlation (R2>=0.85). The reduced list of features was further ranked based on the z-score standardized version of the scores calculated by the multivariate filter RReliefF. The top 5 features as ranked by the standardized z-score were selected for the ensemble learning based classification procedure. In order to avoid over-fitting, the samples were first split into a training (TR1) and test set (TS1) by bootstrap sampling. Another pair of training (TR2) and test set (TS2) were obtained from TR1. While TR2 is used to train the six individual classifiers, TS2 was used to test their accuracies. Random features for the classification were selected before to the feature prioritization.

For the classification of the genetic entities, a stacked model consisting of five different classifiers (Adaboost, Random Forest, Extreme Gradient Boosting, SVM, and Gaussian Naive Bayes) was used. Probabilities generated from each of the classifiers were then used along with a logistic regression model which served as the meta-classifier to discriminate against the best and worse models for each selection based on the probability and thus certain of the classifier's prediction. The mean-decrease-in-impurity importance of a feature was computed by measuring how effective the feature is at reducing uncertainty (classifiers) or variance (regressors) when creating decision trees within random forests. To find the most influential features in the large feature space, Minimum Redundancy Maximum Relevance (mRMR) was used. The classifier was run for 100 iterations using a stratified train/split test configuration, where 75% of the data was used for training and 25% for testing. The same base random seed (42) was set for all 3 of the experiments and for each run, a new random number was set as a round-seed from the seed number.

Pathway and Network Analysis

The R package ReactomePA was used to identify canonical pathways over-represented (p-value <=0.05) within each feature list (Yu, G. & He, Q. Y. Mol Biosyst 12, 477-479 (2016)). Interaction networks were used for interpreting the importance of the discriminatory features. For this purpose, directed protein-protein interactions and transcriptional regulatory interactions were retrieved from OmniPath2 and DoRothEA (Garcia-Alonso, L., et al. Cancer Res 78, 769-780 (2018) and Turei, D., Koresmaros, T. & Saez-Rodriguez, J. Nat Methods 13, 966-967 (2016)). Tissue and cell-type specific networks were generated by pruning the parent OmniPath2 and DoRothEA networks using tissue- and cell-type specific gene expression retrieved from Bgee (Komljenovic, A., Roux, J, Wollbrett, J., Robinson-Rechavi, M & Bastian, F. B. BgeeDB, an R package for retrieval of curated expression datasets and for gene list expression localization enrichment tests. F1000 Res 5, 2748 (2016)). Genes found to be expressed in at least 13 gold-quality datasets were considered to be expressed in colon. For the ileal and mononuclear cells, no gold-quality datasets were found and hence the silver-quality datasets were used. Network topology measures such as the degree were calculated for the features using the R package igraph on the tissue- and cell-type specific networks (Csardi, G. & Nepusz, T. InterJournal Complex Systems, 1695 (2006)). Degree was determined as the total of the outgoing edges. Enrichment with Gene Ontology Biological Process terms of the targets of the discriminatory features was carried out using the hypergeometric enrichment test. Only features with at least four downstream targets were considered for the enrichment. Only terms with a Benjamini-Hochberg corrected hypergeometric enrichment P-value of <=0.05 were considered as significant.

### Example′″ 3 Discussion

This is, to our knowledge, the largest multi-omics study in IBD, integrating genomic, proteomic and multiple transcriptomic (tissue, CD4⁺ T-cells and monocytes) datasets. Additionally, this study is the first to understand molecular pathways involved in the response to anti-TNF agents and vedolizumab therapy, with subsequent identification of potential predictive biomarkers through a multi-omics approach.

In contrast to what one would expect, we identified the genetic and immune cell (CD4⁺ T cells and monocytes) layers, and not the inflamed mucosal tissue, as the dominant -omic layers associated with endoscopic outcome. Likewise, a similar multi-omics approach in ustekinumab treated patients also highlighted the importance of immune cells in understanding molecular mechanisms and prediction of therapeutic success.²³ However, this observation could be explained by a characterisation of purified immune cells, in contrast to the study of bulk tissue transcriptomics which is prone to noise due to the variety of cells represented. Interestingly, the majority of identified features (88.6%) distinguishing responders from non-responders were different between anti-TNF and vedolizumab treated patients, suggesting that both drugs act differently and thus explaining why some patients respond to one class of drugs, but not to the other. However, the minority (11.4%) of similar features might point to a more refractory signature within IBD patients. Additionally, we identified clear differences in features, pathways and networks between CD and UC patients treated with the same compound, suggesting that predictive biomarkers can not necessarily be applied in both diseases.

Using network analysis, we identified several hubs linked to the therapeutic success of anti-TNF and vedolizumab, which provided novel insights in their mode of action. First, the identification SMAD7 as central hub in the vedolizumab UC CD4⁺ T cell network may come as a surprise, especially after the failure of Mongersen, an oral SMAD7 antisense, in the phase III REVOLVE trial in CD (Celgene Corporation. Celgene Provides Update on GED-0301 (mongersen) Inflammatory Bowel Disease Program. (Celgene Corporation, 2017) Oct. 19, 2017 BusinessWire) IBD is indeed characterised by a decrease in TGF-β1 due to increased levels of Smad7 (Monteleone, G., et al. J Clin Invest 108, 601-609 (2001)). As TGF-β1 inhibits T-cell proliferation and differentiation and reduces macrophage activation and dendritic-cell maturation, it has an essential regulatory role in the control of experimental colitis (Boirivant, M, et al. Gastroenterology 131, 1786-1798 (2006)). However, TGF-β1 also controls the formation and maintenance of gut-resident memory T cells by regulating migration and retention through inhibiting the expression of α4β7 (Zhang, N. & Bevan, M J. Immunity 39, 687-696 (2013)). Hence, Smad7 expression in T cells—upregulated in vedolizumab responders—can influence α4β7 expression through TGF-β1, which may explain its central role within the vedolizumab UC network. However, the identification of TNF as second hub within the vedolizumab UC CD4⁺ T cell network may be an even bigger surprise. Being upregulated in vedolizumab responders, an increased TNF burden seems necessary for vedolizumab efficacy. SOCS3 was identified as the most important hub within the anti-TNF UC remission network. Increased SOCS3 expression has been linked with a polarisation towards T_{H}2 cells (Li, Y., et al. Cytokine Growth Factor Rev 23, 127-138 (2012)). Downregulation of SOCS3 in non-responders could result in an activation of STAT3 with subsequent prolonged survival of pathogenic T cell and increased expression of TNFα (Sugimoto, K. World J Gastroenterol 14, 5110-5114 (2008)) Interestingly, an increased colonic TNF burden in anti-TNF non-responders has already earlier been suggested.

In contrast to UC, C-X-C Motif Chemokine Receptor 4 (CXCR4) was identified as the key hub protein in the anti-TNF CD CD4⁺ T cell network.

The monocyte anti-TNF CD network was held together by TNFAIP3, again upregulated in remitters. Being involved in maintaining the intestinal barrier and preventing excessive cytokine production in myeloid cells (Kolodziej, L. E., et al. PLoS One 6, e26352 (2011) and Vereecke, L., et al. Nat Commun 5, 5103 (2014)) TNFAIP3 CD4⁺ T cell expression is inhibited by anti-TNF agents and acts as a master switch in TNFα blockade driven IL-17A expression.

In contrast to the TNF induced NF-kappaB signalling in CD4⁺ T cells within the UC vedolizumab network, NFKBIA was upregulated in the monocyte CD vedolizumab network, resulting in a decreased cellular response to TNF and NF-kB signalling in vedolizumab responders. This rather opposing findings reinforces the importance of using separated cell subsets for discovery science in immune-mediated disease. NF-kappaB is a critical mediator of macrophage inflammatory responses (Pagliari, L. J, et al. Mol Cell Biol 20, 8855-8865 (2000)), with the increased NFKBIA expression in monocytes suggesting a polarization towards M2 macrophages (Gordon, S. & Martinez, F. O. Immunity 32, 593-604 (2010)). Our data suggest that monocytes from vedolizumab remitters already have an a priori tendency towards M2 macrophage differentiation.

Finally, EGR1 played a central role in the vedolizumab network in CD monocytes, being markedly upregulated in remitters. Intriguingly, in the anti-TNF UC monocyte network, EGR1 was also identified the top feature hub, however downregulated in anti-TNF non-responders.

Hence, we report for the first time a regulatory hub which has opposing effects in anti-TNF and vedolizumab treated patients.

Based on our network analysis, it is clear that different biologicals act differently in a cell and disease specific manner. Based on the top -omic layers in terms of the variance contribution to the explanatory LFs identified for each dataset, we designed predictive panels for therapeutic success which were treatment and disease specific, as confirmed through cross-validation. Interestingly, 3 out of the 4 predictive panels included only genetics, which offers translational opportunities due to its stability as opposed to the dynamic nature of transcriptomics.

Despite the use of training and test sets to reduce the risk of over-fitting, the lack of a validation cohorts warrants caution. Furthermore, we do acknowledge the issue of missing data. Missing data is not uncommon, and reflects daily clinical practice, especially if many different specimens (tissue, serum, blood) have to be collected and processed from the same individual. Luckily, the MOFA tool has been designed to cope with this limitation.⁶⁵ Furthermore, we realise we did not cover the entire immunome, but only focused on the CD4⁺ T cell and monocyte compartment.

In conclusion, by applying multi-omics integration, we identified separate key hubs in the immune cell network, affecting therapeutic success of vedolizumab and anti-TNF agents in both CD and UC. Surprisingly, almost all hubs were enriched in treatment-remitters, providing novel insights in their core mechanism of action. we identified predictive markers which now have to be validated within independent cohorts or clinical trials, but which bring personalised medicine in IBD one step closer.

### Examples ′″4 on Biomarker Signature that for a α₄β₇-Integrine Blocker Treatment

### Example 4 a Method

### Example 4 a (i) Patient Selection

This prospective study was carried out at the University Hospitals Leuven (Leuven, Belgium). Independent validation cohorts were recruited in the same center as well as in the IBD center of the Hospital Clinic (Barcelona, Spain). Endoscopy-derived inflamed colonic biopsies were obtained from IBD patients initiating biologic therapy (vedolizumab, adalimumab or infliximab). All patients had endoscopically proven active disease, and they all had to be naïve for the drug that was initiated at inclusion. Patients received vedolizumab 300 mg at baseline, week 2 and week 6, with subsequent administration every 8 weeks. All CD patients received an additional infusion at week 10. In case of anti-TNF therapy, patients received infliximab (CTP-13) 5 mg/kg at baseline, week 2 and week 6, with subsequent administration every 8 weeks. Adalimumab was administered 160 mg subcutaneously (SC) at baseline, 80 mg SC at week 2 with subsequent 40 mg every other week thereafter. To reduce the risk of including treatment failures secondary to immunogenicity (and not drug mechanistic failure) or nondrug-related responders, all anti-TNF treated patients had to have a good drug exposure, defined as a maintenance trough level or >3.0 μg/ml for infliximab and >5.0 g/ml for adalimumab. Due the lack of agreement on the targeted threshold for vedolizumab, if any, we did not include an exposure requirement in the definition of (non-) response for vedolizumab.

All biopsies were taken at the most affected site, at the edge of the ulcerative surface. Biopsies were taken during endoscopy prior to the start of therapy, stored in RNALater buffer (Ambion, Austin, Tex., USA) and preserved at −80° C. Additional biopsies were immediately fixed in formalin's fixative for up to 5 hours and then dehydrated, cleared and paraffin-embedded for histological examination and immunohistochemistry.

### Example 4 a (ii) Endoscopic Outcomes

Outcome was assessed objectively trough ileocolonoscopy at a fixed time point. In CD patients, endoscopic remission was evaluated after 6 months, and defined as a complete absence of ulcerations, (Schnitzler F, Fidder H, Ferrante M, et al. Gut 2009; 58:492-500). whereas in UC it was based as a Mayo endoscopic sub-score ≤1. Due to national reimbursement criteria, all UC patients were endoscopically assessed at week 8 (adalimumab) or week 14 (infliximab and vedolizumab).

### Example 4 a (iii) Isolation of RNA

Total RNA from inflamed biopsies was extracted using the AllPrep DNA/RNA Mini kit (Qiagen, Hilden, Germany) according to the manufacturer's instructions, after tissue lysis using the FastPrep Lysing Matrix D tubes (MP Biomedicals, Brussels, Belgium) with RLT lysis buffer (Qiagen, Hilden, Germany). The integrity and quantity of all RNA was assessed with a 2100 Bioanalyzer (Agilent, Waldbronn, Germany) and a Nanodrop ND-1000 spectrophotometer (Thermo Scientific, Waltham, Mass., USA). Extracted RNA was stored at −80° C. until further processing. For the Barcelona validation cohort, RNA was extracted using the RNEasy Mini Kit (Qiagen, Hilden, Germany).

### Example 4 a (iv) RNA Sequencing

Next-generation 75 base pair single-end sequencing was performed using the Illumina HiSeq 4000NGS, after library preparation using the TruSeq Stranded mRNA protocol (Illumina, San Diego, USA) according to the manufacturer's instructions. Raw RNA-sequencing data were aligned to the reference genome using Hisat2 version 2.1.0, (Kim D, Langmead B, Salzberg S L. Nat Methods 2015; 12:357-60) absolute counts generated using HTSeq, (Anders S, Pyl P T, Huber W. Bioinformatics 2015; 31:166-9) where after counts were normalized, protein coding genes selected according the Ensemble hg 19 reference build, (Yates A, Akanni W, Amode M R, et al. Ensembl 2016. Nucleic Acids Res 2016; 44:D710-6) and differential gene expression assessed using the DESeq2 package. (Love M I, Huber W, Anders S. Genome Biol 2014; 15:550) RNA-sequencing data have been deposited in the ArrayExpress database at EMBL-EBI (www.ebi.ac.uk/arrayexpress) under accession number E-MTAB-7845. Pathway analysis was performed using the Gene Set Enrichment Analysis software (GSEA, Broad Institute, Massachusetts Institute of Technology, and Regents of the University of California, USA), (Mootha V K, Lindgren C M, Eriksson K F, et al. Nat Genet 2003; 34:267-73) and Ingenuity Pathway Analysis (IPA, Aarhus, Denmark). Cell deconvolution was performed using the xCell online tool, which harmonized 1822 pure human cell type transcriptomes allowing the distinction of 64 immune and stromal cell types (Aran D, Hu Z, Butte A J. Genome Biol 2017; 18:220).

### Example 4 a (v) Quantitative RT-PCR

Gene expression (DCHS2, PIWIL1, MAATS1, RGS13) in inflamed colonic biopsies was studied trough quantitative real-time polymerase chain reaction (qPCR) analysis. cDNA was synthesized from 0.500 μg of total RNA using the RevertAid H Minus First Strand cDNA synthesis kit (Fermentas, St. Leon-Rot, Germany) according to the manufacturer's protocol. The primers for the housekeeping @-actin gene were synthesized by Sigma-Genosys (Haverhill, UK) (Table 3′″) and 10 μM stock solution was used to make the reaction mixture (5 μl SybrGreen, 0.2 μM FW & RV primer, 2 μl cDNA sample, 2.8 μl RNAse-free H2O). All samples were amplified in duplicate reactions. Samples were analysed with the Lightcycler 480 (Roche, Basel, Switzerland). The following amplification program was used: 5′ 95° C., 45×(10″ 95° C., 15″ 60° C., 15″ 72° C.), 5″ 95° C., 1′60° C., 4° C.

To determine the expression of all other genes (P/WIL1, MAATS1, DCHS2 and RGS13), validated target-specific primers were used for TaqMan qPCR (Table 4′″). A total reaction volume of 20 μl was made: 10 μl TaqMan fast advanced master mix, 2 μl cDNA sample, 7 μl RNAse-free H₂O, 1 μl TaqMan assay (containing both primers and probe). All samples were amplified in duplicate reactions. Samples were analyzed with the Applied Biosystems 7500 Fast (Applied Biosystems, Foster City, USA). The following amplification program was used: 5′ 95° C., 40×(3″ 95° C., 30″ 60° C.), 4° C. mRNA-levels were normalized to the housekeeping gene p-actin and quantified using the comparative (ΔΔ) Ct method.

### Example 4 a (vi) Immunohistochemistry

To localize the corresponding proteins of the predictive panel in colonic mucosa, immunohistochemical stainings were performed on 5 μm-thick step slides prepared from paraffin formalin-fixed endoscopy-derived inflamed colonic biopsies from IBD patients, taken prior to vedolizumab initiation. Endogenous peroxidase activity was blocked in deparaffined sections by incubating the slides for 20 min in a 0.3% solution of H₂O₂ in methanol. Epitope retrieval was performed by heating the slides for 30 min in Tris/EDTA buffer (pH 9) at 98° C. Specific protocols for each protein are summarized in Table 5′″. All procedures were conducted automatically by the BOND MAX autostainer. The BOND polymer refine Detection kit (Leica Microsystems Ltd, Heerbrugg, Switzerland) was used for visualization of bound primary antibody according to the manufacturer's instructions. An IBD experienced pathologist (GDH) evaluated all stains. Microscopic images were acquired with Leci Application Suite V4.1.0. software using a Leica DFC290 HD camera (Leica Microsystems Ltd, Heerbrugg, Switzerland) mounted on a Leica DM2000 LED bright field microscope.

### Example 4 a (vii) Statistical Analysis

All machine learning based analyses were carried out using R version 3.5.0 (R Development Core Team, Vienna, Austria). Unlike conventional statistics, for machine learning purposes the initial vedolizumab dataset (n=31 samples) was randomly partitioned into a training (⅔) and validation (⅓) set. Predictive modelling was performed using the randomGLM (RGLM) package, which shares the advantages of a random forest (excellent predictive accuracy, feature importance measures, out-of-bag estimates of accuracy) with those of a forward selected generalized linear model (interpretability) (Song L, Langfelder P, Horvath S. Bioinformatics 2013; 14:5). Parameter choices were optimised according to the developers suggestions, with parameters nBags=100, nFeaturesInBag=5, nCandidateCovariates=5. The identified signature was validated in several independent cohorts using ConsensusClusterPlus (Wilkerson M D, Hayes DN. ConsensusClusterPlus: a class discovery tool with confidence assessments and item tracking. Bioinformatics 2010; 26:1572-3) qPCR expression results were used in binary logistic regression analysis, whereupon predicted probabilities were used to assess performance with receiver operating characteristics (ROC) analysis. A false discovery rate (FDR) correction was applied during differential gene expression, to correct for multiple testing. A two-tailed FDR-corrected p-value <0.25 was considered significant. For all other analysis, a two-tailed nominal p-value <0.05 was considered significant.

### Example 4 Results

Patient Characteristics

Thirty-one patients with endoscopically active colonic inflammatory bowel disease (11 CD, 20 UC) with a median (IQR) disease duration of 8.4 (4.0-15.3) years were included prior to their first vedolizumab administration (Table 1′″). One third (n=10, 32.3%) received vedolizumab as first-line biological therapy. In UC patients, an endoscopic remission rate of 65.0% was observed after 14 weeks, whereas 54.5% of CD patients achieved endoscopic remission after 26 weeks. Remitters and non-remitters did not significantly differ in diagnosis, gender, age, concomitant steroids, previous anti-TNF exposure, body mass index or smoking behavior (p>0.05). Baseline features of the validation cohorts are also reported in Table 1′″.

Additionally, colonic biopsies from 20 actively inflamed patients (6 CD, 14 UC) initiating anti-TNF therapy (12 adalimumab, 8 infliximab), of whom 17 (90.0%) were entirely anti-TNF naïve, were collected. None of them had been exposed to vedolizumab before (Table 6′″).

Differential Gene Expression and Deconvolution

Within the inflamed colonic biopsies prior to vedolizumab initiation, 186 genes were differentially expressed between remitters and non-remitters at a nominal p<0.005 level (Table 7′″). Among them, only 44 genes remained significantly different applying a conservative 0.25 FDR threshold of significance. However, just five genes reached the stringent FDR 0.05 cut-off threshold of significance: KRT23, TMEM35, DCHS2, CLDN8 and IFI6 (FIG. 1′″). None of them was differentially expressed between CD and UC samples (p>0.05). Genes previously linked to anti-TNF non-responsiveness, were not differentially expressed between vedolizumab responders and non-responders: OSM (p=0.76), IL13RA2 (p=0.54), TREM1 (p=0.46). Similarly, no significant differential expression was observed in MadCAM-1 (p=0.59), integrin a4 subunit ITGA4 (p=0.97) or integrin b7 subunit ITGB7 (p=0.99) expression levels.

Pathway analysis using IPA revealed rather vague top canonical pathways (granulocyte adhesion and diapedesis (p=6.6×10⁻⁸) and agranulocyte adhesion and diapedesis (p=1.7×10⁻⁴), enriched in non-remitters. Similarly, we performed a more focused gene enrichment analysis using GSEA, looking at GeneOntology (GO) gene sets covering leukocyte migration and cell adhesion in more detail because of the mode of vedolizumab action. The GO leukocyte migration gene set, among many other trafficking and adhesion gene sets, was indeed significantly enriched in non-remitters (p=0.006) (Table 8′″). Predicted upstream regulated in vedolizumab non-remitters included interleukin 1β (p=2.3×10⁻¹⁵), tumor necrosis factor (p=6.7×10⁻¹⁴) and NFκB (p=2.0×10⁻¹³). Deconvolution methods showed a significant enrichment of effector memory CD4 T-cells (CD4 TEM)(p=0.008), monocytes (p=0.005), M1 macrophages (p=0.05) and Tregs (p=0.05) in non-remitters prior to vedolizumab initiation. In contrast, naïve B-cells cells were significantly enriched in colonic biopsies of remitters (p=0.03) (FIG. 2′″).

A Four-Gene Based Model Predicting Endoscopic Response to Vedolizumab Therapy

The initial dataset containing 31 inflamed colonic IBD biopsies prior to vedolizumab therapy, was randomly split by the RGLM R package in a discovery (⅔, n=20) and validation (⅓, n=11) set. Within the dataset of all 44 differentially expressed genes (at the FDR 0.25 level), we identified a 4-gene signature predicting endoscopic remission to vedolizumab using randomized general linear regression. A model containing RGS13, DCHS2, MAATS1 and PIWIL1 expression could accurately (accuracy 80.0%) predict remission in the discovery cohort (9 non-remitters, 11 remitters). Similarly, the same model could accurately differentiate vedolizumab remitters from non-remitters in validation cohort 1 (3 non-remitters, 8 remitters) (Table 2′″).

Subsequently, we recruited another 16 patients initiating vedolizumab (11 non-responders, 5 responders, validation cohort 2) (Table 1′″), in whom we could accurately predict endoscpic remission (81.3%) through unsupervised consensus clustering based on the expression of the 4 identified genes (Table 2′″). Combining validation cohort 1 and 2 together (14 non-responders, 13 responders) ultimately resulted in an 88.9% accuracy (85.7% sensitivity, 92.3% specificity, 92.3% positive predictive value (PPV), 85.7% negative predictive value (NPV), positive likelihood ratio (LR+) 11.1 and negative likelihood ratio (LR−) 0.15). All 4 identified genes were significantly upregulated in remitters, with PIWIL1 not at all expressed in any of the non-remitters (FIG. 5′″).

Validation of the Four-Gene Model in a Publicly Available Dataset from the GEMINI Long-Term Extension Program

Publicly available transcriptomic data in vedolizumab treated patients are very limited.¹¹ Therefore, we could validate our predictive signature only in a small independent cohort of 13 UC patients (9 non-remitters, 4 remitters; validation cohort 3), treated during the GEMINI long-term extension program (Gene Expression Omnibus database GSE73661). In this historic cohort, the 4-gene panel could accurately identify those patients who would not benefit from vedolizumab therapy (NPV 100.0%, LR− 0.0, overall accuracy 76.9%) (Table 2′″).

Importantly, the combination of all validation cohorts (validation cohort 1-2-3) did confirm a predictive accuracy >80.0% in both CD and UC patients separately.

Validation of the Four-Gene Model in an Independent Cohort Using qPCR

This 4-gene panel was subsequently tested in an additional cohort from Belgium and Spain using qPCR. (30 UC, 7 CD) (Table 1′″). Using qPCR, the baseline 4-gene predictive panel could accurately differentiate between remitters and non-remitters with an AUC of 78.6% (95% confidence interval 63.8-93.3%, p=0.003) (FIG. 3′″). In contrast, the predictive accuracy of the individual genes was clearly lower: PIWIL1 AUC 69.6% (p=0.05), MAATS1 AUC 61.2% (p=0.20), RGS13 AUC 49.0% (p=0.69), DCHS2 AUC 50.4% (p=0.80).

A Vedolizumab Specific Signature

In order to confirm the vedolizumab-specificity of our 4-gene panel, its predictive accuracy was tested in a cohort of 20 patients initiating anti-TNF therapy (8 remitters, 12 non-remitters). In contrast to vedolizumab treated patients, this signature could not accurately predict endoscopic outcome in anti-TNF treated patients (accuracy 55.0%, LR+ 1.3, LR− 0.6) (Table 2′″).

Immunohistochemistry

Immunohistochemistry was performed to confirm the presence of the 4 identified genes at a protein level, aiming to get a better understanding of their expression in inflamed colonic biopsies of IBD patients. PIWIL1 expression could not at all be observed in regenerating epithelium (FIG. 6′″), whereas it was clearly expressed in goblet cells and to some extent in stromal cells in inflamed tissue (FIG. 4A′″). In contrast, MAATS1 (C3orf15) was predominantly identified in endothelial cells and only weakly in epithelium and smooth muscle cells (FIG. 4B′″). Likewise, DCHS2 was found in endothelial cells (FIG. 4C′″). Finally, RGS13 was expressed solely in the epithelial barrier, cytoplasmic just above the cell nucleus (FIG. 4D′″).

Despite the therapeutic success of emerging drugs in IBD, (Verstockt B, Ferrante M, Vermeire S, et al. J Gastroenterol 2018; 53:585-590 and Sabino J, Verstockt B, Vermeire S, et al. An update. Therap Adv Gastroenterol. 2019) endoscopic remission rates are still not exceeding 30% (Rutgeerts P, Van Assche G, Sandborn W J, et al. Gastroenterology 2012; 142:1102-1111 e2; Rutgeerts P, Diamond R H, Bala M, et al. Gastrointest Endosc 2006; 63:433-42; quiz 464; Colombel J F, Sandborn W J, Reinisch W, et al. N Engl J Med 2010; 362:1383-95; Danese S, Feagan B, Sandborn W, et al. 13th Congress of ECCO. Vienna, 2018 and Rutgeerts P, Gasink C, Chan D, et al. Efficacy of Ustekinumab in Inducing Endoscopic Healing in Patients with Crohn's Disease. Gastroenterology 2018).

Besides a better patient selection (active endoscopic disease, early disease as opposed to late, . . . ) and individualized dosing scheme using population pharmacokinetic-pharmacodynamic modelling, (Passot C, Pouw M F, Mulleman D, et al. Therapeutic Drug Monitoring of Biopharmaceuticals May Benefit From Pharmacokinetic and Pharmacokinetic-Pharmacodynamic Modeling. Ther Drug Monit 2017; 39:322-326) therapy outcomes could be further improved using predictive biomarkers. In this study, we identified and validated a 4-gene colonic expression panel predicting endoscopic therapeutic success of vedolizumab therapy specifically.

Very little is known about the role of the four identified genes, PIWIL1, MAATS1, RGS13 and DCHS2 in IBD, and even in colonic mucosa to a larger extent. Piwi-like protein 1 (PIWIL1 or HIWI) encodes a member of the PIWI subfamily of Argonaute proteins, (Sasaki T, Shiohama A, Minoshima S, et al. Genomics 2003; 82:323-30) and is known to contribute to stem cell renewal, RNA silencing and translational regulation (Cox D N, Chao A, Baker J, et al. A novel class of evolutionarily conserved genes defined by piwi are essential for stem cell self-renewal. Genes Dev 1998; 12:3715-27 and Ponnusamy M, Yan K W, Liu C Y, et al. PIWI family emerging as a decisive factor of cell fate: An overview. Eur J Cell Biol 2017; 96:746-757). PIWI proteins and PIWI-interacting RNAs (piRNAs) have broader functions in many vital biological processes, including cell proliferation, migration, differentiation, survival and inflammation (Ponnusamy M, Yan K W, Liu C Y, et al. Eur J Cell Biol 2017; 96:746-757 and Ng K W, Anderson C, Marshall E A, et al. Mol Cancer 2016; 15:5). Hence, their involvement in wound healing and tissue regeneration does not come as a surprise (Ponnusamy M, Yan K W, Liu C Y, et al. Eur J Cell Biol 2017; 96:746-757). Although its highest expression is observed in germline tissue, PIWIL1 expression has been reported along the gastro-intestinal tract (Uhlen M, Fagerberg L, Hallstrom B M, et al. Proteomics. Science 2015, 347:1260419). Existing studies mainly focused on the aberrant expression of PIWIL1 in tumors (Ng K W, Anderson C, Marshall E A, et al. Mol Cancer 2016; 15:5), but the biological role of PIWIL1 in IBD has never been elucidated.

As PIWIL1 is upregulated in vedolizumab remitters, it suggests that those patients have an a priori higher likelihood of stem cell renewal as compared to non-responders. PIWIL1 immunohistochemistry on the other hand pointed towards the contribution of goblet cells, which are fully differentiated and hence not expected to represent a more proliferative state. Whether PIWIL1 affects goblet cell function is currently unknown. Nevertheless, PIWI proteins have been linked to play an important role in the unfolded protein response (UPR) which is crucial in alleviating endoplasmic reticulum (ER) stress (Gebert M, Bartoszewska S, Janaszak-Jasiecka A, et al. Sci Rep 2018; 8:16431), and could therefore hypothetically impact goblet cells, which are particularly sensitive to a dysregulation of ER stress (Heazlewood C K, Cook M C, Eri R, et al. PLoS Med 2008; 5:e54). How this is linked to vedolizumab efficacy in particular cannot be answered based on the current study.

In contrast to PIWIL1, MYCBP associated and testis expressed 1 (MAATS1 or C3orf15) and Dachsous Cadherin-related 2 (DCHS2 or Cadherin J) were mainly found on endothelial cells, suggesting that both may interfere with diapedesis and cell migration, key processes in the mode of action of vedolizumab. Overall, MAATS1 is predominantly expressed in the fallopian tube and testis, but expression along the gastrointestinal tract has been reported (Uhlen M, Fagerberg L, Hallstrom B M, et al. Proteomics. Tissue-based map of the human proteome. Science 2015; 347:1260419). However, MAATS1 function is entirely unknown so far. In contrast, DCHS2 is implicated in cell adhesion, considered an unconventional cadherin, and mainly expressed in the reproductive system, the gastrointestinal tract and the brain (Uhlen M, Fagerberg L, Hallstrom B M, et al. Science 2015; 347:1260419 and An C H, Je E M, Yoo N J, et al. Pathol Oncol Res 2015; 21:181-5) Single nucleotide polymorphisms (SNPs) within DCHS2 have been linked to age of onset of Alzheimer's disease and mild cognitive impairment (Kamboh M I, Barmada M M, Demirci F Y, et al. Mol Psychiatry 2012; 17:1340-6 and Vieira R N, Avila R, de Paula J J, et al. Int J Geriatr Psychiatry 2016; 31:1337-1344). Additionally, frameshift mutations within DCHS2 have been reported in colorectal and gastric cancer (An C H, Je E M, Yoo N J, et al. Pathol Oncol Res 2015; 21:181-5).

Finally, Regulator of G-protein signaling 13 (RGS13) was mainly observed in the epithelial barrier. Apart from its abundant expression in innate and adaptive immune cells, it is expressed throughout the digestive system (Uhlen M, Fagerberg L, Hallstrom B M, et al. Proteomics. Science 2015; 347:1260419). Interestingly, RGS13 expression impacts CD4 T cell migration through the RGS13 induced unresponsiveness to CXCL12, despite high levels of its receptor CXCR4 on T cells (Lippert E, Yowe D L, Gonzalo J A, et al. J Immunol 2003; 171:1542-55). Similarly, RGS13 mRNA silencing in human B cell lymphoma enhanced the responsiveness to CXCL12 (Han J I, Huang N N, Kim D U, et al. J Leukoc Biol 2006; 79:1357-68). As CXCL12 and CXCR4 are upregulated and constitutively expressed by intra-epithelial cells (IEC) in patients with active IBD (as opposed to patients with non-IBD inflammation), a positive feedback loop has been suggested: increased expression and secretion of CXCL12 by IEC result in an accumulation of CXCR4⁺ monocytes and T cells, (Dotan I, Werner L, Vigodman S, et al. Inflamm Bowel Dis 2010; 16:583-92 and Sanchez-Martin L, Estecha A, Samaniego R, et al. Blood 2011; 117:88-97) which on their turn contribute to additional CXCL12 expression by IECs (Dotan I, Werner L, Vigodman S, et al. Inflamm Bowel Dis 2010; 16:583-92). But, increased RGS13 expression results in impaired CXCL12 responsiveness, implying less leukocyte trafficking.

Additionally, CXCL12 itself improves the adhesion of α4β7⁺ cells to MadCAM-1 by increasing the α4β7 affinity, without affecting the subcellular distribution of α4β7 (Sun H, Liu J, Zheng Y, et al. Dev Cell 2014; 30:61-70). Whether this also affects vedolizumab efficacy remains unknown. However, as vedolizumab acts on the cellular level by internalizing surface α4β7 and hence impairing the interaction with MadCAM-1, (Rath T, Billmeier U, Ferrazzi F, et al. Front Immunol 2018; 9:1700) one might consider that this disturbance is biologically and clinically most relevant in those settings where there is a high α4β7—MadCAM-1 affinity which would otherwise result in adhesion and diapedesis.

The reduced a priori leukocyte trafficking in vedolizumab remitters, as reflected by an increased RGS13 expression in the 4-gene model, was also observed in our unsupervised transcriptome-wide analysis. This raises the question whether many more escape mechanisms exist to maintain leukocyte trafficking and subsequent intestinal inflammation in non-remitters, regardless of α4β7 blocking. Using deconvolution techniques, we identified a significant enrichment of pro-inflammatory M1 macrophages (M1ϕ) in non-responders, prior to vedolizumab. In contrast to non-classical monocytes essential for intestinal wound healing mediated by M2 ϕ (which are blocked by vedolizumab therapy), (Schleier L, Wiendl M, Binder M-T, et al. OP008 α4β7. Journal of Crohn's and Colitis 2018; 12:S005-S007) classical monocytes can still migrate via the αLβ2-ICAM1 pathway, differentiate in proinflammatory M1ϕ and maintain intestinal inflammation (Schittenhelm L, Hilkens C M, Morrison V L. Front Immunol 2017; 8:1866). Vedolizumab may also s affect the innate immune system, as r described by Zeissig and colleagues (Zeissig S, Rosati E, Dowds C M, et al. Gut 2019; 68:25-39). They demonstrated a switch from a M1 ϕ to a M2 ϕ environment, however only in vedolizumab clinical responders. Our data now demonstrate that endoscopic non-remitters have an a priori abundance of M1 ϕ already, together with an increased proportion of monocytes and CD4+Tₑₘ as compared to remitters. As vedolizumab is not able to reduce the abundance of CD4+Tₑₘ, (Zeissig S, Rosati E, Dowds C M, et al. Gut 2019; 68:25-39) the additional abundance of Tregs in non-responders is not able to dampen the pro-inflammatory environment, despite vedolizumab therapy.

Finally, we observed a significant baseline enrichment of naïve B cells in vedolizumab remitters. The data by Zeissig et al. also pointed towards the B cell compartment, as B cell receptor signaling was significant downregulated upon vedolizumab exposure (Zeissig S, Rosati E, Dowds C M, et al. Gut 2019; 68:25-39). However, the role of the complex B cell biology in IBD pathogenesis is very poorly understood, (Uzzan M, Colombel J F, Cerutti A, et al. Dig Dis Sci 2016; 61:3407-3424) and mainly disregarded after the failure of the anti-CD20 rituximab in randomized trial in UC (Leiper K, Martin K, Ellis A, et al. Gut 2011; 60:1520-6x) Given that B cells are now becoming known as key players in both innate and adaptive immune responses and given that micro-organisms have broad effects on B cell response, (Nothelfer K, Sansonetti P J, Phalipon A. Nat Rev Microbiol 2015, 13:173-84) a better understanding of B cell biology in IBD is absolutely warranted.

Why we observe a significant enrichment of naïve B cells in vedolizumab remitters cannot be fully answered based on our findings, and raises the question whether a vedolizumab induced depletion of this cell population is key to its therapeutic success. Indeed, recent data on a small cohort of HIV-infected IBD patients demonstrated that vedolizumab therapy importantly reduced naïve B cells in intestinal mucosa, (Uzzan M, Tokuyama M, Rosenstein A K, et al. Sci Transl Med 2018; 10) preventing subsequent priming by dendritic cells, who are surveying the mucosal barrier for invading pathogens.

In conclusion, we identified and validated a 4-gene vedolizumab specific signature predicting therapeutic success in IBD, highlighting novel pathways previously unrecognized in vedolizumab efficacy. Additionally, our transcriptome wide unbiased analysis of inflamed colonic biopsies prior to vedolizumab therapy suggested an a priori increased leukocyte trafficking in non-remitters, and provided novel insights in the vedolizumab mode of action, including the involvement of B cell compartment in vedolizumab response.

Particular and preferred aspects of the invention are set out in the accompanying independent and dependent claims. Features from the dependent claims may be combined with features of the independent claims and with features of other dependent claims as appropriate and not merely as explicitly set out in the claims.

Thus, the claims following the detailed description are hereby expressly incorporated into this detailed description, with each claim standing on its own as a separate embodiment of this invention.

### DRAWING DESCRIPTION

### Brief Description of the Drawings

The present invention will become more fully understood from the detailed description given herein below and the accompanying drawings which are given by way of illustration only, and thus are not limitative of the present invention, and wherein:

FIG. 1 is a schematic diagram showing baseline whole blood TREM1 (A), OSM (B), TNF (C) and TNFR2 (D) expression in relation to endoscopic remission later on in both Crohn's disease and ulcerative colitis patients, treated with either adalimumab or infliximab *** p b 0.001, NS=not significant.

FIG. 2 is a schematic diagram showing baseline expression of the different whole blood TREM1 transcripts, including TREM1 mb (A), TREM1×2 (B) and TREM1 sv (C), in relation to endoscopic remission later on in both CD and UC patients, treated with either adalimumab or infliximab. * p b 0.05.

FIG. 3 is a schematic diagram showing baseline expression of the different whole blood TREM1 transcripts baseline whole blood TREM1 expression in relation to endoscopic remission later on in both discovery and validation cohort, visualised by diagnosis (B). ** p b 0.01, *** p b 0.001.

FIG. 4 is a schematic diagram showing baseline mucosal TREM1 (A), OSM (B), TNF (C), IL13RA2 (D) and TNFR2 (E) expression in relation to endoscopic remission later on in both Crohn's disease and ulcerative colitis patients, treated with either adalimumab or infliximab. * p b 0.05, ** p b 0.01.

FIG. 5 is a graphic showing the Fagan nomogram demonstrating the post-test probability of non-response in anti-TNF exposed patients, based on a lower (A) and upper (B) defined threshold of baseline TREM1 expression with a sensitivity and specificity of 90.0% respectively. Pre-test probability representing the non-response rate in the included cohort.

FIG. 6 is a schematic diagram showing baseline whole blood TREM1 expression in relation to endoscopic remission in both Crohn's disease and ulcerative colitis patients, treated with either vedolizumab (A) or ustekinumab (B). Baseline mucosal TREM1 expression in relation to endoscopic remission later on in both Crohn's disease and ulcerative colitis patients, treated with either vedolizumab (C) or ustekinumab (D). NS=not significant

′Figures

FIG. 1′ is showing the variance measures as determined by the MOFA model after integrating the six -omic datasets. (A) shows the variance explained per dataset and (B) provides a graphic representation of the variance contribution of every dataset to the identified Latent Factors (LFs). The variance contributions were measured by MOFA after 15000 iterations. For further details, please refer to the Methods section. TD=transcriptomic dataset.

FIG. 2′ is showing the relative expression profiles of the top ten selected features after normalization, dimensionality reduction and relevance-based filtering steps, linked to biological response (50% decrease in faecal calprotectin by week 8) in the monocyte (A) and colonic (B) transcriptomic datasets. The scaled version, equivalent to a z-score standardization procedure of the score generated by the multi-variate filter RReliefF, implemented in the DaMiRseq package, was used for ranking the features prior to selection.

FIG. 3′: is showing the violin plots displaying the mean accuracy rates from the Ensemble classification approach for the top 10 ranked features selected from among the final list of CD14 transcriptomic features (post normalization, dimensionality reduction and relevance) predictive of 50% faecal calprotectin reduction (A) and 10 randomly selected features from the CD14 transcriptomic dataset post-normalization only (B). Similarly, violin plots displaying the mean accuracy rates from the Ensemble classification approach for the top 10 ranked features selected from among the final list of colonic transcriptomic features (post normalization, dimensionality reduction and relevance) predictive of faecal Calprotectin reduction (C) and 10 randomly selected features from the colonic transcriptomic dataset post-normalization only (D). The scaled version, equivalent to a z-score standardization procedure of the score generated by the multi-variate filter RReliefF, implemented in the DaMiRseq package, was used for ranking the features prior to selection.

FIG. 4′: is showing the violin plots displaying the mean accuracy rates from the Ensemble classification approach for the top 10 ranked features selected from among the final list of CD14 transcriptomic features (post normalization, dimensionality reduction and relevance) predictive of endoscopic response (A) and 10 randomly selected features from the CD14 transcriptomic dataset post-normalization only (B). The scaled version, equivalent to a z-score standardization procedure of the score generated by the multi-variate filter RReliefF, implemented in the DaMiRseq package, was used for ranking the features prior to selection.

FIG. 5′: is showing the future perspectives on how both biomarkers could be implemented after proper validation in independent, randomized cohorts.

FIG. 6′: provides an overview of the -omic datasets from the corresponding samples used for the ustekinumab cohort analysis. n=number of patients

FIG. 7′: provides a multi-dimensional scaling plots which show the separation of samples after the normalization, dimensionality reduction and relevance-based filtering steps (A) for the relationship between CD14 transcriptomic data and faecal calprotectin reduction (B) for the relationship between colonic transcriptomic data and faecal calprotectin reduction.

FIG. 8′: provides an enriched reactome signaling pathways (P-value <=0.05) identified by the stand-alone tool ReactomePA in the colonic response network. The P-value from the hypergeometric enrichment test was adjusted using the Benjamini-Hochberg method.

FIG. 9′: provides a graphical illustration of the largest sub-network with the highest number of differentiating features in the colonic response network. The interactions were retrieved from InnateDB, SIGNOR, and SignaLink2 via the OmniPath webserver while transcriptional regulatory interactions corresponding to the confidence levels A, B and C were downloaded from DoRothEA.

FIG. 10′: Graphical representation of the workflow used to integrate the -omic datasets using a combination of unsupervised (MOFA) and supervised methods (DaMiRseq) followed by the interpretation using biological networks.

### ″ FIGURES

FIG. 1″: Heat maps displaying the variance contributions of every -omic layer to the identified Latent Factors (LFs) in the A) Vedolizumab ulcerative colitis (B) Vedolizumab Crohn's disease (C) Anti-TNF ulcerative colitis (D) Anti-TNF Crohn's disease cohorts. Estimates of variance contributions were determined by using the Multi-Omics Factor Analysis tool. For more detailed information, please refer to the Methods section. GRB=genetic risk burden; TD=transcriptomic dataset.

FIG. 2″: Overlap profile of the CD4+ T cell features distinguishing responders and non-responders in each of the cohorts (A) Overlap profile of the CD14⁺ monocyte features distinguishing responders and non-responders in each of the cohorts (B) Highlighted genes represent overlapping features. The gene expression features were determined by using dimensionality reduction followed by supervised methods with the DaMiRseq R package. VDZ=vedolizumab; CD=Crohn's disease; UC=ulcerative colitis

FIG. 3″: Graphical representation of feature hubs and the associated gene-ontology biological process terms enriched among the downstream interaction targets of the hubs in (A) CD4+ cells and (B) monocytes.

FIG. 4″: Accuracy plots (A) of the top 5 informative and (B) top 5 random CD4+ genes whose expression is predictive of anti-TNF induced endoscopic remission in patients with ulcerative colitis. (C) Relative normalized expression profiles of the top 5 informative CD4+ genes whose expression is predictive of anti-TNF remission in UC patients. Accuracy plots (D) of the top 5 informative and (E) top 5 random CD14+ monocyte genes whose expression is predictive of VDZ induced endoscopic remission in patients with Crohn's disease. (F) Relative normalized expression profiles of the top 5 informative CD14+ genes whose expression is predictive of VDZ remission in CD patients.

FIG. 5″: Summary of the samples per every -omic layer in each of the cohorts. (A) Vedolizumab ulcerative colitis (B) Anti-TNF ulcerative colitis (C) Vedolizumab Crohn's disease (D) Anti-TNF Crohn's disease. n=number of samples.

FIG. 6″: Graphical description of the workflow used in this study.

FIG. 7″: Accuracy plots depicting the performance using individual and stacked classifiers of top 5 informative (A) and random (B) genetic markers predicting vedolizumab induced endoscopic remission in patients with ulcerative colitis. Accuracy plots depicting the performance using individual and stacked classifiers of top 5 informative (C) and random (D) genetic markers predicting anti-TNF induced endoscopic remission in patients with Crohn's disease. Accuracy plots depicting the performance using individual and stacked classifiers of top 5 informative (E) and random (F) genetic markers predicting vedolizumab induced endoscopic remission in patients with Crohn's disease. SVC—Support Vector Classifier. The classifier was run for 100 iterations using a stratified train/split test configuration.

### ′″ FIGURES

FIG. 1 ′″—Top 5 differentially expressed genes

Visual representation of the top 5 baseline differentially expressed genes in mucosal biopsies of patients responding and not responding to vedolizumab therapy. log FC=log fold change; FDR p value=false discovery rate corrected p value. KRT23=keratin 23; TMEM35=Transmembrane protein 35; DCHS2=dachsous cadherin-related 2; CLDN8=claudin 8; IFI6=interferon alpha inducible protein 6

FIG. 2 ′—Cellular deconvolution

Visual representation of the enrichment scores for the individual cells types identified being differentially represented between vedolizumab non-remitters (A) and remitters (B), according to deconvolution techniques on the initial baseline transcriptome.²¹

FIG. 3′″—Receiver operating characteristic statistics

Receiver operating characteristic (ROC) statistics predicting vedolizumab induced endoscopic remission based on the colonic 4-gene predictive panel in an independent Belgian-Spanish validation cohort

FIG. 4 ′″—Immunohistochemistry

Immunohistochemical PIWIL1 staining in inflamed IBD colon (original magnification (OM)×100) (A). Immunohistochemical MAATS1 (C3orf15) staining in inflamed IBD colon (OM×100) (B). Immunohistochemical DCHS2 staining in inflamed IBD colon (OM×200) (C). Immunohistochemical RGS13 staining in inflamed IBD colon (OM×200) (D).

FIG. 5 ′″—Differential expression of the 4 genes in the predictive panel

Visual representation of the differential gene expression in mucosal biopsies of patients responding and not responding to vedolizumab therapy of the 4 genes included in the predictive panel.

log FC=log fold change; FDR p value=false discovery rate corrected p value.

PIWIL1=Piwi-like protein 1; MAATS1=MYCBP associated and testis expressed 1; DCHS2=dachsous cadherin-related 2; RGS13=Regulator of G-protein signaling 13

FIG. 6′″—Immunohistochemistry

Immunohistochemical PIWIL1 staining in regenerating colonic epithelium (original magnification (OM)×50).

### LEGENDS TO THE TABLES IN THIS APPLICATION

Table 1 demonstrates the disease characteristics of the whole blood, anti-TNF treated cohort.

Table 2 demonstrates the correlation between the overall TREM1 expression level and the expression of the different transcripts in whole blood.

Table 1′ demonstrates the baseline disease characteristics of all included patients n=number of patients; y=years; IQR=interquartile range; CD=Crohn's disease

Table 2′ demonstrates the enriched pathways among the inferred feature sets in the blood monocytes. Pathway enrichment was performed using Ingenuity Pathway Analysis.

Table 3′ demonstrates the enriched pathways among the inferred feature sets in the colonic mucosa. Pathway enrichment was performed using Ingenuity Pathway Analysis).

Table 4′ demonstrates the colonic signature biological response

Table 1″—Summary of the functional relevance of the top ranked hubs identified in each of the cohorts. Hubs were defined as the class discriminating features (protein coding genes) with the highest number of downstream targets in cell type and cohort specific networks. Hubs with over-represented biological processes among their downstream targets are indicated.

Table 2″—Summary of the identified biomarkers and associated accuracies in the different cohorts. Accuracies were determined by an ensemble classifier built using multiple individual classifiers.

Table 3″: Clinical features of all included patients

Table 4″: Relationship between the weights assigned to every sample in each latent factor (LF) and vedolizumab induced endoscopic remission in ulcerative colitis. Multiple regression was used to calculate the relationships between the LFs and endoscopic outcome. For the trait of endoscopic remission—1 stands for observed endoscopic remission; 0—stands for no observed endoscopic remission. Only LFs with weight contributions to all the samples in their respective cohorts were considered for further downstream analysis.

Table 5″: Relationship between the weights assigned to every sample in each latent factor (LF) and vedolizumab induced endoscopic remission in Crohn's disease. Multiple regression was used to calculate the relationships between the LFs and endoscopic outcome. For the trait of endoscopic remission—1 stands for observed endoscopic remission; 0—stands for no observed endoscopic remission. Only LFs with weight contributions to all the samples in their respective cohorts were considered for further downstream analysis.

Table 6″: Relationship between the weights assigned to every sample in each latent factor (LF) and anti-TNF induced endoscopic remission in ulcerative colitis. Multiple regression was used to calculate the relationships between the LFs and endoscopic outcome. For the trait of endoscopic remission—1 stands for observed endoscopic remission; 0—stands for no observed endoscopic remission. Only LFs with weight contributions to all the samples in their respective cohorts were considered for further downstream analysis.

Table 7″: Relationship between the weights assigned to every sample in each latent factor (LF) and anti-TNF induced endoscopic remission in Crohn's disease. Multiple regression was used to calculate the relationships between the LFs and endoscopic outcome. For the trait of endoscopic remission—1 stands for observed endoscopic remission; 0—stands for no observed endoscopic remission. Only LFs with weight contributions to all the samples in their respective cohorts were considered for further downstream analysis.

Table 8″: Variance contributions of the -omic layers to the MOFA identified latent factors (LF) in the vedolizumab treated ulcerative colitis cohort.

Table 9″: Variance contributions of the -omic layers to the MOFA identified latent factors (LF) in the vedolizumab treated Crohn's disease cohort.

Table 10″: Variance contributions of the -omic layers to the MOFA identified latent factors (LF) in the anti-TNF treated ulcerative colitis cohort.

Table 11″: Variance contributions of the -omic layers to the MOFA identified latent factors (LF) in the anti-TNF treated Crohn's disease cohort.

Table 12″—Summary of the prominent -omic layers contributing to the explanatory latent factors (LF) in the different cohorts. The -omic layer with the highest variance contribution to the explanatory LF was considered as the dominant one.

Table 13″: Tabular summary of the co-occurrence of the features discovered in each of the cohorts and across cell-/tissue-types. 1 indicates the presence of the feature in the corresponding dataset and 0 its absence.

CD=Crohn's disease, UC=ulcerative colitis

Table 14″: Correlation between the discriminatory features and C-reactive protein levels. The correlation was determined by using the psych package in R.

Table 15″: Adjacency matrices representing the interactions in binary format between the discriminatory features in each of the cohorts and their downstream protein targets. Interactions between the nodes are indicated as being present (1) or absent (0).

CD=Crohn's disease, UC=ulcerative colitis, VDZ=vedolizumab Table 16″: Sources of the interaction networks corresponding to the proteins targeted by the feature sets distinguishing responders and non-responders in each of the cohorts.

Table 17″: List of over-represented gene ontology based biological process terms within the set of proteins targeted by the features distinguishing responders and non-responders in each of the cohorts. Gene Ontology terms were retrieved from UniProt.

CD=Crohn's disease, UC=ulcerative colitis, VDZ=vedolizumab; IFXADM=Anti-TNF agents

Table 18″: Reactome pathways enriched among the set of features distinguishing responders and non-responders in the vedolizumab and anti-TNF cohorts. 1 indicates the enrichment of the pathway in the corresponding dataset and 0 indicates no enrichment. CD=Crohn's disease, UC=ulcerative colitis, VDZ=vedolizumab; IFXADM=Anti-TNF agents

Table 37″ provides an oOverview of antibodies used for fluorescence activated cell sorting

Table 20″provides an overview of all proteins measured with the Proximity Extension Technology.

Table 1′″provides the clinical characteristics of the inception cohort, validation cohort 2 and 4

Table 2′″demonstrates the accuracy of the 4-gene signature in vedolizumab and anti-TNF treated patients

Table 3′″provides details of the forward (Fw) and reverse (Rev) primers used for the beta actin qPCR analysis, including the amplicon length, melt temperature (Tm), 5′-3′ sequence and NCBI accession number.

Table 4′″: provides details of target-specific TaqMan Primers.

Table 5′″: provide an overview of primary antibodies immunohistochemistry

Table 6′″: provides the clinical features of the anti-TNF treated cohort

Table 7′″: provides the baseline differentially expressed genes between vedolizumab responders and non-responders selected based on a nominal 0.005 significance level.

Table 8′″: provides a gene set enrichment analysis (GSEA) results focused on the leukocyte migration and cell adhesion gene ontology (GO) gene sets, derived from the MSigDB. All gene sets are enriched in the non-responder group.

### Abbreviations in this Application

ADM adalimumab

AUC area under the curve

CD Crohn's disease

CLDN8 claudin 8

DCHS2 dachsous cadherin-related 2

FDR false discovery rate

GO gene ontology

GSEA gene set enrichment analysis

IBD inflammatory bowel disease

IEC intra-epithelial cell

IF16 interferon alpha inducible protein 6

IFX infliximab

IL interleukin

IPA ingenuity pathway analysis

IQR inter quartile range

ITGA4 integrin subunit alpha 4

ITGB7 integrin subunit beta 7

KRT23 keratin 23

LR likelihood ratio

MadCAM-1 mucosal vascular addressin cell adhesion molecule 1

MAATS1 MYCBP associated and testis expressed 1

NPV negative predictive value

PIWIL1 piwi-like protein 1

PPV positive predictive value

qPCR real-time polymerase chain reaction

RGS13 regulator of G protein signaling 13

RNA ribonucleic acid

ROC receiver operating characteristics

SNP single nucleotide polymorphism

TMEM35 transmembrane protein 35

TNF tumour necrosis factor

UC ulcerative colitis

Tables to this application

TABLE 1
Disease characteristics of the whole blood, anti-TNF treated cohort.

| | Crohn's | | Ulcerative | |
|---|---|---|---|---|
| | disease | | colitis | |
| Characteristic | n = 24 | | n = 30 | |
| Sex, women, n(%) | 12 | (50.0) | 18 | (60.0) |
| Endoscopic assessment after initiated therapy, n (%) | | | | |
| Endoscopic remission | 13 | (54.2) | 10 | (33.3) |
| No endoscopic remission | 11 | (45.8) | 20 | (66.7) |
| Anti -TNF agent, n (%) | | | | |
| Infliximab | 10 | (41.7) | 12 | (40.0) |
| Adalimumab | 14 | (58.3) | 18 | (60.0) |
| Age. Years median (!QR) | 31.9 | (26.5-51.5) | 43.5 | (29.6-55.7) |
| Disease duration.years, median (!QR) | 7.8 | (2.1-22.2) | 5.1 | (1.7-17.0) |
| (-reactive protein, mg,/L median (!QR) | 5.7 | (0.9-8.5) | 3.9 | (1.1-24.6) |
| Faecal calprotectin, μg/g, median (!QR) | 1190 | (328-1800) | 1361 | (804-1800) |
| Albumin, g/L, median (!QR) | 42.3 | (392-45.2) | 43.6 | (39.4-45.5) |
| Body Mass Index, kg/m², median (!QR) | 22.1 | (20.4-25.1) | 21.6 | (19.7-25.9) |

Disease location Crohn's disease, n(%)

| Ileal disease (Ll) | 6 | (25.0) | N.A |
|---|---|---|---|
| Colonic disease (L2) | 6 | (25.0) | |
| Ileocolonic disease (L3) | 12 | (50.0) | |
| Upper GI involvement (L4) | 1 | (4.2) | |

Disease location ulcerative colitis, n (%)

| | | |
|---|---|---|
| Proctitis (El) | 3 | (10.0) |
| Left - sided colitis (E2) | 19 | (63.3) |
| Extensive colitis (E3) | 8 | (26.7) |

Disease behaviour Crohn's disease, n(%)

| Non stricturing non- penetrating (Bl) | 15 | (62.5) | N.A | |
|---|---|---|---|---|
| Stricturing (B2) | 6 | (25.0) | | |
| Penetrating (B3) | 3 | (12.5) | | |
| Perianal disease (p) | 4 | (16.7) | | |
| Previous IBO related surgery (resection, | 10 | (41.7) | N.A | |
| stricturoplasty) Concomitant | | | | |
| medication, n (%) | | | | |
| To picalor systemic steroids | 8 | (33.3) | 8 | (26.7) |
| Immunomodulators | 12 | (50.0) | 6 | (20.0) |
| Previous biological agents, n (%) | | | | |
| Any | 8 | (33.3) | 10 | (33.3) |
| Infliximab | 4 | (16.7) | 2 | (6.6) |
| Adalimumab | 4 | (16.7) | 3 | (10.0) |
| Vedolizumab | 4 | (16.7) | 7 | (23.3) |
| Ustekinumab | 3 | (12.5) | N.A | |
| Smoking, n (%) | | | | |
| Never | 16 | (66.6) | 18 | (60.0) |
| Active | 4 | (16.7) | 5 | (16.7) |
| Former | 4 | (16.7) | 7 | (23.3) |

TABLE 2
Correlation between the overall TREM1 expression level and
the expression of the different transcripts in whole blood.

| Overall | Overall | | |
|---|---|---|---|
| TREM1 | TREM1 | | |
| signal | signal | TREM1-mb | TREM1-sv |
| TREM1-mb | ρ = 0.55 | | |
| | (p = 1.6 × 10−5) | | |
| TREM1-sv | ρ = 0.52 | ρ = 0.73 | |
| | (p = 1.0 × 10−4) | (p = 3.5 × 10−9) | |
| TREM1-x2 | ρ = 0.72 | ρ = 0.78 | ρ = 0.78 |
| | (p = 2.3 × 10−9) | (p = 9.9 × 10−12) | (p = 4.8 × 10−11) |

TABLE 1
Baseline disease characteristics of all included patients

| | | |
|---|---|---|
| Sex, women, n (%) | 44 | (68.8) |
| Disease duration, y, median (IQR) | 16.5 | (9.7-23.6) |
| Age at initiation of ustekinumab, y, median (IQR) | 39.3 | (31.6-50.9) |
| C-reactive protein, mg/L, median (IQR) | 10.8 | (4.6-19.0) |
| Faecal calprotectin, μg/g, median (IQR) | 1260.3 | (257.9-1800) |
| Serum albumin, g/L, median (IQR) | 40.6 | (38.1-42.5) |
| Harvey Bradshaw Index, median (IQR) | 9.0 | (7.0-11.0) |
| Simple Endoscopic Score for Crohn's disease, | 11.5 | (8.0-18.0) |
| median (IQR) | | |
| Previous biological therapy, n (%) | | |
| Previous anti-TNF exposure | 63 | (98.4) |
| Previous vedolizumab exposure | 51 | (81.3) |
| Disease location, n (%) | | |
| Ileal disease (L1) | 19 | (29.7) |
| Colonic disease (L2) | 5 | (7.8) |
| Ileocolonic disease (L3) | 40 | (62.5) |
| Upper GI involvement (L4) | 11 | (17.2) |
| Disease behaviour, n (%) | | |
| Inflammatory (B1) | 19 | (29.7) |
| Stricturing (B2) | 29 | (45.3) |
| Penetrating (B3) | 16 | (25.0) |
| Perianal disease (p) | 25 | (39.1) |
| History of CD-related surgery, n (%)* | 42 | (65.6) |
| Concomitant medication, n (%) | | |
| Corticosteroids | | |
| Topical steroids | 7 | (10.9) |
| Systemic steroids | 13 | (20.3) |
| Immunomodulators | 1 | (1.6) |
| Smoking status, n (%) | | |
| Active smoking | 16 | (25.0) |
| Previously smoking | 18 | (28.1) |
| Never smoked | 30 | (46.9) |

n = number of patients;
y = years;
IQR = interquartile range;
CD = Crohn's disease

TABLE 2
Enriched pathways among the inferred feature sets
in the blood monocytes. Pathway enrichment was
performed using Ingenuity Pathway Analysis.

| | 50% decrease in | Endoscopic |
|---|---|---|
| | faecal calprotectin | response |
| | by week 8 | after 6 months |
| | Benjamini-Hochberg corrected p-value | |
| OX40 Signaling Pathway | 0.00158489 | 5.37032E−07 |
| Antigen Presentation Pathway | 0.0060256 | 1.99526E−06 |
| Allograft Rejection Signaling | 0.006345654 | 2.75423E−06 |
| Cdc42 Signaling | 0.0107519 | 2.5704E−05 |
| Role of NFAT in Regulation of | 0.017519 | 0.0002884 |
| the Immune Response | | |
| Th1 and Th2 Activation | 0.0144544 | 0.00040738 |
| Pathway | | |
| CXCR4 Signaling | 0.03235937 | / |
| Graft-versus-Host Disease | / | 0.00056234 |
| Signaling | | |
| IL-4 Signaling | / | 0.00758578 |
| Neuroinflammation Signaling | / | 0.00891251 |
| Pathway | | |
| Dendritic Cell Maturation | / | 0.01202264 |
| Type I Diabetes Mellitus | / | 0.012022644 |
| Signaling | | |
| Chemokine Signaling | / | 0.04073803 |

TABLE 3
Enriched pathways among the inferred feature sets
in the colonic mucosa. Pathway enrichment was
performed using Ingenuity Pathway Analysis).

| | 50% decrease in | Endoscopic |
|---|---|---|
| | faecal calprotectin | response |
| | by week 8 | after 6 months |
| | Benjamini-Hochberg corrected p-value | |
| Agranulocyte Adhesion and | 5.7544E-06 | / |
| Diapedesis | | |
| Granulocyte Adhesion and | 0.000660693 | / |
| Diapedesis | | |
| Hepatic Fibrosis/Hepatic Stellate | 0.028840315 | / |
| Cell Activation | | |
| Role of IL-17A in Arthritis | 0.038904514 | / |
| Role of IL-17A in Psoriasis | 0.047863009 | / |

TABLE 4

Colonic signature biological response
CELSR3
HAAO
FAM135B
F2RL2
CMPK2
SLC28A2
RET
CHP2
PITX1
GSTT1
Monocyte CD14 biological response
FCER2
CTSL
PTGFRN
GPRC5C
SLAMF7
NR4A2
GNG2
RHOC
SULT1A1
DSC2
Monocyte CD14 endoscopic response
NEDD4L
ENGASE
GSN
GNLY
CLEC10A
HLA_DRB5
BAG3
ASGR2
HLA_DRB1
PTK2

TABLE 1

| | | | | Number of | | | |
|---|---|---|---|---|---|---|---|
| | | | | features | | Number of | |
| | | | | discriminating | | downstream | |
| | | | Size of | positive or | | target | |
| | | Cell or | feature | negative | Top | proteins | Expression |
| | | tissue | sub-network | endoscopic | feature | in the | trend in |
| Drug | Disease | type | (nodes; edges) | outcome | hubs | network | responders |
| Vedolizumab | Ulcerative | CD4 | 32; 26 | 7 | SMAD7* | 13 | ↑ |
| | colitis | | | | TNF* | 6 | ↑ |
| Vedolizumab | Crohn's | CD14 | 79; 70 | 15 | EGR1* | 33 | ↑ |
| | disease | | | | NFKB1A* | 14 | ↑ |
| Anti-TNF | Ulcerative | Colon | 94; 103 | 15 | JUN (AP-1) | 53 | ↑ |
| agents | colitis | CD4 | 20; 23 | 3 | SOCS3* | 18 | ↑ |
| | | CD14 | 68; 58 | 13 | EGR1* | 33 | ↓ |
| Anti-TNF | Crohn's | CD4 | 17; 21 | 11 | CXCR4 | 11 | ↑ |
| agents | disease | CD14 | 35; 43 | 12 | TNFAIP3* | 8 | ↑ |

*indicates the hubs whose downstream targets are enriched with Gene Ontology Biological Processes

TABLE 2

| | Latent | Dominant -omic | Predictive | |
|---|---|---|---|---|
| Cohort | Factor (s) | layer | biomarkers | Accuracy |
| Vedolizumab | LF 5 | Genomics | FAM129A, ELM01, TRIP13 | 84.2% |
| Ulcerative | | | PTAR1, ASAH1 | |
| colitis | | | | |
| Vedolizumab | LF 3 | Genomics | SKAP2, HAUS1, C3orf67, | 77.2% |
| Crohn's | | | SEC14L6, ATP6V0D1 | |
| disease | LF 8 | Monocyte | ABCG1, ERAP1, ERV3_1 | 98.0% |
| | | transcriptomics | APOL6, STON2 | |
| Anti-TNF | LF 2 | CD4⁺ T cell | ELOVL4, FGL2, CTSW, | 92.0% |
| agents | | transcriptomics | DDX11, LYZ | |
| Ulcerative | | | | |
| colitis | | | | |
| Anti-TNF | LF 2-LF9 | Genomics | TRAPPC4, CDKAL1, | 81.3% |
| agents | | | ACVRL1, TSPAN14, PCNP | |
| Crohn's | LF 5-LF 16 | Monocyte | CITED4, CLEC5A, SGK1, | 98.0% |
| disease | | transcriptomics | ALOX4AP, SGK223 | |

TABLE 3

| | Anti-TNF treated | | Vedolizumab treated | | Anti-TNF treated | | Vedolizumab treated | |
|---|---|---|---|---|---|---|---|---|
| | UC cohort | | UC cohort | | CD cohort | | CD cohort | |
| | n = 55 | | n = 61 | | n = 69 | | n = 70 | |
| Sex, women, n (%) | 27 | (49.1) | 31 | (50.8) | 33 | (47.8) | 42 | (60.0) |
| Disease duration, y, median (IQR) | 3.0 | (1.4-11.2) | 10.4 | (5.0-16.6) | 5.4 | (1.2-20.4) | 14.9 | (5.0-25.3) |
| Age at inclusion, y, median (IQR) | 41.0 | (28.0-53.5) | 45.9 | (35.8-57.0) | 34.8 | (22.9-50.8) | 41.7 | (32.1-50.3) |
| C-reactive protein, mg/L, median (IQR) | 6.0 | (1.9-21.8) | 2.7 | (1.0-11.5) | 6.5 | (2.7-26.9) | 4.4 | (1.9-13.2) |
| Serum albumin, g/L, median (IQR) | 42.4 | (39.9-44.1) | 42.3 | (39.6-44.7) | 41.0 | (38.4-44.4) | 42.2 | (39.7-44.7) |
| Previous biological therapy, n (%) | | | | | | | | |
| Previous anti-TNF exposure | 11 | (20.4) | 47 | (77.0) | 19 | (27.5) | 55 | (78.6) |
| Previous vedolizumab exposure | 5 | (9.1) | 0 | (0.0) | 5 | (7.2) | 0 | (0.0) |
| Disease location, n (%) | | | | | | | | |
| Ileal disease (L1) | N.A. | | N.A. | | 21 | (30.4) | 13 | (18.6) |
| Colonic disease (L2) | | | | | 14 | (20.3) | 11 | (15.7) |
| Ileocolonic disease (L3) | | | | | 34 | (49.3) | 46 | (65.7) |
| Upper GI involvement (L4) | | | | | 5 | (7.2) | 8 | (11.4) |
| Disease behaviour, n (%) | | | | | | | | |
| Inflammatory (B1) | N.A. | | N.A. | | 34 | (49.3) | 28 | (40.0) |
| Stricturing (B2) | | | | | 18 | (26.1) | 25 | (35.7) |
| Penetrating (B3) | | | | | 17 | (24.6) | 17 | (24.3) |
| Perianal disease (p) | | | | | 15 | (21.7) | 34 | (48.6) |
| Disease extent, n (%) | | | | | | | | |
| Proctitis (E1) | 12 | (21.8) | 8 | (13.1) | N.A. | | N.A. | |
| Left-sided colitis (E2) | 28 | (50.9) | 29 | (47.5) | | | | |
| Pancolitis (E3) | 15 | (27.3) | 24 | (39.3) | | | | |
| Concomitant medication, n (%) | | | | | | | | |
| Corticosteroids | | | | | | | | |
| Topical steroids | 11 | (20.0) | 24 | (39.3) | 9 | (13.0) | 18 | (25.7) |
| Systemic steroids | 10 | (18.2) | 11 | (18.0) | 6 | (8.7) | 10 | (14.3) |
| Immunomodulators | 15 | (27.3) | 4 | (6.7) | 21 | (30.9) | 7 | (10.0) |
| Smoking status, n (%) | | | | | | | | |
| Active smoking | 5 | (9.1) | 2 | (3.3) | 14 | (20.3) | 13 | (18.6) |
| Previously smoking | 11 | (20.0) | 21 | (34.4) | 13 | (18.8) | 17 | (24.3) |
| Never smoked | 39 | (70.9) | 38 | (62.3) | 42 | (60.9) | 40 | (57.1) |
| Type of anti-TNF therapy, n (%) | | | | | | | | |
| Adalimumab | 19 | (34.5) | N.A. | | 35 | (49.3) | N.A. | |
| Infliximab | 35 | (65.5) | | | 35 | (50.7) | | |
| Favorable endoscopic outcome, n (%) | 20 | (36.4) | 38 | (62.3) | 38 | (55.1) | 33 | (47.1) |

n = number of patients;
IQR = interquartile range;
N.A. = not applicable;
UC = ulcerative colitis;
CD = Crohn's disease

TABLE 4

| ID | LF1 | LF2 | LF3 | LF4 | LF5 | LF6 | LF7 | LF8 |
|---|---|---|---|---|---|---|---|---|
| 227 | −2.612 | −0.213 | 0.070 | −0.323 | −1.678 | −0.061 | 0.228 | −0.109 |
| 9823 | −2.564 | −1.036 | −0.294 | −0.214 | −0.541 | −0.087 | −0.332 | −0.161 |
| 2949 | −1.942 | −0.338 | −0.658 | 2.486 | −3.123 | −0.331 | −0.794 | 0.368 |
| 5561 | −2.487 | −0.505 | 0.160 | −0.083 | −0.778 | −0.346 | −0.713 | 0.114 |
| 7062 | −2.213 | 0.258 | 0.430 | −2.204 | −1.736 | 0.121 | −0.721 | −0.055 |
| 7936 | −2.273 | −1.379 | 0.258 | −1.107 | −0.858 | 0.056 | −0.203 | −0.199 |
| 8537 | −2.251 | 2.156 | −0.060 | −0.623 | −1.577 | −0.026 | 0.584 | −0.326 |
| 9516 | −2.641 | −0.727 | 0.946 | −0.133 | −0.157 | 0.166 | 3.009 | −0.102 |
| 9652 | −2.389 | −0.261 | 0.511 | 1.250 | −1.466 | −0.237 | −0.011 | −0.006 |
| 9893 | 2.663 | 0.236 | −4.536 | 0.202 | 1.194 | −0.582 | 0.515 | −0.270 |
| 12378 | −2.160 | 2.634 | 0.856 | 0.374 | 1.659 | 0.130 | −0.168 | −0.019 |
| 12637 | −1.954 | 0.202 | 0.525 | 0.369 | −5.408 | 0.078 | 0.242 | −0.014 |
| 12688 | −1.799 | −0.730 | 0.471 | −0.741 | 16.107 | −0.143 | 0.406 | 0.120 |
| 4748 | −2.538 | −0.132 | 0.006 | −0.075 | −1.905 | −1.078 | −0.675 | 0.535 |
| 5468 | −3.067 | 1.261 | −0.129 | 0.487 | 0.063 | −0.418 | −0.617 | 0.075 |
| 5520 | −2.510 | −1.171 | −0.035 | −0.350 | −1.213 | 0.609 | −0.154 | −0.095 |
| 7270 | −2.513 | −1.130 | 0.011 | 1.493 | −1.666 | −0.523 | 0.404 | 0.175 |
| 7682 | −2.646 | −1.118 | 0.035 | −0.389 | −0.327 | −0.160 | −0.452 | −0.120 |
| 8554 | −2.364 | −0.960 | 0.021 | −0.702 | −1.318 | 0.974 | 0.411 | −3.352 |
| 8646 | −2.150 | 0.481 | 0.010 | −0.327 | −2.524 | −0.747 | −0.187 | −0.397 |
| 11057 | 6.928 | 0.268 | 0.420 | 1.869 | 0.664 | −0.317 | 0.133 | −0.070 |
| 11615 | 24.304 | 0.307 | 0.309 | −1.455 | −1.672 | −0.216 | −0.368 | −0.020 |
| 12204 | −0.824 | 0.361 | 0.178 | 0.429 | 0.576 | 3.723 | −0.253 | 0.085 |
| 12484 | −3.743 | 1.985 | −0.080 | −0.608 | −2.902 | −0.083 | 0.584 | −0.119 |
| 12755 | −3.825 | −0.709 | −0.153 | −0.544 | 0.523 | −0.173 | −0.590 | −0.056 |
| 12942 | 9.386 | −1.132 | 0.756 | 1.009 | 0.851 | −0.159 | 0.187 | −0.129 |
| 287 | −2.015 | 2.036 | 0.000 | 0.024 | −2.694 | 0.000 | −0.325 | −0.825 |
| 687 | −2.837 | −0.188 | 0.000 | −0.027 | −0.257 | 0.000 | 1.219 | 2.042 |
| 2500 | −2.312 | −0.201 | 0.000 | −0.020 | −1.166 | 0.000 | −2.255 | −0.099 |
| 2634 | −2.792 | −0.552 | 0.000 | 0.029 | −1.225 | 0.000 | 0.265 | −0.328 |
| 3678 | −2.944 | 0.947 | 0.000 | −0.014 | −0.174 | 0.000 | 2.073 | 2.454 |
| 4439 | −2.568 | 0.033 | 0.000 | −0.013 | −1.292 | 0.000 | 0.064 | 0.747 |
| 4928 | −2.396 | 0.806 | 0.000 | −0.038 | −1.388 | 0.000 | −0.982 | −0.942 |
| 5283 | −2.031 | 0.153 | 0.000 | −0.040 | −1.959 | 0.000 | 0.812 | 0.728 |
| 5513 | −2.563 | −0.341 | 0.000 | 0.025 | −1.041 | 0.000 | 0.019 | −0.093 |
| 6009 | −2.486 | −0.491 | −0.001 | −0.094 | −0.292 | 0.000 | −1.882 | −1.303 |
| 6772 | −2.585 | −2.557 | 0.000 | −0.005 | −1.089 | 0.002 | 1.319 | −1.329 |
| 6962 | −2.563 | 0.407 | 0.000 | −0.048 | −0.965 | 0.000 | 3.139 | −2.390 |
| 7120 | −2.414 | −0.478 | 0.000 | −0.063 | −0.380 | 0.000 | −1.768 | −0.519 |
| 7154 | −2.535 | 0.689 | 0.000 | −0.040 | −0.289 | 0.000 | −0.603 | −0.812 |
| 7495 | −3.001 | −0.132 | 0.000 | 0.029 | 0.177 | 0.000 | 0.010 | −1.756 |
| 7915 | −2.278 | −0.276 | 0.000 | −0.035 | −1.447 | 0.000 | −0.117 | 0.610 |
| 7976 | −2.408 | 3.225 | 0.000 | 0.031 | −1.157 | 0.000 | 0.374 | −2.791 |
| 7987 | −2.641 | 1.427 | 0.000 | −0.044 | −0.237 | 0.000 | −1.281 | 1.149 |
| 8051 | −2.652 | −0.856 | 0.000 | −0.010 | 0.088 | 0.000 | 0.975 | −0.371 |
| 8747 | −2.381 | −0.397 | 0.000 | −0.031 | −1.208 | 0.000 | −1.738 | −1.202 |
| 8798 | −2.370 | 0.426 | 0.000 | −0.045 | −0.927 | 0.000 | −1.980 | −0.120 |
| 8978 | −2.582 | 0.357 | 0.000 | −0.019 | −1.071 | 0.000 | −0.773 | 1.587 |
| 9732 | −2.557 | −0.193 | 0.000 | 0.027 | −1.242 | 0.000 | −0.293 | −0.872 |
| 10833 | 0.018 | −0.208 | 0.009 | −0.783 | 0.455 | −0.908 | 0.284 | 0.928 |
| 11128 | 1.925 | 1.220 | 0.004 | −0.783 | 2.517 | 0.257 | −0.649 | −0.999 |
| 11383 | 3.588 | −0.191 | 0.062 | 0.145 | −0.327 | −2.482 | −0.072 | −0.537 |
| 11508 | 5.928 | −0.077 | −0.005 | −0.795 | 1.943 | −1.204 | −0.835 | 0.537 |
| 11605 | 0.326 | −1.318 | −0.011 | −0.368 | −4.526 | 2.909 | −0.073 | 1.073 |
| 12129 | −1.760 | 0.384 | 0.025 | −0.777 | 0.763 | −0.473 | 0.088 | 1.012 |
| 12194 | 0.598 | −0.042 | 0.002 | 4.617 | 1.463 | −0.427 | −0.097 | −0.133 |
| 12792 | 12.090 | 0.184 | 0.014 | −0.347 | −1.458 | 0.372 | 0.355 | 0.654 |
| 12505 | −2.319 | 0.175 | −0.868 | −0.470 | −2.726 | −0.486 | 0.733 | 0.882 |
| 12874 | 1.603 | −0.628 | 0.194 | −0.017 | 0.720 | −1.388 | −0.737 | −0.153 |
| 8724 | −2.397 | −0.142 | 0.477 | 0.452 | −1.259 | −0.130 | 0.076 | 0.010 |
| 12135 | −2.214 | 0.026 | 0.187 | 0.272 | 0.945 | −0.179 | −0.522 | 0.124 |
| R2 | 0.050 | −0.089 | −0.140 | 0.124 | −0.251 | 0.123 | −0.078 | −0.074 |
| P-value | 0.700 | 0.496 | 0.282 | 0.340 | 0.051 | 0.344 | 0.551 | 0.573 |

| | | | | | | | Endoscopic_im- |
|---|---|---|---|---|---|---|---|
| ID | LF9 | LF10 | LF11 | LF12 | LF13 | LF14 | provement |
| 227 | 0.838 | 0.524 | −0.093 | −0.130 | −0.007 | 0.533 | 0.000 |
| 9823 | 0.640 | −0.691 | 0.109 | −0.553 | 0.266 | −0.184 | 1.000 |
| 2949 | 0.215 | −0.127 | 4.487 | −0.707 | 0.059 | 1.277 | 1.000 |
| 5561 | −0.859 | −0.306 | −1.601 | 0.022 | −0.016 | 0.764 | 1.000 |
| 7062 | −0.272 | −0.440 | 1.298 | 0.415 | −0.043 | 1.148 | 1.000 |
| 7936 | −0.287 | −0.736 | 1.415 | 2.667 | 0.215 | −0.141 | 1.000 |
| 8537 | 1.659 | −0.160 | −0.096 | 0.442 | 0.172 | 0.042 | 1.000 |
| 9516 | −0.206 | −0.451 | 0.949 | −1.959 | 0.016 | −0.162 | 0.000 |
| 9652 | −0.231 | −0.319 | −1.009 | −0.222 | −0.072 | 0.042 | 1.000 |
| 9893 | −0.779 | 0.943 | −0.321 | 0.446 | −0.141 | −0.242 | 1.000 |
| 12378 | −0.519 | 0.153 | 1.301 | 0.720 | −0.221 | −0.011 | 0.000 |
| 12637 | −0.960 | 0.718 | −0.427 | 0.111 | 0.318 | −0.296 | 1.000 |
| 12688 | −0.612 | 0.150 | −0.001 | −0.138 | 0.059 | 0.101 | 0.000 |
| 4748 | 0.531 | 0.391 | 0.029 | 0.315 | NA | NA | 0.000 |
| 5468 | 0.090 | 1.847 | −1.797 | −1.422 | 0.068 | 0.721 | 1.000 |
| 5520 | −0.619 | 0.146 | 0.431 | −1.683 | NA | NA | 1.000 |
| 7270 | 4.676 | 0.115 | 0.525 | −0.104 | NA | NA | 0.000 |
| 7682 | 0.546 | −1.769 | −0.808 | −0.758 | −0.126 | 0.322 | 0.000 |
| 8554 | −0.345 | −0.402 | 0.100 | 0.647 | NA | NA | 1.000 |
| 8646 | −0.267 | −0.344 | −0.267 | 0.128 | NA | NA | 1.000 |
| 11057 | −0.218 | −6.901 | −0.948 | 1.708 | 0.063 | 0.120 | 1.000 |
| 11615 | −0.185 | 0.212 | 0.353 | −1.010 | 0.121 | 0.032 | 1.000 |
| 12204 | 0.461 | 0.929 | −0.629 | −0.236 | −0.223 | −0.066 | 1.000 |
| 12484 | −0.199 | −0.546 | −0.100 | −2.011 | NA | −0.044 | 0.000 |
| 12755 | 0.836 | −0.845 | −0.686 | −0.917 | −0.127 | 0.336 | 1.000 |
| 12942 | 0.164 | 5.299 | −0.565 | 2.400 | −0.231 | 0.385 | 1.000 |
| 287 | 0.000 | 0.032 | 0.000 | −0.015 | NA | NA | 1.000 |
| 687 | 0.000 | −0.076 | 0.001 | 0.001 | NA | NA | 0.000 |
| 2500 | 0.000 | −0.446 | 0.000 | −0.046 | NA | NA | 1.000 |
| 2634 | −0.001 | 0.517 | 0.000 | 0.042 | NA | NA | 1.000 |
| 3678 | 0.000 | 0.281 | −0.002 | 0.029 | NA | NA | 1.000 |
| 4439 | 0.000 | −0.146 | 0.000 | −0.015 | NA | NA | 1.000 |
| 4928 | 0.000 | −0.304 | 0.001 | −0.009 | NA | NA | 0.000 |
| 5283 | 0.000 | −0.538 | 0.001 | −0.034 | NA | NA | 1.000 |
| 5513 | −0.001 | 0.385 | −0.002 | 0.020 | NA | NA | 1.000 |
| 6009 | 0.000 | −1.128 | 0.006 | −0.040 | NA | NA | 1.000 |
| 6772 | 0.000 | −0.121 | 0.007 | −0.013 | NA | NA | 0.000 |
| 6962 | 0.000 | −0.440 | 0.002 | −0.006 | NA | NA | 1.000 |
| 7120 | 0.000 | −0.579 | 0.001 | −0.024 | NA | NA | 1.000 |
| 7154 | 0.000 | −0.475 | 0.002 | −0.019 | NA | NA | 0.000 |
| 7495 | 0.001 | 0.004 | 0.000 | −0.002 | NA | NA | 1.000 |
| 7915 | 0.000 | −0.339 | 0.000 | −0.011 | NA | NA | 0.000 |
| 7976 | 0.000 | 0.090 | −0.001 | −0.009 | NA | NA | 0.000 |
| 7987 | 0.000 | −0.644 | 0.001 | −0.025 | NA | NA | 1.000 |
| 8051 | 0.000 | −0.377 | 0.001 | −0.005 | NA | NA | 0.000 |
| 8747 | 0.000 | −0.254 | 0.001 | −0.006 | NA | NA | 1.000 |
| 8798 | 0.000 | −0.737 | 0.001 | −0.050 | NA | NA | 0.000 |
| 8978 | 0.000 | −0.076 | −0.001 | 0.003 | NA | NA | 0.000 |
| 9732 | 0.000 | −0.127 | −0.001 | −0.003 | NA | NA | 1.000 |
| 10833 | −1.137 | −0.424 | 0.179 | 0.564 | NA | NA | 1.000 |
| 11128 | 1.201 | −0.616 | −0.499 | 0.918 | NA | NA | 0.000 |
| 11383 | −0.953 | 0.900 | 0.146 | 0.047 | NA | NA | 1.000 |
| 11508 | 0.414 | −0.083 | −0.226 | −0.836 | NA | NA | 0.000 |
| 11605 | −1.103 | −0.715 | 0.609 | 1.077 | NA | NA | 1.000 |
| 12129 | −0.233 | −0.577 | −0.429 | 0.145 | NA | NA | 0.000 |
| 12194 | −1.673 | −0.760 | −0.318 | −0.100 | NA | NA | 1.000 |
| 12792 | 0.139 | −0.373 | 0.092 | −0.539 | NA | NA | 0.000 |
| 12505 | 0.143 | −0.311 | −0.474 | 1.177 | −0.064 | 0.130 | 1.000 |
| 12874 | 0.168 | 0.896 | 0.258 | −0.041 | 0.053 | NA | 0.000 |
| 8724 | −0.437 | −0.316 | −0.524 | −0.590 | 0.006 | −0.213 | 1.000 |
| 12135 | −0.036 | −0.322 | 0.191 | −0.171 | −0.143 | −5.712 | 0.000 |
| R2 | −0.273 | 0.023 | −0.040 | 0.193 | 0.231 | 0.340 | |
| P-value | 0.033 | 0.860 | 0.758 | 0.136 | 0.277 | 0.104 | |

TABLE 5

| ID | LF1 | LF2 | LF3 | LF4 | LF5 | LF6 | LF7 | LF8 |
|---|---|---|---|---|---|---|---|---|
| 2863 | 0.351 | 0.467 | −2.259 | −0.736 | −0.255 | −0.226 | 0.059 | 0.041 |
| 4550 | −3.102 | −0.514 | −1.490 | −0.753 | 0.009 | 0.274 | −0.312 | −0.641 |
| 8030 | −3.808 | 0.117 | −1.572 | 1.619 | −0.060 | 0.471 | −0.032 | 0.446 |
| 426 | −3.802 | 0.770 | −1.998 | −0.607 | −3.954 | 0.364 | 0.256 | 0.777 |
| 2737 | −2.801 | −0.106 | −1.550 | 1.461 | 0.275 | 0.280 | −0.656 | −0.347 |
| 6365 | −2.712 | 0.009 | −2.013 | 0.215 | 0.212 | 0.553 | −0.275 | 0.520 |
| 7298 | −1.967 | −0.593 | −1.935 | 0.576 | 0.114 | 0.094 | −0.412 | −0.744 |
| 10739 | −3.842 | 0.814 | −1.919 | −2.840 | 0.580 | −0.070 | −0.417 | 0.376 |
| 10795 | 0.082 | −0.569 | 12.126 | 1.169 | −0.017 | 0.045 | −0.013 | 0.013 |
| 10899 | 0.714 | 0.341 | 1.573 | −0.184 | −0.071 | 0.178 | −2.965 | −0.447 |
| 11157 | −0.193 | −0.155 | −2.617 | 1.463 | 0.034 | 0.516 | −0.679 | 0.486 |
| 11193 | −1.685 | −0.473 | −2.837 | 0.438 | −0.104 | 0.582 | 0.004 | −0.683 |
| 11440 | 0.503 | −0.561 | −2.694 | 0.022 | 0.303 | 0.068 | −0.619 | −0.603 |
| 488 | 0.131 | −2.458 | −1.694 | 1.015 | 0.014 | 0.227 | 0.724 | 0.182 |
| 1628 | 0.449 | −0.807 | −1.985 | 0.516 | 0.050 | 0.046 | 1.404 | −0.140 |
| 1706 | −1.858 | −0.911 | −2.013 | 0.111 | −0.186 | 0.862 | 0.025 | −1.027 |
| 1856 | 2.387 | −0.752 | 9.442 | −6.895 | −0.177 | −0.026 | −0.240 | −2.159 |
| 2372 | −0.797 | −0.262 | −1.967 | −0.097 | −0.057 | 0.739 | −0.450 | 0.150 |
| 4699 | −3.342 | −0.089 | −1.250 | 0.204 | 0.148 | 0.229 | 0.182 | −1.659 |
| 5587 | −0.246 | −1.809 | −1.453 | −0.388 | 0.241 | 0.134 | −1.797 | 0.484 |
| 6490 | −5.336 | −0.466 | 1.200 | 0.154 | 0.274 | 0.407 | 3.230 | 3.985 |
| 7129 | −2.125 | −0.582 | −0.901 | −0.338 | 0.624 | 0.360 | 0.086 | 0.164 |
| 7413 | 0.541 | −3.127 | −0.985 | −0.372 | −0.242 | 0.243 | 0.064 | −0.189 |
| 8304 | −1.474 | −0.916 | −1.315 | −0.112 | −1.049 | −0.578 | 0.026 | 0.658 |
| 8462 | −0.992 | −0.475 | 0.465 | 0.984 | −0.006 | 0.166 | −0.553 | −0.753 |
| 10038 | −1.705 | −1.272 | −2.063 | −0.435 | 1.880 | 0.983 | 0.096 | 0.674 |
| 10147 | 0.519 | −1.294 | −1.441 | −0.276 | −0.576 | −0.668 | 0.051 | −0.018 |
| 10283 | 3.646 | −0.566 | −6.490 | 1.592 | 0.296 | 0.037 | 0.592 | −0.699 |
| 10825 | 0.234 | −0.434 | −1.522 | 0.242 | 0.108 | 0.516 | 0.056 | 0.067 |
| 11820 | −3.240 | 0.676 | 9.835 | −1.295 | 0.172 | 0.582 | 0.682 | 3.162 |
| 11952 | 0.059 | 0.121 | −1.733 | −0.314 | 0.140 | −2.148 | −0.396 | 0.296 |
| 12128 | 3.575 | 0.955 | −8.548 | −0.471 | 0.458 | 0.184 | 0.055 | −1.899 |
| 12138 | −0.902 | 0.416 | 1.870 | −0.210 | 0.381 | 0.182 | −0.074 | 2.019 |
| 12601 | 17.634 | −0.224 | 1.577 | 0.957 | 0.189 | 0.531 | 1.259 | 2.502 |
| 12674 | 0.166 | 0.266 | 0.523 | −0.140 | −0.242 | −5.334 | 0.016 | 0.591 |
| 13035 | 1.934 | 0.154 | 7.906 | 0.405 | 0.276 | −0.044 | −0.159 | 0.157 |
| 13244 | 1.524 | 0.877 | −3.141 | 1.103 | 0.827 | 0.022 | 1.463 | −9.039 |
| 158 | −1.898 | −1.668 | −1.145 | 0.265 | −1.002 | 0.383 | −1.203 | −0.591 |
| 608 | −0.283 | −1.792 | −1.386 | 0.301 | −0.777 | −0.412 | 0.123 | 0.206 |
| 2630 | −0.432 | −1.535 | −1.092 | −0.455 | 1.318 | −0.028 | 0.263 | −0.033 |
| 2769 | −3.922 | −0.693 | −1.453 | −0.909 | −1.011 | −0.075 | −1.204 | −0.663 |
| 2852 | −1.583 | −1.257 | −1.180 | 0.174 | 1.005 | 0.664 | 0.868 | 0.461 |
| 2958 | −0.210 | −1.046 | −1.440 | 0.195 | −0.512 | −0.113 | −2.182 | 1.338 |
| 3089 | −2.047 | −1.717 | −1.155 | 0.188 | 1.792 | 0.080 | 2.671 | 1.169 |
| 3623 | −1.411 | −1.539 | −1.358 | −0.889 | 1.951 | 0.505 | 0.062 | 0.289 |
| 3930 | −2.048 | −0.925 | −1.476 | −0.022 | 2.661 | 0.400 | −2.371 | 1.526 |
| 4173 | −0.673 | −1.222 | −1.961 | 0.220 | 2.076 | −0.717 | −1.079 | 0.261 |
| 4698 | −2.338 | −1.482 | −1.541 | 0.367 | −0.498 | 0.337 | 0.025 | 0.587 |
| 4929 | −2.212 | −1.432 | −1.002 | 0.265 | −1.630 | 0.122 | 1.501 | 1.135 |
| 5476 | −1.957 | −1.870 | −0.901 | 0.273 | −1.208 | 0.173 | −1.405 | 0.951 |
| 5559 | −0.259 | −2.017 | −1.153 | 0.210 | −0.506 | −0.051 | −2.009 | −0.931 |
| 6086 | 0.043 | −1.477 | −1.378 | 0.205 | 1.191 | −0.145 | −1.460 | −0.727 |
| 6103 | −0.877 | −0.646 | −1.764 | −0.005 | 1.885 | 0.072 | −2.276 | −0.619 |
| 7052 | −0.958 | −0.767 | −1.791 | 0.072 | 1.878 | 0.710 | −0.333 | 0.329 |
| 7505 | −1.206 | −1.590 | −1.384 | 0.092 | 1.040 | 0.475 | 1.845 | −0.319 |
| 7690 | −1.636 | −1.312 | −1.028 | −0.082 | 1.751 | 0.209 | 0.562 | 0.580 |
| 7691 | −1.930 | −1.868 | −1.147 | 0.029 | 0.698 | −0.507 | 0.010 | 1.495 |
| 7855 | −3.716 | −1.032 | −1.310 | 0.244 | −1.781 | −0.436 | 1.387 | 0.112 |
| 8431 | −0.630 | −1.133 | −1.749 | 0.430 | 2.079 | 0.213 | −0.242 | −1.751 |
| 9415 | −1.474 | 0.109 | 2.645 | −0.685 | 0.048 | 0.111 | 1.215 | 0.171 |
| 10409 | 0.352 | 3.014 | 5.624 | −0.501 | 0.545 | 0.866 | 0.033 | −1.646 |
| 10777 | 6.417 | −0.081 | 0.081 | 1.488 | 0.694 | 0.957 | −0.007 | 0.688 |
| 11463 | 0.187 | 2.112 | −1.798 | −0.109 | 0.607 | −1.843 | −0.166 | 0.555 |
| 11641 | 1.110 | 2.912 | −1.523 | −0.709 | −0.422 | 1.401 | −0.037 | 3.778 |
| 11655 | 0.493 | 0.317 | −1.081 | 0.341 | −0.095 | 0.027 | 2.155 | 0.074 |
| 11766 | 0.364 | −0.029 | 2.004 | −1.031 | −0.077 | 0.062 | −3.626 | −0.237 |
| 11881 | 0.374 | 0.048 | −0.150 | −0.149 | 0.173 | −0.028 | 3.674 | −0.418 |
| 12011 | 0.003 | −3.983 | −0.406 | −0.010 | −0.480 | −0.743 | 0.048 | −0.100 |
| 12316 | −0.229 | 2.237 | 3.984 | 0.149 | −1.293 | 1.496 | 0.007 | −1.467 |
| 12459 | −0.072 | −0.085 | 3.196 | −1.043 | 0.073 | 0.070 | 0.757 | 0.149 |
| R2 | 0.048 | −0.141 | −0.157 | 0.119 | 0.018 | 0.052 | −0.005 | −0.159 |
| P- | 0.692 | 0.243 | 0.195 | 0.325 | 0.882 | 0.670 | 0.964 | 0.188 |
| value | | | | | | | | |

| ID | LF9 | LF10 | LF11 | LF12 | LF13 | LF14 | LF15 | |
|---|---|---|---|---|---|---|---|---|
| 2863 | 0.305 | −0.995 | 0.722 | −0.561 | −1.078 | 0.252 | −4.360 | 0 |
| 4550 | 1.020 | 0.404 | −0.077 | −0.111 | −1.308 | −0.034 | −0.135 | 1 |
| 8030 | −0.051 | 0.183 | −0.446 | −0.485 | −0.784 | −1.745 | −0.243 | 0 |
| 426 | 0.009 | 0.034 | 0.324 | 0.320 | −0.933 | 0.192 | 1.009 | 1 |
| 2737 | 0.977 | −0.473 | 0.073 | 1.321 | −0.609 | 0.317 | 0.245 | 0 |
| 6365 | −1.947 | 0.131 | −0.790 | 0.393 | −0.831 | −0.150 | −0.349 | 1 |
| 7298 | −2.164 | 0.146 | 0.567 | 0.479 | −0.641 | −0.132 | 0.358 | 0 |
| 10739 | 0.065 | −0.264 | 0.553 | −0.301 | −0.774 | −0.205 | 0.667 | 0 |
| 10795 | −0.072 | 0.022 | 0.350 | 0.216 | −2.179 | 0.061 | 0.035 | 0 |
| 10899 | 0.041 | 0.619 | 0.131 | −2.844 | 2.432 | 0.486 | 0.585 | 0 |
| 11157 | 0.287 | 0.191 | −0.639 | 0.548 | −0.661 | −0.022 | −0.178 | 0 |
| 11193 | 0.855 | 0.113 | −0.828 | 0.787 | 0.855 | −0.227 | −0.617 | 0 |
| 11440 | −0.354 | −0.410 | 0.516 | 0.893 | −3.428 | 0.543 | 0.543 | 1 |
| 488 | 0.073 | 0.291 | −0.303 | 1.475 | −0.568 | 0.748 | 1.066 | 0 |
| 1628 | −0.176 | −0.814 | −0.368 | −1.165 | −0.584 | 0.301 | −0.188 | 0 |
| 1706 | −3.365 | 0.058 | 1.123 | 0.398 | −0.369 | −0.572 | 0.127 | 1 |
| 1856 | −0.218 | −0.116 | 0.829 | −0.065 | 0.223 | −0.362 | 0.315 | 0 |
| 2372 | −3.515 | −0.513 | 0.229 | −0.154 | −0.473 | −0.284 | −0.042 | 1 |
| 4699 | −0.121 | −0.283 | 0.113 | −1.094 | −1.169 | −0.143 | 0.139 | 1 |
| 5587 | 0.616 | −0.207 | 2.322 | −0.941 | −0.609 | −1.922 | −0.354 | 0 |
| 6490 | −0.890 | 0.120 | −0.477 | −2.496 | −1.221 | 0.586 | 0.149 | 0 |
| 7129 | 1.684 | −0.495 | 1.815 | 0.000 | −1.246 | 1.341 | 0.027 | 1 |
| 7413 | 0.186 | 0.277 | 1.879 | −0.119 | −0.946 | −1.176 | 2.887 | 1 |
| 8304 | −2.101 | −0.189 | −2.812 | −0.024 | −0.939 | 0.670 | 0.436 | 1 |
| 8462 | −0.598 | 0.293 | 0.310 | 0.203 | −1.016 | −0.055 | 0.176 | 1 |
| 10038 | −0.737 | 0.594 | −1.700 | −0.331 | −0.385 | 1.058 | 0.125 | 1 |
| 10147 | 0.788 | −0.742 | −0.086 | 0.035 | −0.800 | −0.047 | 0.706 | 1 |
| 10283 | 0.720 | −0.704 | 0.314 | −0.278 | 13.825 | 0.012 | 0.179 | 1 |
| 10825 | 0.110 | −0.919 | −2.188 | −3.501 | −1.128 | −0.015 | 0.370 | 1 |
| 11820 | −1.010 | 0.730 | −1.131 | 1.613 | 5.676 | 0.297 | −0.249 | 0 |
| 11952 | 0.391 | 0.328 | 1.544 | −0.470 | 1.339 | −0.104 | −1.611 | 1 |
| 12128 | 0.617 | 0.050 | 0.197 | −0.036 | −3.467 | −0.100 | 0.182 | 0 |
| 12138 | 1.354 | 0.200 | −0.049 | 0.034 | −2.397 | 0.095 | 0.217 | 0 |
| 12601 | 0.145 | 0.350 | −0.844 | 0.993 | 0.001 | 0.014 | −0.306 | 1 |
| 12674 | 0.458 | −0.430 | −0.734 | −0.010 | −1.854 | 0.069 | 0.494 | 0 |
| 13035 | −0.008 | −0.551 | 0.590 | 0.466 | 1.278 | −0.107 | 0.536 | 1 |
| 13244 | 0.366 | 0.011 | 0.375 | −0.419 | −0.539 | 0.259 | 0.575 | 1 |
| 158 | 0.001 | 0.047 | 0.000 | 0.001 | −0.892 | 0.000 | 0.000 | 0 |
| 608 | 0.001 | −0.018 | 0.000 | 0.001 | −0.525 | 0.000 | 0.000 | 1 |
| 2630 | −0.002 | −0.302 | −0.001 | 0.000 | −0.890 | 0.001 | 0.000 | 1 |
| 2769 | −0.004 | 0.380 | −0.001 | −0.002 | −0.679 | 0.000 | 0.000 | 0 |
| 2852 | 0.001 | −0.001 | −0.001 | 0.000 | −0.741 | 0.000 | 0.000 | 0 |
| 2958 | 0.001 | −0.410 | 0.000 | 0.001 | −0.915 | 0.000 | 0.000 | 0 |
| 3089 | 0.000 | 0.396 | 0.000 | 0.000 | −0.608 | 0.000 | −0.001 | 1 |
| 3623 | 0.002 | 0.224 | 0.000 | 0.000 | −0.723 | −0.001 | 0.000 | 0 |
| 3930 | 0.002 | 0.220 | 0.000 | 0.000 | −0.677 | 0.000 | 0.000 | 1 |
| 4173 | 0.002 | −0.081 | 0.001 | 0.000 | −0.376 | 0.000 | 0.001 | 0 |
| 4698 | 0.001 | 0.420 | 0.000 | 0.000 | −0.479 | 0.000 | −0.001 | 1 |
| 4929 | 0.001 | 0.257 | 0.000 | 0.000 | −1.063 | 0.000 | 0.000 | 0 |
| 5476 | 0.002 | 0.232 | 0.000 | 0.000 | −0.835 | 0.000 | −0.001 | 1 |
| 5559 | 0.001 | −0.357 | 0.000 | 0.001 | −0.628 | 0.000 | 0.000 | 0 |
| 6086 | 0.001 | −0.582 | 0.000 | 0.000 | −0.571 | 0.000 | −0.001 | 1 |
| 6103 | 0.000 | −0.450 | 0.000 | 0.000 | −0.467 | 0.000 | 0.000 | 1 |
| 7052 | 0.003 | −0.219 | 0.000 | 0.000 | −0.553 | 0.000 | 0.000 | 0 |
| 7505 | −0.001 | −0.033 | −0.001 | 0.000 | −0.797 | 0.000 | −0.001 | 0 |
| 7690 | −0.001 | −0.124 | 0.000 | 0.000 | −0.888 | 0.000 | 0.000 | 1 |
| 7691 | 0.001 | 0.383 | 0.000 | 0.000 | −0.579 | 0.000 | −0.001 | 1 |
| 7855 | −0.001 | 0.430 | −0.001 | 0.000 | −0.641 | 0.000 | −0.001 | 1 |
| 8431 | 0.002 | −0.426 | −0.001 | 0.000 | −0.524 | 0.000 | −0.001 | 1 |
| 9415 | 0.738 | 0.010 | 0.335 | −1.004 | 0.085 | 2.473 | 1.799 | 0 |
| 10409 | 0.153 | −0.026 | −0.983 | 0.214 | 3.123 | −3.175 | 0.400 | 0 |
| 10777 | 0.620 | −0.020 | −0.633 | −0.133 | 1.690 | 0.339 | −1.112 | 0 |
| 11463 | −1.163 | −0.026 | 1.249 | −0.065 | 4.973 | 2.003 | 1.324 | 0 |
| 11641 | 0.251 | 0.024 | 0.163 | 0.224 | −1.125 | −0.473 | −1.077 | 0 |
| 11655 | 0.059 | −0.065 | 0.446 | −0.173 | 5.367 | −0.414 | 0.820 | 0 |
| 11766 | 0.095 | −0.049 | −0.077 | 3.037 | 1.026 | 1.573 | 0.111 | 1 |
| 11881 | −0.286 | 0.078 | −0.464 | 1.292 | 0.315 | 0.031 | −0.924 | 1 |
| 12011 | 0.245 | 0.050 | 0.313 | 0.197 | 3.686 | −0.374 | −0.029 | 0 |
| 12316 | 0.380 | 0.048 | −0.496 | −0.170 | 0.533 | 2.166 | 1.045 | 1 |
| 12459 | 0.244 | 0.056 | −2.492 | 1.334 | 0.837 | −1.886 | −0.406 | 0 |
| R2 | −0.158 | −0.052 | 0.007 | 0.051 | −0.049 | 0.146 | 0.104 | |
| P- | 0.193 | 0.670 | 0.956 | 0.674 | 0.689 | 0.228 | 0.390 | |
| value | | | | | | | | |

* = Endoscopic remission

TABLE 6

| LF1 | LF2 | LF3 | LF4 | LF5 | LF6 | LF7 | LF8 | LF9 | LF10 | LF11 | LF12 | LF13 | LF14 | LF15 | |
|---|---|---|---|---|---|---|---|---|---|---|---|---|---|---|---|
| 1.3 | 0.0005 | −0.0002 | −0.0771 | −0.0003 | 0.0001 | 0.0644 | −0.0005 | 0.0003 | 0.0003 | 0.0004 | NA | NA | −0.0090 | 0.0002 | 0 |
| 1.2 | 0.0001 | −0.0004 | −1.5738 | 0.0002 | 0.0004 | 0.0664 | −0.0007 | −0.0001 | 0.0001 | 0.0003 | NA | NA | 0.0003 | 0.0000 | 1 |
| 1.3 | 0.0003 | 0.0004 | −1.5222 | 0.0011 | 0.0001 | 0.0568 | −0.0026 | −0.0005 | −0.0009 | 0.0002 | NA | NA | −0.0260 | −0.0005 | 1 |
| 1.4 | −0.0234 | −0.9551 | 0.7069 | 0.2537 | 0.7710 | 0.1267 | −0.4931 | −0.9769 | 2.1486 | −0.0830 | 0.4952 | 0.0645 | −0.4808 | −0.1989 | 0 |
| 1.2 | −1.1162 | 0.3452 | −1.4871 | 0.0543 | −0.5874 | 0.0979 | 0.0659 | 0.0240 | 1.1795 | −0.1716 | 1.3374 | NA | −0.2017 | −0.0160 | 0 |
| 1.3 | 0.0000 | −0.0001 | −0.5494 | 0.0003 | 0.0002 | 0.0700 | −0.0005 | 0.0000 | −0.0003 | 0.0002 | NA | NA | −0.0052 | 0.0000 | 0 |
| 1.4 | 0.0001 | 0.0001 | −0.5215 | −0.0003 | −0.0004 | 0.0777 | 0.0007 | 0.0005 | 0.0002 | 0.0001 | NA | NA | 0.0054 | 0.0000 | 1 |
| 1.4 | −0.0003 | 0.0001 | 0.0106 | 0.0005 | −0.0006 | −0.0289 | −0.0006 | −0.0002 | 0.0000 | 0.0001 | NA | NA | −0.0060 | 0.0002 | 1 |
| 1.2 | −0.0003 | −0.0001 | −1.4938 | −0.0002 | 0.0008 | 0.0866 | −0.0009 | −0.0004 | −0.0001 | −0.0001 | NA | NA | −0.0137 | −0.0003 | 1 |
| 1.3 | 0.0064 | −0.0784 | −1.1556 | −1.4151 | 0.2896 | 0.0508 | 0.3608 | 0.2423 | −1.7521 | −0.8247 | 0.4460 | 0.2005 | −3.0759 | −0.1960 | 0 |
| 1.2 | 3.0497 | 0.5460 | −1.4887 | −1.7992 | 0.8908 | 0.2040 | −0.1347 | 1.4749 | −1.0829 | 0.2892 | NA | NA | −0.2874 | 0.3448 | 1 |
| 1.2 | −0.0001 | −0.0004 | −1.0991 | −0.0001 | 0.0004 | 0.0936 | −0.0007 | −0.0006 | 0.0014 | 0.0001 | NA | NA | −0.0080 | −0.0005 | 0 |
| 1.3 | −0.0007 | −0.0004 | −0.8424 | 0.0006 | −0.0004 | 0.0555 | −0.0009 | −0.0005 | 0.0000 | 0.0002 | NA | NA | −0.0077 | 0.0001 | 0 |
| 1.3 | −0.0001 | 0.0002 | −0.5456 | 0.0002 | 0.0006 | −0.3167 | 0.0016 | −0.0009 | 0.0007 | 0.0000 | NA | NA | 0.0332 | 0.0002 | 0 |
| 1.4 | −0.0002 | 0.0004 | −0.4269 | −0.0006 | 0.0002 | −0.3469 | 0.0045 | 0.0000 | 0.0011 | −0.0005 | NA | NA | 0.0582 | −0.0001 | 0 |
| 1.4 | 2.0316 | −0.0359 | −0.3986 | 3.5765 | 0.5415 | −1.0002 | −0.3330 | −0.1636 | −0.1681 | 0.0057 | NA | NA | 0.1763 | −1.2483 | 0 |
| 1.4 | 0.0002 | −0.0003 | −0.5070 | 0.0001 | 0.0006 | 0.1167 | −0.0008 | 0.0000 | 0.0007 | 0.0003 | NA | NA | −0.0062 | −0.0004 | 0 |
| 1.3 | −0.0005 | −0.0003 | −0.5079 | 0.0004 | −0.0002 | 0.0555 | −0.0014 | −0.0005 | 0.0005 | 0.0002 | NA | NA | −0.0077 | 0.0000 | 1 |
| 1.3 | 0.0001 | −0.0001 | −0.6650 | 0.0006 | 0.0003 | 0.0829 | −0.0018 | −0.0005 | 0.0000 | 0.0000 | NA | NA | −0.0141 | −0.0003 | 0 |
| 1.4 | 0.0000 | 0.0000 | −0.6076 | −0.0004 | 0.0002 | −0.1194 | 0.0015 | −0.0003 | 0.0008 | −0.0001 | NA | NA | 0.0124 | 0.0000 | 0 |
| 1.4 | 0.0002 | 0.0002 | 0.1603 | −0.0003 | 0.0000 | −0.0397 | 0.0007 | 0.0008 | −0.0009 | −0.0001 | NA | NA | −0.0030 | 0.0000 | 1 |
| 1.2 | 0.0002 | −0.0002 | −1.4678 | 0.0003 | 0.0009 | 0.0492 | −0.0006 | −0.0003 | −0.0002 | 0.0001 | NA | NA | −0.0141 | −0.0004 | 1 |
| 1.3 | −0.0003 | −0.0003 | −1.0010 | 0.0002 | −0.0001 | 0.0723 | −0.0013 | −0.0003 | 0.0001 | 0.0001 | NA | NA | −0.0114 | 0.0001 | 0 |
| 1.4 | −0.0002 | 0.0002 | 0.2470 | −0.0013 | −0.0003 | −0.1682 | 0.0022 | 0.0003 | 0.0010 | −0.0002 | NA | NA | 0.0236 | 0.0003 | 1 |
| 1.3 | −0.0005 | −0.0005 | −1.0524 | 0.0003 | 0.0000 | −0.0837 | 0.0013 | −0.0002 | 0.0011 | 0.0004 | NA | NA | 0.0148 | 0.0004 | 0 |
| 1.4 | −0.0005 | −0.0002 | −0.2712 | 0.0006 | 0.0000 | 0.0934 | −0.0005 | −0.0002 | −0.0001 | 0.0002 | NA | NA | −0.0070 | 0.0001 | 1 |
| 1.3 | −0.0533 | 1.3686 | −1.1999 | −0.8468 | −0.2763 | 0.1436 | 0.0432 | −0.0115 | 0.1172 | −0.3391 | 0.6898 | −0.9841 | 5.0796 | −0.1132 | 1 |
| 1.2 | −0.0634 | 1.9727 | −1.0041 | 0.5381 | −1.9784 | −0.0136 | 0.0322 | 0.1907 | −1.5757 | −0.5731 | −0.2276 | 0.8325 | −0.2363 | −0.8291 | 0 |
| 1.3 | −0.0001 | −0.0004 | −1.3443 | −0.0001 | 0.0005 | 0.0976 | −0.0010 | −0.0002 | 0.0002 | 0.0002 | NA | NA | −0.0119 | 0.0001 | 1 |
| 1.3 | 0.0000 | −0.0003 | −0.7000 | 0.0002 | 0.0003 | 0.1008 | 0.0002 | −0.0001 | −0.0002 | 0.0000 | NA | NA | −0.0098 | 0.0000 | 0 |
| 1.3 | −0.0476 | −0.1825 | −0.9309 | 0.0496 | −2.2545 | 0.6324 | 0.0549 | 0.1852 | 0.6129 | 0.2149 | −0.0063 | 0.0492 | 0.0608 | 0.7924 | 0 |
| −0.4 | −0.1083 | −0.1214 | 1.2137 | −0.1197 | −1.4563 | 0.7550 | 0.1090 | −0.7889 | 0.1529 | 0.2146 | NA | −3.0377 | 0.0624 | −0.9715 | 0 |
| −0.2 | −2.3877 | −0.1773 | −0.5165 | 1.7600 | 0.3733 | 1.2657 | −0.0451 | 1.2144 | −0.3345 | −0.0591 | NA | NA | −0.2153 | −2.6085 | 0 |
| 1.4 | −0.0741 | 0.1651 | 0.5111 | 0.1997 | 0.4572 | −1.0591 | −5.3544 | −0.3748 | −0.4254 | −0.0659 | 0.4517 | −0.3886 | 0.1237 | 0.1859 | 1 |
| −8.7 | 0.0229 | 0.2332 | −0.2680 | 2.0546 | −0.4987 | 0.2180 | −0.0891 | −0.0744 | −0.1318 | −0.0461 | NA | NA | −0.1460 | 1.9151 | 1 |
| −4.4 | 1.0760 | 0.0189 | −0.2979 | −3.2580 | −0.6061 | −0.6507 | −0.1300 | −0.5437 | −0.3992 | −0.1340 | NA | NA | 0.1016 | −3.0659 | 0 |
| 1.6 | −4.0276 | −0.2449 | 0.2949 | −0.9688 | −1.5791 | −1.2861 | −0.1350 | −0.6237 | −1.4166 | 0.1120 | NA | NA | −0.0366 | 1.6773 | 0 |
| 1.1 | −0.0498 | −0.3401 | −0.1717 | 0.1835 | −0.8683 | 1.5232 | 0.1101 | 1.3572 | −0.9751 | −0.1699 | −0.2981 | 0.1064 | 0.0862 | 0.1680 | 0 |
| 1.2 | −0.0460 | −7.2270 | 0.5807 | 0.2942 | 0.0585 | 0.9622 | 0.1109 | 0.7600 | −0.6694 | −0.2605 | −0.3273 | 0.2484 | 0.3275 | −0.0488 | 1 |
| −1.8 | −3.3253 | 0.2353 | −0.1187 | −0.9833 | 2.2811 | 0.1181 | 0.1985 | 0.6957 | 0.0990 | 0.2323 | NA | NA | 0.2834 | −0.4775 | 0 |
| 1.2 | −0.0460 | 0.2792 | −0.1175 | 0.1276 | 0.0508 | −1.1744 | −0.0130 | −0.3056 | −0.3946 | −0.2199 | −0.1280 | 0.2342 | 0.4192 | 0.0756 | 0 |
| 0.1 | −0.0248 | 0.0818 | 0.4584 | −0.0404 | −0.0542 | 0.1387 | −0.1866 | 0.1852 | 0.0323 | 5.3884 | −0.0841 | 0.4700 | −0.2316 | −0.1484 | 0 |
| 1.2 | −0.1121 | 0.2444 | 1.6120 | −0.1644 | −0.0938 | 0.6370 | 0.2790 | 1.0513 | −1.0150 | −0.3925 | −0.4563 | −1.3927 | −0.1475 | 0.6688 | 0 |
| 2.2 | 0.2437 | 0.0913 | 0.7605 | 1.4226 | −0.9547 | −0.6853 | 0.1692 | −0.0926 | 0.1027 | 0.1072 | NA | NA | 0.0085 | 0.1420 | 1 |
| 1.4 | −0.1041 | 1.0921 | 2.3911 | 0.2968 | 1.2370 | −0.4285 | 0.7010 | 0.8171 | −0.9773 | −0.4065 | −0.3061 | −0.3258 | −0.4137 | 0.0672 | 0 |
| 0.3 | −0.0380 | 0.0467 | ###### | 0.2996 | 0.8821 | 1.3686 | −0.0973 | 0.7370 | 1.4209 | 0.0724 | −1.2162 | 0.0999 | 0.1454 | 0.0281 | 0 |
| 1.2 | −0.0119 | 0.3473 | 1.3175 | 0.0609 | 0.9598 | 1.1139 | −0.0700 | −4.2244 | 0.0370 | −0.0918 | −0.3937 | 0.4785 | −0.1592 | 0.2683 | 0 |
| 1.3 | −0.0705 | 0.7913 | 1.1672 | 0.4102 | 0.5131 | −2.8439 | 1.7720 | −0.4831 | −1.0932 | −0.2408 | −0.3010 | 0.6789 | −0.0248 | 0.0991 | 0 |
| 1.2 | 0.0030 | −1.7178 | −0.1692 | −0.2837 | −0.8568 | −0.2905 | −0.3240 | −0.5163 | 0.6668 | −0.2445 | 0.1758 | 1.0153 | 0.0456 | −0.0198 | 1 |
| −23.6 | −0.0700 | 0.6366 | 0.5211 | 0.1650 | 0.0819 | −0.1602 | 0.5734 | 0.3342 | −0.4146 | −0.4776 | 0.1047 | 0.0168 | −0.0785 | 0.0780 | 0 |
| 0.8 | −0.0286 | 0.0253 | 2.8252 | −0.3363 | −0.4345 | −0.7354 | −0.2637 | −0.1533 | 2.5915 | −0.1780 | −0.7898 | 0.2335 | −0.2528 | −0.2388 | 1 |
| 0.9 | −0.0067 | −0.6106 | 3.5841 | −0.3427 | 0.8155 | −1.4132 | 0.5047 | 0.0999 | 0.7847 | −0.0291 | −0.9797 | 0.4178 | −0.1908 | −0.0553 | 1 |
| 1.1 | 0.0317 | 2.5989 | 2.8101 | 0.2057 | 1.1203 | 1.8133 | 0.5176 | 1.1069 | −0.6948 | −0.4111 | −0.7006 | 1.6598 | −0.1109 | −0.1577 | 0 |
| −0.2 | 1.5875 | −0.7774 | 0.0189 | −1.0454 | 1.8129 | −0.3624 | −0.3025 | −0.1678 | −0.5344 | 0.0196 | NA | NA | 0.0587 | 2.1804 | 0 |
| 1.8 | 0.9526 | 0.4213 | −2.0907 | 0.3068 | 0.1867 | −0.7847 | 0.1870 | 0.4209 | 0.6713 | 0.0318 | 2.5091 | −0.5047 | 0.0214 | −0.0014 | 1 |
| 0.1 | 0.1975 | −0.2062 | 0.0323 | 0.0177 | −0.0446 | −0.1244 | −0.2052 | 0.0495 | 0.1950 | −0.0603 | 0.2147 | −0.0018 | 0.2020 | 0.1347 | |
| 0.5 | 0.1480 | 0.1310 | 0.8150 | 0.8980 | 0.7470 | 0.3650 | 0.1330 | 0.7200 | 0.1540 | 0.6620 | 0.3373 | 0.9935 | 0.1390 | 0.3270 | |

* = Endoscopic remission

TABLE 7

| LF8 | LF9 | LF10 | LF11 | LF12 | LF13 | LF14 | LF15 | LF16 | LF17 | LF18 | LF19 | LF20 | LF21 | |
|---|---|---|---|---|---|---|---|---|---|---|---|---|---|---|
| −0.13 | −1.0655 | 0.0004 | −0.0001 | 0.0001 | NA | NA | −0.0003 | −0.0005 | 0.0000 | NA | 0.0000 | 0.0002 | NA | 1 |
| 0.18 | −0.9945 | −0.0001 | −0.0003 | −0.0002 | NA | NA | 0.0001 | −0.0002 | −0.0002 | NA | −0.0004 | 0.0001 | NA | 0 |
| −0.36 | −1.2829 | −0.0014 | −0.0004 | −0.0009 | NA | NA | −0.0003 | 0.0012 | 0.0001 | NA | 0.0009 | 0.0001 | NA | 0 |
| −0.09 | −1.1664 | −0.0002 | −0.0001 | −0.0003 | NA | NA | 0.0001 | 0.0005 | 0.0000 | NA | 0.0000 | 0.0000 | NA | 1 |
| 0.69 | −0.9659 | −0.6857 | 0.0629 | 0.4525 | NA | NA | −0.4935 | −0.8644 | 0.8301 | NA | −0.5158 | 2.6816 | NA | 0 |
| 0.44 | −0.3847 | −0.7390 | 0.7145 | 0.3756 | −0.7884 | 1.2173 | −0.2850 | 2.9499 | 0.1687 | −0.0587 | 0.1267 | −0.2870 | −0.3209 | 0 |
| −0.31 | −1.4948 | −1.0498 | −2.8897 | 0.0304 | −0.4199 | 0.0747 | 0.7711 | −0.9647 | −0.5248 | 0.3931 | −1.0751 | −0.0432 | −0.1870 | 1 |
| 0.46 | −1.1302 | −0.4967 | 0.8120 | −0.2666 | NA | NA | −0.0731 | −0.4687 | −0.7250 | NA | 3.2179 | −0.4825 | NA | 1 |
| −0.10 | −0.8123 | −0.4796 | 0.6374 | −0.1975 | NA | NA | 0.4449 | 0.9334 | 0.0733 | NA | −1.6412 | −1.3094 | NA | 0 |
| −0.53 | −1.4447 | −2.0167 | 1.6603 | 0.7312 | −1.1717 | −1.0437 | −0.1610 | −0.0593 | 0.5134 | 0.0162 | 0.6920 | −0.1829 | −0.3992 | 1 |
| −0.53 | −1.0596 | 0.0001 | −0.0003 | −0.0007 | NA | NA | 0.0000 | −0.0004 | −0.0001 | NA | 0.0001 | 0.0003 | NA | 0 |
| 0.58 | −0.8939 | 0.0005 | 0.0003 | −0.0003 | NA | NA | −0.0002 | 0.0002 | 0.0004 | NA | −0.0005 | 0.0002 | NA | 1 |
| 1.09 | −0.7523 | −3.3496 | −1.2362 | 0.0969 | 0.2402 | −1.3002 | −0.7260 | −0.5881 | −0.2630 | −4.4986 | −0.1508 | 0.0576 | 0.2127 | 1 |
| −0.46 | −1.4550 | 0.9717 | −0.0208 | 0.0628 | 0.4153 | −0.2110 | 0.1518 | −0.7275 | 0.1285 | 0.3430 | −0.1190 | 0.0742 | 0.3411 | 1 |
| −0.50 | −1.3333 | 0.0005 | 0.0000 | −0.0005 | NA | NA | −0.0002 | −0.0001 | 0.0001 | NA | 0.0000 | 0.0003 | NA | 0 |
| −0.94 | −0.8281 | −0.0005 | −0.0002 | −0.0005 | NA | NA | 0.0000 | 0.0004 | 0.0002 | NA | −0.0003 | 0.0001 | NA | 0 |
| −0.93 | −1.0619 | −2.6753 | −2.6890 | −10.0013 | −0.0553 | 0.3335 | −0.4459 | −0.2001 | −1.6829 | 0.3316 | −1.0388 | 0.0752 | 0.1066 | 0 |
| −0.35 | −0.7838 | 1.5200 | 1.6013 | 1.1265 | 0.9643 | −0.2442 | 0.3450 | 0.8661 | −0.7480 | −0.0456 | −0.2642 | 0.2646 | −0.2143 | 1 |
| −0.11 | −1.1000 | 0.5101 | 1.0776 | 0.4054 | 0.6806 | −0.2101 | 1.0115 | −0.0863 | −0.4039 | 0.2189 | 1.3330 | −0.4786 | 0.4537 | 1 |
| −0.75 | −1.1438 | −0.0004 | −0.0003 | −0.0006 | NA | NA | 0.0003 | 0.0001 | −0.0002 | NA | 0.0007 | 0.0001 | NA | 0 |
| −0.58 | −0.7059 | −0.0001 | −0.0002 | 0.0006 | NA | NA | 0.0000 | −0.0001 | −0.0002 | NA | −0.0003 | 0.0001 | NA | 0 |
| −0.32 | −0.8475 | −0.4928 | 0.7024 | 0.4201 | NA | NA | 0.3201 | −2.0278 | −0.3487 | NA | 0.2459 | −0.4683 | NA | 1 |
| −1.58 | −1.2599 | −0.0043 | 0.0082 | −4.6757 | NA | NA | 0.0018 | 0.0147 | −0.0617 | NA | 0.0364 | −0.0179 | NA | 0 |
| −0.60 | −1.1165 | −0.0020 | 0.0036 | 3.7329 | NA | NA | −0.0007 | −0.0013 | 0.1267 | NA | −0.0074 | −0.0012 | NA | 0 |
| −0.62 | −1.2382 | 0.0007 | 0.0003 | 0.0009 | NA | NA | −0.0005 | 0.0003 | 0.0006 | NA | −0.0002 | 0.0002 | NA | 1 |
| −0.25 | −1.1152 | −0.0001 | 0.0002 | 0.0001 | NA | NA | −0.0001 | −0.0003 | 0.0001 | NA | 0.0000 | 0.0000 | NA | 0 |
| −0.78 | −0.1123 | 4.8370 | −0.9087 | 1.0102 | −0.8367 | −0.2083 | 0.0095 | 0.0091 | 0.4612 | 0.4946 | 0.5069 | −0.1351 | 0.1281 | 0 |
| −0.57 | −0.9683 | −0.0004 | −0.0004 | −0.0012 | NA | NA | −0.0002 | −0.0002 | −0.0001 | NA | 0.0006 | 0.0002 | NA | 1 |
| 0.05 | −1.2958 | −1.5998 | −3.3128 | −0.2725 | NA | NA | 0.6233 | 0.4254 | −0.5007 | NA | −0.2111 | 0.1739 | NA | 0 |
| 0.06 | −0.9454 | −1.1361 | 0.8879 | 0.7020 | −0.5309 | 0.1104 | 0.5221 | −0.3278 | 0.2310 | −0.0873 | −0.0221 | −0.2594 | 0.3665 | 0 |
| −3.94 | 2.0603 | 0.0004 | −0.0008 | 1.7260 | NA | NA | 0.0005 | 0.0092 | 0.7718 | NA | 0.0274 | −0.0068 | NA | 0 |
| −0.23 | −0.6989 | 0.0000 | 0.0001 | 0.0003 | NA | NA | −0.0002 | 0.0005 | −0.0002 | NA | 0.0006 | 0.0001 | NA | 0 |
| 2.16 | 2.4730 | 0.6370 | −0.3090 | −0.7059 | NA | NA | −0.5477 | −0.5773 | 0.0265 | NA | −1.1842 | −0.3751 | NA | 1 |
| 0.58 | 0.0063 | 0.5013 | 0.4893 | 0.2564 | NA | NA | 1.3168 | 0.0606 | −1.1926 | NA | −0.1919 | 1.2841 | NA | 0 |
| −0.52 | −1.0384 | 0.0003 | 0.0003 | −0.0004 | NA | NA | 0.0000 | 0.0003 | 0.0004 | NA | 0.0002 | 0.0002 | NA | 0 |
| −0.60 | −0.8615 | −0.0001 | 0.0000 | 0.0002 | NA | NA | −0.0001 | 0.0002 | 0.0003 | NA | −0.0003 | 0.0001 | NA | 1 |
| −0.86 | −1.0056 | −0.0002 | 0.0001 | −0.0001 | NA | NA | −0.0002 | 0.0002 | 0.0001 | NA | 0.0003 | 0.0001 | NA | 1 |
| −1.05 | 2.2650 | −0.4772 | 0.3809 | −0.0447 | −0.5909 | −0.9023 | 0.5977 | −0.9736 | 0.6487 | 0.0421 | −0.5154 | 0.4914 | −0.3444 | 1 |
| −0.40 | −1.3260 | −3.9472 | 0.3884 | 0.7955 | 0.0727 | −2.0010 | 2.0954 | 0.1590 | 2.6226 | 0.2451 | 0.7849 | −0.1379 | −0.2074 | 0 |
| −0.57 | −2.9982 | −2.4609 | 2.4095 | 0.2554 | 0.2964 | 0.4693 | −2.5146 | −0.2499 | 0.2804 | −0.2214 | 0.0939 | −0.1148 | −0.2861 | 1 |
| 0.68 | 0.3402 | −0.3872 | 0.1055 | 0.0675 | NA | NA | 0.0015 | 1.7510 | 1.0939 | NA | 0.3737 | −0.3013 | NA | 0 |
| 7.70 | 0.3498 | 0.0004 | −0.0005 | −0.6107 | NA | NA | 0.0010 | 0.0031 | −0.1426 | NA | 0.0087 | −0.0020 | NA | 1 |
| 1.06 | 0.4635 | −0.9050 | −0.3898 | −0.3701 | NA | NA | 0.1511 | −1.1379 | 0.2515 | NA | 0.3973 | −0.4331 | NA | 1 |
| 1.23 | 12.0198 | −0.3782 | 1.0512 | 0.5078 | 0.1859 | 2.4513 | 0.0449 | −0.5293 | −0.2711 | −0.2043 | 0.3021 | −0.2013 | −0.1588 | 1 |
| 0.37 | −0.2502 | 0.5712 | 1.3420 | −4.5686 | −0.5416 | −1.1736 | 2.0088 | 1.2550 | −0.5547 | −0.4223 | 0.9265 | 0.1416 | −0.3990 | 1 |
| −0.06 | 1.1362 | −0.2150 | 0.2570 | −0.0893 | NA | NA | −0.0913 | 0.3859 | 0.4874 | NA | 1.9796 | −0.3562 | NA | 1 |
| −0.38 | −1.7879 | 0.8948 | 0.4178 | −0.5749 | NA | NA | −0.5049 | −0.8034 | −0.9075 | NA | −0.9886 | 1.8059 | NA | 1 |
| 0.29 | −0.5132 | −0.3914 | 0.6647 | 0.3593 | NA | NA | 0.2104 | 0.4256 | 0.6320 | NA | −0.2573 | 0.7161 | NA | 1 |
| −0.70 | 0.8566 | 1.6224 | 0.2506 | −0.9982 | NA | NA | 0.2745 | 0.5925 | −0.8857 | NA | −0.3552 | −0.1142 | NA | 0 |
| 0.79 | −0.5415 | −0.8498 | −2.5259 | 0.4881 | 0.4328 | 0.8364 | 0.7550 | 0.0296 | −0.9278 | 0.0123 | 0.1001 | 0.1259 | −0.2476 | 1 |
| −0.60 | −0.3650 | −0.2905 | −0.0512 | −0.6415 | −0.0682 | −0.0383 | −0.2986 | −0.9179 | 2.0054 | 0.2084 | 0.7068 | 0.3643 | −0.0931 | 1 |
| −5.55 | 3.1902 | 0.0109 | −0.0189 | −3.0709 | NA | NA | −0.0009 | −0.0094 | 0.1030 | NA | −0.0078 | 0.0214 | NA | 1 |
| −0.50 | −1.8686 | −0.0050 | 0.0083 | −3.1003 | NA | NA | −0.0003 | −0.0033 | −2.6155 | NA | −0.0230 | −0.0013 | NA | 0 |
| −0.12 | −0.0772 | −0.6854 | 0.3003 | 0.8149 | NA | NA | −0.0190 | 0.6278 | −0.5377 | NA | 0.0148 | 0.2127 | NA | 1 |
| −0.01 | −1.0528 | 0.3664 | 0.2201 | 0.4967 | NA | NA | −0.2511 | −1.1761 | −0.1006 | NA | −1.1162 | −3.2650 | NA | 0 |
| 1.06 | −1.3482 | 0.3135 | −2.1930 | 0.3140 | 0.4224 | 0.9803 | 0.1261 | −0.1612 | −0.8296 | 0.0136 | −0.0489 | −0.1196 | −0.3487 | 1 |
| −0.95 | −1.4507 | 3.0651 | 1.3884 | −1.0154 | 0.7140 | 0.3134 | −1.2719 | 0.5091 | 3.1311 | −0.0805 | −2.8353 | −0.0846 | −0.1052 | 1 |
| −0.86 | −0.2576 | 0.3737 | −0.2376 | −1.3131 | NA | NA | −0.4174 | 1.4257 | −0.5199 | NA | −0.4053 | −0.0390 | NA | 0 |
| −1.06 | 12.1430 | 4.4910 | 0.1009 | 0.2154 | 0.6651 | −0.5935 | 0.1190 | 0.0007 | 0.2277 | 0.4380 | 0.7631 | 0.0011 | 0.8869 | 1 |
| −0.07 | −3.6470 | 2.1785 | 1.5038 | −0.2582 | 0.2246 | 0.2257 | 0.1405 | −0.5706 | 0.5513 | 0.4398 | 0.7799 | 0.1964 | 0.9424 | 0 |
| −1.41 | 2.0627 | −1.3872 | −0.8062 | 1.2210 | 0.1897 | −0.1009 | −0.9688 | −0.6420 | −0.9602 | 0.5106 | −0.1875 | −0.1635 | −0.2265 | 1 |
| −0.34 | 0.7758 | −2.5132 | −1.4033 | 3.7513 | 0.6468 | 0.9315 | −0.3926 | 1.4862 | −0.9600 | −0.0248 | −1.8116 | 0.4234 | 0.1220 | 1 |
| 0.62 | −1.9197 | −0.2983 | 1.1532 | 0.2476 | 0.2393 | −0.3394 | 0.6279 | −0.5883 | −0.5737 | 0.1359 | −0.0774 | −0.2618 | −0.3510 | 1 |
| −0.36 | −2.7296 | 1.9929 | −0.8686 | 0.8273 | −0.6502 | 0.1133 | −0.1410 | −0.4138 | −0.6330 | 0.0016 | −1.3996 | −0.0937 | −0.1552 | 1 |
| 1.54 | −4.9226 | 0.0318 | 3.8192 | 0.5531 | 0.5636 | 2.0236 | 0.0601 | 0.1436 | −0.4260 | −0.4011 | 0.4416 | 0.1913 | 0.2049 | 1 |
| 3.16 | 2.7551 | 0.4895 | −0.6657 | 1.2019 | 0.3243 | −2.0970 | −1.9554 | 0.6722 | 0.5185 | 1.9875 | 2.6677 | 0.0445 | 0.1644 | 1 |
| −0.25 | −3.5416 | 4.4666 | −3.5602 | 1.3178 | −1.5877 | 0.5184 | 0.1228 | 0.0728 | −1.0598 | 0.2278 | −0.5587 | 0.1155 | 0.1297 | 0 |
| 0.19 | 0.1986 | −0.0543 | 0.1399 | 0.1078 | 0.4662 | −0.0131 | −0.1413 | −0.2229 | 0.0067 | −0.1357 | 0.1119 | 0.0579 | −0.2671 | |
| 0.13 | 0.1070 | 0.6630 | 0.2590 | 0.3850 | 0.0124 | 0.9470 | 0.2540 | 0.0699 | 0.9570 | 0.4910 | 0.3670 | 0.6420 | 0.1690 | |

* = Endoscopic remission

TABLE 8

| | CD14 | CD4 | Colonic | | |
|---|---|---|---|---|---|
| | transcriptomics | transcriptomics | transcriptomics | Genomics | Proteomics |
| LF1 | 0.105273974 | 0.088385213 | 0.003020753 | 0.950280698 | 0.00059792 |
| LF2 | 9.79E−06 | 0.021578921 | 0.359994242 | 7.53E−09 | 0.014131716 |
| LF3 | 0.168740926 | 0.180140734 | 7.54E−07 | 7.11E−10 | 4.14E−07 |
| LF4 | 0.04953133 | 0.021118877 | 0.169837798 | 3.60E−09 | 0.000186971 |
| LF5 | 0.020767421 | 0.015356318 | 0.001364137 | 0.175277505 | 0.012267772 |
| LF6 | 0.203808131 | 3.80E−06 | 0.011145948 | 1.38E−09 | 6.04E−07 |
| LF7 | 0.007917863 | 0.027104104 | 0.018025908 | 9.59E−09 | 0.129881764 |
| LF8 | 0.049873998 | 0.075545791 | 0.008884538 | 8.64E−09 | 0.037829629 |
| LF9 | 0.005778834 | 0.089055019 | 0.06924577 | 5.07E−09 | 7.45E−07 |
| LF10 | 0.002831743 | 0.019490559 | 0.019680736 | 0.061263467 | 0.002262467 |
| LF11 | 0.033345742 | 0.026426239 | 0.036621108 | 5.25E−09 | 5.47E−06 |
| LF12 | 0.026443226 | 0.036596012 | 0.027253031 | 3.21E−09 | 7.14E−05 |
| LF13 | 0.000284343 | 0.073086147 | 4.22E−05 | 1.04E−10 | 9.70E−08 |
| LF14 | 0.033559388 | 0.002761335 | 0.004343051 | 1.94E−09 | 1.65E−07 |

TABLE 9

| | CD14 | CD4 | Colonic | | Ileal | |
|---|---|---|---|---|---|---|
| | transcriptomics | transcriptomics | transcriptomics | Genomics | transcriptomics | Proteomics |
| LF1 | 0.020150505 | 0.003058299 | 0.000846118 | 0.334258023 | 0.495806409 | 2.11E−06 |
| LF2 | 0.107734145 | 0.046613838 | 0.00479919 | 0.452931764 | 3.11E−06 | 5.80E−06 |
| LF3 | 6.09E−06 | 0.046999362 | 0.004739375 | 0.524176396 | 0.022252521 | 4.05E−06 |
| LF4 | 0.026229796 | 8.00E−06 | 0.497343231 | 3.51E−09 | 0.018303664 | 0.00165458 |
| LF5 | 0.037831659 | 0.105712983 | 0.004057203 | 7.20E−09 | 0.015250092 | 0.12256421 |
| LF6 | 0.004332774 | 0.238432617 | 0.007804865 | 0.033322902 | 1.53E−06 | 2.62E−06 |
| LF7 | 0.073274257 | 0.016133792 | 0.101671539 | 9.49E−09 | 0.010775876 | 0.05717292 |
| LF8 | 0.074763911 | 4.27E−06 | 0.019791162 | 0.012119188 | 0.030863568 | 0.02554424 |
| LF9 | 1.75E−06 | 0.029411629 | 0.002378223 | 5.12E−09 | 0.126107277 | 3.47E−06 |
| LF10 | 0.1039055 | 2.22E−06 | 1.56E−07 | 0.045663968 | 2.99E−07 | 7.12E−07 |
| LF11 | 3.40E−06 | 0.111258636 | 0.003463474 | 4.79E−09 | 0.034760745 | 1.05E−06 |
| LF12 | 0.100361605 | 0.00477009 | 0.027440728 | 4.67E−09 | 0.003723176 | 7.69E−07 |
| LF13 | 0.011756012 | 0.020174663 | 0.008019835 | 0.089616176 | 0.003114438 | 0.00333921 |
| LF14 | 0.035499941 | 0.021397798 | 0.044367602 | 2.61E−09 | 0.00313375 | 6.84E−07 |
| LF15 | 0.011464998 | 0.06266972 | 0.009527294 | 3.02E−09 | 0.004044323 | 1.26E−06 |

TABLE 10

| | CD14 | CD4 | Colonic | | |
|---|---|---|---|---|---|
| | transcriptomics | transcriptomics | transcriptomics | Genomics | Proteomics |
| LF1 | 0.442632084 | 5.33E−06 | 0.007531549 | 0.982202051 | 1.57E−06 |
| LF2 | 0.005086355 | 0.325089094 | 0.02583928 | 3.20E−09 | 3.66E−07 |
| LF3 | 0.00333871 | 0.014214092 | 0.334368288 | 1.89E−09 | 1.28E−06 |
| LF4 | 0.045498059 | 0.000311435 | 1.76E−06 | 0.108273583 | 0.009734834 |
| LF5 | 0.102532335 | 0.050208257 | 0.004298854 | 4.27E−09 | 1.61E−06 |
| LF6 | 0.026804598 | 0.011217684 | 0.107876292 | 3.61E−09 | 6.60E−07 |
| LF7 | 2.14E−06 | 0.086136381 | 0.030769008 | 1.21E−09 | 0.000171567 |
| LF8 | 2.81E−06 | 0.048805876 | 0.05816833 | 4.01E−09 | 2.60E−06 |
| LF9 | 0.013884364 | 0.069446956 | 0.005171907 | 1.23E−09 | 9.22E−07 |
| LF10 | 0.045592335 | 0.031843017 | 0.007515848 | 3.18E−09 | 1.12E−06 |
| LF11 | 0.055844894 | 1.26E−05 | 0.025234355 | 6.19E−10 | 3.42E−07 |
| LF12 | 0.077704623 | 2.22E−06 | 0.002306699 | 9.74E−10 | 2.81E−07 |
| LF13 | 1.86E−06 | 0.076106163 | 7.50E−07 | 6.86E−10 | 5.18E−07 |
| LF14 | 0.015243691 | 8.77E−05 | 0.043536692 | 4.86E−09 | 4.46E−05 |
| LF15 | 0.014899092 | 0.026819286 | 0.013584118 | 1.15E−09 | 4.99E−07 |

TABLE 11

| | CD14 | CD4 | Colonic | | Ileal | |
|---|---|---|---|---|---|---|
| | transcriptomics | transcriptomics | transcriptomics | Genomics | transcriptomics | Proteomics |
| LF1 | 1.75E−06 | 8.67E−06 | 6.98E−08 | 2.84E−09 | 0.004638137 | 0.954307756 |
| LF2 | 2.32E−06 | 0.022701945 | 0.078441858 | 0.834271439 | 0.005266775 | 2.41E−05 |
| LF3 | 0.048774509 | 4.66E−06 | 0.007605199 | 0.01058122 | 0.345318237 | 1.91E−07 |
| LF4 | 0.008617638 | 0.017423723 | 5.74E−08 | 0.359833042 | 3.51E−10 | 0.007421826 |
| LF5 | 0.1812121 | 0.148223702 | 0.018473985 | 3.41E−09 | 0.002591473 | 3.64E−08 |
| LF6 | 0.029239849 | 2.84E−06 | 0.247929687 | 3.67E−09 | 0.06754099 | 9.45E−08 |
| LF7 | 0.134358638 | 1.11E−05 | 0.166522802 | 5.21E−09 | 0.002596748 | 0 |
| LF8 | 1.85E−06 | 0.190583538 | 0.016226277 | 0.078432602 | 0.004980627 | 0.000157842 |
| LF9 | 2.60E−06 | 0.026639018 | 0.012440982 | 0.104642669 | 0.006781351 | 0 |
| LF10 | 0.024241853 | 2.92E−06 | 7.17E−08 | 5.66E−09 | 0.12481585 | 0 |
| LF11 | 1.97E−06 | 0.022741538 | 7.25E−08 | 4.23E−09 | 0.078348768 | 4.25E−08 |
| LF12 | 0.035699754 | 0.003652557 | 0.037520329 | 6.94E−09 | 0.02318372 | 9.93E−08 |
| LF13 | 0.092620236 | 4.45E−06 | 0.001607794 | 1.13E−09 | 0.001651592 | 2.45E−08 |
| LF14 | 2.00E−06 | 0.087368741 | 0.003380707 | 8.43E−10 | 0.000214822 | 0 |
| LF15 | 0.012721971 | 0.065161833 | 3.13E−08 | 1.29E−09 | 0.004013418 | 6.60E−09 |
| LF16 | 0.063446827 | 0.012745593 | 2.79E−08 | 2.83E−09 | 0.00257053 | 1.68E−08 |
| LF17 | 0.011917856 | 2.73E−06 | 0.03637788 | 2.23E−09 | 0.02768804 | 9.90E−10 |
| LF18 | 3.63E−06 | 0.068279999 | 5.20E−08 | 2.73E−09 | 9.33E−05 | 0 |
| LF19 | 0.031015424 | 1.57E−06 | 8.06E−08 | 2.99E−09 | 0.028866491 | 1.09E−08 |
| LF20 | 0.033015328 | 0.021404602 | 4.49E−08 | 8.99E−10 | 0.001514128 | 7.05E−09 |
| LF21 | 1.96E−06 | 0.030069691 | 1.56E−09 | 1.14E−10 | 1.45E−11 | 0.000439666 |

TABLE 12

| Drug | Disease | Latent Factor | Dominant -omic layer |
|---|---|---|---|
| Vedolizumab | Ulcerative | LF 5 | Genomics |
| | colitis | LF 9-LF 12 | CD4⁺ T cell transcriptomics |
| Vedolizumab | Crohn's | LF 3 | Genomics |
| | disease | LF 8 | Monocyte transcriptomics |
| | | LF9 | Ileal tissue transcriptomics |
| Anti-TNF | Ulcerative | LF2 | CD4⁺ T cell transcriptomics |
| agents | colitis | LF 3-LF | Colonic tissue |
| | | 8-LF 14 | transcriptomics |
| | | LF 10 | Monocyte transcriptomics |
| Anti-TNF | Crohn's | LF 2-LF9 | Genomics |
| agents | disease | LF 5-LF 16 | Monocyte transcriptomics |
| | | LF 8 | CD4⁺ T cell transcriptomics |

TABLE 13

| | | | | Anti-TNF | Anti-TNF | Anti-TNF | Anti-TNF | Anti-TNF |
|---|---|---|---|---|---|---|---|---|
| | Vedolizumab - | Vedolizumab - | Vedolizumab - | agents - UC - | agents - UC - | agents - | agents - | agents - CD - |
| | UC - CD4 T cell | CD- CD14 | CD- Ileal | CD4 T cell | Colonic | UC - CD14 | CD - CD14 | CD4 T cell |
| | transcrip- | transcrip- | transcrip- | transcrip- | transcrip- | transcrip- | transcrip- | transcrip- |
| Feature | tomics | tomics | tomics | tomics | tomics | tomics | tomics | tomics |
| NPDC1 | 1 | 0 | 0 | 0 | 0 | 0 | 0 | 0 |
| KRT73 | 1 | 0 | 0 | 0 | 0 | 0 | 0 | 0 |
| ERAP2 | 1 | 0 | 1 | 1 | 0 | 0 | 0 | 1 |
| AFAP1 | 1 | 0 | 0 | 0 | 0 | 0 | 0 | 0 |
| MOB1B | 1 | 0 | 0 | 0 | 0 | 0 | 0 | 0 |
| TP53INP1 | 1 | 0 | 0 | 0 | 0 | 0 | 0 | 0 |
| IGFBP3 | 1 | 0 | 0 | 0 | 0 | 0 | 0 | 0 |
| NKG7 | 1 | 0 | 0 | 1 | 0 | 0 | 0 | 0 |
| PLEK | 1 | 0 | 0 | 0 | 0 | 0 | 0 | 0 |
| LRRC16A | 1 | 0 | 0 | 0 | 0 | 0 | 0 | 0 |
| NLRP2 | 1 | 0 | 1 | 0 | 1 | 0 | 0 | 0 |
| RNF207 | 1 | 0 | 0 | 0 | 0 | 0 | 0 | 0 |
| ZNF879 | 1 | 0 | 0 | 0 | 0 | 0 | 0 | 0 |
| CCL5 | 1 | 0 | 0 | 0 | 0 | 0 | 0 | 0 |
| RPS17 | 1 | 0 | 0 | 0 | 0 | 0 | 0 | 0 |
| NELL2 | 1 | 0 | 0 | 0 | 0 | 0 | 0 | 0 |
| FAM118A | 1 | 0 | 0 | 0 | 0 | 0 | 0 | 0 |
| CD101 | 1 | 0 | 0 | 0 | 0 | 0 | 0 | 0 |
| TNF | 1 | 0 | 0 | 0 | 0 | 0 | 0 | 0 |
| RPS26 | 1 | 0 | 0 | 0 | 0 | 0 | 0 | 0 |
| PAXIP1_AS2 | 1 | 0 | 0 | 0 | 0 | 0 | 0 | 0 |
| SMAD7 | 1 | 0 | 0 | 0 | 0 | 0 | 0 | 0 |
| BLK | 1 | 0 | 0 | 0 | 0 | 0 | 0 | 0 |
| GNLY | 1 | 0 | 0 | 1 | 0 | 0 | 0 | 1 |
| MDGA1 | 1 | 0 | 0 | 1 | 0 | 0 | 0 | 1 |
| MIDN | 1 | 0 | 0 | 0 | 0 | 0 | 0 | 0 |
| IFIT3 | 1 | 0 | 0 | 0 | 0 | 0 | 0 | 0 |
| NCF2 | 1 | 0 | 0 | 0 | 0 | 0 | 0 | 0 |
| CPT1A | 1 | 0 | 0 | 1 | 0 | 0 | 0 | 0 |
| DACT1 | 1 | 0 | 0 | 0 | 0 | 0 | 0 | 0 |
| STON2 | 0 | 1 | 0 | 0 | 0 | 0 | 0 | 0 |
| ABCG1 | 0 | 1 | 0 | 0 | 0 | 1 | 0 | 0 |
| ERAP1 | 0 | 1 | 0 | 0 | 0 | 0 | 0 | 0 |
| APOL6 | 0 | 1 | 0 | 0 | 0 | 0 | 0 | 0 |
| ERV3_1 | 0 | 1 | 0 | 0 | 0 | 0 | 0 | 0 |
| ANKH | 0 | 1 | 0 | 0 | 0 | 0 | 0 | 0 |
| ARHGEF17 | 0 | 1 | 0 | 0 | 0 | 0 | 0 | 0 |
| TRIB3 | 0 | 1 | 0 | 0 | 0 | 0 | 0 | 0 |
| MAFF | 0 | 1 | 0 | 0 | 0 | 1 | 0 | 0 |
| RIMKLB | 0 | 1 | 0 | 0 | 0 | 0 | 0 | 0 |
| PLB1 | 0 | 1 | 1 | 0 | 0 | 0 | 0 | 0 |
| PCDH12 | 0 | 1 | 0 | 0 | 0 | 0 | 0 | 0 |
| SLC7A5 | 0 | 1 | 0 | 0 | 0 | 0 | 0 | 1 |
| PRMT6 | 0 | 1 | 0 | 0 | 0 | 0 | 0 | 0 |
| ID1 | 0 | 1 | 0 | 0 | 0 | 0 | 0 | 0 |
| ZNF844 | 0 | 1 | 0 | 0 | 0 | 0 | 0 | 0 |
| RPH3A | 0 | 1 | 0 | 0 | 0 | 1 | 0 | 0 |
| HLA_DQB1 | 0 | 1 | 0 | 1 | 1 | 0 | 1 | 0 |
| KCNN4 | 0 | 1 | 0 | 0 | 0 | 0 | 0 | 0 |
| CDC42EP1 | 0 | 1 | 0 | 0 | 0 | 0 | 0 | 0 |
| SPATA20 | 0 | 1 | 0 | 0 | 0 | 1 | 0 | 0 |
| CCR1 | 0 | 1 | 0 | 0 | 0 | 0 | 0 | 0 |
| GPR183 | 0 | 1 | 0 | 0 | 0 | 1 | 0 | 0 |
| EMR3 | 0 | 1 | 0 | 0 | 0 | 0 | 0 | 0 |
| SPTBN5 | 0 | 1 | 0 | 0 | 0 | 0 | 0 | 0 |
| PVRL2 | 0 | 1 | 0 | 0 | 0 | 0 | 0 | 0 |
| SOD2 | 0 | 1 | 0 | 0 | 0 | 0 | 0 | 0 |
| PPP1R15B | 0 | 1 | 0 | 0 | 0 | 0 | 0 | 0 |
| CDKN1B | 0 | 1 | 0 | 0 | 0 | 0 | 0 | 0 |
| EIF4A3 | 0 | 1 | 0 | 0 | 0 | 0 | 0 | 0 |
| ABCA1 | 0 | 1 | 0 | 0 | 0 | 1 | 1 | 1 |
| DDIT3 | 0 | 1 | 0 | 0 | 0 | 0 | 0 | 0 |
| FCGR3A | 0 | 1 | 0 | 0 | 0 | 0 | 0 | 0 |
| RASD1 | 0 | 1 | 0 | 0 | 1 | 0 | 0 | 0 |
| HSPE1 | 0 | 1 | 0 | 0 | 0 | 0 | 0 | 0 |
| NFKBIA | 0 | 1 | 0 | 0 | 0 | 0 | 0 | 0 |
| MAP3K7CL | 0 | 1 | 0 | 0 | 0 | 0 | 0 | 0 |
| FAM26F | 0 | 1 | 0 | 0 | 0 | 0 | 1 | 0 |
| EMR1 | 0 | 1 | 0 | 0 | 0 | 0 | 0 | 0 |
| RRAD | 0 | 1 | 0 | 0 | 0 | 0 | 1 | 0 |
| ANKRD28 | 0 | 1 | 0 | 0 | 0 | 0 | 0 | 0 |
| FOSL1 | 0 | 1 | 0 | 0 | 0 | 0 | 0 | 0 |
| H1F0 | 0 | 1 | 0 | 0 | 0 | 0 | 0 | 0 |
| KIF23 | 0 | 1 | 0 | 0 | 0 | 0 | 0 | 0 |
| CDK11A | 0 | 1 | 0 | 0 | 0 | 0 | 0 | 0 |
| KLF9 | 0 | 1 | 0 | 0 | 0 | 0 | 1 | 0 |
| PIM2 | 0 | 1 | 0 | 0 | 0 | 0 | 0 | 0 |
| DNAJA4 | 0 | 1 | 0 | 0 | 0 | 0 | 0 | 0 |
| JUP | 0 | 1 | 0 | 0 | 0 | 1 | 0 | 0 |
| APOBEC3A | 0 | 1 | 0 | 0 | 0 | 0 | 0 | 0 |
| GSTM4 | 0 | 1 | 0 | 0 | 0 | 0 | 0 | 0 |
| LILRA3 | 0 | 1 | 0 | 0 | 0 | 0 | 0 | 0 |
| EGR1 | 0 | 1 | 0 | 0 | 0 | 1 | 0 | 0 |
| HSPD1 | 0 | 1 | 0 | 0 | 0 | 0 | 0 | 0 |
| PDK4 | 0 | 1 | 0 | 0 | 0 | 1 | 0 | 0 |
| OLR1 | 0 | 1 | 0 | 0 | 0 | 0 | 0 | 0 |
| SFMBT1 | 0 | 1 | 0 | 0 | 0 | 0 | 0 | 0 |
| NRP1 | 0 | 1 | 0 | 0 | 0 | 1 | 0 | 0 |
| CC2D2A | 0 | 1 | 0 | 0 | 0 | 0 | 0 | 0 |
| LIF | 0 | 1 | 0 | 0 | 0 | 1 | 0 | 0 |
| CKS2 | 0 | 1 | 0 | 0 | 0 | 0 | 0 | 0 |
| IL1R2 | 0 | 1 | 0 | 0 | 0 | 0 | 1 | 0 |
| C15orf38 | 0 | 1 | 0 | 0 | 0 | 0 | 0 | 0 |
| HLA_DRB5 | 0 | 1 | 1 | 0 | 0 | 1 | 0 | 0 |
| PHLDA1 | 0 | 1 | 0 | 0 | 0 | 1 | 0 | 0 |
| G0S2 | 0 | 1 | 0 | 0 | 0 | 0 | 1 | 0 |
| INSIG1 | 0 | 1 | 0 | 0 | 0 | 0 | 0 | 0 |
| IL8 | 0 | 1 | 0 | 0 | 1 | 1 | 0 | 0 |
| TRIB1 | 0 | 1 | 0 | 0 | 0 | 0 | 0 | 0 |
| LYZ | 0 | 1 | 0 | 1 | 0 | 0 | 0 | 0 |
| MFGE8 | 0 | 1 | 0 | 0 | 0 | 0 | 1 | 0 |
| SEMA6B | 0 | 1 | 0 | 0 | 0 | 0 | 1 | 0 |
| ZNF83 | 0 | 1 | 0 | 0 | 0 | 0 | 0 | 0 |
| DHCR7 | 0 | 1 | 0 | 0 | 0 | 0 | 0 | 0 |
| VSTM1 | 0 | 1 | 0 | 0 | 0 | 0 | 0 | 0 |
| MAFB | 0 | 1 | 0 | 0 | 0 | 0 | 0 | 0 |
| PRDX2 | 0 | 1 | 0 | 0 | 0 | 0 | 0 | 0 |
| FCER2 | 0 | 1 | 0 | 0 | 0 | 1 | 0 | 0 |
| EGR3 | 0 | 1 | 0 | 0 | 0 | 0 | 0 | 0 |
| EMP1 | 0 | 1 | 0 | 0 | 0 | 0 | 0 | 0 |
| S1PR3 | 0 | 1 | 0 | 0 | 0 | 0 | 1 | 0 |
| SEH1L | 0 | 1 | 0 | 0 | 0 | 0 | 0 | 0 |
| C3AR1 | 0 | 1 | 0 | 0 | 0 | 0 | 0 | 0 |
| HLA_DQA2 | 0 | 1 | 0 | 0 | 0 | 0 | 1 | 0 |
| PROK2 | 0 | 1 | 0 | 0 | 0 | 0 | 1 | 0 |
| LIPA | 0 | 1 | 0 | 0 | 0 | 1 | 0 | 0 |
| FCGR2B | 0 | 1 | 0 | 0 | 0 | 0 | 0 | 0 |
| LMNA | 0 | 1 | 0 | 0 | 0 | 0 | 1 | 0 |
| SDC3 | 0 | 1 | 0 | 0 | 0 | 0 | 0 | 0 |
| MRPL41 | 0 | 1 | 0 | 0 | 0 | 0 | 0 | 0 |
| TMTC1 | 0 | 1 | 0 | 0 | 0 | 0 | 0 | 0 |
| TRIM66 | 0 | 1 | 0 | 0 | 0 | 0 | 0 | 0 |
| CXCL13 | 0 | 0 | 1 | 0 | 1 | 0 | 0 | 0 |
| HLA_G | 0 | 0 | 1 | 0 | 0 | 0 | 0 | 0 |
| HLA_DOB | 0 | 0 | 1 | 0 | 0 | 0 | 0 | 0 |
| TRPM6 | 0 | 0 | 1 | 0 | 0 | 0 | 0 | 0 |
| HLA_DQB2 | 0 | 0 | 1 | 0 | 1 | 0 | 0 | 0 |
| PDE4C | 0 | 0 | 1 | 0 | 0 | 0 | 0 | 0 |
| SCNN1B | 0 | 0 | 1 | 0 | 0 | 0 | 0 | 0 |
| PI15 | 0 | 0 | 1 | 0 | 1 | 0 | 0 | 0 |
| ESPL1 | 0 | 0 | 1 | 0 | 0 | 0 | 0 | 0 |
| LCT | 0 | 0 | 1 | 0 | 0 | 0 | 0 | 0 |
| SLC4A4 | 0 | 0 | 1 | 0 | 0 | 0 | 0 | 0 |
| CEACAM7 | 0 | 0 | 1 | 0 | 0 | 0 | 0 | 0 |
| CCL13 | 0 | 0 | 1 | 0 | 0 | 0 | 0 | 0 |
| FAM189A1 | 0 | 0 | 1 | 0 | 0 | 0 | 0 | 0 |
| C17orf78 | 0 | 0 | 1 | 0 | 0 | 0 | 0 | 0 |
| AMPD1 | 0 | 0 | 1 | 0 | 0 | 0 | 0 | 0 |
| LDHD | 0 | 0 | 1 | 0 | 0 | 0 | 0 | 0 |
| STEAP3 | 0 | 0 | 1 | 0 | 0 | 0 | 0 | 0 |
| TNFSF11 | 0 | 0 | 1 | 0 | 0 | 0 | 0 | 0 |
| SMLR1 | 0 | 0 | 1 | 0 | 0 | 0 | 0 | 0 |
| ABCA4 | 0 | 0 | 1 | 0 | 0 | 0 | 0 | 0 |
| CA4 | 0 | 0 | 1 | 0 | 0 | 0 | 0 | 0 |
| SELL | 0 | 0 | 1 | 0 | 0 | 0 | 0 | 0 |
| AIFM3 | 0 | 0 | 1 | 0 | 0 | 0 | 0 | 0 |
| SLC16A10 | 0 | 0 | 1 | 0 | 0 | 0 | 0 | 0 |
| TFPI2 | 0 | 0 | 1 | 0 | 0 | 0 | 0 | 0 |
| CCL19 | 0 | 0 | 1 | 0 | 0 | 0 | 0 | 0 |
| SLC5A12 | 0 | 0 | 1 | 0 | 0 | 0 | 0 | 0 |
| GREM2 | 0 | 0 | 1 | 0 | 1 | 0 | 0 | 0 |
| NDRG4 | 0 | 0 | 1 | 0 | 0 | 0 | 0 | 0 |
| REG3G | 0 | 0 | 1 | 0 | 0 | 0 | 0 | 0 |
| CUBN | 0 | 0 | 1 | 0 | 0 | 0 | 0 | 0 |
| CTD_2228K2_5 | 0 | 0 | 1 | 0 | 0 | 0 | 0 | 0 |
| NXPE4 | 0 | 0 | 1 | 0 | 1 | 0 | 0 | 0 |
| PKIB | 0 | 0 | 1 | 0 | 0 | 0 | 0 | 0 |
| SLC26A2 | 0 | 0 | 1 | 0 | 0 | 0 | 0 | 0 |
| FSIP2 | 0 | 0 | 1 | 0 | 0 | 0 | 0 | 0 |
| ADORA2B | 0 | 0 | 1 | 0 | 0 | 0 | 0 | 0 |
| MUC12 | 0 | 0 | 1 | 0 | 0 | 0 | 0 | 0 |
| CYR61 | 0 | 0 | 1 | 0 | 0 | 0 | 0 | 0 |
| COL22A1 | 0 | 0 | 1 | 0 | 0 | 0 | 0 | 0 |
| KCNJ16 | 0 | 0 | 1 | 0 | 0 | 0 | 0 | 0 |
| CLC | 0 | 0 | 1 | 0 | 0 | 0 | 0 | 0 |
| WNT11 | 0 | 0 | 1 | 0 | 0 | 0 | 0 | 0 |
| CA1 | 0 | 0 | 1 | 0 | 1 | 0 | 0 | 0 |
| ABCC2 | 0 | 0 | 1 | 0 | 0 | 0 | 0 | 0 |
| DPEP1 | 0 | 0 | 1 | 0 | 0 | 0 | 0 | 0 |
| AKR1B15 | 0 | 0 | 1 | 0 | 0 | 0 | 0 | 0 |
| GJB3 | 0 | 0 | 1 | 0 | 0 | 0 | 0 | 0 |
| DEFB1 | 0 | 0 | 1 | 0 | 0 | 0 | 0 | 0 |
| CA7 | 0 | 0 | 1 | 0 | 1 | 0 | 0 | 0 |
| VSIG2 | 0 | 0 | 1 | 0 | 0 | 0 | 0 | 0 |
| CA12 | 0 | 0 | 1 | 0 | 0 | 0 | 0 | 0 |
| HMGCS2 | 0 | 0 | 1 | 0 | 0 | 0 | 0 | 0 |
| MT1H | 0 | 0 | 1 | 0 | 0 | 0 | 0 | 0 |
| MYEOV | 0 | 0 | 1 | 0 | 0 | 0 | 0 | 0 |
| FAM134B | 0 | 0 | 1 | 0 | 0 | 0 | 0 | 0 |
| DHRS9 | 0 | 0 | 1 | 0 | 0 | 0 | 1 | 0 |
| SLC13A1 | 0 | 0 | 1 | 0 | 0 | 0 | 0 | 0 |
| CNGA1 | 0 | 0 | 1 | 0 | 0 | 0 | 0 | 0 |
| DUOX2 | 0 | 0 | 1 | 0 | 0 | 0 | 0 | 0 |
| DAB1 | 0 | 0 | 1 | 0 | 0 | 0 | 0 | 0 |
| CA9 | 0 | 0 | 1 | 0 | 0 | 0 | 0 | 0 |
| GPR15 | 0 | 0 | 1 | 0 | 0 | 0 | 0 | 1 |
| TMPRSS15 | 0 | 0 | 1 | 0 | 0 | 0 | 0 | 0 |
| SLC26A3 | 0 | 0 | 1 | 0 | 0 | 0 | 0 | 0 |
| CA2 | 0 | 0 | 1 | 0 | 0 | 0 | 0 | 0 |
| PIGZ | 0 | 0 | 1 | 0 | 0 | 0 | 0 | 0 |
| ITGA11 | 0 | 0 | 1 | 0 | 0 | 0 | 0 | 0 |
| GSTT1 | 0 | 0 | 1 | 0 | 0 | 0 | 0 | 0 |
| SDR16C5 | 0 | 0 | 1 | 0 | 0 | 0 | 0 | 0 |
| CXCL11 | 0 | 0 | 1 | 0 | 0 | 0 | 0 | 0 |
| TM4SF4 | 0 | 0 | 1 | 0 | 0 | 0 | 0 | 0 |
| FMO1 | 0 | 0 | 1 | 0 | 0 | 0 | 0 | 0 |
| C10orf99 | 0 | 0 | 1 | 0 | 0 | 0 | 0 | 0 |
| REG1B | 0 | 0 | 1 | 0 | 0 | 0 | 0 | 0 |
| ATP10B | 0 | 0 | 1 | 0 | 0 | 0 | 0 | 0 |
| SLC16A9 | 0 | 0 | 1 | 0 | 0 | 0 | 0 | 0 |
| FAM151A | 0 | 0 | 1 | 0 | 0 | 0 | 0 | 0 |
| SLC9A2 | 0 | 0 | 1 | 0 | 0 | 0 | 0 | 0 |
| SFRP2 | 0 | 0 | 1 | 0 | 1 | 0 | 0 | 0 |
| CLCA4 | 0 | 0 | 1 | 0 | 0 | 0 | 0 | 0 |
| NAT8 | 0 | 0 | 1 | 0 | 0 | 0 | 0 | 0 |
| PTPRR | 0 | 0 | 1 | 0 | 0 | 0 | 0 | 0 |
| SLC9A3 | 0 | 0 | 1 | 0 | 0 | 0 | 0 | 0 |
| ENPP7 | 0 | 0 | 1 | 0 | 0 | 0 | 0 | 0 |
| RGS13 | 0 | 0 | 1 | 0 | 0 | 0 | 0 | 0 |
| MS4A10 | 0 | 0 | 1 | 0 | 0 | 0 | 0 | 0 |
| CYP2B6 | 0 | 0 | 1 | 0 | 0 | 0 | 0 | 0 |
| APOC3 | 0 | 0 | 1 | 0 | 0 | 0 | 0 | 0 |
| GSTA1 | 0 | 0 | 1 | 0 | 0 | 0 | 0 | 0 |
| GSTA2 | 0 | 0 | 1 | 0 | 0 | 0 | 0 | 0 |
| SULT2A1 | 0 | 0 | 1 | 0 | 0 | 0 | 0 | 0 |
| AADAC | 0 | 0 | 1 | 0 | 0 | 0 | 0 | 0 |
| DEFA5 | 0 | 0 | 1 | 0 | 0 | 0 | 0 | 0 |
| CCL18 | 0 | 0 | 1 | 0 | 0 | 0 | 0 | 0 |
| C12orf36 | 0 | 0 | 1 | 0 | 1 | 0 | 0 | 0 |
| LYPD8 | 0 | 0 | 1 | 0 | 0 | 0 | 0 | 0 |
| SLC30A10 | 0 | 0 | 1 | 0 | 0 | 0 | 0 | 0 |
| ZG16 | 0 | 0 | 1 | 0 | 1 | 0 | 0 | 0 |
| CEACAM1 | 0 | 0 | 1 | 0 | 0 | 0 | 0 | 0 |
| MMP1 | 0 | 0 | 1 | 0 | 1 | 0 | 0 | 0 |
| TRIM31 | 0 | 0 | 1 | 0 | 0 | 0 | 0 | 0 |
| TSPAN1 | 0 | 0 | 1 | 0 | 0 | 0 | 0 | 0 |
| CCL25 | 0 | 0 | 1 | 0 | 0 | 0 | 0 | 0 |
| OASL | 0 | 0 | 1 | 0 | 0 | 0 | 0 | 0 |
| DHDH | 0 | 0 | 1 | 0 | 0 | 0 | 0 | 0 |
| AKR1C1 | 0 | 0 | 1 | 0 | 0 | 0 | 0 | 0 |
| RETNLB | 0 | 0 | 1 | 0 | 1 | 0 | 0 | 0 |
| CKB | 0 | 0 | 1 | 0 | 1 | 0 | 0 | 0 |
| ELOVL4 | 0 | 0 | 0 | 1 | 0 | 0 | 0 | 0 |
| DDX11 | 0 | 0 | 0 | 1 | 0 | 0 | 0 | 0 |
| FGL2 | 0 | 0 | 0 | 1 | 0 | 0 | 0 | 0 |
| CTSW | 0 | 0 | 0 | 1 | 0 | 0 | 0 | 0 |
| DST | 0 | 0 | 0 | 1 | 0 | 0 | 0 | 0 |
| RPL21 | 0 | 0 | 0 | 1 | 0 | 0 | 0 | 1 |
| ZBTB16 | 0 | 0 | 0 | 1 | 0 | 0 | 0 | 0 |
| HBB | 0 | 0 | 0 | 1 | 1 | 0 | 0 | 0 |
| SLC7A8 | 0 | 0 | 0 | 1 | 0 | 0 | 0 | 1 |
| SMDT1 | 0 | 0 | 0 | 1 | 0 | 0 | 0 | 0 |
| PARVB | 0 | 0 | 0 | 1 | 0 | 0 | 0 | 0 |
| MARCKSL1 | 0 | 0 | 0 | 1 | 0 | 0 | 0 | 0 |
| DPYSL4 | 0 | 0 | 0 | 1 | 0 | 0 | 0 | 0 |
| ENTPD1 | 0 | 0 | 0 | 1 | 0 | 0 | 0 | 0 |
| GOLGA8B | 0 | 0 | 0 | 1 | 0 | 0 | 0 | 0 |
| FCN1 | 0 | 0 | 0 | 1 | 0 | 0 | 0 | 0 |
| RPS4Y1 | 0 | 0 | 0 | 1 | 0 | 0 | 1 | 0 |
| SOCS3 | 0 | 0 | 0 | 1 | 0 | 0 | 0 | 0 |
| SLAMF7 | 0 | 0 | 0 | 1 | 0 | 0 | 0 | 0 |
| FOSB | 0 | 0 | 0 | 1 | 1 | 0 | 0 | 0 |
| SESN1 | 0 | 0 | 0 | 1 | 0 | 0 | 0 | 0 |
| FKBP5 | 0 | 0 | 0 | 1 | 0 | 0 | 0 | 0 |
| FADS2 | 0 | 0 | 0 | 1 | 1 | 1 | 1 | 0 |
| PLAT | 0 | 0 | 0 | 0 | 1 | 0 | 0 | 0 |
| CDH3 | 0 | 0 | 0 | 0 | 1 | 0 | 0 | 0 |
| MESDC1 | 0 | 0 | 0 | 0 | 1 | 0 | 0 | 0 |
| PTPRCAP | 0 | 0 | 0 | 0 | 1 | 0 | 0 | 0 |
| TMEM160 | 0 | 0 | 0 | 0 | 1 | 0 | 0 | 0 |
| SLC13A2 | 0 | 0 | 0 | 0 | 1 | 0 | 0 | 0 |
| PHGR1 | 0 | 0 | 0 | 0 | 1 | 0 | 0 | 0 |
| LEPREL1 | 0 | 0 | 0 | 0 | 1 | 0 | 0 | 0 |
| ZNF219 | 0 | 0 | 0 | 0 | 1 | 0 | 0 | 0 |
| JUND | 0 | 0 | 0 | 0 | 1 | 0 | 0 | 0 |
| TCN1 | 0 | 0 | 0 | 0 | 1 | 0 | 0 | 0 |
| KLK10 | 0 | 0 | 0 | 0 | 1 | 0 | 0 | 0 |
| IER5L | 0 | 0 | 0 | 0 | 1 | 0 | 0 | 0 |
| HES4 | 0 | 0 | 0 | 0 | 1 | 0 | 0 | 0 |
| SFRP1 | 0 | 0 | 0 | 0 | 1 | 0 | 0 | 0 |
| AMN | 0 | 0 | 0 | 0 | 1 | 0 | 0 | 0 |
| UGT2B17 | 0 | 0 | 0 | 0 | 1 | 0 | 0 | 0 |
| PLN | 0 | 0 | 0 | 0 | 1 | 0 | 0 | 0 |
| TPO | 0 | 0 | 0 | 0 | 1 | 0 | 0 | 0 |
| FAM135B | 0 | 0 | 0 | 0 | 1 | 0 | 0 | 0 |
| AHNAK2 | 0 | 0 | 0 | 0 | 1 | 0 | 0 | 0 |
| B3GNT7 | 0 | 0 | 0 | 0 | 1 | 0 | 0 | 0 |
| GLDN | 0 | 0 | 0 | 0 | 1 | 0 | 0 | 0 |
| IL1RN | 0 | 0 | 0 | 0 | 1 | 0 | 0 | 0 |
| CTSE | 0 | 0 | 0 | 0 | 1 | 0 | 0 | 0 |
| SERPINB3 | 0 | 0 | 0 | 0 | 1 | 0 | 0 | 0 |
| VSIG1 | 0 | 0 | 0 | 0 | 1 | 0 | 0 | 0 |
| C4B | 0 | 0 | 0 | 0 | 1 | 0 | 0 | 0 |
| AOX1 | 0 | 0 | 0 | 0 | 1 | 0 | 0 | 0 |
| CLDN5 | 0 | 0 | 0 | 0 | 1 | 0 | 0 | 0 |
| THBS2 | 0 | 0 | 0 | 0 | 1 | 0 | 0 | 0 |
| SOX18 | 0 | 0 | 0 | 0 | 1 | 0 | 0 | 0 |
| SLC6A14 | 0 | 0 | 0 | 0 | 1 | 0 | 0 | 0 |
| HIC1 | 0 | 0 | 0 | 0 | 1 | 0 | 0 | 0 |
| AZGP1 | 0 | 0 | 0 | 0 | 1 | 0 | 0 | 0 |
| CYP1B1 | 0 | 0 | 0 | 0 | 1 | 0 | 0 | 0 |
| PLA2G2A | 0 | 0 | 0 | 0 | 1 | 0 | 0 | 0 |
| MT_ATP8 | 0 | 0 | 0 | 0 | 1 | 1 | 0 | 0 |
| GREM1 | 0 | 0 | 0 | 0 | 1 | 0 | 0 | 0 |
| CFD | 0 | 0 | 0 | 0 | 1 | 0 | 0 | 0 |
| APOE | 0 | 0 | 0 | 0 | 1 | 0 | 0 | 0 |
| SOCS1 | 0 | 0 | 0 | 0 | 1 | 0 | 0 | 0 |
| CCL11 | 0 | 0 | 0 | 0 | 1 | 0 | 0 | 0 |
| CHI3L2 | 0 | 0 | 0 | 0 | 1 | 0 | 0 | 0 |
| VIP | 0 | 0 | 0 | 0 | 1 | 0 | 0 | 0 |
| REG3A | 0 | 0 | 0 | 0 | 1 | 0 | 0 | 0 |
| JUN | 0 | 0 | 0 | 0 | 1 | 0 | 0 | 0 |
| MMP7 | 0 | 0 | 0 | 0 | 1 | 0 | 0 | 0 |
| HCAR3 | 0 | 0 | 0 | 0 | 1 | 0 | 1 | 0 |
| L1TD1 | 0 | 0 | 0 | 0 | 1 | 0 | 0 | 0 |
| SERPINB7 | 0 | 0 | 0 | 0 | 1 | 0 | 0 | 0 |
| GUCA2A | 0 | 0 | 0 | 0 | 1 | 0 | 0 | 0 |
| MT1G | 0 | 0 | 0 | 0 | 1 | 0 | 0 | 0 |
| SLC15A1 | 0 | 0 | 0 | 0 | 1 | 0 | 0 | 0 |
| DEFA6 | 0 | 0 | 0 | 0 | 1 | 0 | 0 | 0 |
| GAS1 | 0 | 0 | 0 | 0 | 1 | 0 | 0 | 0 |
| MEDAG | 0 | 0 | 0 | 0 | 1 | 0 | 0 | 0 |
| AQP8 | 0 | 0 | 0 | 0 | 1 | 0 | 0 | 0 |
| FAP | 0 | 0 | 0 | 0 | 1 | 0 | 0 | 0 |
| NTN1 | 0 | 0 | 0 | 0 | 1 | 0 | 0 | 0 |
| MUC2 | 0 | 0 | 0 | 0 | 1 | 0 | 0 | 0 |
| KCNJ10 | 0 | 0 | 0 | 0 | 1 | 0 | 0 | 0 |
| AC142381_1 | 0 | 0 | 0 | 0 | 1 | 0 | 0 | 0 |
| ADM | 0 | 0 | 0 | 0 | 1 | 0 | 0 | 0 |
| ST3GAL1 | 0 | 0 | 0 | 0 | 1 | 0 | 0 | 0 |
| PYY | 0 | 0 | 0 | 0 | 1 | 0 | 0 | 0 |
| CNN1 | 0 | 0 | 0 | 0 | 1 | 0 | 0 | 0 |
| TRIM40 | 0 | 0 | 0 | 0 | 1 | 0 | 0 | 0 |
| CTSG | 0 | 0 | 0 | 0 | 1 | 0 | 0 | 0 |
| LY6D | 0 | 0 | 0 | 0 | 1 | 0 | 0 | 0 |
| LILRA5 | 0 | 0 | 0 | 0 | 1 | 0 | 0 | 0 |
| SST | 0 | 0 | 0 | 0 | 1 | 0 | 0 | 0 |
| DMBT1 | 0 | 0 | 0 | 0 | 1 | 0 | 0 | 0 |
| GBP3 | 0 | 0 | 0 | 0 | 1 | 0 | 0 | 0 |
| TNIP3 | 0 | 0 | 0 | 0 | 1 | 0 | 0 | 0 |
| CXCL9 | 0 | 0 | 0 | 0 | 1 | 0 | 0 | 0 |
| IL24 | 0 | 0 | 0 | 0 | 1 | 0 | 0 | 0 |
| FOS | 0 | 0 | 0 | 0 | 1 | 0 | 0 | 0 |
| IL11 | 0 | 0 | 0 | 0 | 1 | 0 | 0 | 0 |
| CCL21 | 0 | 0 | 0 | 0 | 1 | 0 | 0 | 0 |
| WNT2 | 0 | 0 | 0 | 0 | 1 | 0 | 0 | 0 |
| NOS2 | 0 | 0 | 0 | 0 | 1 | 0 | 0 | 0 |
| MASP1 | 0 | 0 | 0 | 0 | 1 | 0 | 0 | 0 |
| EGF | 0 | 0 | 0 | 0 | 1 | 0 | 0 | 0 |
| SAA1 | 0 | 0 | 0 | 0 | 1 | 0 | 0 | 0 |
| CD8A | 0 | 0 | 0 | 0 | 1 | 0 | 0 | 0 |
| GABRP | 0 | 0 | 0 | 0 | 1 | 0 | 0 | 0 |
| CXCL5 | 0 | 0 | 0 | 0 | 1 | 0 | 0 | 0 |
| TNC | 0 | 0 | 0 | 0 | 1 | 0 | 0 | 0 |
| NAF1 | 0 | 0 | 0 | 0 | 0 | 1 | 0 | 0 |
| MAP7D3 | 0 | 0 | 0 | 0 | 0 | 1 | 0 | 0 |
| GPR155 | 0 | 0 | 0 | 0 | 0 | 1 | 0 | 0 |
| NRG1 | 0 | 0 | 0 | 0 | 0 | 1 | 0 | 0 |
| SPRY1 | 0 | 0 | 0 | 0 | 0 | 1 | 0 | 0 |
| C1QB | 0 | 0 | 0 | 0 | 0 | 1 | 0 | 0 |
| LYPD3 | 0 | 0 | 0 | 0 | 0 | 1 | 0 | 0 |
| SLFN5 | 0 | 0 | 0 | 0 | 0 | 1 | 1 | 0 |
| ADCY3 | 0 | 0 | 0 | 0 | 0 | 1 | 0 | 0 |
| PDE4B | 0 | 0 | 0 | 0 | 0 | 1 | 1 | 0 |
| CEP44 | 0 | 0 | 0 | 0 | 0 | 1 | 0 | 0 |
| MSR1 | 0 | 0 | 0 | 0 | 0 | 1 | 0 | 0 |
| PLA2G16 | 0 | 0 | 0 | 0 | 0 | 1 | 0 | 0 |
| CPED1 | 0 | 0 | 0 | 0 | 0 | 1 | 0 | 0 |
| ICOSLG | 0 | 0 | 0 | 0 | 0 | 1 | 0 | 0 |
| TUBG2 | 0 | 0 | 0 | 0 | 0 | 1 | 0 | 0 |
| SNAI1 | 0 | 0 | 0 | 0 | 0 | 1 | 0 | 0 |
| CCL20 | 0 | 0 | 0 | 0 | 0 | 1 | 0 | 0 |
| IL6 | 0 | 0 | 0 | 0 | 0 | 1 | 0 | 0 |
| OSM | 0 | 0 | 0 | 0 | 0 | 1 | 0 | 0 |
| CCR2 | 0 | 0 | 0 | 0 | 0 | 1 | 1 | 0 |
| APOBEC3B | 0 | 0 | 0 | 0 | 0 | 1 | 0 | 0 |
| LGALS2 | 0 | 0 | 0 | 0 | 0 | 1 | 0 | 0 |
| ANPEP | 0 | 0 | 0 | 0 | 0 | 1 | 0 | 0 |
| BHLHE40 | 0 | 0 | 0 | 0 | 0 | 1 | 0 | 0 |
| NR4A1 | 0 | 0 | 0 | 0 | 0 | 1 | 0 | 1 |
| CXCR4 | 0 | 0 | 0 | 0 | 0 | 1 | 0 | 1 |
| MTRNR2L12 | 0 | 0 | 0 | 0 | 0 | 1 | 0 | 0 |
| PPBP | 0 | 0 | 0 | 0 | 0 | 1 | 0 | 0 |
| TMEM176B | 0 | 0 | 0 | 0 | 0 | 1 | 0 | 0 |
| ARL4C | 0 | 0 | 0 | 0 | 0 | 1 | 0 | 0 |
| RGS1 | 0 | 0 | 0 | 0 | 0 | 1 | 0 | 0 |
| NBPF14 | 0 | 0 | 0 | 0 | 0 | 1 | 0 | 0 |
| TAGAP | 0 | 0 | 0 | 0 | 0 | 1 | 0 | 0 |
| ACKR3 | 0 | 0 | 0 | 0 | 0 | 1 | 0 | 0 |
| COLEC12 | 0 | 0 | 0 | 0 | 0 | 1 | 0 | 0 |
| CLU | 0 | 0 | 0 | 0 | 0 | 1 | 1 | 0 |
| SULT1A2 | 0 | 0 | 0 | 0 | 0 | 1 | 0 | 0 |
| GRASP | 0 | 0 | 0 | 0 | 0 | 1 | 0 | 0 |
| SIK1 | 0 | 0 | 0 | 0 | 0 | 1 | 0 | 0 |
| NR4A2 | 0 | 0 | 0 | 0 | 0 | 1 | 0 | 0 |
| CLEC5A | 0 | 0 | 0 | 0 | 0 | 0 | 1 | 0 |
| SGK223 | 0 | 0 | 0 | 0 | 0 | 0 | 1 | 0 |
| SGK1 | 0 | 0 | 0 | 0 | 0 | 0 | 1 | 0 |
| CITED4 | 0 | 0 | 0 | 0 | 0 | 0 | 1 | 0 |
| ALOX5AP | 0 | 0 | 0 | 0 | 0 | 0 | 1 | 0 |
| ALOX15B | 0 | 0 | 0 | 0 | 0 | 0 | 1 | 0 |
| RAPH1 | 0 | 0 | 0 | 0 | 0 | 0 | 1 | 0 |
| DNAJB1 | 0 | 0 | 0 | 0 | 0 | 0 | 1 | 0 |
| MMP19 | 0 | 0 | 0 | 0 | 0 | 0 | 1 | 0 |
| STEAP4 | 0 | 0 | 0 | 0 | 0 | 0 | 1 | 0 |
| TLR7 | 0 | 0 | 0 | 0 | 0 | 0 | 1 | 0 |
| SEL1L3 | 0 | 0 | 0 | 0 | 0 | 0 | 1 | 0 |
| NFXL1 | 0 | 0 | 0 | 0 | 0 | 0 | 1 | 0 |
| PPP1R16B | 0 | 0 | 0 | 0 | 0 | 0 | 1 | 0 |
| KIAA0226L | 0 | 0 | 0 | 0 | 0 | 0 | 1 | 0 |
| KCNJ15 | 0 | 0 | 0 | 0 | 0 | 0 | 1 | 0 |
| UNC5B | 0 | 0 | 0 | 0 | 0 | 0 | 1 | 0 |
| ZNF714 | 0 | 0 | 0 | 0 | 0 | 0 | 1 | 0 |
| HSPA1A | 0 | 0 | 0 | 0 | 0 | 0 | 1 | 0 |
| ADK | 0 | 0 | 0 | 0 | 0 | 0 | 1 | 0 |
| CAMKK1 | 0 | 0 | 0 | 0 | 0 | 0 | 1 | 0 |
| TTC39B | 0 | 0 | 0 | 0 | 0 | 0 | 1 | 0 |
| GPRIN3 | 0 | 0 | 0 | 0 | 0 | 0 | 1 | 0 |
| DUSP2 | 0 | 0 | 0 | 0 | 0 | 0 | 1 | 0 |
| LPAR6 | 0 | 0 | 0 | 0 | 0 | 0 | 1 | 0 |
| CX3CR1 | 0 | 0 | 0 | 0 | 0 | 0 | 1 | 0 |
| ADAMTS2 | 0 | 0 | 0 | 0 | 0 | 0 | 1 | 0 |
| CCL2 | 0 | 0 | 0 | 0 | 0 | 0 | 1 | 0 |
| TNFAIP3 | 0 | 0 | 0 | 0 | 0 | 0 | 1 | 0 |
| CACNB4 | 0 | 0 | 0 | 0 | 0 | 0 | 1 | 0 |
| TBKBP1 | 0 | 0 | 0 | 0 | 0 | 0 | 1 | 0 |
| TPST1 | 0 | 0 | 0 | 0 | 0 | 0 | 1 | 0 |
| NT5C3B | 0 | 0 | 0 | 0 | 0 | 0 | 1 | 0 |
| CD69 | 0 | 0 | 0 | 0 | 0 | 0 | 1 | 0 |
| IFIT5 | 0 | 0 | 0 | 0 | 0 | 0 | 1 | 0 |
| LOXHD1 | 0 | 0 | 0 | 0 | 0 | 0 | 1 | 0 |
| FPR2 | 0 | 0 | 0 | 0 | 0 | 0 | 1 | 0 |
| HSPA6 | 0 | 0 | 0 | 0 | 0 | 0 | 1 | 0 |
| CD180 | 0 | 0 | 0 | 0 | 0 | 0 | 1 | 0 |
| HES1 | 0 | 0 | 0 | 0 | 0 | 0 | 1 | 0 |
| CTC_534A2_2 | 0 | 0 | 0 | 0 | 0 | 0 | 1 | 0 |
| GBP1 | 0 | 0 | 0 | 0 | 0 | 0 | 1 | 0 |
| BAG3 | 0 | 0 | 0 | 0 | 0 | 0 | 1 | 0 |
| IFFO2 | 0 | 0 | 0 | 0 | 0 | 0 | 1 | 0 |
| TUBA1A | 0 | 0 | 0 | 0 | 0 | 0 | 1 | 0 |
| PLP2 | 0 | 0 | 0 | 0 | 0 | 0 | 1 | 1 |
| FAM20A | 0 | 0 | 0 | 0 | 0 | 0 | 1 | 0 |
| CCL3L1 | 0 | 0 | 0 | 0 | 0 | 0 | 1 | 0 |
| MGLL | 0 | 0 | 0 | 0 | 0 | 0 | 1 | 0 |
| UAP1L1 | 0 | 0 | 0 | 0 | 0 | 0 | 1 | 0 |
| THBS1 | 0 | 0 | 0 | 0 | 0 | 0 | 1 | 0 |
| EGR2 | 0 | 0 | 0 | 0 | 0 | 0 | 1 | 0 |
| SH3PXD2B | 0 | 0 | 0 | 0 | 0 | 0 | 1 | 0 |
| DUSP6 | 0 | 0 | 0 | 0 | 0 | 0 | 0 | 1 |
| SBNO2 | 0 | 0 | 0 | 0 | 0 | 0 | 0 | 1 |
| ATHL1 | 0 | 0 | 0 | 0 | 0 | 0 | 0 | 1 |
| ZNF514 | 0 | 0 | 0 | 0 | 0 | 0 | 0 | 1 |
| PC | 0 | 0 | 0 | 0 | 0 | 0 | 0 | 1 |
| PDGFB | 0 | 0 | 0 | 0 | 0 | 0 | 0 | 1 |
| GAS7 | 0 | 0 | 0 | 0 | 0 | 0 | 0 | 1 |
| KLF11 | 0 | 0 | 0 | 0 | 0 | 0 | 0 | 1 |
| COL18A1 | 0 | 0 | 0 | 0 | 0 | 0 | 0 | 1 |
| SDK2 | 0 | 0 | 0 | 0 | 0 | 0 | 0 | 1 |
| DCHS1 | 0 | 0 | 0 | 0 | 0 | 0 | 0 | 1 |
| TMEM8A | 0 | 0 | 0 | 0 | 0 | 0 | 0 | 1 |
| KRT72 | 0 | 0 | 0 | 0 | 0 | 0 | 0 | 1 |
| CCDC163P | 0 | 0 | 0 | 0 | 0 | 0 | 0 | 1 |
| ZNF772 | 0 | 0 | 0 | 0 | 0 | 0 | 0 | 1 |
| NR4A3 | 0 | 0 | 0 | 0 | 0 | 0 | 0 | 1 |
| JUNB | 0 | 0 | 0 | 0 | 0 | 0 | 0 | 1 |
| DDX3Y | 0 | 0 | 0 | 0 | 0 | 0 | 0 | 1 |
| SYNM | 0 | 0 | 0 | 0 | 0 | 0 | 0 | 1 |

TABLE 14

| feature | CRPcorr | CRPcorr.pval | CRPcorr.adjpval |
|---|---|---|---|
| ENPP7 | −0.41 | 0.08 | 1 |
| CLC | 0.38 | 0.11 | 1 |
| MYEOV | −0.38 | 0.11 | 1 |
| AKR1C1 | −0.36 | 0.13 | 1 |
| SLC16A10 | −0.35 | 0.14 | 1 |
| C10orf99 | 0.34 | 0.16 | 1 |
| SLC16A9 | 0.34 | 0.16 | 1 |
| APOC3 | −0.32 | 0.18 | 1 |
| NLRP2 | 0.3 | 0.22 | 1 |
| ITGA11 | 0.28 | 0.24 | 1 |
| KCNJ16 | −0.28 | 0.25 | 1 |
| SMLR1 | −0.26 | 0.28 | 1 |
| AIFM3 | 0.26 | 0.28 | 1 |
| COL22A1 | 0.26 | 0.29 | 1 |
| C17orf78 | −0.25 | 0.31 | 1 |
| ABCC2 | −0.24 | 0.31 | 1 |
| AADAC | −0.25 | 0.31 | 1 |
| DHDH | −0.25 | 0.31 | 1 |
| MMP1 | −0.23 | 0.33 | 1 |
| CA12 | 0.23 | 0.34 | 1 |
| CCL19 | 0.23 | 0.35 | 1 |
| REG3G | −0.23 | 0.35 | 1 |
| LCT | −0.22 | 0.37 | 1 |
| FAM134B | −0.21 | 0.38 | 1 |
| CA9 | −0.22 | 0.38 | 1 |
| ADORA2B | 0.21 | 0.39 | 1 |
| DPEP1 | −0.2 | 0.41 | 1 |
| SDR16C5 | −0.19 | 0.43 | 1 |
| TM4SF4 | −0.19 | 0.43 | 1 |
| PLB1 | −0.19 | 0.43 | 1 |
| DAB1 | −0.18 | 0.45 | 1 |
| FAM189A1 | 0.18 | 0.46 | 1 |
| STEAP3 | 0.18 | 0.46 | 1 |
| CCL18 | −0.18 | 0.46 | 1 |
| ABCA4 | −0.18 | 0.47 | 1 |
| AKR1B15 | −0.18 | 0.47 | 1 |
| CYR61 | −0.17 | 0.48 | 1 |
| WNT11 | −0.17 | 0.48 | 1 |
| MS4A10 | −0.17 | 0.49 | 1 |
| SLC30A10 | −0.17 | 0.49 | 1 |
| TMPRSS15 | −0.16 | 0.5 | 1 |
| SULT2A1 | −0.17 | 0.5 | 1 |
| RETNLB | 0.17 | 0.5 | 1 |
| MT1H | −0.15 | 0.53 | 1 |
| RGS13 | 0.15 | 0.53 | 1 |
| CCL25 | −0.15 | 0.53 | 1 |
| DUOX2 | −0.15 | 0.54 | 1 |
| SLC5A12 | −0.15 | 0.55 | 1 |
| CA2 | −0.14 | 0.56 | 1 |
| CEACAM1 | −0.14 | 0.56 | 1 |
| TRIM31 | −0.14 | 0.56 | 1 |
| PI15 | −0.14 | 0.57 | 1 |
| PKIB | −0.14 | 0.57 | 1 |
| LDHD | −0.13 | 0.58 | 1 |
| CLCA4 | −0.13 | 0.58 | 1 |
| GSTA1 | −0.14 | 0.58 | 1 |
| SLC4A4 | −0.13 | 0.59 | 1 |
| SFRP2 | 0.13 | 0.6 | 1 |
| TRPM6 | −0.13 | 0.61 | 1 |
| GSTT1 | 0.12 | 0.61 | 1 |
| GREM2 | 0.12 | 0.63 | 1 |
| CXCL13 | −0.11 | 0.64 | 1 |
| HMGCS2 | 0.12 | 0.64 | 1 |
| FMO1 | −0.11 | 0.66 | 1 |
| PTPRR | −0.11 | 0.66 | 1 |
| GSTA2 | −0.11 | 0.66 | 1 |
| PDE4C | −0.1 | 0.68 | 1 |
| GJB3 | 0.1 | 0.68 | 1 |
| C12orf36 | 0.1 | 0.68 | 1 |
| CUBN | −0.1 | 0.69 | 1 |
| SLC9A2 | −0.1 | 0.69 | 1 |
| CCL13 | −0.1 | 0.7 | 1 |
| CA1 | −0.09 | 0.7 | 1 |
| NAT8 | −0.1 | 0.7 | 1 |
| ERAP2 | −0.09 | 0.72 | 1 |
| DEFBI | −0.09 | 0.72 | 1 |
| GPR15 | 0.09 | 0.72 | 1 |
| ZG16 | −0.09 | 0.72 | 1 |
| AMPD1 | 0.08 | 0.73 | 1 |
| NXPE4 | 0.08 | 0.73 | 1 |
| TSPAN1 | −0.08 | 0.73 | 1 |
| CEACAM7 | 0.07 | 0.78 | 1 |
| FAM151A | 0.07 | 0.78 | 1 |
| CA4 | −0.06 | 0.79 | 1 |
| TFPI2 | −0.06 | 0.79 | 1 |
| CKB | 0.06 | 0.8 | 1 |
| SLC9A3 | −0.05 | 0.83 | 1 |
| LYPD8 | −0.05 | 0.85 | 1 |
| MUC12 | −0.04 | 0.86 | 1 |
| CNGA1 | 0.04 | 0.86 | 1 |
| ESPL1 | −0.04 | 0.87 | 1 |
| SLC13A1 | −0.04 | 0.87 | 1 |
| PIGZ | 0.04 | 0.88 | 1 |
| SCNN1B | 0.03 | 0.89 | 1 |
| TNFSF11 | −0.03 | 0.89 | 1 |
| DEFA5 | 0.03 | 0.89 | 1 |
| REG1B | 0.03 | 0.91 | 1 |
| CYP2B6 | −0.03 | 0.91 | 1 |
| SELL | −0.01 | 0.95 | 1 |
| FSIP2 | 0.02 | 0.95 | 1 |
| CA7 | −0.02 | 0.95 | 1 |
| SLC26A2 | −0.01 | 0.96 | 1 |
| CXCL11 | −0.01 | 0.96 | 1 |
| ATP10B | 0.01 | 0.97 | 1 |
| OASL | 0.01 | 0.98 | 1 |
| NDRG4 | 0 | 0.99 | 1 |
| VSIG2 | 0 | 0.99 | 1 |
| SLC26A3 | 0 | 0.99 | 1 |
| DHRS9 | 0 | 1 | 1 |

TABLE 15

| | Cohort | | | | | | | | | | | | |
|---|---|---|---|---|---|---|---|---|---|---|---|---|---|
| | Anti- | Anti- | Anti- | Anti- | Anti- | Anti- | Anti- | Anti- | Anti- | Anti- | Anti- | Anti- | Anti- |
| | TNF | TNF | TNF | TNF | TNF | TNF | TNF | TNF | TNF | TNF | TNF | TNF | TNF |
| | UC | UC | UC | UC | UC | UC | UC | UC | UC | UC | UC | UC | UC |
| | Feature | | | | | | | | | | | | |
| Target | NLRP2 | HIC1 | THBS2 | IL11 | IL1RN | JUND | WNT2 | JUN | APOE | VIP | EGF | FOS | SOCS1 |
| PYCARD | 1 | 0 | 0 | 0 | 0 | 0 | 0 | 0 | 0 | 0 | 0 | 0 | 0 |
| IKBKB | 1 | 0 | 0 | 0 | 0 | 0 | 0 | 0 | 0 | 0 | 0 | 0 | 0 |
| ACKR3 | 0 | 1 | 0 | 0 | 0 | 0 | 0 | 0 | 0 | 0 | 0 | 0 | 0 |
| STAT3 | 0 | 1 | 0 | 0 | 0 | 0 | 0 | 0 | 0 | 0 | 0 | 0 | 0 |
| CD47 | 0 | 0 | 1 | 0 | 0 | 0 | 0 | 0 | 0 | 0 | 0 | 0 | 0 |
| IL6ST | 0 | 0 | 0 | 1 | 0 | 0 | 0 | 0 | 0 | 0 | 0 | 0 | 0 |
| IL1R2 | 0 | 0 | 0 | 0 | 1 | 0 | 0 | 0 | 0 | 0 | 0 | 0 | 0 |
| IL1R1 | 0 | 0 | 0 | 0 | 1 | 0 | 0 | 0 | 0 | 0 | 0 | 0 | 0 |
| SOX4 | 0 | 0 | 0 | 0 | 0 | 1 | 0 | 0 | 0 | 0 | 0 | 0 | 0 |
| F3 | 0 | 0 | 0 | 0 | 0 | 1 | 0 | 0 | 0 | 0 | 0 | 0 | 0 |
| CLU | 0 | 0 | 0 | 0 | 0 | 1 | 0 | 0 | 0 | 0 | 0 | 1 | 0 |
| ITGAV | 0 | 0 | 0 | 0 | 0 | 1 | 0 | 0 | 0 | 0 | 0 | 0 | 0 |
| CCL5 | 0 | 0 | 0 | 0 | 0 | 1 | 0 | 1 | 0 | 0 | 0 | 0 | 0 |
| GADD45A | 0 | 0 | 0 | 0 | 0 | 1 | 0 | 0 | 0 | 0 | 0 | 0 | 0 |
| PLAUR | 0 | 0 | 0 | 0 | 0 | 1 | 0 | 1 | 0 | 0 | 0 | 1 | 0 |
| MYB | 0 | 0 | 0 | 0 | 0 | 0 | 1 | 1 | 0 | 0 | 0 | 0 | 0 |
| LRP6 | 0 | 0 | 0 | 0 | 0 | 0 | 0 | 1 | 0 | 0 | 0 | 0 | 0 |
| CDKN1A | 0 | 0 | 0 | 0 | 0 | 0 | 0 | 1 | 0 | 0 | 0 | 0 | 0 |
| NQO1 | 0 | 0 | 0 | 0 | 0 | 0 | 0 | 1 | 0 | 0 | 0 | 1 | 0 |
| PLAT | 0 | 0 | 0 | 0 | 0 | 0 | 0 | 1 | 0 | 0 | 0 | 0 | 0 |
| VCAM1 | 0 | 0 | 0 | 0 | 0 | 0 | 0 | 1 | 0 | 0 | 0 | 0 | 0 |
| PDHA1 | 0 | 0 | 0 | 0 | 0 | 0 | 0 | 1 | 0 | 0 | 0 | 1 | 0 |
| ATF3 | 0 | 0 | 0 | 0 | 0 | 0 | 0 | 1 | 0 | 0 | 0 | 0 | 0 |
| MYC | 0 | 0 | 0 | 0 | 0 | 0 | 0 | 1 | 0 | 0 | 0 | 1 | 0 |
| CTSL | 0 | 0 | 0 | 0 | 0 | 0 | 0 | 1 | 0 | 0 | 0 | 0 | 0 |
| GCLC | 0 | 0 | 0 | 0 | 0 | 0 | 0 | 1 | 0 | 0 | 0 | 0 | 0 |
| TXN | 0 | 0 | 0 | 0 | 0 | 0 | 0 | 1 | 0 | 0 | 0 | 0 | 0 |
| APP | 0 | 0 | 0 | 0 | 0 | 0 | 0 | 1 | 0 | 0 | 0 | 0 | 0 |
| UGT2B15 | 0 | 0 | 0 | 0 | 0 | 0 | 0 | 1 | 0 | 0 | 0 | 0 | 0 |
| SMAD7 | 0 | 0 | 0 | 0 | 0 | 0 | 0 | 1 | 0 | 0 | 0 | 1 | 0 |
| ETS2 | 0 | 0 | 0 | 0 | 0 | 0 | 0 | 1 | 0 | 0 | 0 | 0 | 0 |
| GJA1 | 0 | 0 | 0 | 0 | 0 | 0 | 0 | 1 | 0 | 0 | 0 | 0 | 0 |
| VEGFA | 0 | 0 | 0 | 0 | 0 | 0 | 0 | 1 | 0 | 0 | 0 | 0 | 0 |
| RHOB | 0 | 0 | 0 | 0 | 0 | 0 | 0 | 1 | 0 | 0 | 0 | 0 | 0 |
| TNC | 0 | 0 | 0 | 0 | 0 | 0 | 0 | 1 | 0 | 0 | 0 | 0 | 0 |
| NAT1 | 0 | 0 | 0 | 0 | 0 | 0 | 0 | 1 | 0 | 0 | 0 | 1 | 0 |
| EGFR | 0 | 0 | 0 | 0 | 0 | 0 | 0 | 1 | 0 | 0 | 1 | 0 | 0 |
| EZR | 0 | 0 | 0 | 0 | 0 | 0 | 0 | 1 | 0 | 0 | 0 | 1 | 0 |
| VDR | 0 | 0 | 0 | 0 | 0 | 0 | 0 | 1 | 0 | 0 | 0 | 1 | 0 |
| CD82 | 0 | 0 | 0 | 0 | 0 | 0 | 0 | 1 | 0 | 0 | 0 | 0 | 0 |
| TIMP2 | 0 | 0 | 0 | 0 | 0 | 0 | 0 | 1 | 0 | 0 | 0 | 0 | 0 |
| FAS | 0 | 0 | 0 | 0 | 0 | 0 | 0 | 1 | 0 | 0 | 0 | 1 | 0 |
| PTN | 0 | 0 | 0 | 0 | 0 | 0 | 0 | 1 | 0 | 0 | 0 | 0 | 0 |
| ABCB1 | 0 | 0 | 0 | 0 | 0 | 0 | 0 | 1 | 0 | 0 | 0 | 0 | 0 |
| MMP2 | 0 | 0 | 0 | 0 | 0 | 0 | 0 | 1 | 0 | 0 | 0 | 0 | 0 |
| MAT2A | 0 | 0 | 0 | 0 | 0 | 0 | 0 | 1 | 0 | 0 | 0 | 0 | 0 |
| CYP2J2 | 0 | 0 | 0 | 0 | 0 | 0 | 0 | 1 | 0 | 0 | 0 | 0 | 0 |
| TFF1 | 0 | 0 | 0 | 0 | 0 | 0 | 0 | 1 | 0 | 0 | 0 | 0 | 0 |
| VIM | 0 | 0 | 0 | 0 | 0 | 0 | 0 | 1 | 0 | 0 | 0 | 0 | 0 |
| CCND1 | 0 | 0 | 0 | 0 | 0 | 0 | 0 | 1 | 0 | 0 | 0 | 2 | 0 |
| HEY1 | 0 | 0 | 0 | 0 | 0 | 0 | 0 | 1 | 0 | 0 | 0 | 0 | 0 |
| POLD2 | 0 | 0 | 0 | 0 | 0 | 0 | 0 | 1 | 0 | 0 | 0 | 0 | 0 |
| IGF1 | 0 | 0 | 0 | 0 | 0 | 0 | 0 | 1 | 0 | 0 | 0 | 0 | 0 |
| NR3C1 | 0 | 0 | 0 | 0 | 0 | 0 | 0 | 1 | 0 | 0 | 0 | 1 | 0 |
| MGP | 0 | 0 | 0 | 0 | 0 | 0 | 0 | 1 | 0 | 0 | 0 | 0 | 0 |
| CREB1 | 0 | 0 | 0 | 0 | 0 | 0 | 0 | 1 | 0 | 0 | 0 | 0 | 0 |
| FOS | 0 | 0 | 0 | 0 | 0 | 0 | 0 | 1 | 0 | 0 | 0 | 0 | 0 |
| ATF2 | 0 | 0 | 0 | 0 | 0 | 0 | 0 | 1 | 0 | 0 | 0 | 1 | 0 |
| CREBBP | 0 | 0 | 0 | 0 | 0 | 0 | 0 | 1 | 0 | 0 | 0 | 0 | 0 |
| ABL1 | 0 | 0 | 0 | 0 | 0 | 0 | 0 | 1 | 0 | 0 | 0 | 0 | 0 |
| NFE2L2 | 0 | 0 | 0 | 0 | 0 | 0 | 0 | 1 | 0 | 0 | 0 | 0 | 0 |
| EP300 | 0 | 0 | 0 | 0 | 0 | 0 | 0 | 1 | 0 | 0 | 0 | 1 | 0 |
| SMAD4 | 0 | 0 | 0 | 0 | 0 | 0 | 0 | 1 | 0 | 0 | 0 | 1 | 0 |
| TCF7L2 | 0 | 0 | 0 | 0 | 0 | 0 | 0 | 1 | 0 | 0 | 0 | 0 | 0 |
| SMAD3 | 0 | 0 | 0 | 0 | 0 | 0 | 0 | 1 | 0 | 0 | 0 | 0 | 0 |
| TCF4 | 0 | 0 | 0 | 0 | 0 | 0 | 0 | 1 | 0 | 0 | 0 | 0 | 0 |
| SORL1 | 0 | 0 | 0 | 0 | 0 | 0 | 0 | 0 | 1 | 0 | 0 | 0 | 0 |
| VIPR1 | 0 | 0 | 0 | 0 | 0 | 0 | 0 | 0 | 0 | 1 | 0 | 0 | 0 |
| ERBB2 | 0 | 0 | 0 | 0 | 0 | 0 | 0 | 0 | 0 | 0 | 1 | 0 | 0 |
| BCL2L1 | 0 | 0 | 0 | 0 | 0 | 0 | 0 | 0 | 0 | 0 | 0 | 1 | 0 |
| HSPH1 | 0 | 0 | 0 | 0 | 0 | 0 | 0 | 0 | 0 | 0 | 0 | 1 | 0 |
| MAF | 0 | 0 | 0 | 0 | 0 | 0 | 0 | 0 | 0 | 0 | 0 | 1 | 0 |
| GSTP1 | 0 | 0 | 0 | 0 | 0 | 0 | 0 | 0 | 0 | 0 | 0 | 1 | 0 |
| SP3 | 0 | 0 | 0 | 0 | 0 | 0 | 0 | 0 | 0 | 0 | 0 | 1 | 0 |
| CDH1 | 0 | 0 | 0 | 0 | 0 | 0 | 0 | 0 | 0 | 0 | 0 | 1 | 0 |
| CTNNB1 | 0 | 0 | 0 | 0 | 0 | 0 | 0 | 0 | 0 | 0 | 0 | 1 | 0 |
| STAT1 | 0 | 0 | 0 | 0 | 0 | 0 | 0 | 0 | 0 | 0 | 0 | 1 | 0 |
| JUN | 0 | 0 | 0 | 0 | 0 | 0 | 0 | 0 | 0 | 0 | 0 | 1 | 0 |
| IFNGR1 | 0 | 0 | 0 | 0 | 0 | 0 | 0 | 0 | 0 | 0 | 0 | 0 | 1 |
| MAP3K5 | 0 | 0 | 0 | 0 | 0 | 0 | 0 | 0 | 0 | 0 | 0 | 0 | 1 |
| JAK1 | 0 | 0 | 0 | 0 | 0 | 0 | 0 | 0 | 0 | 0 | 0 | 0 | 1 |
| IRAK1 | 0 | 0 | 0 | 0 | 0 | 0 | 0 | 0 | 0 | 0 | 0 | 0 | 1 |
| RASA1 | 0 | 0 | 0 | 0 | 0 | 0 | 0 | 0 | 0 | 0 | 0 | 0 | 1 |

TABLE 16

| Feature.Uniprot ID | Target.Uniprot ID | Feature.gene_name | Target.gene_name | Stimulatory? | Inhibitory? |
|---|---|---|---|---|---|
| Q9NX02 | Q9ULZ3 | NLRP2 | PYCARD | 0 | 0 |
| Q9NX02 | O14920 | NLRP2 | IKBKB | 0 | 0 |
| Q96A56 | Q9GZQ8 | TP53INP1 | MAP1LC3B | 1 | 0 |
| Q96A56 | P60520 | TP53INP1 | GABARAPL2 | 1 | 0 |
| P13501 | P51681 | CCL5 | CCR5 | 1 | 0 |
| P13501 | P32246 | CCL5 | CCR1 | 1 | 0 |
| P01375 | P20333 | TNF | TNFRSF1B | 1 | 0 |
| P01375 | P31749 | TNF | AKT1 | 1 | 0 |
| P01375 | P42336 | TNF | PIK3CA | 1 | 0 |
| P01375 | Q15628 | TNF | TRADD | 0 | 1 |
| P01375 | Q13490 | TNF | BIRC2 | 0 | 0 |
| P01375 | P19438 | TNF | TNFRSF1A | 1 | 0 |
| O15105 | O43318 | SMAD7 | MAP3K7 | 1 | 0 |
| O15105 | Q13233 | SMAD7 | MAP3K1 | 0 | 1 |
| O15105 | Q9Y3F4 | SMAD7 | STRAP | 1 | 0 |
| O15105 | O75807 | SMAD7 | PPP1R15A | 1 | 0 |
| O15105 | Q15796 | SMAD7 | SMAD2 | 1 | 1 |
| O15105 | Q9NYJ8 | SMAD7 | TAB2 | 1 | 0 |
| O15105 | P62136 | SMAD7 | PPP1CA | 1 | 0 |
| O15105 | Q13485 | SMAD7 | SMAD4 | 1 | 0 |
| O15105 | P84022 | SMAD7 | SMAD3 | 1 | 1 |
| O15105 | Q04771 | SMAD7 | ACVR1 | 1 | 0 |
| O15105 | Q9HAU4 | SMAD7 | SMURF2 | 1 | 0 |
| O14879 | Q9UHD2 | IFIT3 | TBK1 | 0 | 0 |
| O15105 | P01106 | SMAD7 | MYC | 1 | 0 |
| O15105 | P09601 | SMAD7 | HMOX1 | 0 | 0 |

TABLE 17

| | | | | Number of |
|---|---|---|---|---|
| | | | Number of | nodes with |
| | | | nodes in | BPGO term |
| | | | feature | in feature |
| Set | Feature | Biological Process Gene Ontology (BPGO) term | sub-network | sub-network |
| IFXADM_UC_CD4 | SOCS3 | cytokine-mediated_signaling_pathway_[GO:0019221] | 18 | 7 |
| IFXADM_UC_CD4 | SOCS3 | post-translational_protein_modification_[GO:0043687] | 18 | 6 |
| IFXADM_UC_CD4 | SOCS3 | interleukin-35-mediated_signaling_pathway_[GO:0070757] | 18 | 4 |
| IFXADM_UC_CD4 | SOCS3 | receptor_signaling_pathway_via_JAK-STAT_[GO:0007259] | 18 | 4 |
| VDZ_UC_CD4 | SMAD7 | protein_deubiquitination_[GO:0016579] | 13 | 6 |
| VDZ_UC_CD4 | SMAD7 | transforming_growth_factor_beta_receptor_signaling_pathway_[GO:0007179] | 13 | 5 |
| VDZ_UC_CD4 | SMAD7 | regulation_of_transforming_growth_factor_beta_receptor_signaling_path- | 13 | 4 |
| | | way_[GO:0017015] | | |
| VDZ_UC_CD4 | TNF | I-kappaB_kinase/NF-kappaB_signaling_[GO:0007249] | 6 | 4 |
| VDZ_UC_CD4 | TNF | tumor_necrosis_factor-mediated_signaling_pathway_[GO:0033209] | 6 | 4 |
| VDZ_UC_CD4 | SMAD7 | negative_regulation_of_trans- | 13 | 4 |
| | | forming_growth_factor_beta_receptor_signaling_pathway_[GO:0030512] | | |
| VDZ_UC_CD4 | TNF | cytokine-mediated_signaling_pathway_[GO:0019221] | 6 | 4 |
| VDZ_CD_CD14 | NFKBIA | interleukin-1-mediated_signaling_pathway_[GO:0070498] | 14 | 6 |
| VDZ_CD_CD14 | EGR1 | negative_regulation_of_apoptotic_process_[GO:0043066] | 33 | 12 |
| VDZ_CD_CD14 | EGR1 | cytokine-mediated_signaling_pathway_[GO:0019221] | 33 | 8 |
| VDZ_CD_CD14 | NFKBIA | negative_regulation_of_transcription_by_RNA_polymerase_II_[GO:0000122] | 14 | 7 |
| VDZ_CD_CD14 | EGR1 | negative_regulation_of_gene_expression_[GO:0010629] | 33 | 6 |
| VDZ_CD_CD14 | NFKBIA | positive_regulation_of_NF-kappaB_transcrip- | 14 | 5 |
| | | tion_factor_activity_[GO:0051092] | | |
| VDZ_CD_CD14 | NFKBIA | stress-activated_MAPK_cascade_[GO:0051403] | 14 | 4 |
| VDZ_CD_CD14 | NFKBIA | cellular_response_to_tumor_necrosis_factor_[GO:0071356] | 14 | 4 |
| VDZ_CD_CD14 | NFKBIA | cellular_response_to_lipopolysaccharide_[GO:0071222] | 14 | 4 |
| IFXADM_UC_CD14 | EGR1 | negative_regulation_of_apoptotic_process_[GO:0043066] | 33 | 12 |
| IFXADM_UC_CD14 | EGR1 | cytokine-mediated_signaling_pathway_[GO:0019221] | 33 | 8 |
| IFXADM_UC_CD14 | EGR1 | negative_regulation_of_gene_expression_[GO:0010629] | 33 | 6 |
| IFXADM_UC_CD14 | EGR1 | aging_[GO:0007568] | 33 | 5 |
| IFXADM_CD_CD14 | TNFAIP3 | positive_regulation_of_transcription_by_RNA_polymerase_II_[GO:0045944] | 8 | 6 |
| IFXADM_CD_CD14 | TNFAIP3 | TRIF-dependent_toll-like_receptor_signaling_pathway_[GO:0035666] | 8 | 4 |
| IFXADM_CD_CD14 | TNFAIP3 | I-kappaB_kinase/NF-kappaB_signaling_[GO:0007249] | 8 | 4 |
| IFXADM_CD_CD14 | TNFAIP3 | inflammatory_response_[GO:0006954] | 8 | 4 |
| IFXADM_UC_COLON | JUN | positive_regulation_of_transcription_by_RNA_polymerase_II_[GO:0045944] | 53 | 23 |
| IFXADM_UC_COLON | JUN | negative_regulation_of_transcription_by_RNA_polymerase_II_[GO:0000122] | 53 | 19 |
| IFXADM_UC_COLON | JUN | positive_regulation_of_transcription,_DNA-templated_[GO:0045893] | 53 | 14 |
| IFXADM_UC_COLON | FOS | negative_regulation_of_transcription_by_RNA_polymerase_II_[GO:0000122] | 24 | 13 |
| IFXADM_UC_COLON | FOS | positive_regulation_of_transcription_by_RNA_polymerase_II_[GO:0045944] | 24 | 11 |
| IFXADM_UC_COLON | JUN | response_to_hypoxia_[GO:0001666] | 53 | 10 |
| IFXADM_UC_COLON | JUN | positive_regulation_of_gene_expression_[GO:0010628] | 53 | 10 |
| IFXADM_UC_COLON | JUN | cytokine-mediated_signaling_pathway_[GO:0019221] | 53 | 10 |
| IFXADM_UC_COLON | JUN | regulation_of_transcription_by_RNA_polymerase_II_[GO:0006357] | 53 | 10 |
| IFXADM_UC_COLON | FOS | positive_regulation_of_transcription,_DNA-templated_[GO:0045893] | 24 | 8 |
| IFXADM_UC_COLON | JUN | aging_[GO:0007568] | 53 | 7 |
| IFXADM UC COLON | JUN | positive_regulation_of_protein_phosphorylation_[GO:0001934] | 53 | 7 |
| IFXADM_UC_COLON | FOS | negative_regulation_of_apoptotic_process_[GO:0043066] | 24 | 7 |
| IFXADM_UC_COLON | JUN | positive_regulation_of_epithelial_cell_proliferation_[GO:0050679] | 53 | 5 |
| IFXADM_UC_COLON | JUN | cellular_response_to_hydrogen_peroxide_[GO:0070301] | 53 | 5 |
| IFXADM_UC_COLON | FOS | positive_regulation_of_neuron_apoptotic_process_[GO:0043525] | 24 | 4 |
| IFXADM_UC_COLON | JUN | positive_regulation_of_phosphorylation_[GO:0042327] | 53 | 4 |
| IFXADM_UC_COLON | JUN | negative_regulation_of_mitotic_cell_cycle_[GO:0045930] | 53 | 4 |
| IFXADM_UC_COLON | JUN | beta-catenin-TCF_complex_assembly_[GO:1904837] | 53 | 4 |
| IFXADM_UC_COLON | FOS | response_to_estradiol_[GO:0032355] | 24 | 4 |
| IFXADM_UC_COLON | JUN | xenobiotic_metabolic_process_[GO:0006805] | 53 | 4 |
| IFXADM_UC_COLON | JUN | SMAD_protein_signal_transduction_[GO:0060395] | 53 | 4 |
| IFXADM_UC_COLON | JUN | fat_cell_differentiation_[GO:0045444] | 53 | 4 |

| Set | Feature | Biological Process Gene Ontology (BPGO) term | P-value | Adj. Pval |
|---|---|---|---|---|
| IFXADM_UC_CD4 | SOCS3 | cytokine-mediated_signaling_pathway_[GO:0019221] | 9.37E−06 | 2.34E−04 |
| IFXADM_UC_CD4 | SOCS3 | post-translational_protein_modification_[GO:0043687] | 7.22E−06 | 1.73E−04 |
| IFXADM_UC_CD4 | SOCS3 | interleukin-35-mediated_signaling_pathway_[GO:0070757] | 3.45E−08 | 6.90E−08 |
| IFXADM_UC_CD4 | SOCS3 | receptor_signaling_pathway_via_JAK-STAT_[GO:0007259] | 1.50E−06 | 1.35E−05 |
| VDZ_UC_CD4 | SMAD7 | protein_deubiquitination_[GO:0016579] | 2.59E−07 | 1.55E−06 |
| VDZ_UC_CD4 | SMAD7 | transforming_growth_factor_beta_receptor_signaling_pathway_[GO:0007179] | 8.77E−07 | 7.02E−06 |
| VDZ_UC_CD4 | SMAD7 | regulation_of_transforming_growth_factor_beta_receptor_signaling_path- | 1.75E−08 | 1.75E−08 |
| | | way_[GO:0017015] | | |
| VDZ_UC_CD4 | TNF | I-kappaB_kinase/NF-kappaB_signaling_[GO:0007249] | 1.75E−07 | 6.98E−07 |
| VDZ_UC_CD4 | TNF | tumor_necrosis_factor-mediated_signaling_pathway_[GO:0033209] | 2.17E−07 | 1.08E−06 |
| VDZ_UC_CD4 | SMAD7 | negative_regulation_of_trans- | 3.77E−06 | 6.41E−05 |
| | | forming_growth_factor_beta_receptor_signaling_pathway_[GO:0030512] | | |
| VDZ_UC_CD4 | TNF | cytokine-mediated_signaling_pathway_[GO:0019221] | 6.72E−05 | 3.23E−03 |
| VDZ_CD_CD14 | NFKBIA | interleukin-1-mediated_signaling_pathway_[GO:0070498] | 1.56E−09 | 1.5624E−09 |
| VDZ_CD_CD14 | EGR1 | negative_regulation_of_apoptotic_process_[GO:0043066] | 3.16E−07 | 6.3245E−07 |
| VDZ_CD_CD14 | EGR1 | cytokine-mediated_signaling_pathway_[GO:0019221] | 9.76E−05 | 0.00204928 |
| VDZ_CD_CD14 | NFKBIA | negative_regulation_of_transcription_by_RNA_polymerase_II_[GO:0000122] | 9.88E−05 | 0.00217378 |
| VDZ_CD_CD14 | EGR1 | negative_regulation_of_gene_expression_[GO:0010629] | 0.000119 | 0.00333989 |
| VDZ_CD_CD14 | NFKBIA | positive_regulation_of_NF-kappaB_transcrip- | 1.5E−05 | 0.0001351 |
| | | tion_factor_activity_[GO:0051092] | | |
| VDZ_CD_CD14 | NFKBIA | stress-activated_MAPK_cascade_[GO:0051403] | 5.84E−07 | 2.3378E−06 |
| VDZ_CD_CD14 | NFKBIA | cellular_response_to_tumor_necrosis_factor_[GO:0071356] | 7.19E−05 | 0.00143882 |
| VDZ_CD_CD14 | NFKBIA | cellular_response_to_lipopolysaccharide_[GO:0071222] | 0.000193 | 0.00657612 |
| IFXADM_UC_CD14 | EGR1 | negative_regulation_of_apoptotic_process_[GO:0043066] | 3.16E−07 | 3.1622E−07 |
| IFXADM_UC_CD14 | EGR1 | cytokine-mediated_signaling_pathway_[GO:0019221] | 9.76E−05 | 0.00117101 |
| IFXADM_UC_CD14 | EGR1 | negative_regulation_of_gene_expression_[GO:0010629] | 0.000119 | 0.00178922 |
| IFXADM_UC_CD14 | EGR1 | aging_[GO:0007568] | 0.000726 | 0.03630687 |
| IFXADM_CD_CD14 | TNFAIP3 | positive_regulation_of_transcription_by_RNA_polymerase_II_[GO:0045944] | 0.00011 | 0.00253128 |
| IFXADM_CD_CD14 | TNFAIP3 | TRIF-dependent_toll-like_receptor_signaling_pathway_[GO:0035666] | 2.13E−08 | 2.1254E−08 |
| IFXADM_CD_CD14 | TNFAIP3 | I-kappaB_kinase/NF-kappaB_signaling_[GO:0007249] | 7.98E−07 | 3.193E−06 |
| IFXADM_CD_CD14 | TNFAIP3 | inflammatory_response_[GO:0006954] | 0.000308 | 0.01294716 |
| IFXADM_UC_COLON | JUN | positive_regulation_of_transcription_by_RNA_polymerase_II_[GO:0045944] | 4.35E−08 | 8.70E−08 |
| IFXADM_UC_COLON | JUN | negative_regulation_of_transcription_by_RNA_polymerase_II_[GO:0000122] | 8.27E−08 | 2.48E−07 |
| IFXADM_UC_COLON | JUN | positive_regulation_of_transcription,_DNA-templated_[GO:0045893] | 2.17E−05 | 1.96E−04 |
| IFXADM_UC_COLON | FOS | negative_regulation_of_transcription_by_RNA_polymerase_II_[GO:0000122] | 2.81E−08 | 2.81E−08 |
| IFXADM_UC_COLON | FOS | positive_regulation_of_transcription_by_RNA_polymerase_II_[GO:0045944] | 8.07E−05 | 1.61E−03 |
| IFXADM_UC_COLON | JUN | response_to_hypoxia_[GO:0001666] | 4.39E−07 | 1.76E−06 |
| IFXADM_UC_COLON | JUN | positive_regulation_of_gene_expression_[GO:0010628] | 0.000117 | 3.27E−03 |
| IFXADM_UC_COLON | JUN | cytokine-mediated_signaling_pathway_[GO:0019221] | 0.000125 | 3.75E−03 |
| IFXADM_UC_COLON | JUN | regulation_of_transcription_by_RNA_polymerase_II_[GO:0006357] | 0.000142 | 4.84E−03 |
| IFXADM_UC_COLON | FOS | positive_regulation_of_transcription,_DNA-templated_[GO:0045893] | 0.000227 | 1.16E−02 |
| IFXADM_UC_COLON | JUN | aging_[GO:0007568] | 0.000152 | 5.64E−03 |
| IFXADM UC COLON | JUN | positive_regulation_of_protein_phosphorylation_[GO:0001934] | 0.000207 | 9.71E−03 |
| IFXADM_UC_COLON | FOS | negative_regulation_of_apoptotic_process_[GO:0043066] | 0.000442 | 3.71E−02 |
| IFXADM_UC_COLON | JUN | positive_regulation_of_epithelial_cell_proliferation_[GO:0050679] | 9.07E−05 | 2.09E−03 |
| IFXADM_UC_COLON | JUN | cellular_response_to_hydrogen_peroxide_[GO:0070301] | 0.000148 | 5.34E−03 |
| IFXADM_UC_COLON | FOS | positive_regulation_of_neuron_apoptotic_process_[GO:0043525] | 3.57E−05 | 3.92E−04 |
| IFXADM_UC_COLON | JUN | positive_regulation_of_phosphorylation_[GO:0042327] | 4.23E−05 | 5.50E−04 |
| IFXADM_UC_COLON | JUN | negative_regulation_of_mitotic_cell_cycle_[GO:0045930] | 4.23E−05 | 5.93E−04 |
| IFXADM_UC_COLON | JUN | beta-catenin-TCF_complex_assembly_[GO:1904837] | 7.52E−05 | 1.35E−03 |
| IFXADM_UC_COLON | FOS | response_to_estradiol_[GO:0032355] | 0.000357 | 2.11E−02 |
| IFXADM_UC_COLON | JUN | xenobiotic_metabolic_process_[GO:0006805] | 0.000396 | 3.09E−02 |
| IFXADM_UC_COLON | JUN | SMAD_protein_signal_transduction_[GO:0060395] | 0.000441 | 3.62E−02 |
| IFXADM_UC_COLON | JUN | fat_cell_differentiation_[GO:0045444] | 0.000441 | 3.66E−02 |

TABLE 18

| Reactome_path- | Reactome_path- | VDZ_{—} | VDZ_{—} | VDZ_{—} | IFXADM_{—} | IFXADM_{—} | IFXADM_{—} | IFXADM_{—} | IFXADM_{—} |
|---|---|---|---|---|---|---|---|---|---|
| way_id | way_name | UC_cd4 | CD_cd14 | CD_ileum | UC_colon | UC_cd4 | UC_cd14 | CD_cd4 | CD_cd14 |
| R-HSA- | Interleukin-10 | — | 1 | — | — | — | 1 | — | 1 |
| 6783783 | signaling | | | | | | | | |
| R-HSA- | Translocation of | — | 1 | — | — | — | — | — | — |
| 202430 | ZAP-70 to | | | | | | | | |
| | Immunological | | | | | | | | |
| | synapse | | | | | | | | |
| R-HSA- | Phosphorylation | — | 1 | — | — | — | — | — | — |
| 202427 | of CD3 and | | | | | | | | |
| | TCR zeta chains | | | | | | | | |
| R-HSA- | PD-1 signaling | — | 1 | — | — | — | — | — | — |
| 389948 | | | | | | | | | |
| R-HSA- | Deregulated CDK5 | — | 1 | — | — | — | — | — | — |
| 8862803 | triggers multiple | | | | | | | | |
| | neurodegenerative | | | | | | | | |
| | pathways in | | | | | | | | |
| | Alzheimer's | | | | | | | | |
| | disease models | | | | | | | | |
| R-HSA- | Neurodegenerative | — | 1 | — | — | — | — | — | — |
| 8863678 | Diseases | | | | | | | | |
| R-HSA- | Costimulation by | — | 1 | — | — | — | — | — | — |
| 388841 | the CD28 family | | | | | | | | |
| R-HSA- | Generation of | — | 1 | — | — | — | — | — | — |
| 202433 | second messenger | | | | | | | | |
| | molecules | | | | | | | | |
| R-HSA- | Interferon Signaling | — | 1 | — | — | — | — | — | — |
| 913531 | | | | | | | | | |
| R-HSA- | Reversible hydration | — | — | 1 | — | — | — | — | — |
| 1475029 | of carbon dioxide | | | | | | | | |
| R-HSA- | G alpha (i) | — | — | 1 | 1 | — | 1 | — | — |
| 418594 | signalling events | | | | | | | | |
| R-HSA- | Transport of | — | — | 1 | — | — | — | — | — |
| 425393 | inorganic cations/ | | | | | | | | |
| | anions and | | | | | | | | |
| | amino acids/ | | | | | | | | |
| | oligopeptides | | | | | | | | |
| R-HSA- | Chemokine | — | — | 1 | 1 | — | 1 | — | — |
| 380108 | receptors bind | | | | | | | | |
| | chemokines | | | | | | | | |
| R-HSA- | Biological | — | — | 1 | — | — | — | — | — |
| 211859 | oxidations | | | | | | | | |
| R-HSA- | Erythrocytes take | — | — | 1 | — | — | — | — | — |
| 1237044 | up carbon dioxide | | | | | | | | |
| | and release oxygen | | | | | | | | |
| R-HSA- | O2/CO2 exchange | — | — | 1 | — | — | — | — | — |
| 1480926 | in erythrocytes | | | | | | | | |
| R-HSA- | SLC-mediated | — | — | 1 | — | — | — | — | — |
| 425407 | transmembrane | | | | | | | | |
| | transport | | | | | | | | |
| R-HSA- | Visual | — | — | 1 | — | — | — | — | — |
| 2187338 | phototransduction | | | | | | | | |
| R-HSA- | Interferon gamma | — | — | 1 | — | — | — | — | — |
| 877300 | signaling | | | | | | | | |
| R-HSA- | Phase II - | — | — | 1 | — | — | — | — | — |
| 156580 | Conjugation | | | | | | | | |
| | of compounds | | | | | | | | |
| R-HSA- | Glutathione | — | — | 1 | — | — | — | — | — |
| 156590 | conjugation | | | | | | | | |
| R-HSA- | Interleukin-4 and | — | — | — | 1 | — | 1 | — | — |
| 6785807 | Interleukin-13 | | | | | | | | |
| | signaling | | | | | | | | |
| R-HSA- | Peptide ligand- | — | — | — | 1 | — | 1 | — | — |
| 375276 | binding receptors | | | | | | | | |
| R-HSA- | GPCR ligand | — | — | — | 1 | — | 1 | — | — |
| 500792 | binding | | | | | | | | |
| R-HSA- | Signaling by | — | — | — | 1 | — | 1 | — | — |
| 449147 | Interleukins | | | | | | | | |
| R-HSA- | Binding and | — | — | — | 1 | — | — | — | — |
| 2173782 | Uptake of | | | | | | | | |
| | Ligands by | | | | | | | | |
| | Scavenger | | | | | | | | |
| | Receptors | | | | | | | | |
| R-HSA- | Class A/1 | — | — | — | 1 | — | 1 | — | — |
| 373076 | (Rhodopsin-like | | | | | | | | |
| | receptors) | | | | | | | | |
| R-HSA- | Initial | — | — | — | 1 | — | — | — | — |
| 166663 | triggering of | | | | | | | | |
| | complement | | | | | | | | |
| R-HSA- | Activation of | — | — | — | 1 | — | — | — | — |
| 1592389 | Matrix | | | | | | | | |
| | Metalloproteinases | | | | | | | | |
| R-HSA- | Regulation of | — | — | — | 1 | — | — | — | — |
| 381426 | Insulin-like | | | | | | | | |
| | Growth Factor | | | | | | | | |
| | (IGF) transport | | | | | | | | |
| | and uptake by | | | | | | | | |
| | Insulin-like | | | | | | | | |
| | Growth Factor | | | | | | | | |
| | Binding Proteins | | | | | | | | |
| | (IGFBPs) | | | | | | | | |
| R-HSA- | Activation of the | — | — | — | 1 | — | — | — | — |
| 450341 | AP-1 family of | | | | | | | | |
| | transcription | | | | | | | | |
| | factors | | | | | | | | |
| R-HSA- | Interleukin-6 | — | — | — | — | — | 1 | — | — |
| 6783589 | family signaling | | | | | | | | |
| R-HSA- | IL-6-type cytokine | — | — | — | — | — | 1 | — | — |
| 6788467 | receptor ligand | | | | | | | | |
| | interactions | | | | | | | | |
| R-HSA- | Scavenging by | — | — | — | — | — | 1 | — | — |
| 3000480 | Class A Receptors | | | | | | | | |
| R-HSA- | Plasma lipoprotein | — | — | — | — | — | 1 | — | — |
| 174824 | assembly, | | | | | | | | |
| | remodeling, | | | | | | | | |
| | and clearance | | | | | | | | |
| R-HSA- | Regulation of | — | — | — | — | — | — | — | 1 |
| 3371453 | HSF1-mediated heat | | | | | | | | |
| | shock response | | | | | | | | |

TABLE 19

| | | | Catalog |
|---|---|---|---|
| Target | Fluorophore | Company | number |
| CD3 | APC | Thermo Fisher | 17-0038 |
| | | Scientific | |
| CD4 | BB515 | BD | 564419 |
| | | Biosciences | |
| CD8 | APC- | Thermo Fisher | 47-0087 |
| | eFluor780 | Scientific | |
| CD14 | PerCP-Cy5.5 | Thermo Fisher | 45-0149 |
| | | Scientific | |
| CD19 | PE | Thermo Fisher | 12-0199-80 |
| | | Scientific | |
| CD45 | AF700 | Thermo Fisher | 56-9459 |
| | | Scientific | |

TABLE 20

| | | |
|---|---|---|
| T cell surface glycoprotein CD6 isoform (CD6) | Q8WWJ7 | INFLAMMATION |
| T-cell surface glycoprotein CD5 (CD5) | P06127 | INFLAMMATION |
| T-cell surface glycoprotein CD8 alpha chain (CD8A) | P01732 | INFLAMMATION |
| Thymic stromal lymphopoietin (TSLP) | Q969D9 | INFLAMMATION |
| TNF-beta (TNFB) | P01374 | INFLAMMATION |
| TNF-related activation-induced cytokine (TRANCE) | O14788 | INFLAMMATION |
| TNF-related apoptosis-inducing ligand (TRAIL) | P50591 | INFLAMMATION |
| Transforming growth factor alpha (TGF-alpha) | P01135 | INFLAMMATION |
| Tumor necrosis factor (Ligand) superfamily, member 12 (TWEAK) | O43508 | INFLAMMATION |
| Tumor necrosis factor (TNF) | P01375 | INFLAMMATION |
| Tumor necrosis factor ligand superfamily member 14 (TNFSF14) | O43557 | INFLAMMATION |
| Tumor necrosis factor receptor superfamily member 9 (TNFRSF9) | Q07011 | INFLAMMATION |
| Urokinase-type plasminogen activator (uPA) | P00749 | INFLAMMATION |
| Vascular endothelial growth factor A (VEGF-A) | P15692 | INFLAMMATION |

TABLE 21

| | Latent | Dominant - | Predictive | |
|---|---|---|---|---|
| Cohort | Factor (s) | omic layer | biomarkers | Accuracy |
| Vedolizumab | LF 5 | Genomics | FAM129A, | 84.2% |
| Ulcerative | | | ELM01, | |
| colitis | | | TRIP13 | |
| | | | PTAR1, | |
| | | | ASAH1 | |
| Vedolizumab | LF 3 | Genomics | SKAP2, | 77.2% |
| Crohn's | | Monocyte | HAUS1, | |
| disease | | transcriptomics | C3orf67, | |
| | | | SEC14L6, | |
| | | | ATP6V0D1 | |
| | LF 8 | | ABCG1, | 98.0% |
| | | | ERAP1, | |
| | | | ERV3_1 | |
| | | | APOL6, | |
| | | | STON2 | |
| Anti-TNF | LF 2 | CD4⁺ T cell | ELOVL4, | 92.0% |
| agents | | transcriptomics | FGL2, CTSW, | |
| Ulcerative | | | DDX11, LYZ | |
| colitis | | | | |
| Anti-TNF | LF 2-LF9 | Genomics | TRAPPC4, | 81.3% |
| agents | | | CDKAL1, | |
| Crohn's | | | ACVRL1, | |
| disease | | | TSPAN14, | |
| | | | PCNP | |
| | | | CITED4, | |
| | | | CLEC5A, | |
| | | | SGK1, | |
| | LF 5-LF | Monocyte | ALOX5AP, | 98.0% |
| | 16 | transcriptomics | SGK223 | |

TABLE 1
Clinical characteristics of the inception cohort, validation cohort 2 and 4

| | INCEPTION | | | | | |
|---|---|---|---|---|---|---|
| | COHORT | | VALIDATION 2 | | | |
| | DISCOVERY + | | RNA - | | VALIDATION 4 | |
| | VALIDATION 1 | | Sequencing | | qPCR | |
| | n = 31 | | n = 16 | | n = 37 | |
| Diagnosis, n, % | | | | | | |
| Ulcerative colitis | 20 | (64.5) | 7 | (43.8) | 30 | (81.1) |
| Crohn's disease | 11 | (35.5) | 9 | (56.3) | 7 | (18.9) |
| Gender, n women, % | 17 | (54.8) | 44.2 | (26.0-55.8) | 38.2 | (31.0-48.0) |
| Age, years, (median, | 45.3 | (29.6-56.3) | 7 | (43.8) | 25 | (67.6) |
| IQR) | | | | | | |
| Disease duration, | 8.4 | (4.0-15.3) | 3.7 | (1.6-20.7) | 6.9 | (1.7-11.7) |
| years, (median, IQR) | | | | | | |
| Disease location*, n, % | | | | | | |
| L1 | 0 | (0) | 0 | (0) | 0 | (0) |
| L2 | 2 | (18.2) | 4 | (44.4) | 3 | (42.9) |
| L3 | 9 | (81.8) | 5 | (56.6) | 4 | (57.1) |
| L4 modifier | 2 | (18.2) | 2 | (22.2) | 0 | (0) |
| E1 | 3 | (15.0) | 1 | (14.3) | 8 | (26.7) |
| E2 | 10 | (50.0) | 2 | (28.6) | 9 | (30.0) |
| E3 | 7 | (35.0) | 4 | (57.1) | 13 | (43.3) |
| Disease behavior*, n, % | | | | | | |
| B1 | 6 | (54.5) | 4 | (44.4) | 6 | (85.7) |
| B2 | 3 | (27.3) | 2 | (22.2) | 0 | (0.0) |
| B3 | 2 | (18.2) | 3 | (33.3) | 1 | (14.3) |
| perianal | 5 | (45.5) | 2 | (22.2) | 2 | (28.6) |
| involvement | | | | | | |
| Steroid use during | | | | | | |
| induction, n, % | | | | | | |
| Topical | 10 | (32.3) | 5 | (31.3) | 15 | (40.1) |
| Systemic | 8 | (25.8) | 6 | (37.5) | 7 | (16.2) |
| Previous anti-TNF | | | | | | |
| exposure, n, % | | | | | | |
| naïve | 10 | (32.3) | 4 | (25.0) | 26 | (70.3) |
| exposed | 21 | (67.7) | 12 | (75.0) | 13 | (29.7) |
| C-reactive protein, | 2.0 | (0.9-6.7) | 3.8 | (1.4-7.2) | 1.8 | (0.7-6.0) |
| mg/L, (median, IQR) | | | | | | |
| Endoscopic remission, | | | | | | |
| n, % | | | | | | |
| Yes | 19 | (61.3) | 5 | (31.1) | 14 | (37.8) |
| No | 12 | (38.7) | 11 | (68.9) | 23 | (62.2) |

n = number of patients;
IQR = interquartile range
*Montreal classification⁵³

TABLE 2
Accuracy of the 4-gene signature in vedolizumab and anti-TNF treated patients

| | Discovery | Validation | Validation | Validation | Anti-TNF |
|---|---|---|---|---|---|
| | dataset | dataset 1 | dataset 2 | dataset 3 | dataset |
| | RNA-seq | RNA-seq | RNA-seq | Microarray | RNA-seq |
| | n = 20 | n = 11 | n = 16 | n = 13 | n = 20 |
| | 9 NR, 11 R | 3 NR, 8 R | 11 NR, 5 R | 9 NR, 4 R | NR 12, R 8 |
| Accuracy | 80.0% | 100.0% | 81.3% | 76.9% | 55.0% |
| Sensitivity | 81.8% | 100.0% | 66.7% | 100.0% | 75.0% |
| Specificity | 77.8% | 100.0% | 90.0% | 70.0% | 41.7% |
| Positive | 81.8% | 100.0% | 80.0% | 50.0% | 46.2% |
| predictive | | | | | |
| value | | | | | |
| Negative | 77.8% | 100.0% | 81.8% | 100.0% | 71.4% |
| predictive | | | | | |
| value | | | | | |
| Positive | 3.7 | ∞ | 6.67 | 3.3 | 1.3 |
| likelihood | | | | | |
| ratio | | | | | |
| Negative | 0.2 | 0 | 0.3 | 0 | 0.6 |
| likelihood | | | | | |
| ratio | | | | | |

NR = non responder;
R = responder;
n = number of patients

TABLE 3
Details of the forward (Fw) and reverse (Rev) primers used for the beta actin qPCR analysis,
including the amplicon length, melt temperature (Tm), 5′-3′ sequence and NCBI accession
number.

| | Amplicon | | | | Accession | |
|---|---|---|---|---|---|---|
| Fw/Rev | Length | Tm (° C.) | Sequence (5’-3’) | Gene | number | Reference |
| Fw | 108 | 59.9 | ACAATGTGGCCGAGGACTTT | Beta Actin | NM_001101.3 | Own design |
| Rev | | 59.7 | TGGGGTGGCTTTTAGGATGG | | | primer BLAST |

TABLE 4
Details of target-specific TaqMan Primers.

| Gene | Target-specific primer | Company |
|---|---|---|
| PIWIL1 | Hs01041737_m1 | ThermoFisher Scientific |
| MAATS1 | Hs00398573_m1 | ThermoFisher Scientific |
| DCHS2 | Hs03006670_m1 | ThermoFisher Scientific |
| RGS13 | Hs00243182_m1 | ThermoFisher Scientific |

TABLE 5
Overview primary antibodies immunohistochemistry

| | Dilution | | Incubation |
|---|---|---|---|
| | primary | | details primary |
| Protein | antibody | Primary antibody | antibody |
| PIWIL1 | 1:2000 | rabbit polyclonal anti-PIWIL1 Ab - HPA018798 - Sigma Aldrich | 30 min at RT |
| MAATS1 | 1:800 | mouse monoclonal anti-MAATS1/C3orf15 Ab - MA5-26540 - Invitrogen | 30 min at RT |
| DCHS2 | 1:400 | rabbit polyclonal anti-DCHS2 Ab - HPA064159 - Sigma Aldrich | 30 min at RT |
| RGS13 | 1:200 | rabbit polyclonal anti-RGS13 Ab - HPA044952 - Sigma Aldrich | 30 min at RT |

Ab = antibody;
RT = room temperature

TABLE 6
Clinical features of the anti-TNF treated cohort

| | | |
|---|---|---|
| Diagnosis, n, % | | |
| Ulcerative colitis | 12 | (60.0) |
| Crohn's disease | 8 | (40.0) |
| Gender, n women, % | 12 | (60.0) |
| Age, years, (median, IQR) | 33.7 | (21.6-48.0) |
| Disease duration, years, | 1.4 | (0.2-4.8) |
| (median, IQR) | | |
| Disease location*, n, % | | |
| L1 | 0 | (0.0) |
| L2 | 1 | (16.7) |
| L3 | 5 | (83.3) |
| L4 modifier | 1 | (16.7) |
| E1 | 0 | (0.0) |
| E2 | 11 | (83.3) |
| E3 | 3 | (21.4) |
| Disease behavior*, n, % | | |
| B1 | 3 | (50.0) |
| B2 | 3 | (50.0) |
| B3 | 0 | (0.0) |
| perianal involvement | 1 | (16.7) |
| Steroid use during | | |
| induction, n, % | | |
| Topical | 3 | (15.0) |
| Systemic | 7 | (35.0) |
| Immunomodulators during | 1 | (5.0) |
| induction, n, % | | |
| C-reactive protein, mg/L, | 5.1 | (1.4-14.2) |
| (median, IQR) | | |
| Endoscopic remission, n, % | | |

| | | |
|---|---|---|
| GO Leukocyte | 0.006 | 0.017 |
| migration | | |
| GO Leukocyte | 0.032 | 0.090 |
| adhesion to vascular | | |
| endothelial cells | | |
| GO Cellular | 0.038 | 0.060 |
| Extravasation | | |
| GO Leukocyte Cell Cell | 0.089 | 0.098 |
| adhesion | | |
| GO integrin mediated | 0.117 | 0.145 |
| signalling pathway | | |
| GO Integrin binding | 0.171 | 0.166 |
| GO cell adhesion | 0.201 | 0.221 |
| molecule binding | | |
| GO cell substrate | 0.232 | 0.208 |
| adhesion | | |

FDR = false discovery rate

TABLE 7
Baseline differentially expressed genes between vedolizumab responders and
non-responders selected based on a nominal 0.005 significance level.

| | | | | FDR |
|---|---|---|---|---|
| | Base | | Nominal | adjusted |
| Gene | Mean | log2FoldChange | pvalue | pvalue |
| KRT23 | 16.71808585 | −2.024205179 | 1.09E−08 | 0.000168888 |
| TMEM35 | 22.00731444 | 1.023871908 | 5.24E−07 | 0.004044778 |
| DCHS2 | 14.21680319 | 1.568224721 | 2.75E−06 | 0.014128976 |
| CLDN8 | 90.06084218 | 4.396104381 | 4.96E−06 | 0.019127255 |
| IFI6 | 334.6292867 | −0.74725992 | 6.36E−06 | 0.019615927 |
| APOBEC3A | 70.46242613 | −1.681358164 | 2.93E−05 | 0.075378873 |
| PCOLCE2 | 7.461678684 | 2.224767485 | 3.85E−05 | 0.084915322 |
| CXCL6 | 367.0654431 | −1.860149522 | 5.42E−05 | 0.096168704 |
| P2RX2 | 3.988036794 | 2.337362091 | 5.61E−05 | 0.096168704 |
| HEPHL1 | 7.197847735 | −2.712063282 | 6.93E−05 | 0.106875332 |
| GZMB | 179.6966807 | −1.235947978 | 0.000105257 | 0.126064653 |
| CCL3 | 110.8862223 | −1.370797965 | 0.000106858 | 0.126064653 |
| DCBLD1 | 335.8001475 | −0.433965692 | 0.000109094 | 0.126064653 |
| IL18RAP | 88.25256523 | −0.945525735 | 0.000114389 | 0.126064653 |
| IL32 | 1239.138654 | −0.516138001 | 0.000191042 | 0.196506067 |
| RGS13 | 26.71377804 | 1.005581305 | 0.000209322 | 0.196608037 |
| C16orf89 | 27.46766449 | 0.798707147 | 0.000216627 | 0.196608037 |
| RASGRP4 | 66.48234435 | −0.86694788 | 0.000229439 | 0.196667853 |
| GLRA2 | 12.88014457 | 2.035443889 | 0.000259165 | 0.207049407 |
| NCF2 | 451.2453884 | −0.812766366 | 0.000276283 | 0.207049407 |
| PPP1R3C | 50.46721132 | 0.777591784 | 0.000281809 | 0.207049407 |
| PTGER1 | 5.95195593 | −1.818433719 | 0.000319712 | 0.209795607 |
| AFF3 | 85.79594968 | 0.720825474 | 0.000327476 | 0.209795607 |
| SERPINA9 | 5.578816375 | 3.073114165 | 0.000328424 | 0.209795607 |
| ITPRIPL2 | 719.9640658 | −0.322518253 | 0.000339937 | 0.209795607 |
| SHC1 | 1492.183764 | −0.276223273 | 0.000370729 | 0.212595281 |
| MAATS1 | 16.84982693 | 1.359399253 | 0.000393874 | 0.212595281 |
| PRF1 | 175.5627542 | −0.778342102 | 0.000408969 | 0.212595281 |
| RIPK2 | 223.2224243 | −0.488962188 | 0.000415642 | 0.212595281 |
| C7 | 206.9021406 | 1.132514881 | 0.000431099 | 0.212595281 |
| VASN | 134.7232446 | −0.923967057 | 0.000469609 | 0.212595281 |
| LILRB2 | 378.0288889 | −1.034543392 | 0.00050092 | 0.212595281 |
| OCA2 | 5.77409323 | 1.797972428 | 0.00050103 | 0.212595281 |
| GDF6 | 5.727696335 | 1.73964914 | 0.000509621 | 0.212595281 |
| HMG20A | 436.3906394 | 0.224406505 | 0.000515674 | 0.212595281 |
| ARRB2 | 608.6137097 | −0.453860183 | 0.000518642 | 0.212595281 |
| MMP1 | 3428.229662 | −2.00236066 | 0.000527115 | 0.212595281 |
| IFNG | 39.99709331 | −1.72185823 | 0.00053709 | 0.212595281 |
| C21orf88 | 75.04395766 | 2.489433131 | 0.000537379 | 0.212595281 |
| CEBPB | 427.6936466 | −0.897826636 | 0.000657776 | 0.24482901 |
| CSF2 | 22.91065713 | −1.739282902 | 0.000659524 | 0.24482901 |
| ZNF587B | 108.0999501 | 0.322149108 | 0.000668663 | 0.24482901 |
| PIWIL1 | 1.671388529 | 3.855039524 | 0.000682457 | 0.24482901 |
| EVA1B | 59.27235971 | −0.927452555 | 0.000698197 | 0.24482901 |
| SLC13A2 | 65.21677719 | 1.487452862 | 0.000751791 | 0.254142023 |
| HSD11B1 | 70.37426993 | −1.04799772 | 0.000783544 | 0.254142023 |
| SHISA3 | 12.42272354 | 1.422845205 | 0.000796262 | 0.254142023 |
| NTRK3 | 10.77519023 | 1.203351121 | 0.00079719 | 0.254142023 |
| S100A3 | 32.14898349 | −1.151271376 | 0.000810185 | 0.254142023 |
| NPTX2 | 112.143113 | −1.739417902 | 0.000823586 | 0.254142023 |
| CMIP | 1060.938128 | −0.24995589 | 0.000847772 | 0.256475996 |
| WBSCR27 | 13.62748883 | 1.304689938 | 0.000930061 | 0.275959815 |
| APOL1 | 3402.486088 | −0.800049872 | 0.000968958 | 0.282076494 |
| LILRA5 | 142.3459388 | −1.210971756 | 0.001013086 | 0.289461169 |
| TENM2 | 4.012797652 | 2.203730979 | 0.001039446 | 0.29159288 |
| TBC1D1 | 837.9063125 | −0.229263126 | 0.00112296 | 0.299758073 |
| KCNH2 | 74.39915275 | 0.642044407 | 0.001154201 | 0.299758073 |
| HSPA12B | 77.76903555 | −0.768198612 | 0.001154923 | 0.299758073 |
| PFKFB4 | 177.1101779 | −0.634147022 | 0.001188816 | 0.299758073 |
| SOX18 | 75.69686921 | −1.037860294 | 0.001217785 | 0.299758073 |
| FCGR2A | 590.6782548 | −1.007841239 | 0.001242733 | 0.299758073 |
| OSCAR | 51.81121286 | −0.694437399 | 0.00124279 | 0.299758073 |
| RP11-812E19.9 | 21.33158313 | −1.080040039 | 0.001243239 | 0.299758073 |
| MMP3 | 4282.898556 | −1.863898029 | 0.001298899 | 0.299758073 |
| ADNP2 | 329.918377 | 0.193727231 | 0.001305613 | 0.299758073 |
| PDPN | 555.8842075 | −0.933770736 | 0.001322834 | 0.299758073 |
| PTAFR | 604.2748337 | −0.609657379 | 0.001323439 | 0.299758073 |
| PLAU | 1885.135502 | −1.002161766 | 0.001329836 | 0.299758073 |
| MMP14 | 2445.673642 | −0.525351269 | 0.001344761 | 0.299758073 |
| HCAR2 | 118.6397201 | −1.66801409 | 0.001360294 | 0.299758073 |
| FFAR2 | 158.9609359 | −1.263390496 | 0.001379404 | 0.299758073 |
| SLC43A2 | 499.956189 | −0.49681542 | 0.001422941 | 0.301417508 |
| GNAI1 | 286.2872531 | −0.361791712 | 0.001436971 | 0.301417508 |
| GBP5 | 1201.697296 | −1.045603479 | 0.001458182 | 0.301417508 |
| KRTAP13-2 | 7.313850486 | 4.807195155 | 0.001478671 | 0.301417508 |
| ZNF525 | 93.38275607 | 0.443027871 | 0.001579396 | 0.301417508 |
| CLGN | 10.73315268 | 1.037888533 | 0.001585583 | 0.301417508 |
| WARS | 7144.39929 | −0.830127459 | 0.001626954 | 0.301417508 |
| PXDN | 1131.818794 | −0.620587799 | 0.001628192 | 0.301417508 |
| NRCAM | 80.95633993 | −1.163930343 | 0.001639771 | 0.301417508 |
| TBX2 | 211.9814703 | −0.576759114 | 0.00165562 | 0.301417508 |
| CCR1 | 328.7571071 | −0.766553241 | 0.001700304 | 0.301417508 |
| GPRASP1 | 96.65151048 | 0.437566455 | 0.001703022 | 0.301417508 |
| TIMM10 | 242.7560124 | 0.413713665 | 0.001710192 | 0.301417508 |
| FCN3 | 84.27508311 | −1.407743033 | 0.001752829 | 0.301417508 |
| DRAXIN | 3.824098101 | −1.486830398 | 0.001762212 | 0.301417508 |
| RAB31 | 1034.096128 | −0.515186815 | 0.001763017 | 0.301417508 |
| IL7R | 1452.388087 | −0.626916046 | 0.001778229 | 0.301417508 |
| FAM26F | 223.730022 | −0.927787816 | 0.00179398 | 0.301417508 |
| CA1 | 2884.818511 | 2.076657265 | 0.001794694 | 0.301417508 |
| PRELP | 149.2603821 | 1.068878913 | 0.001826146 | 0.301417508 |
| RGS3 | 603.1198191 | −0.313215009 | 0.001894436 | 0.301417508 |
| GSDMC | 13.68277036 | −1.325067842 | 0.001897953 | 0.301417508 |
| TYMP | 1464.768981 | −0.696167395 | 0.001937775 | 0.301417508 |
| MYO1F | 606.4251332 | −0.532393196 | 0.001947213 | 0.301417508 |
| EDN1 | 151.1367903 | −0.485183623 | 0.00195317 | 0.301417508 |
| MT2A | 754.0649999 | −0.800885696 | 0.001957318 | 0.301417508 |
| CD300C | 38.64801845 | −0.687888133 | 0.001965729 | 0.301417508 |
| CNTN1 | 34.82071639 | 1.202507422 | 0.001979141 | 0.301417508 |
| SLC9A9 | 151.5146286 | 0.390117741 | 0.00198252 | 0.301417508 |
| APOL2 | 1380.803074 | −0.61816297 | 0.001991339 | 0.301417508 |
| GLT1D1 | 30.55404476 | −1.335459397 | 0.001992649 | 0.301417508 |
| KIAA2022 | 8.39545959 | 1.110659808 | 0.002032918 | 0.303055283 |
| KANK4 | 11.99966942 | 1.242845228 | 0.002050125 | 0.303055283 |
| EMR2 | 368.7178652 | −0.826542898 | 0.002062402 | 0.303055283 |
| CHMP4C | 264.8618808 | 0.501371898 | 0.00211717 | 0.307508524 |
| CXXC4 | 27.2122769 | 0.522500707 | 0.002132569 | 0.307508524 |
| PDGFB | 178.670281 | −0.620897944 | 0.002153947 | 0.307715287 |
| PRKCDBP | 113.3674965 | −0.794751614 | 0.002195238 | 0.310736989 |
| HCAR3 | 166.6445715 | −1.797143179 | 0.002252748 | 0.312805681 |
| MEFV | 69.9932486 | −1.079552144 | 0.002255787 | 0.312805681 |
| ITIH1 | 3.765114851 | 1.801203883 | 0.002270674 | 0.312805681 |
| ZNF180 | 96.81099378 | 0.284407631 | 0.00232086 | 0.31688978 |
| ACSL1 | 769.3567693 | −0.629204746 | 0.002375655 | 0.321526177 |
| SERPINH1 | 1448.642851 | −0.435782479 | 0.002401686 | 0.322222743 |
| KCNJ15 | 74.21697188 | −1.388441195 | 0.00247467 | 0.329152443 |
| FPR2 | 152.2665921 | −1.561931781 | 0.002610446 | 0.343491034 |
| PML | 856.7650377 | −0.450426176 | 0.002626997 | 0.343491034 |
| CRIP3 | 9.858462532 | 1.131413542 | 0.002653799 | 0.344079565 |
| PAK3 | 14.29951182 | 0.871163812 | 0.002741388 | 0.352095927 |
| S100A8 | 516.1026144 | −1.44896929 | 0.002761268 | 0.352095927 |
| AGTRAP | 303.5954041 | −0.366155738 | 0.002851262 | 0.357943211 |
| NRBF2 | 254.2046332 | −0.217973831 | 0.00285909 | 0.357943211 |
| LCN8 | 2.661740817 | 4.526324201 | 0.002886973 | 0.357943211 |
| LCP2 | 700.8022057 | −0.529213276 | 0.002903855 | 0.357943211 |
| CD97 | 1624.920088 | −0.29376518 | 0.002923122 | 0.357943211 |
| RNF149 | 966.8684308 | −0.385556934 | 0.003016884 | 0.359954883 |
| FAM20C | 554.4572542 | −0.729110504 | 0.003027376 | 0.359954883 |
| IL6 | 137.4603638 | −1.872812541 | 0.003039422 | 0.359954883 |
| MAMDC2 | 77.14364178 | 1.077587002 | 0.00304496 | 0.359954883 |
| FRMD5 | 42.45662616 | −0.551789098 | 0.003056199 | 0.359954883 |
| PRSS23 | 1269.574378 | −0.518664008 | 0.003105654 | 0.36300863 |
| ADAMTS2 | 404.6682717 | −0.823644527 | 0.003170801 | 0.363452743 |
| CXCR2 | 198.0697661 | −1.504911179 | 0.003175255 | 0.363452743 |
| CGNL1 | 107.0674399 | 0.554566817 | 0.003210693 | 0.363452743 |
| SLC26A7 | 7.979381157 | 1.155365714 | 0.003215226 | 0.363452743 |
| MAPK4 | 8.790190651 | 1.599861787 | 0.003274881 | 0.363452743 |
| GPR68 | 145.2882826 | −0.63197975 | 0.003320694 | 0.363452743 |
| CCDC85B | 70.38711199 | −1.072235946 | 0.003334462 | 0.363452743 |
| TFB2M | 208.4541531 | 0.305081722 | 0.003341185 | 0.363452743 |
| FPR3 | 538.077495 | −0.491211879 | 0.003355985 | 0.363452743 |
| IL10 | 27.97631112 | −0.748586007 | 0.00336366 | 0.363452743 |
| GZMH | 45.67425824 | −0.978458514 | 0.003368575 | 0.363452743 |
| TWIST1 | 22.06253863 | −1.168607671 | 0.003429279 | 0.367432956 |
| KYNU | 313.5307864 | −0.887143876 | 0.003465275 | 0.368729158 |
| P2RY6 | 118.969069 | −0.682542667 | 0.003508725 | 0.370795351 |
| DUOXA1 | 41.78447289 | 1.164986485 | 0.003605027 | 0.375315993 |
| THNSL1 | 71.40192978 | 0.481084394 | 0.003614439 | 0.375315993 |
| GAS1 | 52.07464884 | −1.302056746 | 0.003624479 | 0.375315993 |
| AJAP1 | 22.61295569 | −0.903346358 | 0.003684437 | 0.376071973 |
| TARBP1 | 393.023524 | 0.273797525 | 0.003688553 | 0.376071973 |
| ECEL1 | 6.278329523 | −1.744911015 | 0.003711289 | 0.376071973 |
| TRPM5 | 25.12588897 | 0.868271644 | 0.003729277 | 0.376071973 |
| TAP1 | 4573.546961 | −0.432425555 | 0.003804006 | 0.381116896 |
| XIRP1 | 5.95863417 | −1.915459539 | 0.003852525 | 0.381611932 |
| SPON2 | 436.7821724 | −0.682099861 | 0.003868974 | 0.381611932 |
| RND3 | 742.3046393 | −0.304031374 | 0.003883147 | 0.381611932 |
| PI15 | 236.0123091 | −1.64216232 | 0.003944315 | 0.383224026 |
| GBP1 | 2082.256606 | −0.688433984 | 0.003961081 | 0.383224026 |
| CDC25B | 1152.129405 | −0.457393671 | 0.003974065 | 0.383224026 |
| COL19A1 | 14.0999984 | 1.161531971 | 0.004058959 | 0.383658079 |
| UCN2 | 15.82232094 | −1.706643873 | 0.004067746 | 0.383658079 |
| KIAA1199 | 892.9038839 | −1.464035577 | 0.004114575 | 0.383658079 |
| FAM65C | 246.6649576 | −0.805227997 | 0.004122964 | 0.383658079 |
| TNFAIP6 | 103.975943 | −1.23855828 | 0.004124454 | 0.383658079 |
| SPI1 | 442.2024738 | −0.606434313 | 0.004127762 | 0.383658079 |
| IL22 | 12.11840649 | −1.555283495 | 0.004226837 | 0.39051419 |
| LOX | 233.7338087 | −0.500770995 | 0.00427652 | 0.392752577 |
| CLEC4E | 72.03668222 | −1.087601128 | 0.004314872 | 0.392778101 |
| HSD3B2 | 9.889031223 | 2.615679647 | 0.004327713 | 0.392778101 |
| HAL | 17.07758535 | −1.177931464 | 0.004384927 | 0.395643489 |
| GAPT | 86.19781481 | 0.577299495 | 0.004463871 | 0.396779869 |
| SNAP29 | 538.2244988 | −0.205047055 | 0.004487674 | 0.396779869 |
| SEC22A | 156.8551614 | 0.214707333 | 0.004515121 | 0.396779869 |
| SPHK1 | 149.208726 | −0.854051265 | 0.004534048 | 0.396779869 |
| FCGR3A | 666.4526877 | −0.819413262 | 0.004563509 | 0.396779869 |
| CENPF | 452.425366 | 0.363691565 | 0.004563724 | 0.396779869 |
| KLF10 | 769.779455 | −0.301948619 | 0.004608633 | 0.396779869 |
| TWIST2 | 6.123232934 | −1.243975465 | 0.004640758 | 0.396779869 |
| CYP27C1 | 11.76547844 | 0.875786123 | 0.004652416 | 0.396779869 |
| TMEM132A | 295.0309403 | −0.774756412 | 0.004654686 | 0.396779869 |
| CEBPD | 375.6933995 | −0.854411874 | 0.004682067 | 0.396920927 |
| TNFAIP3 | 1311.603345 | −0.535092443 | 0.004740862 | 0.398333069 |
| TMEM255A | 37.62852252 | 0.840794889 | 0.004750359 | 0.398333069 |
| FHL1 | 1126.475823 | 0.526990709 | 0.004880715 | 0.407051615 |
| MNDA | 354.0706377 | −0.921457857 | 0.004912218 | 0.40747638 |

TABLE 8
Gene set enrichment analysis (GSEA) results focused
on the leukocyte migration and cell adhesion gene ontology
(GO) gene sets, derived from the MSigDB. All gene sets
are enriched in the non-responder group.

| | Nominal | FDR corrected |
|---|---|---|
| GO gene set | p-value | p-value |
| GO Leukocyte | 0.006 | 0.017 |
| migration | | |
| GO Leukocyte | 0.032 | 0.090 |
| adhesion to vascular | | |
| endothelial cells | | |
| GO Cellular | 0.038 | 0.060 |
| Extravasation | | |
| GO Leukocyte Cell Cell | 0.089 | 0.098 |
| adhesion | | |
| GO integrin mediated | 0.117 | 0.145 |
| signalling pathway | | |
| GO Integrin binding | 0.171 | 0.166 |
| GO cell adhesion | 0.201 | 0.221 |
| molecule binding | | |
| GO cell substrate | 0.232 | 0.208 |
| adhesion | | |

FDR = false discovery rate

1.-21. (canceled)

22. A method of determining to which treatment a patient suffering of inflammatory bowel diseases is likely to respond or of determining to which treatment a patient suffering of inflammatory bowel diseases is likely to respond best, the method comprising:
(i) (a) detecting expression of the genes of a first group consisting of CELSR3, HAAO, FAM135B, F2RL2, CMPK2, SLC28A2, RET, CHP2, PITX1 and GSTT1 in a biological sample from a patient and expression of the genes of a second group consisting of FCER2, CTSL, PTGFRN, GPRC5C, SLAMF7, NR4A2, GNG2, RHOC, SULT1A1, DSC2, NEDD4L, ENGASE, GSN, GNLY, CLEC10A, HLA_DRB5, BAG3, ASGR2, HLA_DRB1 and PTK2 in a biological sample from the patient, and
(b) detecting expression of the genes of a third group consisting of FAM129A, ELM01, TRIP13, PTAR1, ASAH1, SKAP2, HAUS1, C3orf67, SEC14L6, ATP6V0D1, ABCG1, ERAP1, ERV3_1, APOL6 and STON2 in a biological sample from the patient
(c) detecting expression of the TREM1 gene in a biological sample from the patient
(d) comparing the expression level of the genes to a reference expression level,
wherein downregulation of TREM1 expression indicates the future anti-TNF healers or identifies a patient who is likely to respond to treatment with a TNF antagonist,
wherein a different expression level of each of the genes of the first group of genes and of each of the genes of the second group of genes compared to a reference expression level indicates the future anti-IL-12/23 pathway healers or identifies a patient who is likely to respond to treatment with a IL-12/23 pathway antagonist, and
wherein a different expression level of each of the genes the third group of genes compared to a reference expression level indicates the future anti-α₄β₇-integrin healers or identifies a patient who is likely to respond to treatment with a α₄β₇-integrin antagonist, or
(ii) (a′) detecting expression of the genes of a first group consisting of CELSR3, HAAO, FAM135B, F2RL2, CMPK2, SLC28A2, RET, CHP2, PITX1 and GSTT1 in a biological sample from a patient and expression of the genes of a second group consisting of FCER2, CTSL, PTGFRN, GPRC5C, SLAMF7, NR4A2, GNG2, RHOC, SULT1A1, DSC2, NEDD4L, ENGASE, GSN, GNLY, CLEC10A, HLA_DRB5, BAG3, ASGR2, HLA_DRB1 and PTK2 in a biological sample from the patient, and
(b′) detecting expression of the genes of a third group consisting of PIWIL1, MAATS1, RGS13 and DCHS2 in a biological sample from the patient.
(c′) detecting expression of the TREM1 gene in a biological sample from the patient.
(d′) comparing the expression level of the genes to a reference expression level,
wherein downregulation of TREM1 expression indicates the future anti-TNF healers or identifies a patient who is likely to respond to treatment with a TNF antagonist,
wherein a different expression level each of the genes of the first group of genes and of each of the genes of the second group of genes compared to a reference expression level indicates the future anti-IL-12/23 pathway healers or identifies a patient who is likely to respond to treatment with a IL-12/23 pathway antagonist, and wherein a decreased expression level of each of the genes the third group of genes compared to a reference expression level indicates the future anti-α₄β₇-integrin healers or identifies a patient who is likely to respond to treatment with a α₄β₇-integrin antagonist.

23. The method according to claim 22, wherein the method comprises:
(i) (a) detecting expression of the genes of a first group consisting of CELSR3, HAAO, FAM135B, F2RL2, CMPK2, SLC28A2, RET, CHP2, PITX1 and GSTT1 in a colonic tissue sample and expression of the genes of a second group consisting of FCER2, CTSL, PTGFRN, GPRC5C, SLAMF7, NR4A2, GNG2, RHOC, SULT1A1, DSC2, NEDD4L, ENGASE, GSN, GNLY, CLEC10A, HLA_DRB5, BAG3, ASGR2, HLA_DRB1 and PTK2 in a blood sample from the patient; and
(b) detecting expression of the genes of a third group consisting of FAM129A, ELM01, TRIP13, PTAR1, ASAH1, SKAP2, HAUS1, C3orf67, SEC14L6, ATP6V0D1, ABCG1, ERAP1, ERV31, APOL6 and STON2 in a blood sample from the patient
(c) detecting expression of the TREM1 gene in a blood sample from the patient
(d) comparing the expression level of the genes to a reference expression level,
wherein downregulation of TREM1 expression indicates the future anti-TNF healers or identifies a patient who is likely to respond to treatment with a TNF antagonist,
wherein a different expression level each of the genes of the first group of genes and of each of the genes of the second group of genes compared to a reference expression level indicates the future anti-IL-12/23 pathway healers or identifies a patient who is likely to respond to treatment with a IL-12/23 pathway antagonist and
wherein a different expression level of each of the genes the third group of genes compared to a reference expression level indicates the future anti-α₄β₇-integrin healers or identifies a patient who is likely to respond to treatment with a α₄β₇-integrin antagonist, or
(ii) (a′) detecting expression of the genes of a first group consisting of CELSR3, HAAO, FAM135B, F2RL2, CMPK2, SLC28A2, RET, CHP2, PITX1 and GSTT1 in a colonic tissue sample and expression of the genes of a second group consisting of FCER2, CTSL, PTGFRN, GPRC5C, SLAMF7, NR4A2, GNG2, RHOC, SULT1A1, DSC2, NEDD4L, ENGASE, GSN, GNLY, CLEC10A, HLA_DRB5, BAG3, ASGR2, HLA_DRB1 and PTK2 in a blood sample from the; and
(b′) detecting expression of the genes of a third group consisting of PIWIL1, MAATS1, RGS13 and DCHS2 in sample of colonic tissue sample from the patient
(c′) detecting expression of the TREM1 gene in a blood sample from the patient
(d′) comparing the expression level of the genes to a reference expression level,
wherein downregulation of TREM1 expression indicates the future anti-TNF healers or identifies a patient who is likely to respond to treatment with a TNF antagonist,
wherein a different expression level each of the genes of the first group of genes and of each of the genes of the second group of genes compared to a reference expression level indicates the future anti-IL-12/23 pathway healers or identifies a patient who is likely to respond to treatment with a IL-12/23 pathway antagonist and
wherein a decreased expression level of each of the genes the third group of genes compared to a reference expression level indicates the future anti-α₄β₇-integrin healers or identifies a patient who is likely to respond to treatment with a α₄β₇-integrin antagonist.

24. The method according to claim 22, wherein the method comprises:
(a) detecting expression of the genes of a first group consisting of CELSR3, HAAO, FAM135B, F2RL2, CMPK2, SLC28A2, RET, CHP2, PITX1 and GSTT1 in a colonic mucosal biopsy sample and expression of the genes of a second group consisting of FCER2, CTSL, PTGFRN, GPRC5C, SLAMF7, NR4A2, GNG2, RHOC, SULT1A1, DSC2, NEDD4L, ENGASE, GSN, GNLY, CLEC10A, HLA_DRB5, BAG3, ASGR2, HLA_DRB1 and PTK2 in CD14+ monocytes from the patient; and
(b) detecting expression of the genes of a third group consisting of FAM129A, ELM01, TRIP13, PTAR1, ASAH1, SKAP2, HAUS1, C3orf67, SEC14L6, ATP6V0D1, ABCG1, ERAP1, ERV31, APOL6 and STON2 in CD14+ monocytes from the patient
(c) detecting expression of the TREM1 gene in a whole blood cell sample from the patient
(d) comparing the expression level of the genes to a reference expression level, wherein downregulation of TREM1 expression indicates the future anti-TNF healer or identifies a patient who is likely to respond to treatment with a TNF antagonist,
wherein a different expression level of each of the genes the first group of genes and of each of the genes of the second group of genes compared to a reference expression level indicates the future anti-IL-12/23 pathway healers or identifies a patient who is likely to respond to treatment with a IL-12/23 pathway antagonist, and
wherein a different expression level of each of the genes the third group of genes compared to a reference expression level indicates the future anti-α₄β₇-integrin healer or identifies a patient who is likely to respond to treatment with a α₄β₇-integrin antagonist, or
(ii) (a′) detecting expression of the genes of a first group consisting of CELSR3, HAAO, FAM135B, F2RL2, CMPK2, SLC28A2, RET, CHP2, PITX1 and GSTT1 in a colonic mucosal biopsy sample and expression of the genes of a second group consisting of FCER2, CTSL, PTGFRN, GPRC5C, SLAMF7, NR4A2, GNG2, RHOC, SULT1A1, DSC2, NEDD4L, ENGASE, GSN, GNLY, CLEC10A, HLA_DRB5, BAG3, ASGR2, HLA_DRB1 and PTK2 in CD14+ monocytes from the patient; and
(b′) detecting expression of the genes of a third group consisting of PIWIL1, MAATS1, RGS13 and DCHS2 in a colonic mucosal biopsy sample from the patient
(c′) detecting expression of the TREM1 gene in a whole blood cell sample of the patient.
(d′) comparing the expression level of the genes to a reference expression level,
wherein downregulation of TREM1 expression indicates the future anti-TNF healer or identifies a patient who is likely to respond to treatment with a TNF antagonist,
wherein a different expression level of each of the genes the first group of genes and of each of the genes of the second group of genes compared to a reference expression level indicates the future anti-IL-12/23 pathway healers or identifies a patient who is likely to respond to treatment with a IL-12/23 pathway antagonist and
wherein a decreased expression level of each of the genes the third group of genes compared to a reference expression level indicates the future anti-α₄β₇-integrin healers or identifies a patient who is likely to respond to treatment with a α₄β₇-integrin antagonist.

25. The method according to claim 22, wherein the expression of the genes or the expression product of the genes in a sample obtained from the patient is a nucleic acid molecule selected from the group consisting of mRNA and cDNA mRNA or polypeptides derived therefrom.

26. The method according to claim 22, wherein the expression of the genes in a sample obtained from the patient is detected by measuring mRNA.

27. The method according to claim 22, wherein the inflammatory bowel disease is ulcerative colitis.

28. The method according to claim 22, wherein the inflammatory bowel disease is Crohn's disease.

29. The method according to claim 22, wherein the patient is a human.

30. The method according to claim 22, wherein the sample analysed for gene expression is obtained from the patient prior to any administration of any of the antagonists to the patient.

31. The method according to claim 22, wherein the sample analysed for gene expression is obtained from the patient after an initial treatment with a selected TNF, α₄β₇-integrin or IL-12/23 antagonists to the patient.

32. The method according to claim 22, wherein each sample is from the patient removed sample or biopsy sample and the analysis is in vitro.

33. The method according to claim 22, wherein a TNF antagonist is infliximab or adalimumab.

34. The method according to claim 22, wherein a α₄β₇-integrin antagonist is an anti-α₄β₇-integrin antibody therapy that blocks action of α₄β₇-integrin by preventing Integrin α₄β₇ forming a complex with the T-cell surface Cd4.

35. The method according to claim 22, wherein a α₄β₇-integrin antagonist is an anti-α₄β₇-integrin antibody therapy that blocks the action of α₄β₇-integrin by preventing α₄β₇-integrin of interacting with MadCAM-1.

36. The method according to claim 22, wherein a α₄β₇-integrin antagonist is an antibody of the group consisting of a Natalizumab, Vedolizumab, Etrolizumab and AMG-18.

37. The method according to claim 22, wherein an IL-12/23 pathway antagonist is an anti-P40 antibody.

38. The method according to claim 22, wherein an IL-12/23 pathway antagonist is ustekinumab.

39. The method according to claim 22, wherein each sample is a from the patient removed sample or is a biopsy sample and the analysis is in vitro.

40. A monoclonal anti-TNF antibody or an antigen-binding fragment thereof for use in the treatment of an inflammatory bowel disease, comprising claim 22 identifying the patient as more likely to respond to a therapy comprising the monoclonal anti-TNF antibody or an antigen-binding fragment thereof and administering the monoclonal anti-TNF antibody or an antigen-binding fragment thereof when the level of mRNA expression measured indicate the patient is a future anti-TNF healer.

41. A monoclonal anti-α₄β₇-integrin antibody or an antigen-binding fragment thereof for use in the treatment of an inflammatory bowel disease, comprising claim 22 identifying the patient as more likely to respond to a therapy comprising the monoclonal anti-α₄β₇-integrin antibody or an antigen-binding fragment thereof and administering the monoclonal anti-α₄β₇-integrin antibody or an antigen-binding fragment thereof when the level of mRNA expression measured indicate the patient is a future anti-α₄β₇-integrin healer.

42. A monoclonal directed against IL12 and IL23 or an antibody binds to the p-40 subunit of both IL-12 and IL-23 or the antigen-binding fragment thereof for use in the treatment of an inflammatory bowel disease, comprising claim 22 identifying the patient as more likely to respond to a therapy comprising the monoclonal antibody or an antigen-binding fragment thereof and administering the monoclonal antibody or an antigen-binding fragment thereof when the level of mRNA expression measured indicate the patient is a future anti-IL-12/23 pathway healer.
